(19) United States
(12) Patent Application Publication
(10) Pub. No.: US 2023/0416732 A1
(43) Pub. Date: Dec. 28, 2023

(54) COMPOSITIONS COMPRISING AN RNA GUIDE TARGETING BCL11A AND USES THEREOF

(71) Applicant: Arbor Biotechnologies, Inc., Cambridge, MA (US)

(72) Inventors: Quinton Norman Wessells, Cambridge, MA (US); Jeffrey Raymond Haswell, Newton, MA (US); Tia Marie Ditommaso, Waltham, MA (US); Noah Michael Jakimo, Cambridge, MA (US)

(21) Appl. No.: 18/251,183

(22) PCT Filed: Oct. 29, 2021
(86) PCT No.: PCT/US2021/057426

§ 371 Date: Apr. 28, 2023

Related U.S. Application Data
(60) Provisional application No. 63/252,832, filed on Oct. 6, 2021.
(60) Provisional application No. 63/108,110, filed on Oct. 30, 2020.

(51) Int. Cl. C12N 15/11; C12N 9/22; C12N 15/10; C12N 15/90

(52) U.S. Cl. CPC C12N 15/11; C12N 9/22; C12N 15/102; C12N 15/907; C12N 2310/20

(57) ABSTRACT

The present invention relates to compositions comprising RNA guides targeting BCL11A, processes for characterizing the compositions, cells comprising the compositions, and methods of using the compositions.

### SEQUENCE LISTING

The instant application contains a Sequence Listing which has been submitted electronically in ASCII format and is hereby incorporated by reference in its entirety. Said ASCII copy, created on Oct. 28, 2021, is named 51451-017WO3_Sequence_Listing_10_28_21_ST25, and is 682,314 bytes in size.

### BACKGROUND

Clustered Regularly Interspaced Short Palindromic Repeats (CRISPR) and CRISPR-associated (Cas) genes, collectively known as CRISPR-Cas or CRISPR/Cas systems, are adaptive immune systems in archaea and bacteria that defend particular species against foreign genetic elements.

### SUMMARY OF THE INVENTION

It is against the above background that the present invention provides certain advantages and advancements over the prior art. Although this invention disclosed herein is not limited to specific advantages or functionalities, the invention provides a composition comprising an RNA guide, wherein the RNA guide comprises (i) a spacer sequence that is substantially complementary to a target sequence within a BCL11A gene and (ii) a direct repeat sequence; wherein the target sequence is adjacent to a protospacer adjacent motif (PAM) comprising the sequence 5′-NTTN-3′.

In one aspect of the composition, the target sequence is within exon 1, exon 2, exon 3, exon 4, or the enhancer region of the BCL11A gene.

In another aspect of the composition, the BCL11A gene comprises the sequence of SEQ ID NO: 2635, the reverse complement of SEQ ID NO: 2635, a variant of SEQ ID NO: 2635, or the reverse complement of a variant of SEQ ID NO: 2635.

In another aspect of the composition, the spacer sequence comprises: a. nucleotide 1 through nucleotide 16 of a sequence that is at least 90% identical to a sequence of any one of SEQ ID NOs: 1322-2632; b. nucleotide 1 through nucleotide 17 of a sequence that is at least 90% identical to a sequence of any one of SEQ ID NOs: 1322-2632; c. nucleotide 1 through nucleotide 18 of a sequence that is at least 90% identical to a sequence of any one of SEQ ID NOs: 1322-2632; d. nucleotide 1 through nucleotide 19 of a sequence that is at least 90% identical to a sequence of any one of SEQ ID NOs: 1322-2632; e. nucleotide 1 through nucleotide 20 of a sequence that is at least 90% identical to a sequence of any one of SEQ ID NOs: 1322-2632; f. nucleotide 1 through nucleotide 21 of a sequence that is at least 90% identical to a sequence of any one of SEQ ID NOs: 1322-2632; g. nucleotide 1 through nucleotide 22 of a sequence that is at least 90% identical to a sequence of any one of SEQ ID NOs: 1322-2632; h. nucleotide 1 through nucleotide 23 of a sequence that is at least 90% identical to a sequence of any one of SEQ ID NOs: 1322-2632; i. nucleotide 1 through nucleotide 24 of a sequence that is at least 90% identical to a sequence of any one of SEQ ID NOs: 1322-2632; j. nucleotide 1 through nucleotide 25 of a sequence that is at least 90% identical to a sequence of any one of SEQ ID NOs: 1322-2632; k. nucleotide 1 through nucleotide 26 of a sequence that is at least 90% identical to a sequence of any one of SEQ ID NOs: 1322-2632; 1. nucleotide 1 through nucleotide 27 of a sequence that is at least 90% identical to a sequence of any one of SEQ ID NOs: 1322-2632; m. nucleotide 1 through nucleotide 28 of a sequence that is at least 90% identical to a sequence of any one of SEQ ID NOs: 1322-2632; n. nucleotide 1 through nucleotide 29 of a sequence that is at least 90% identical to a sequence of any one of SEQ ID NOs: 1322-1425 and 1427-2632; or o. nucleotide 1 through nucleotide 30 of a sequence that is at least 90% identical to a sequence of any one of SEQ ID NOs: 1322-1425 and 1427-2632.

In another aspect of the composition, the spacer sequence comprises: a. nucleotide 1 through nucleotide 16 of any one of SEQ ID NOs: 1322-2632; b. nucleotide 1 through nucleotide 17 of any one of SEQ ID NOs: 1322-2632; c. nucleotide 1 through nucleotide 18 of any one of SEQ ID NOs: 1322-2632; d. nucleotide 1 through nucleotide 19 of any one of SEQ ID NOs: 1322-2632; e. nucleotide 1 through nucleotide 20 of any one of SEQ ID NOs: 1322-2632; f. nucleotide 1 through nucleotide 21 of any one of SEQ ID NOs: 1322-2632; g. nucleotide 1 through nucleotide 22 of any one of SEQ ID NOs: 1322-2632; h. nucleotide 1 through nucleotide 23 of any one of SEQ ID NOs: 1322-2632; i. nucleotide 1 through nucleotide 24 of any one of SEQ ID NOs: 1322-2632; j. nucleotide 1 through nucleotide 25 of any one of SEQ ID NOs: 1322-2632; k. nucleotide 1 through nucleotide 26 of any one of SEQ ID NOs: 1322-2632; 1. nucleotide 1 through nucleotide 27 of any one of SEQ ID NOs: 1322-2632; m. nucleotide 1 through nucleotide 28 of any one of SEQ ID NOs: 1322-2632; n. nucleotide 1 through nucleotide 29 of any one of SEQ ID NOs: 1322-1425 and 1427-2632; or o. nucleotide 1 through nucleotide 30 of any one of SEQ ID NOs: 1322-1425 and 1427-2632.

In another aspect of the composition, the direct repeat comprises: a. nucleotide 1 through nucleotide 36 of a sequence that is at least 90% identical to a sequence of any one of SEQ ID NOs: 1-8; b. nucleotide 2 through nucleotide 36 of a sequence that is at least 90% identical to a sequence of any one of SEQ ID NOs: 1-8; c. nucleotide 3 through nucleotide 36 of a sequence that is at least 90% identical to a sequence of any one of SEQ ID NOs: 1-8; d. nucleotide 4 through nucleotide 36 of a sequence that is at least 90% identical to a sequence of any one of SEQ ID NOs: 1-8; e. nucleotide 5 through nucleotide 36 of a sequence that is at least 90% identical to a sequence of any one of SEQ ID NOs: 1-8; f. nucleotide 6 through nucleotide 36 of a sequence that is at least 90% identical to a sequence of any one of SEQ ID NOs: 1-8; g. nucleotide 7 through nucleotide 36 of a sequence that is at least 90% identical to a sequence of any one of SEQ ID NOs: 1-8; h. nucleotide 8 through nucleotide 36 of a sequence that is at least 90% identical to a sequence of any one of SEQ ID NOs: 1-8; i. nucleotide 9 through nucleotide 36 of a sequence that is at least 90% identical to a sequence of any one of SEQ ID NOs: 1-8; j. nucleotide 10 through nucleotide 36 of a sequence that is at least 90% identical to a sequence of any one of SEQ ID NOs: 1-8; k. nucleotide 11 through nucleotide 36 of a sequence that is at least 90% identical to a sequence of any one of SEQ ID NOs: 1-8; 1. nucleotide 12 through nucleotide 36 of a sequence that is at least 90% identical to a sequence of any one of SEQ ID NOs: 1-8; m. nucleotide 13 through nucleotide 36 of a sequence that is at least 90% identical to a sequence of any one of SEQ ID NOs: 1-8; n. nucleotide 14 through nucleotide 36 of a sequence that is at least 90% identical to a sequence of any one of SEQ ID NOs: 1-8; o. nucleotide 1 through nucleotide 34 of a sequence that is at least 90% identical to a sequence of SEQ ID NO: 9; p. nucleotide 2 through nucleotide 34 of a sequence that is at least 90% identical to a sequence of SEQ ID NO: 9; q. nucleotide 3 through nucleotide 34 of a sequence that is at least 90% identical to a sequence of SEQ ID NO: 9; r. nucleotide 4 through nucleotide 34 of a sequence that is at least 90% identical to a sequence of SEQ ID NO: 9; s. nucleotide 5 through nucleotide 34 of a sequence that is at least 90% identical to a sequence of SEQ ID NO: 9; t. nucleotide 6 through nucleotide 34 of a sequence that is at least 90% identical to a sequence of SEQ ID NO: 9; u. nucleotide 7 through nucleotide 34 of a sequence that is at least 90% identical to a sequence of SEQ ID NO: 9; v. nucleotide 8 through nucleotide 34 of a sequence that is at least 90% identical to a sequence of SEQ ID NO: 9; w. nucleotide 9 through nucleotide 34 of a sequence that is at least 90% identical to a sequence of SEQ ID NO: 9; x. nucleotide 10 through nucleotide 34 of a sequence that is at least 90% identical to a sequence of SEQ ID NO: 9; y. nucleotide 11 through nucleotide 34 of a sequence that is at least 90% identical to a sequence of SEQ ID NO: 9; z. nucleotide 12 through nucleotide 34 of a sequence that is at least 90% identical to a sequence of SEQ ID NO: 9; or aa. a sequence that is at least 90% identical to a sequence of SEQ ID NO: 10 or a portion thereof.

In another aspect of the composition, the direct repeat comprises: a. nucleotide 1 through nucleotide 36 of any one of SEQ ID NOs: 1-8; b. nucleotide 2 through nucleotide 36 of any one of SEQ ID NOs: 1-8; c. nucleotide 3 through nucleotide 36 of any one of SEQ ID NOs: 1-8; d. nucleotide 4 through nucleotide 36 of any one of SEQ ID NOs: 1-8; e. nucleotide 5 through nucleotide 36 of any one of SEQ ID NOs: 1-8; f. nucleotide 6 through nucleotide 36 of any one of SEQ ID NOs: 1-8; g. nucleotide 7 through nucleotide 36 of any one of SEQ ID NOs: 1-8; h. nucleotide 8 through nucleotide 36 of any one of SEQ ID NOs: 1-8; i. nucleotide 9 through nucleotide 36 of any one of SEQ ID NOs: 1-8; j. nucleotide 10 through nucleotide 36 of any one of SEQ ID NOs: 1-8; k. nucleotide 11 through nucleotide 36 of any one of SEQ ID NOs: 1-8; 1. nucleotide 12 through nucleotide 36 of any one of SEQ ID NOs: 1-8; m. nucleotide 13 through nucleotide 36 of any one of SEQ ID NOs: 1-8; n. nucleotide 14 through nucleotide 36 of any one of SEQ ID NOs: 1-8; o. nucleotide 1 through nucleotide 34 of SEQ ID NO: 9; p. nucleotide 2 through nucleotide 34 of SEQ ID NO: 9; q. nucleotide 3 through nucleotide 34 of SEQ ID NO: 9; r. nucleotide 4 through nucleotide 34 of SEQ ID NO: 9; s. nucleotide 5 through nucleotide 34 of SEQ ID NO: 9; t. nucleotide 6 through nucleotide 34 of SEQ ID NO: 9; u. nucleotide 7 through nucleotide 34 of SEQ ID NO: 9; v. nucleotide 8 through nucleotide 34 of SEQ ID NO: 9; w. nucleotide 9 through nucleotide 34 of SEQ ID NO: 9; x. nucleotide 10 through nucleotide 34 of SEQ ID NO: 9; y. nucleotide 11 through nucleotide 34 of SEQ ID NO: 9; z. nucleotide 12 through nucleotide 34 of SEQ ID NO: 9; or aa. SEQ ID NO: 10 or a portion thereof.

In another aspect of the composition, the direct repeat comprises: a. nucleotide 1 through nucleotide 36 of a sequence that is at least 90% identical to a sequence of any one of SEQ ID NOs: 2652-2669; b. nucleotide 2 through nucleotide 36 of a sequence that is at least 90% identical to a sequence of any one of SEQ ID NOs: 2652-2669; c. nucleotide 3 through nucleotide 36 of a sequence that is at least 90% identical to a sequence of any one of SEQ ID NOs: 2652-2669; d. nucleotide 4 through nucleotide 36 of a sequence that is at least 90% identical to a sequence of any one of SEQ ID NOs: 2652-2669; e. nucleotide 5 through nucleotide 36 of a sequence that is at least 90% identical to a sequence of any one of SEQ ID NOs: 2652-2669; f. nucleotide 6 through nucleotide 36 of a sequence that is at least 90% identical to a sequence of any one of SEQ ID NOs: 2652-2669; g. nucleotide 7 through nucleotide 36 of a sequence that is at least 90% identical to a sequence of any one of SEQ ID NOs: 2652-2669; h. nucleotide 8 through nucleotide 36 of a sequence that is at least 90% identical to a sequence of any one of SEQ ID NOs: 2652-2669; i. nucleotide 9 through nucleotide 36 of a sequence that is at least 90% identical to a sequence of any one of SEQ ID NOs: 2652-2669; j. nucleotide 10 through nucleotide 36 of a sequence that is at least 90% identical to a sequence of any one of SEQ ID NOs: 2652-2669; k. nucleotide 11 through nucleotide 36 of a sequence that is at least 90% identical to a sequence of any one of SEQ ID NOs: 2652-2669; 1. nucleotide 12 through nucleotide 36 of a sequence that is at least 90% identical to a sequence of any one of SEQ ID NOs: 2652-2669; m. nucleotide 13 through nucleotide 36 of a sequence that is at least 90% identical to a sequence of any one of SEQ ID NOs: 2652-2669; n. nucleotide 14 through nucleotide 36 of a sequence that is at least 90% identical to a sequence of any one of SEQ ID NOs: 2652-2669; or o. a sequence that is at least 90% identical to a sequence of SEQ ID NO: 2670 or a portion thereof.

In another aspect of the composition, the direct repeat comprises: a. nucleotide 1 through nucleotide 36 of any one of SEQ ID NOs: 2652-2669; b. nucleotide 2 through nucleotide 36 of any one of SEQ ID NOs: 2652-2669; c. nucleotide 3 through nucleotide 36 of any one of SEQ ID NOs: 2652-2669; d. nucleotide 4 through nucleotide 36 of any one of SEQ ID NOs: 2652-2669; e. nucleotide 5 through nucleotide 36 of any one of SEQ ID NOs: 2652-2669; f. nucleotide 6 through nucleotide 36 of any one of SEQ ID NOs: 2652-2669; g. nucleotide 7 through nucleotide 36 of any one of SEQ ID NOs: 2652-2669; h. nucleotide 8 through nucleotide 36 of any one of SEQ ID NOs: 2652-2669; i. nucleotide 9 through nucleotide 36 of any one of SEQ ID NOs: 2652-2669; j. nucleotide 10 through nucleotide 36 of any one of SEQ ID NOs: 2652-2669; k. nucleotide 11 through nucleotide 36 of any one of SEQ ID NOs: 2652-2669; 1. nucleotide 12 through nucleotide 36 of any one of SEQ ID NOs: 2652-2669; m. nucleotide 13 through nucleotide 36 of any one of SEQ ID NOs: 2652-2669; n. nucleotide 14 through nucleotide 36 of any one of SEQ ID NOs: 2652-2669; or o. SEQ ID NO: 2670 or a portion thereof.

In another aspect of the composition, the direct repeat comprises: a. nucleotide 1 through nucleotide 36 of a sequence that is at least 90% identical to SEQ ID NO: 2671; b. nucleotide 2 through nucleotide 36 of a sequence that is at least 90% identical to SEQ ID NO: 2671; c. nucleotide 3 through nucleotide 36 of a sequence that is at least 90% identical to SEQ ID NO: 2671; d. nucleotide 4 through nucleotide 36 of a sequence that is at least 90% identical to SEQ ID NO: 2671; e. nucleotide 5 through nucleotide 36 of a sequence that is at least 90% identical to SEQ ID NO: 2671; f. nucleotide 6 through nucleotide 36 of a sequence that is at least 90% identical to SEQ ID NO: 2671; g. nucleotide 7 through nucleotide 36 of a sequence that is at least 90% identical to SEQ ID NO: 2671; h. nucleotide 8 through nucleotide 36 of a sequence that is at least 90% identical to SEQ ID NO: 2671; i. nucleotide 9 through nucleotide 36 of a sequence that is at least 90% identical to SEQ ID NO: 2671; j. nucleotide 10 through nucleotide 36 of a sequence that is at least 90% identical to SEQ ID NO: 2671; k. nucleotide 11 through nucleotide 36 of a sequence that is at least 90% identical to SEQ ID NO: 2671; 1. nucleotide 12 through nucleotide 36 of a sequence that is at least 90% identical to SEQ ID NO: 2671; m. nucleotide 13 through nucleotide 36 of a sequence that is at least 90% identical to SEQ ID NO: 2671; n. nucleotide 14 through nucleotide 36 of a sequence that is at least 90% identical to SEQ ID NO: 2671; or o. a sequence that is at least 90% identical to a sequence of SEQ ID NO: 2672 or SEQ ID NO: 2673 or a portion thereof.

In another aspect of the composition, the direct repeat comprises: a. nucleotide 1 through nucleotide 36 of SEQ ID NO: 2671; b. nucleotide 2 through nucleotide 36 of SEQ ID NO: 2671; c. nucleotide 3 through nucleotide 36 of SEQ ID NO: 2671; d. nucleotide 4 through nucleotide 36 of SEQ ID NO: 2671; e. nucleotide 5 through nucleotide 36 of SEQ ID NO: 2671; f. nucleotide 6 through nucleotide 36 of SEQ ID NO: 2671; g. nucleotide 7 through nucleotide 36 of SEQ ID NO: 2671; h. nucleotide 8 through nucleotide 36 of SEQ ID NO: 2671; i. nucleotide 9 through nucleotide 36 of SEQ ID NO: 2671; j. nucleotide 10 through nucleotide 36 of SEQ ID NO: 2671; k. nucleotide 11 through nucleotide 36 of SEQ ID NO: 2671; 1. nucleotide 12 through nucleotide 36 of SEQ ID NO: 2671; m. nucleotide 13 through nucleotide 36 of SEQ ID NO: 2671; n. nucleotide 14 through nucleotide 36 of SEQ ID NO: 2671; or o. SEQ ID NO: 2672 or SEQ ID NO: 2673 or a portion thereof.

In another aspect of the composition, the direct repeat comprises: a. nucleotide 1 through nucleotide 36 of a sequence that is at least 90% identical to a sequence of SEQ ID NO: 2674 or SEQ ID NO: 2675; b. nucleotide 2 through nucleotide 36 of a sequence that is at least 90% identical to a sequence of SEQ ID NO: 2674 or SEQ ID NO: 2675; c. nucleotide 3 through nucleotide 36 of a sequence that is at least 90% identical to a sequence of SEQ ID NO: 2674 or SEQ ID NO: 2675; d. nucleotide 4 through nucleotide 36 of a sequence that is at least 90% identical to a sequence of SEQ ID NO: 2674 or SEQ ID NO: 2675; e. nucleotide 5 through nucleotide 36 of a sequence that is at least 90% identical to a sequence of SEQ ID NO: 2674 or SEQ ID NO: 2675; f. nucleotide 6 through nucleotide 36 of a sequence that is at least 90% identical to a sequence of SEQ ID NO: 2674 or SEQ ID NO: 2675; g. nucleotide 7 through nucleotide 36 of a sequence that is at least 90% identical to a sequence of SEQ ID NO: 2674 or SEQ ID NO: 2675; h. nucleotide 8 through nucleotide 36 of a sequence that is at least 90% identical to a sequence of SEQ ID NO: 2674 or SEQ ID NO: 2675; i. nucleotide 9 through nucleotide 36 of a sequence that is at least 90% identical to a sequence of SEQ ID NO: 2674 or SEQ ID NO: 2675; j. nucleotide 10 through nucleotide 36 of a sequence that is at least 90% identical to a sequence of SEQ ID NO: 2674 or SEQ ID NO: 2675; k. nucleotide 11 through nucleotide 36 of a sequence that is at least 90% identical to a sequence of SEQ ID NO: 2674 or SEQ ID NO: 2675; 1. nucleotide 12 through nucleotide 36 of a sequence that is at least 90% identical to a sequence of SEQ ID NO: 2674 or SEQ ID NO: 2675; m. nucleotide 13 through nucleotide 36 of a sequence that is at least 90% identical to a sequence of SEQ ID NO: 2674 or SEQ ID NO: 2675; n. nucleotide 14 through nucleotide 36 of a sequence that is at least 90% identical to a sequence of SEQ ID NO: 2674 or SEQ ID NO: 2675; o. nucleotide 15 through nucleotide 36 of a sequence that is at least 90% identical to a sequence of SEQ ID NO: 2674 or SEQ ID NO: 2675; or p. a sequence that is at least 90% identical to a sequence of SEQ ID NO: 2676 or a portion thereof.

In another aspect of the composition, the direct repeat comprises: a. nucleotide 1 through nucleotide 36 of SEQ ID NO: 2674 or SEQ ID NO: 2675; b. nucleotide 2 through nucleotide 36 of SEQ ID NO: 2674 or SEQ ID NO: 2675; c. nucleotide 3 through nucleotide 36 of SEQ ID NO: 2674 or SEQ ID NO: 2675; d. nucleotide 4 through nucleotide 36 of SEQ ID NO: 2674 or SEQ ID NO: 2675; e. nucleotide 5 through nucleotide 36 of SEQ ID NO: 2674 or SEQ ID NO: 2675; f. nucleotide 6 through nucleotide 36 of SEQ ID NO: 2674 or SEQ ID NO: 2675; g. nucleotide 7 through nucleotide 36 of SEQ ID NO: 2674 or SEQ ID NO: 2675; h. nucleotide 8 through nucleotide 36 of SEQ ID NO: 2674 or SEQ ID NO: 2675; i. nucleotide 9 through nucleotide 36 of SEQ ID NO: 2674 or SEQ ID NO: 2675; j. nucleotide 10 through nucleotide 36 of SEQ ID NO: 2674 or SEQ ID NO: 2675; k. nucleotide 11 through nucleotide 36 of SEQ ID NO: 2674 or SEQ ID NO: 2675; 1. nucleotide 12 through nucleotide 36 of SEQ ID NO: 2674 or SEQ ID NO: 2675; m. nucleotide 13 through nucleotide 36 of SEQ ID NO: 2674 or SEQ ID NO: 2675; n. nucleotide 14 through nucleotide 36 of SEQ ID NO: 2674 or SEQ ID NO: 2675; o. nucleotide 15 through nucleotide 36 of SEQ ID NO: 2674 or SEQ ID NO: 2675; or p. SEQ ID NO: 2676 or a portion thereof.

In another aspect of the composition, the spacer sequence is substantially complementary to the complement of a sequence of any one of SEQ ID NOs: 11-1321.

In another aspect of the composition, the PAM comprises the sequence 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′.

In another aspect of the composition, the target sequence is immediately adjacent to the PAM sequence.

In another aspect of the composition, the composition further comprises a Cas12i polypeptide.

In another aspect of the composition, the Cas12i polypeptide is: a. a Cas12i2 polypeptide comprising a sequence that is at least 90% identical to the sequence of SEQ ID NO: 2634, SEQ ID NO: 2641, SEQ ID NO: 2642, SEQ ID NO: 2643, SEQ ID NO: 2644, or SEQ ID NO: 2645; b. a Cas12i4 polypeptide comprising a sequence that is at least 90% identical to the sequence of SEQ ID NO: 2647, SEQ ID NO: 2648, or SEQ ID NO: 2649; c. a Cas12i1 polypeptide comprising a sequence that is at least 90% identical to the sequence of SEQ ID NO: 2650; or d. a Cas12i3 polypeptide comprising a sequence that is at least 90% identical to the sequence of SEQ ID NO: 2651.

In another aspect of the composition, the Cas12i polypeptide is: a. a Cas12i2 polypeptide comprising a sequence of SEQ ID NO: 2634, SEQ ID NO: 2641, SEQ ID NO: 2642, SEQ ID NO: 2643, SEQ ID NO: 2644, or SEQ ID NO: 2645; b. a Cas12i4 polypeptide comprising a sequence of SEQ ID NO: 2647, SEQ ID NO: 2648, or SEQ ID NO: 2649; c. a Cas12i1 polypeptide comprising a sequence of SEQ ID NO: 2650; or d. a Cas12i3 polypeptide comprising a sequence of SEQ ID NO: 2651.

In another aspect of the composition, the RNA guide and the Cas12i polypeptide form a ribonucleoprotein complex.

In another aspect of the composition, the ribonucleoprotein complex binds a target nucleic acid.

In another aspect of the composition, the composition is present within a cell.

In another aspect of the composition, the RNA guide and the Cas12i polypeptide are encoded in a vector, e.g., expression vector. In another aspect of the composition, the RNA guide and the Cas12i polypeptide are encoded in a single vector or the RNA guide is encoded in a first vector and the Cas12i polypeptide is encoded in a second vector.

The invention further provides a vector system comprising one or more vectors encoding an RNA guide disclosed herein and a Cas12i polypeptide. In an embodiment, the vector system comprises a first vector encoding an RNA guide disclosed herein and a second vector encoding a Cas12i polypeptide. The vectors may be expression vectors.

The invention further provides a composition comprising an RNA guide and a Cas12i polypeptide, wherein the RNA guide comprises (i) a spacer sequence that is substantially complementary to a target sequence within a BCL11A gene and (ii) a direct repeat sequence.

In one aspect of the composition, the target sequence is within exon 1, exon 2, exon 3, exon 4, or the enhancer region of the BCL11A gene.

In another aspect of the composition, the BCL11A gene comprises the sequence of SEQ ID NO: 2635, the reverse complement of SEQ ID NO: 2635, a variant of SEQ ID NO: 2635, or the reverse complement of a variant of SEQ ID NO: 2635.

In another aspect of the composition, the spacer sequence comprises: a. nucleotide 1 through nucleotide 16 of a sequence that is at least 90% identical to a sequence of any one of SEQ ID NOs: 1322-2632; b. nucleotide 1 through nucleotide 17 of a sequence that is at least 90% identical to a sequence of any one of SEQ ID NOs: 1322-2632; c. nucleotide 1 through nucleotide 18 of a sequence that is at least 90% identical to a sequence of any one of SEQ ID NOs: 1322-2632; d. nucleotide 1 through nucleotide 19 of a sequence that is at least 90% identical to a sequence of any one of SEQ ID NOs: 1322-2632; e. nucleotide 1 through nucleotide 20 of a sequence that is at least 90% identical to a sequence of any one of SEQ ID NOs: 1322-2632; f. nucleotide 1 through nucleotide 21 of a sequence that is at least 90% identical to a sequence of any one of SEQ ID NOs: 1322-2632; g. nucleotide 1 through nucleotide 22 of a sequence that is at least 90% identical to a sequence of any one of SEQ ID NOs: 1322-2632; h. nucleotide 1 through nucleotide 23 of a sequence that is at least 90% identical to a sequence of any one of SEQ ID NOs: 1322-2632; i. nucleotide 1 through nucleotide 24 of a sequence that is at least 90% identical to a sequence of any one of SEQ ID NOs: 1322-2632; j. nucleotide 1 through nucleotide 25 of a sequence that is at least 90% identical to a sequence of any one of SEQ ID NOs: 1322-2632; k. nucleotide 1 through nucleotide 26 of a sequence that is at least 90% identical to a sequence of any one of SEQ ID NOs: 1322-2632; 1. nucleotide 1 through nucleotide 27 of a sequence that is at least 90% identical to a sequence of any one of SEQ ID NOs: 1322-2632; m. nucleotide 1 through nucleotide 28 of a sequence that is at least 90% identical to a sequence of any one of SEQ ID NOs: 1322-2632; n. nucleotide 1 through nucleotide 29 of a sequence that is at least 90% identical to a sequence of any one of SEQ ID NOs: 1322-1425 and 1427-2632; or o. nucleotide 1 through nucleotide 30 of a sequence that is at least 90% identical to a sequence of any one of SEQ ID NOs: 1322-1425 and 1427-2632.

In another aspect of the composition, the spacer sequence comprises: a. nucleotide 1 through nucleotide 16 of any one of SEQ ID NOs: 1322-2632; b. nucleotide 1 through nucleotide 17 of any one of SEQ ID NOs: 1322-2632; c. nucleotide 1 through nucleotide 18 of any one of SEQ ID NOs: 1322-2632; d. nucleotide 1 through nucleotide 19 of any one of SEQ ID NOs: 1322-2632; e. nucleotide 1 through nucleotide 20 of any one of SEQ ID NOs: 1322-2632; f. nucleotide 1 through nucleotide 21 of any one of SEQ ID NOs: 1322-2632; g. nucleotide 1 through nucleotide 22 of any one of SEQ ID NOs: 1322-2632; h. nucleotide 1 through nucleotide 23 of any one of SEQ ID NOs: 1322-2632; i. nucleotide 1 through nucleotide 24 of any one of SEQ ID NOs: 1322-2632; j. nucleotide 1 through nucleotide 25 of any one of SEQ ID NOs: 1322-2632; k. nucleotide 1 through nucleotide 26 of any one of SEQ ID NOs: 1322-2632; 1. nucleotide 1 through nucleotide 27 of any one of SEQ ID NOs: 1322-2632; m. nucleotide 1 through nucleotide 28 of any one of SEQ ID NOs: 1322-2632; n. nucleotide 1 through nucleotide 29 of any one of SEQ ID NOs: 1322-1425 and 1427-2632; or o. nucleotide 1 through nucleotide 30 of any one of SEQ ID NOs: 1322-1425 and 1427-2632.

In another aspect of the composition, the direct repeat comprises: a. nucleotide 1 through nucleotide 36 of a sequence that is at least 90% identical to a sequence of any one of SEQ ID NOs: 1-8; b. nucleotide 2 through nucleotide 36 of a sequence that is at least 90% identical to a sequence of any one of SEQ ID NOs: 1-8; c. nucleotide 3 through nucleotide 36 of a sequence that is at least 90% identical to a sequence of any one of SEQ ID NOs: 1-8; d. nucleotide 4 through nucleotide 36 of a sequence that is at least 90% identical to a sequence of any one of SEQ ID NOs: 1-8; e. nucleotide 5 through nucleotide 36 of a sequence that is at least 90% identical to a sequence of any one of SEQ ID NOs: 1-8; f. nucleotide 6 through nucleotide 36 of a sequence that is at least 90% identical to a sequence of any one of SEQ ID NOs: 1-8; g. nucleotide 7 through nucleotide 36 of a sequence that is at least 90% identical to a sequence of any one of SEQ ID NOs: 1-8; h. nucleotide 8 through nucleotide 36 of a sequence that is at least 90% identical to a sequence of any one of SEQ ID NOs: 1-8; i. nucleotide 9 through nucleotide 36 of a sequence that is at least 90% identical to a sequence of any one of SEQ ID NOs: 1-8; j. nucleotide 10 through nucleotide 36 of a sequence that is at least 90% identical to a sequence of any one of SEQ ID NOs: 1-8; k. nucleotide 11 through nucleotide 36 of a sequence that is at least 90% identical to a sequence of any one of SEQ ID NOs: 1-8; 1. nucleotide 12 through nucleotide 36 of a sequence that is at least 90% identical to a sequence of any one of SEQ ID NOs: 1-8; m. nucleotide 13 through nucleotide 36 of a sequence that is at least 90% identical to a sequence of any one of SEQ ID NOs: 1-8; n. nucleotide 14 through nucleotide 36 of a sequence that is at least 90% identical to a sequence of any one of SEQ ID NOs: 1-8; o. nucleotide 1 through nucleotide 34 of a sequence that is at least 90% identical to a sequence of SEQ ID NO: 9; p. nucleotide 2 through nucleotide 34 of a sequence that is at least 90% identical to a sequence of SEQ ID NO: 9; q. nucleotide 3 through nucleotide 34 of a sequence that is at least 90% identical to a sequence of SEQ ID NO: 9; r. nucleotide 4 through nucleotide 34 of a sequence that is at least 90% identical to a sequence of SEQ ID NO: 9; s. nucleotide 5 through nucleotide 34 of a sequence that is at least 90% identical to a sequence of SEQ ID NO: 9; t. nucleotide 6 through nucleotide 34 of a sequence that is at least 90% identical to a sequence of SEQ ID NO: 9; u. nucleotide 7 through nucleotide 34 of a sequence that is at least 90% identical to a sequence of SEQ ID NO: 9; v. nucleotide 8 through nucleotide 34 of a sequence that is at least 90% identical to a sequence of SEQ ID NO: 9; w. nucleotide 9 through nucleotide 34 of a sequence that is at least 90% identical to a sequence of SEQ ID NO: 9; x. nucleotide 10 through nucleotide 34 of a sequence that is at least 90% identical to a sequence of SEQ ID NO: 9; y. nucleotide 11 through nucleotide 34 of a sequence that is at least 90% identical to a sequence of SEQ ID NO: 9; z. nucleotide 12 through nucleotide 34 of a sequence that is at least 90% identical to a sequence of SEQ ID NO: 9; or aa. a sequence that is at least 90% identical to a sequence of SEQ ID NO: 10 or a portion thereof.

In another aspect of the composition, the direct repeat comprises: a. nucleotide 1 through nucleotide 36 of any one of SEQ ID NOs: 1-8; b. nucleotide 2 through nucleotide 36 of any one of SEQ ID NOs: 1-8; c. nucleotide 3 through nucleotide 36 of any one of SEQ ID NOs: 1-8; d. nucleotide 4 through nucleotide 36 of any one of SEQ ID NOs: 1-8; e. nucleotide 5 through nucleotide 36 of any one of SEQ ID NOs: 1-8; f. nucleotide 6 through nucleotide 36 of any one of SEQ ID NOs: 1-8; g. nucleotide 7 through nucleotide 36 of any one of SEQ ID NOs: 1-8; h. nucleotide 8 through nucleotide 36 of any one of SEQ ID NOs: 1-8; i. nucleotide 9 through nucleotide 36 of any one of SEQ ID NOs: 1-8; j. nucleotide 10 through nucleotide 36 of any one of SEQ ID NOs: 1-8; k. nucleotide 11 through nucleotide 36 of any one of SEQ ID NOs: 1-8; 1. nucleotide 12 through nucleotide 36 of any one of SEQ ID NOs: 1-8; m. nucleotide 13 through nucleotide 36 of any one of SEQ ID NOs: 1-8; n. nucleotide 14 through nucleotide 36 of any one of SEQ ID NOs: 1-8; o. nucleotide 1 through nucleotide 34 of SEQ ID NO: 9; p. nucleotide 2 through nucleotide 34 of SEQ ID NO: 9; q. nucleotide 3 through nucleotide 34 of SEQ ID NO: 9; r. nucleotide 4 through nucleotide 34 of SEQ ID NO: 9; s. nucleotide 5 through nucleotide 34 of SEQ ID NO: 9; t. nucleotide 6 through nucleotide 34 of SEQ ID NO: 9; u. nucleotide 7 through nucleotide 34 of SEQ ID NO: 9; v. nucleotide 8 through nucleotide 34 of SEQ ID NO: 9; w. nucleotide 9 through nucleotide 34 of SEQ ID NO: 9; x. nucleotide 10 through nucleotide 34 of SEQ ID NO: 9; y. nucleotide 11 through nucleotide 34 of SEQ ID NO: 9; z. nucleotide 12 through nucleotide 34 of SEQ ID NO: 9; or aa. SEQ ID NO: 10 or a portion thereof.

In another aspect of the composition, the direct repeat comprises: a. nucleotide 1 through nucleotide 36 of a sequence that is at least 90% identical to a sequence of any one of SEQ ID NOs: 2652-2669; b. nucleotide 2 through nucleotide 36 of a sequence that is at least 90% identical to a sequence of any one of SEQ ID NOs: 2652-2669; c. nucleotide 3 through nucleotide 36 of a sequence that is at least 90% identical to a sequence of any one of SEQ ID NOs: 2652-2669; d. nucleotide 4 through nucleotide 36 of a sequence that is at least 90% identical to a sequence of any one of SEQ ID NOs: 2652-2669; e. nucleotide 5 through nucleotide 36 of a sequence that is at least 90% identical to a sequence of any one of SEQ ID NOs: 2652-2669; f. nucleotide 6 through nucleotide 36 of a sequence that is at least 90% identical to a sequence of any one of SEQ ID NOs: 2652-2669; g. nucleotide 7 through nucleotide 36 of a sequence that is at least 90% identical to a sequence of any one of SEQ ID NOs: 2652-2669; h. nucleotide 8 through nucleotide 36 of a sequence that is at least 90% identical to a sequence of any one of SEQ ID NOs: 2652-2669; i. nucleotide 9 through nucleotide 36 of a sequence that is at least 90% identical to a sequence of any one of SEQ ID NOs: 2652-2669; j. nucleotide 10 through nucleotide 36 of a sequence that is at least 90% identical to a sequence of any one of SEQ ID NOs: 2652-2669; k. nucleotide 11 through nucleotide 36 of a sequence that is at least 90% identical to a sequence of any one of SEQ ID NOs: 2652-2669; 1. nucleotide 12 through nucleotide 36 of a sequence that is at least 90% identical to a sequence of any one of SEQ ID NOs: 2652-2669; m. nucleotide 13 through nucleotide 36 of a sequence that is at least 90% identical to a sequence of any one of SEQ ID NOs: 2652-2669; n. nucleotide 14 through nucleotide 36 of a sequence that is at least 90% identical to a sequence of any one of SEQ ID NOs: 2652-2669; or o. a sequence that is at least 90% identical to a sequence of SEQ ID NO: 2670 or a portion thereof.

In another aspect of the composition, the direct repeat comprises: a. nucleotide 1 through nucleotide 36 of any one of SEQ ID NOs: 2652-2669; b. nucleotide 2 through nucleotide 36 of any one of SEQ ID NOs: 2652-2669; c. nucleotide 3 through nucleotide 36 of any one of SEQ ID NOs: 2652-2669; d. nucleotide 4 through nucleotide 36 of any one of SEQ ID NOs: 2652-2669; e. nucleotide 5 through nucleotide 36 of any one of SEQ ID NOs: 2652-2669; f. nucleotide 6 through nucleotide 36 of any one of SEQ ID NOs: 2652-2669; g. nucleotide 7 through nucleotide 36 of any one of SEQ ID NOs: 2652-2669; h. nucleotide 8 through nucleotide 36 of any one of SEQ ID NOs: 2652-2669; i. nucleotide 9 through nucleotide 36 of any one of SEQ ID NOs: 2652-2669; j. nucleotide 10 through nucleotide 36 of any one of SEQ ID NOs: 2652-2669; k. nucleotide 11 through nucleotide 36 of any one of SEQ ID NOs: 2652-2669; 1. nucleotide 12 through nucleotide 36 of any one of SEQ ID NOs: 2652-2669; m. nucleotide 13 through nucleotide 36 of any one of SEQ ID NOs: 2652-2669; n. nucleotide 14 through nucleotide 36 of any one of SEQ ID NOs: 2652-2669; or o. SEQ ID NO: 2670 or a portion thereof.

In another aspect of the composition, the direct repeat comprises: a. nucleotide 1 through nucleotide 36 of a sequence that is at least 90% identical to SEQ ID NO: 2671; b. nucleotide 2 through nucleotide 36 of a sequence that is at least 90% identical to SEQ ID NO: 2671; c. nucleotide 3 through nucleotide 36 of a sequence that is at least 90% identical to SEQ ID NO: 2671; d. nucleotide 4 through nucleotide 36 of a sequence that is at least 90% identical to SEQ ID NO: 2671; e. nucleotide 5 through nucleotide 36 of a sequence that is at least 90% identical to SEQ ID NO: 2671; f. nucleotide 6 through nucleotide 36 of a sequence that is at least 90% identical to SEQ ID NO: 2671; g. nucleotide 7 through nucleotide 36 of a sequence that is at least 90% identical to SEQ ID NO: 2671; h. nucleotide 8 through nucleotide 36 of a sequence that is at least 90% identical to SEQ ID NO: 2671; i. nucleotide 9 through nucleotide 36 of a sequence that is at least 90% identical to SEQ ID NO: 2671; j. nucleotide 10 through nucleotide 36 of a sequence that is at least 90% identical to SEQ ID NO: 2671; k. nucleotide 11 through nucleotide 36 of a sequence that is at least 90% identical to SEQ ID NO: 2671; 1. nucleotide 12 through nucleotide 36 of a sequence that is at least 90% identical to SEQ ID NO: 2671; m. nucleotide 13 through nucleotide 36 of a sequence that is at least 90% identical to SEQ ID NO: 2671; n. nucleotide 14 through nucleotide 36 of a sequence that is at least 90% identical to SEQ ID NO: 2671; or o. a sequence that is at least 90% identical to a sequence of SEQ ID NO: 2672 or SEQ ID NO: 2673 or a portion thereof.

In another aspect of the composition, the direct repeat comprises: a. nucleotide 1 through nucleotide 36 of SEQ ID NO: 2671; b. nucleotide 2 through nucleotide 36 of SEQ ID NO: 2671; c. nucleotide 3 through nucleotide 36 of SEQ ID NO: 2671; d. nucleotide 4 through nucleotide 36 of SEQ ID NO: 2671; e. nucleotide 5 through nucleotide 36 of SEQ ID NO: 2671; f. nucleotide 6 through nucleotide 36 of SEQ ID NO: 2671; g. nucleotide 7 through nucleotide 36 of SEQ ID NO: 2671; h. nucleotide 8 through nucleotide 36 of SEQ ID NO: 2671; i. nucleotide 9 through nucleotide 36 of SEQ ID NO: 2671; j. nucleotide 10 through nucleotide 36 of SEQ ID NO: 2671; k. nucleotide 11 through nucleotide 36 of SEQ ID NO: 2671; 1. nucleotide 12 through nucleotide 36 of SEQ ID NO: 2671; m. nucleotide 13 through nucleotide 36 of SEQ ID NO: 2671; n. nucleotide 14 through nucleotide 36 of SEQ ID NO: 2671; or o. SEQ ID NO: 2672 or SEQ ID NO: 2673 or a portion thereof.

In another aspect of the composition, the direct repeat comprises: a. nucleotide 1 through nucleotide 36 of a sequence that is at least 90% identical to a sequence of SEQ ID NO: 2674 or SEQ ID NO: 2675; b. nucleotide 2 through nucleotide 36 of a sequence that is at least 90% identical to a sequence of SEQ ID NO: 2674 or SEQ ID NO: 2675; c. nucleotide 3 through nucleotide 36 of a sequence that is at least 90% identical to a sequence of SEQ ID NO: 2674 or SEQ ID NO: 2675; d. nucleotide 4 through nucleotide 36 of a sequence that is at least 90% identical to a sequence of SEQ ID NO: 2674 or SEQ ID NO: 2675; e. nucleotide 5 through nucleotide 36 of a sequence that is at least 90% identical to a sequence of SEQ ID NO: 2674 or SEQ ID NO: 2675; f. nucleotide 6 through nucleotide 36 of a sequence that is at least 90% identical to a sequence of SEQ ID NO: 2674 or SEQ ID NO: 2675; g. nucleotide 7 through nucleotide 36 of a sequence that is at least 90% identical to a sequence of SEQ ID NO: 2674 or SEQ ID NO: 2675; h. nucleotide 8 through nucleotide 36 of a sequence that is at least 90% identical to a sequence of SEQ ID NO: 2674 or SEQ ID NO: 2675; i. nucleotide 9 through nucleotide 36 of a sequence that is at least 90% identical to a sequence of SEQ ID NO: 2674 or SEQ ID NO: 2675; j. nucleotide 10 through nucleotide 36 of a sequence that is at least 90% identical to a sequence of SEQ ID NO: 2674 or SEQ ID NO: 2675; k. nucleotide 11 through nucleotide 36 of a sequence that is at least 90% identical to a sequence of SEQ ID NO: 2674 or SEQ ID NO: 2675; 1. nucleotide 12 through nucleotide 36 of a sequence that is at least 90% identical to a sequence of SEQ ID NO: 2674 or SEQ ID NO: 2675; m. nucleotide 13 through nucleotide 36 of a sequence that is at least 90% identical to a sequence of SEQ ID NO: 2674 or SEQ ID NO: 2675; n. nucleotide 14 through nucleotide 36 of a sequence that is at least 90% identical to a sequence of SEQ ID NO: 2674 or SEQ ID NO: 2675; o. nucleotide 15 through nucleotide 36 of a sequence that is at least 90% identical to a sequence of SEQ ID NO: 2674 or SEQ ID NO: 2675; or p. a sequence that is at least 90% identical to a sequence of SEQ ID NO: 2676 or a portion thereof.

In another aspect of the composition, the direct repeat comprises: a. nucleotide 1 through nucleotide 36 of SEQ ID NO: 2674 or SEQ ID NO: 2675; b. nucleotide 2 through nucleotide 36 of SEQ ID NO: 2674 or SEQ ID NO: 2675; c. nucleotide 3 through nucleotide 36 of SEQ ID NO: 2674 or SEQ ID NO: 2675; d. nucleotide 4 through nucleotide 36 of SEQ ID NO: 2674 or SEQ ID NO: 2675; e. nucleotide 5 through nucleotide 36 of SEQ ID NO: 2674 or SEQ ID NO: 2675; f. nucleotide 6 through nucleotide 36 of SEQ ID NO: 2674 or SEQ ID NO: 2675; g. nucleotide 7 through nucleotide 36 of SEQ ID NO: 2674 or SEQ ID NO: 2675; h. nucleotide 8 through nucleotide 36 of SEQ ID NO: 2674 or SEQ ID NO: 2675; i. nucleotide 9 through nucleotide 36 of SEQ ID NO: 2674 or SEQ ID NO: 2675; j. nucleotide 10 through nucleotide 36 of SEQ ID NO: 2674 or SEQ ID NO: 2675; k. nucleotide 11 through nucleotide 36 of SEQ ID NO: 2674 or SEQ ID NO: 2675; 1. nucleotide 12 through nucleotide 36 of SEQ ID NO: 2674 or SEQ ID NO: 2675; m. nucleotide 13 through nucleotide 36 of SEQ ID NO: 2674 or SEQ ID NO: 2675; n. nucleotide 14 through nucleotide 36 of SEQ ID NO: 2674 or SEQ ID NO: 2675; o. nucleotide 15 through nucleotide 36 of SEQ ID NO: 2674 or SEQ ID NO: 2675; or p. SEQ ID NO: 2676 or a portion thereof.

In another aspect of the composition, the spacer sequence is substantially complementary to the complement of a sequence of any one of SEQ ID NOs: 11-1321.

In another aspect of the composition, the target sequence is adjacent to a protospacer adjacent motif (PAM) comprising the sequence 5′-NTTN-3′.

In another aspect of the composition, the PAM comprises the sequence 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′.

In another aspect of the composition, the target sequence is immediately adjacent to the PAM sequence.

In another aspect of the composition, the target sequence is within 1, 2, 3, 4, or 5 nucleotides of the PAM sequence.

In another aspect of the composition, the Cas12i polypeptide is: a. a Cas12i2 polypeptide comprising a sequence that is at least 90% identical to the sequence of SEQ ID NO: 2634, SEQ ID NO: 2641, SEQ ID NO: 2642, SEQ ID NO: 2643, SEQ ID NO: 2644, or SEQ ID NO: 2645; b. a Cas12i4 polypeptide comprising a sequence that is at least 90% identical to the sequence of SEQ ID NO: 2647, SEQ ID NO: 2648, or SEQ ID NO: 2649; c. a Cas12i1 polypeptide comprising a sequence that is at least 90% identical to the sequence of SEQ ID NO: 2650; or d. a Cas12i3 polypeptide comprising a sequence that is at least 90% identical to the sequence of SEQ ID NO: 2651.

In another aspect of the composition, the Cas12i polypeptide is: a. a Cas12i2 polypeptide comprising a sequence of SEQ ID NO: 2634, SEQ ID NO: 2641, SEQ ID NO: 2642, SEQ ID NO: 2643, SEQ ID NO: 2644, or SEQ ID NO: 2645; b. a Cas12i4 polypeptide comprising a sequence of SEQ ID NO: 2647, SEQ ID NO: 2648, or SEQ ID NO: 2649; c. a Cas12i1 polypeptide comprising a sequence of SEQ ID NO: 2650; or d. a Cas12i3 polypeptide comprising a sequence of SEQ ID NO: 2651.

In another aspect of the composition, the RNA guide and the Cas12i polypeptide form a ribonucleoprotein complex.

In another aspect of the composition, the ribonucleoprotein complex binds a target nucleic acid.

In another aspect of the composition, the composition is present within a cell.

In another aspect of the composition, the RNA guide and the Cas12i polypeptide are encoded in a vector, e.g., expression vector. In another aspect of the composition, the RNA guide and the Cas12i polypeptide are encoded in a single vector or the RNA guide is encoded in a first vector and the Cas12i polypeptide is encoded in a second vector.

The invention further provides a vector system comprising one or more vectors encoding an RNA guide disclosed herein and a Cas12i polypeptide. In an embodiment, the vector system comprises a first vector encoding an RNA guide disclosed herein and a second vector encoding a Cas12i polypeptide. The vectors may be expression vectors.

The invention yet further provides an RNA guide comprising (i) a spacer sequence that is substantially complementary to a target sequence within a BCL11A gene and (ii) a direct repeat sequence.

In one aspect of the RNA guide, the target sequence is within exon 1, exon 2, exon 3, exon 4, or the enhancer region of the BCL11A gene.

In another aspect of the RNA guide, the BCL11A gene comprises the sequence of SEQ ID NO: 2635, the reverse complement of SEQ ID NO: 2635, a variant of SEQ ID NO: 2635, or the reverse complement of a variant of SEQ ID NO: 2635.

In another aspect of the RNA guide, the spacer sequence comprises: a. nucleotide 1 through nucleotide 16 of a sequence that is at least 90% identical to a sequence of any one of SEQ ID NOs: 1322-2632; b. nucleotide 1 through nucleotide 17 of a sequence that is at least 90% identical to a sequence of any one of SEQ ID NOs: 1322-2632; c. nucleotide 1 through nucleotide 18 of a sequence that is at least 90% identical to a sequence of any one of SEQ ID NOs: 1322-2632; d. nucleotide 1 through nucleotide 19 of a sequence that is at least 90% identical to a sequence of any one of SEQ ID NOs: 1322-2632; e. nucleotide 1 through nucleotide 20 of a sequence that is at least 90% identical to a sequence of any one of SEQ ID NOs: 1322-2632; f. nucleotide 1 through nucleotide 21 of a sequence that is at least 90% identical to a sequence of any one of SEQ ID NOs: 1322-2632; g. nucleotide 1 through nucleotide 22 of a sequence that is at least 90% identical to a sequence of any one of SEQ ID NOs: 1322-2632; h. nucleotide 1 through nucleotide 23 of a sequence that is at least 90% identical to a sequence of any one of SEQ ID NOs: 1322-2632; i. nucleotide 1 through nucleotide 24 of a sequence that is at least 90% identical to a sequence of any one of SEQ ID NOs: 1322-2632; j. nucleotide 1 through nucleotide 25 of a sequence that is at least 90% identical to a sequence of any one of SEQ ID NOs: 1322-2632; k. nucleotide 1 through nucleotide 26 of a sequence that is at least 90% identical to a sequence of any one of SEQ ID NOs: 1322-2632; 1. nucleotide 1 through nucleotide 27 of a sequence that is at least 90% identical to a sequence of any one of SEQ ID NOs: 1322-2632; m. nucleotide 1 through nucleotide 28 of a sequence that is at least 90% identical to a sequence of any one of SEQ ID NOs: 1322-2632; n. nucleotide 1 through nucleotide 29 of a sequence that is at least 90% identical to a sequence of any one of SEQ ID NOs: 1322-1425 and 1427-2632; or o. nucleotide 1 through nucleotide 30 of a sequence that is at least 90% identical to a sequence of any one of SEQ ID NOs: 1322-1425 and 1427-2632.

In another aspect of the composition, the spacer sequence comprises: a. nucleotide 1 through nucleotide 16 of any one of SEQ ID NOs: 1322-2632; b. nucleotide 1 through nucleotide 17 of any one of SEQ ID NOs: 1322-2632; c. nucleotide 1 through nucleotide 18 of any one of SEQ ID NOs: 1322-2632; d. nucleotide 1 through nucleotide 19 of any one of SEQ ID NOs: 1322-2632; e. nucleotide 1 through nucleotide 20 of any one of SEQ ID NOs: 1322-2632; f. nucleotide 1 through nucleotide 21 of any one of SEQ ID NOs: 1322-2632; g. nucleotide 1 through nucleotide 22 of any one of SEQ ID NOs: 1322-2632; h. nucleotide 1 through nucleotide 23 of any one of SEQ ID NOs: 1322-2632; i. nucleotide 1 through nucleotide 24 of any one of SEQ ID NOs: 1322-2632; j. nucleotide 1 through nucleotide 25 of any one of SEQ ID NOs: 1322-2632; k. nucleotide 1 through nucleotide 26 of any one of SEQ ID NOs: 1322-2632; 1. nucleotide 1 through nucleotide 27 of any one of SEQ ID NOs: 1322-2632; m. nucleotide 1 through nucleotide 28 of any one of SEQ ID NOs: 1322-2632; n. nucleotide 1 through nucleotide 29 of any one of SEQ ID NOs: 1322-1425 and 1427-2632; or o. nucleotide 1 through nucleotide 30 of any one of SEQ ID NOs: 1322-1425 and 1427-2632.

In another aspect of the RNA guide, the direct repeat comprises: a. nucleotide 1 through nucleotide 36 of a sequence that is at least 90% identical to a sequence of any one of SEQ ID NOs: 1-8; b. nucleotide 2 through nucleotide 36 of a sequence that is at least 90% identical to a sequence of any one of SEQ ID NOs: 1-8; c. nucleotide 3 through nucleotide 36 of a sequence that is at least 90% identical to a sequence of any one of SEQ ID NOs: 1-8; d. nucleotide 4 through nucleotide 36 of a sequence that is at least 90% identical to a sequence of any one of SEQ ID NOs: 1-8; e. nucleotide 5 through nucleotide 36 of a sequence that is at least 90% identical to a sequence of any one of SEQ ID NOs: 1-8; f. nucleotide 6 through nucleotide 36 of a sequence that is at least 90% identical to a sequence of any one of SEQ ID NOs: 1-8; g. nucleotide 7 through nucleotide 36 of a sequence that is at least 90% identical to a sequence of any one of SEQ ID NOs: 1-8; h. nucleotide 8 through nucleotide 36 of a sequence that is at least 90% identical to a sequence of any one of SEQ ID NOs: 1-8; i. nucleotide 9 through nucleotide 36 of a sequence that is at least 90% identical to a sequence of any one of SEQ ID NOs: 1-8; j. nucleotide 10 through nucleotide 36 of a sequence that is at least 90% identical to a sequence of any one of SEQ ID NOs: 1-8; k. nucleotide 11 through nucleotide 36 of a sequence that is at least 90% identical to a sequence of any one of SEQ ID NOs: 1-8; 1. nucleotide 12 through nucleotide 36 of a sequence that is at least 90% identical to a sequence of any one of SEQ ID NOs: 1-8; m. nucleotide 13 through nucleotide 36 of a sequence that is at least 90% identical to a sequence of any one of SEQ ID NOs: 1-8; n. nucleotide 14 through nucleotide 36 of a sequence that is at least 90% identical to a sequence of any one of SEQ ID NOs: 1-8; o. nucleotide 1 through nucleotide 34 of a sequence that is at least 90% identical to a sequence of SEQ ID NO: 9; p. nucleotide 2 through nucleotide 34 of a sequence that is at least 90% identical to a sequence of SEQ ID NO: 9; q. nucleotide 3 through nucleotide 34 of a sequence that is at least 90% identical to a sequence of SEQ ID NO: 9; r. nucleotide 4 through nucleotide 34 of a sequence that is at least 90% identical to a sequence of SEQ ID NO: 9; s. nucleotide 5 through nucleotide 34 of a sequence that is at least 90% identical to a sequence of SEQ ID NO: 9; t. nucleotide 6 through nucleotide 34 of a sequence that is at least 90% identical to a sequence of SEQ ID NO: 9; u. nucleotide 7 through nucleotide 34 of a sequence that is at least 90% identical to a sequence of SEQ ID NO: 9; v. nucleotide 8 through nucleotide 34 of a sequence that is at least 90% identical to a sequence of SEQ ID NO: 9; w. nucleotide 9 through nucleotide 34 of a sequence that is at least 90% identical to a sequence of SEQ ID NO: 9; x. nucleotide 10 through nucleotide 34 of a sequence that is at least 90% identical to a sequence of SEQ ID NO: 9; y. nucleotide 11 through nucleotide 34 of a sequence that is at least 90% identical to a sequence of SEQ ID NO: 9; z. nucleotide 12 through nucleotide 34 of a sequence that is at least 90% identical to a sequence of SEQ ID NO: 9; or aa. a sequence that is at least 90% identical to a sequence of SEQ ID NO: 10 or a portion thereof.

In another aspect of the RNA guide, the direct repeat comprises: a. nucleotide 1 through nucleotide 36 of any one of SEQ ID NOs: 1-8; b. nucleotide 2 through nucleotide 36 of any one of SEQ ID NOs: 1-8; c. nucleotide 3 through nucleotide 36 of any one of SEQ ID NOs: 1-8; d. nucleotide 4 through nucleotide 36 of any one of SEQ ID NOs: 1-8; e. nucleotide 5 through nucleotide 36 of any one of SEQ ID NOs: 1-8; f. nucleotide 6 through nucleotide 36 of any one of SEQ ID NOs: 1-8; g. nucleotide 7 through nucleotide 36 of any one of SEQ ID NOs: 1-8; h. nucleotide 8 through nucleotide 36 of any one of SEQ ID NOs: 1-8; i. nucleotide 9 through nucleotide 36 of any one of SEQ ID NOs: 1-8; j. nucleotide 10 through nucleotide 36 of any one of SEQ ID NOs: 1-8; k. nucleotide 11 through nucleotide 36 of any one of SEQ ID NOs: 1-8; 1. nucleotide 12 through nucleotide 36 of any one of SEQ ID NOs: 1-8; m. nucleotide 13 through nucleotide 36 of any one of SEQ ID NOs: 1-8; n. nucleotide 14 through nucleotide 36 of any one of SEQ ID NOs: 1-8; o. nucleotide 1 through nucleotide 34 of SEQ ID NO: 9; p. nucleotide 2 through nucleotide 34 of SEQ ID NO: 9; q. nucleotide 3 through nucleotide 34 of SEQ ID NO: 9; r. nucleotide 4 through nucleotide 34 of SEQ ID NO: 9; s. nucleotide 5 through nucleotide 34 of SEQ ID NO: 9; t. nucleotide 6 through nucleotide 34 of SEQ ID NO: 9; u. nucleotide 7 through nucleotide 34 of SEQ ID NO: 9; v. nucleotide 8 through nucleotide 34 of SEQ ID NO: 9; w. nucleotide 9 through nucleotide 34 of SEQ ID NO: 9; x. nucleotide 10 through nucleotide 34 of SEQ ID NO: 9; y. nucleotide 11 through nucleotide 34 of SEQ ID NO: 9; z. nucleotide 12 through nucleotide 34 of SEQ ID NO: 9; or aa. SEQ ID NO: 10 or a portion thereof.

In another aspect of the RNA guide, the direct repeat comprises: a. nucleotide 1 through nucleotide 36 of a sequence that is at least 90% identical to a sequence of any one of SEQ ID NOs: 2652-2669; b. nucleotide 2 through nucleotide 36 of a sequence that is at least 90% identical to a sequence of any one of SEQ ID NOs: 2652-2669; c. nucleotide 3 through nucleotide 36 of a sequence that is at least 90% identical to a sequence of any one of SEQ ID NOs: 2652-2669; d. nucleotide 4 through nucleotide 36 of a sequence that is at least 90% identical to a sequence of any one of SEQ ID NOs: 2652-2669; e. nucleotide 5 through nucleotide 36 of a sequence that is at least 90% identical to a sequence of any one of SEQ ID NOs: 2652-2669; f. nucleotide 6 through nucleotide 36 of a sequence that is at least 90% identical to a sequence of any one of SEQ ID NOs: 2652-2669; g. nucleotide 7 through nucleotide 36 of a sequence that is at least 90% identical to a sequence of any one of SEQ ID NOs: 2652-2669; h. nucleotide 8 through nucleotide 36 of a sequence that is at least 90% identical to a sequence of any one of SEQ ID NOs: 2652-2669; i. nucleotide 9 through nucleotide 36 of a sequence that is at least 90% identical to a sequence of any one of SEQ ID NOs: 2652-2669; j. nucleotide 10 through nucleotide 36 of a sequence that is at least 90% identical to a sequence of any one of SEQ ID NOs: 2652-2669; k. nucleotide 11 through nucleotide 36 of a sequence that is at least 90% identical to a sequence of any one of SEQ ID NOs: 2652-2669; 1. nucleotide 12 through nucleotide 36 of a sequence that is at least 90% identical to a sequence of any one of SEQ ID NOs: 2652-2669; m. nucleotide 13 through nucleotide 36 of a sequence that is at least 90% identical to a sequence of any one of SEQ ID NOs: 2652-2669; n. nucleotide 14 through nucleotide 36 of a sequence that is at least 90% identical to a sequence of any one of SEQ ID NOs: 2652-2669; or o. a sequence that is at least 90% identical to a sequence of SEQ ID NO: 2670 or a portion thereof.

In another aspect of the RNA guide, the direct repeat comprises: a. nucleotide 1 through nucleotide 36 of any one of SEQ ID NOs: 2652-2669; b. nucleotide 2 through nucleotide 36 of any one of SEQ ID NOs: 2652-2669; c. nucleotide 3 through nucleotide 36 of any one of SEQ ID NOs: 2652-2669; d. nucleotide 4 through nucleotide 36 of any one of SEQ ID NOs: 2652-2669; e. nucleotide 5 through nucleotide 36 of any one of SEQ ID NOs: 2652-2669; f. nucleotide 6 through nucleotide 36 of any one of SEQ ID NOs: 2652-2669; g. nucleotide 7 through nucleotide 36 of any one of SEQ ID NOs: 2652-2669; h. nucleotide 8 through nucleotide 36 of any one of SEQ ID NOs: 2652-2669; i. nucleotide 9 through nucleotide 36 of any one of SEQ ID NOs: 2652-2669; j. nucleotide 10 through nucleotide 36 of any one of SEQ ID NOs: 2652-2669; k. nucleotide 11 through nucleotide 36 of any one of SEQ ID NOs: 2652-2669; 1. nucleotide 12 through nucleotide 36 of any one of SEQ ID NOs: 2652-2669; m. nucleotide 13 through nucleotide 36 of any one of SEQ ID NOs: 2652-2669; n. nucleotide 14 through nucleotide 36 of any one of SEQ ID NOs: 2652-2669; or o. SEQ ID NO: 2670 or a portion thereof.

In another aspect of the RNA guide, the direct repeat comprises: a. nucleotide 1 through nucleotide 36 of a sequence that is at least 90% identical to SEQ ID NO: 2671; b. nucleotide 2 through nucleotide 36 of a sequence that is at least 90% identical to SEQ ID NO: 2671; c. nucleotide 3 through nucleotide 36 of a sequence that is at least 90% identical to SEQ ID NO: 2671; d. nucleotide 4 through nucleotide 36 of a sequence that is at least 90% identical to SEQ ID NO: 2671; e. nucleotide 5 through nucleotide 36 of a sequence that is at least 90% identical to SEQ ID NO: 2671; f. nucleotide 6 through nucleotide 36 of a sequence that is at least 90% identical to SEQ ID NO: 2671; g. nucleotide 7 through nucleotide 36 of a sequence that is at least 90% identical to SEQ ID NO: 2671; h. nucleotide 8 through nucleotide 36 of a sequence that is at least 90% identical to SEQ ID NO: 2671; i. nucleotide 9 through nucleotide 36 of a sequence that is at least 90% identical to SEQ ID NO: 2671; j. nucleotide 10 through nucleotide 36 of a sequence that is at least 90% identical to SEQ ID NO: 2671; k. nucleotide 11 through nucleotide 36 of a sequence that is at least 90% identical to SEQ ID NO: 2671; 1. nucleotide 12 through nucleotide 36 of a sequence that is at least 90% identical to SEQ ID NO: 2671; m. nucleotide 13 through nucleotide 36 of a sequence that is at least 90% identical to SEQ ID NO: 2671; n. nucleotide 14 through nucleotide 36 of a sequence that is at least 90% identical to SEQ ID NO: 2671; or o. a sequence that is at least 90% identical to a sequence of SEQ ID NO: 2672 or SEQ ID NO: 2673 or a portion thereof.

In another aspect of the RNA guide, the direct repeat comprises: a. nucleotide 1 through nucleotide 36 of SEQ ID NO: 2671; b. nucleotide 2 through nucleotide 36 of SEQ ID NO: 2671; c. nucleotide 3 through nucleotide 36 of SEQ ID NO: 2671; d. nucleotide 4 through nucleotide 36 of SEQ ID NO: 2671; e. nucleotide 5 through nucleotide 36 of SEQ ID NO: 2671; f. nucleotide 6 through nucleotide 36 of SEQ ID NO: 2671; g. nucleotide 7 through nucleotide 36 of SEQ ID NO: 2671; h. nucleotide 8 through nucleotide 36 of SEQ ID NO: 2671; i. nucleotide 9 through nucleotide 36 of SEQ ID NO: 2671; j. nucleotide 10 through nucleotide 36 of SEQ ID NO: 2671; k. nucleotide 11 through nucleotide 36 of SEQ ID NO: 2671; 1. nucleotide 12 through nucleotide 36 of SEQ ID NO: 2671; m. nucleotide 13 through nucleotide 36 of SEQ ID NO: 2671; n. nucleotide 14 through nucleotide 36 of SEQ ID NO: 2671; or o. SEQ ID NO: 2672 or SEQ ID NO: 2673 or a portion thereof.

In another aspect of the RNA guide, the direct repeat comprises: a. nucleotide 1 through nucleotide 36 of a sequence that is at least 90% identical to a sequence of SEQ ID NO: 2674 or SEQ ID NO: 2675; b. nucleotide 2 through nucleotide 36 of a sequence that is at least 90% identical to a sequence of SEQ ID NO: 2674 or SEQ ID NO: 2675; c. nucleotide 3 through nucleotide 36 of a sequence that is at least 90% identical to a sequence of SEQ ID NO: 2674 or SEQ ID NO: 2675; d. nucleotide 4 through nucleotide 36 of a sequence that is at least 90% identical to a sequence of SEQ ID NO: 2674 or SEQ ID NO: 2675; e. nucleotide 5 through nucleotide 36 of a sequence that is at least 90% identical to a sequence of SEQ ID NO: 2674 or SEQ ID NO: 2675; f. nucleotide 6 through nucleotide 36 of a sequence that is at least 90% identical to a sequence of SEQ ID NO: 2674 or SEQ ID NO: 2675; g. nucleotide 7 through nucleotide 36 of a sequence that is at least 90% identical to a sequence of SEQ ID NO: 2674 or SEQ ID NO: 2675; h. nucleotide 8 through nucleotide 36 of a sequence that is at least 90% identical to a sequence of SEQ ID NO: 2674 or SEQ ID NO: 2675; i. nucleotide 9 through nucleotide 36 of a sequence that is at least 90% identical to a sequence of SEQ ID NO: 2674 or SEQ ID NO: 2675; j. nucleotide 10 through nucleotide 36 of a sequence that is at least 90% identical to a sequence of SEQ ID NO: 2674 or SEQ ID NO: 2675; k. nucleotide 11 through nucleotide 36 of a sequence that is at least 90% identical to a sequence of SEQ ID NO: 2674 or SEQ ID NO: 2675; 1. nucleotide 12 through nucleotide 36 of a sequence that is at least 90% identical to a sequence of SEQ ID NO: 2674 or SEQ ID NO: 2675; m. nucleotide 13 through nucleotide 36 of a sequence that is at least 90% identical to a sequence of SEQ ID NO: 2674 or SEQ ID NO: 2675; n. nucleotide 14 through nucleotide 36 of a sequence that is at least 90% identical to a sequence of SEQ ID NO: 2674 or SEQ ID NO: 2675; o. nucleotide 15 through nucleotide 36 of a sequence that is at least 90% identical to a sequence of SEQ ID NO: 2674 or SEQ ID NO: 2675; or p. a sequence that is at least 90% identical to a sequence of SEQ ID NO: 2676 or a portion thereof.

In another aspect of the RNA guide, the direct repeat comprises: a. nucleotide 1 through nucleotide 36 of SEQ ID NO: 2674 or SEQ ID NO: 2675; b. nucleotide 2 through nucleotide 36 of SEQ ID NO: 2674 or SEQ ID NO: 2675; c. nucleotide 3 through nucleotide 36 of SEQ ID NO: 2674 or SEQ ID NO: 2675; d. nucleotide 4 through nucleotide 36 of SEQ ID NO: 2674 or SEQ ID NO: 2675; e. nucleotide 5 through nucleotide 36 of SEQ ID NO: 2674 or SEQ ID NO: 2675; f. nucleotide 6 through nucleotide 36 of SEQ ID NO: 2674 or SEQ ID NO: 2675; g. nucleotide 7 through nucleotide 36 of SEQ ID NO: 2674 or SEQ ID NO: 2675; h. nucleotide 8 through nucleotide 36 of SEQ ID NO: 2674 or SEQ ID NO: 2675; i. nucleotide 9 through nucleotide 36 of SEQ ID NO: 2674 or SEQ ID NO: 2675; j. nucleotide 10 through nucleotide 36 of SEQ ID NO: 2674 or SEQ ID NO: 2675; k. nucleotide 11 through nucleotide 36 of SEQ ID NO: 2674 or SEQ ID NO: 2675; 1. nucleotide 12 through nucleotide 36 of SEQ ID NO: 2674 or SEQ ID NO: 2675; m. nucleotide 13 through nucleotide 36 of SEQ ID NO: 2674 or SEQ ID NO: 2675; n. nucleotide 14 through nucleotide 36 of SEQ ID NO: 2674 or SEQ ID NO: 2675; o. nucleotide 15 through nucleotide 36 of SEQ ID NO: 2674 or SEQ ID NO: 2675; or p. SEQ ID NO: 2676 or a portion thereof.

In another aspect of the RNA guide, the spacer sequence is substantially complementary to the complement of a sequence of any one of SEQ ID NOs: 11-1321.

In another aspect of the RNA guide, the target sequence is adjacent to a protospacer adjacent motif (PAM) comprising the sequence 5′-NTTN-3′, wherein N is any nucleotide.

In another aspect of the RNA guide, the PAM comprises the sequence 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′.

In another aspect of the RNA guide, the target sequence is immediately adjacent to the PAM sequence.

In another aspect of the RNA guide, the target sequence is within 1, 2, 3, 4, or 5 nucleotides of the PAM sequence.

The invention yet further provides a nucleic acid encoding an RNA guide as described herein.

The invention yet further provides a vector comprising such an RNA guide as described herein.

The invention yet further provides a cell comprising a composition, an RNA guide, a nucleic acid, or a vector as described herein.

In one aspect of the cell, the cell is a eukaryotic cell, an animal cell, a mammalian cell, a human cell, a primary cell, a cell line, a stem cell, or a T cell.

The invention yet further provides a kit comprising a composition, an RNA guide, a nucleic acid, or a vector as described herein.

The invention yet further provides a method of editing a BCL11A sequence, the method comprising contacting a BCL11A sequence with a composition or an RNA guide as described herein. In an embodiment, the method is carried out in vitro. In an embodiment, the method is carried out ex vivo.

In one aspect of the method, the BCL11A sequence is in a cell.

In one aspect of the method, the composition or the RNA guide induces a deletion in the BCL11A sequence.

In one aspect of the method, the deletion is adjacent to a 5′-NTTN-3′ sequence, wherein N is any nucleotide.

In one aspect of the method, the deletion is downstream of the 5′-NTTN-3′ sequence.

In one aspect of the method, the deletion is up to about 40 nucleotides in length.

In one aspect of the method, the deletion is from about 4 nucleotides to 40 nucleotides in length.

In one aspect of the method, the deletion is from about 4 nucleotides to 25 nucleotides in length.

In one aspect of the method, the deletion is from about 10 nucleotides to 25 nucleotides in length.

In one aspect of the method, the deletion is from about 10 nucleotides to 15 nucleotides in length.

In one aspect of the method, the deletion starts within about 5 nucleotides to about 15 nucleotides of the 5′-NTTN-3′ sequence.

In one aspect of the method, the deletion starts within about 5 nucleotides to about 10 nucleotides of the 5′-NTTN-3′ sequence.

In one aspect of the method, the deletion starts within about 10 nucleotides to about 15 nucleotides of the 5′-NTTN-3′ sequence.

In one aspect of the method, the deletion starts within about 5 nucleotides to about 15 nucleotides downstream of the 5′-NTTN-3′ sequence.

In one aspect of the method, the deletion starts within about 5 nucleotides to about 10 nucleotides downstream of the 5′-NTTN-3′ sequence.

In one aspect of the method, the deletion starts within about 10 nucleotides to about 15 nucleotides downstream of the 5′-NTTN-3′ sequence.

In one aspect of the method, the deletion ends within about 20 nucleotides to about 30 nucleotides of the 5′-NTTN-3′ sequence.

In one aspect of the method, the deletion ends within about 20 nucleotides to about 25 nucleotides of the 5′-NTTN-3′ sequence.

In one aspect of the method, the deletion ends within about 25 nucleotides to about 30 nucleotides of the 5′-NTTN-3′ sequence.

In one aspect of the method, the deletion ends within about 20 nucleotides to about 30 nucleotides downstream of the 5′-NTTN-3′ sequence.

In one aspect of the method, the deletion ends within about 20 nucleotides to about 25 nucleotides downstream of the 5′-NTTN-3′ sequence.

In one aspect of the method, the deletion ends within about 25 nucleotides to about 30 nucleotides downstream of the 5′-NTTN-3′ sequence.

In one aspect of the method, the deletion starts within about 5 nucleotides to about 15 nucleotides downstream of the 5′-NTTN-3′ sequence and ends within about 20 nucleotides to about 30 nucleotides downstream of the 5′-NTTN-3′ sequence.

In one aspect of the method, the deletion starts within about 5 nucleotides to about 15 nucleotides downstream of the 5′-NTTN-3′ sequence and ends within about 20 nucleotides to about 25 nucleotides downstream of the 5′-NTTN-3′ sequence.

In one aspect of the method, the deletion starts within about 5 nucleotides to about 15 nucleotides downstream of the 5′-NTTN-3′ sequence and ends within about 25 nucleotides to about 30 nucleotides downstream of the 5′-NTTN-3′ sequence.

In one aspect of the method, the deletion starts within about 5 nucleotides to about 10 nucleotides downstream of the 5′-NTTN-3′ sequence and ends within about 20 nucleotides to about 30 nucleotides downstream of the 5′-NTTN-3′ sequence.

In one aspect of the method, the deletion starts within about 5 nucleotides to about 10 nucleotides downstream of the 5′-NTTN-3′ sequence and ends within about 20 nucleotides to about 25 nucleotides downstream of the 5′-NTTN-3′ sequence.

In one aspect of the method, the deletion starts within about 5 nucleotides to about 10 nucleotides downstream of the 5′-NTTN-3′ sequence and ends within about 25 nucleotides to about 30 nucleotides downstream of the 5′-NTTN-3′ sequence.

In one aspect of the method, the deletion starts within about 10 nucleotides to about 15 nucleotides downstream of the 5′-NTTN-3′ sequence and ends within about 20 nucleotides to about 30 nucleotides downstream of the 5′-NTTN-3′ sequence.

In one aspect of the method, the deletion starts within about 10 nucleotides to about 15 nucleotides downstream of the 5′-NTTN-3′ sequence and ends within about 20 nucleotides to about 25 nucleotides downstream of the 5′-NTTN-3′ sequence.

In one aspect of the method, the deletion starts within about 10 nucleotides to about 15 nucleotides 5 downstream of the 5′-NTTN-3′ sequence and ends within about 25 nucleotides to about 30 nucleotides downstream of the 5′-NTTN-3′ sequence.

In one aspect of the method, the 5′-NTTN-3′ sequence is 5′-CTTT-3′, 5′-CTTC-3′, 5′-GTTT-3′, 5′-GTTC-3′, 5′-TTTC-3′, 5′-GTTA-3′, or 5′-GTTG-3′.

In one aspect of the method, the deletion overlaps with a mutation in the gene.

In one aspect of the method, the deletion overlaps with an insertion in the gene.

In one aspect of the method, the deletion removes a repeat expansion of the gene or a portion thereof.

In one aspect of the method, the deletion disrupts one or both alleles of the gene.

In one aspect of the method, the deletion disrupts a GATAA motif of an enhancer region of the BCL11A gene.

In one aspect of the composition, RNA guide, nucleic acid, vector, cell, kit or method described herein, the composition, RNA guide, nucleic acid, vector, cell, kit or method disrupts a GATAA motif of an enhancer region of the BCL11A gene.

In one aspect of the composition, cell, kit or method described herein, the composition, cell, kit or method comprises at least two RNA guides targeting a GATAA motif of an enhancer region of the BCL11A gene.

In one aspect of the composition, cell, kit or method described herein, the at least two RNA guides comprise at least 90% identity to:

(SEQ ID NO: 2677)
AGAAAUCCGUCUUUCAUUGACGGGAAGCUAGUCUAGUGCAAGC;
(SEQ ID NO: 2678)
AGAAAUCCGUCUUUCAUUGACGGCUGGAGCCUGUGAUAAAAGC;
and/or
(SEQ ID NO: 66)
AGAAAUCCGUCUUUCAUUGACGGUACCCCACCCACGCCCCCAC.

In one aspect of the composition, cell, kit or method described herein, the at least two RNA guides comprise at least 95% identity to:

(SEQ ID NO: 2677)
AGAAAUCCGUCUUUCAUUGACGGGAAGCUAGUCUAGUGCAAGC;
(SEQ ID NO: 2678)
AGAAAUCCGUCUUUCAUUGACGGCUGGAGCCUGUGAUAAAAGC;
and/or
(SEQ ID NO: 2679)
AGAAAUCCGUCUUUCAUUGACGGUACCCCACCCACGCCCCCAC.

In one aspect of the composition, cell, kit or method described herein, the at least two RNA guides comprise at least two sequences of:

(SEQ ID NO: 2677)
AGAAAUCCGUCUUUCAUUGACGGGAAGCUAGUCUAGUGCAAGC;
(SEQ ID NO: 2678)
AGAAAUCCGUCUUUCAUUGACGGCUGGAGCCUGUGAUAAAAGC;
and
(SEQ ID NO: 2679)
AGAAAUCCGUCUUUCAUUGACGGUACCCCACCCACGCCCCCAC.

In one aspect of the composition, RNA guide, nucleic acid, vector, cell, kit or method described herein, the RNA guide consists of the sequence of:

(SEQ ID NO: 2677)
AGAAAUCCGUCUUUCAUUGACGGGAAGCUAGUCUAGUGCAAGC;
(SEQ ID NO: 2678)
AGAAAUCCGUCUUUCAUUGACGGCUGGAGCCUGUGAUAAAAGC;
or
(SEQ ID NO: 2679)
AGAAAUCCGUCUUUCAUUGACGGUACCCCACCCACGCCCCCAC.

In one aspect of the composition, RNA guide, nucleic acid, vector, cell, kit or method described herein, the RNA guide does not consist of the sequence of:

(SEQ ID NO: 2677)
AGAAAUCCGUCUUUCAUUGACGGGAAGCUAGUCUAGUGCAAGC;
(SEQ ID NO: 2678)
AGAAAUCCGUCUUUCAUUGACGGCUGGAGCCUGUGAUAAAAGC;
or
(SEQ ID NO: 2679)
AGAAAUCCGUCUUUCAUUGACGGUACCCCACCCACGCCCCCAC.

### Definitions

The present invention will be described with respect to particular embodiments, but the invention is not limited thereto but only by the claims. Terms as set forth hereinafter are generally to be understood in their common sense unless indicated otherwise.

As used herein, the term “activity” refers to a biological activity. In some embodiments, activity includes enzymatic activity, e.g., catalytic ability of an effector. For example, activity can include nuclease activity.

As used herein the term “BCL11A” refers to “B-cell lymphoma/leukemia 11A.” BCL11A plays a role in hematopoietic development and may also function as a leukemia disease gene. SEQ ID NO: 2635 as set forth herein provides an example of a BCL11A gene sequence. It is understood that spacer sequences described herein can target SEQ ID NO: 2635 or the reverse complement thereof, depending upon whether they are indicated as “+” or “−” as set forth in Table 5. The target sequences listed in Table 5 are on the non-target strand of the BCL11A gene.

As used herein, the term “Cas12i polypeptide” (also referred to herein as Cas12i) refers to a polypeptide that binds to a target sequence on a target nucleic acid specified by an RNA guide, wherein the polypeptide has at least some amino acid sequence homology to a wild-type Cas12i polypeptide. In some embodiments, the Cas12i polypeptide comprises at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99% or 100% sequence identity with any one of SEQ ID NOs: 1-5 and 11-18 of U.S. Pat. No. 10,808,245, which is incorporated by reference herein in its entirety. In some embodiments, a Cas12i polypeptide comprises at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99% or 100% sequence identity with any one of SEQ ID NO: 3 (Cas12i1), SEQ ID NO: 5 (Cas12i2), SEQ ID NO: 14 (Cas12i3), or SEQ ID NO: 16 (Cas12i4) of U.S. Pat. No. 10,808,245, corresponding to SEQ ID NOs: 2650, 2634, 2651, and 2647 of the present application. In some embodiments, a Cas12i polypeptide of the disclosure is a Cas12i1 polypeptide or Cas12i2 polypeptide as described in PCT/US2021/025257. In some embodiments, the Cas12i polypeptide cleaves a target nucleic acid (e.g., as a nick or a double strand break).

As used herein, the term “complex” refers to a grouping of two or more molecules. In some embodiments, the complex comprises a polypeptide and a nucleic acid molecule interacting with (e.g., binding to, coming into contact with, adhering to) one another. As used herein, the term “complex” can refer to a grouping of an RNA guide and a polypeptide (e.g., a Cas12i polypeptide). As used herein, the term “complex” can refer to a grouping of an RNA guide, a polypeptide, and a target sequence. As used herein, the term “complex” can refer to a grouping of a BCL11A-targeting RNA guide and a Cas12i polypeptide.

As used herein, the term “protospacer adjacent motif” or “PAM” refers to a DNA sequence adjacent to a target sequence (e.g., a BCL11A target sequence) to which a complex comprising an RNA guide (e.g., a BCL11A-targeting RNA guide) and a Cas12i polypeptide binds. In the case of a double-stranded target, the RNA guide binds to a first strand of the target (e.g., the target strand or the spacer-complementary strand), and a PAM sequence as described herein is present in the second, complementary strand (e.g., the non-target strand or the non-spacer-complementary strand). As used herein, the term “adjacent” includes instances in which the RNA guide of a complex comprising an RNA guide and a Cas12i polypeptide specifically binds, interacts, or associates with a target sequence that is immediately adjacent to a PAM. In such instances, there are no nucleotides between the target sequence and the PAM. The term “adjacent” also includes instances in which there are a small number (e.g., 1, 2, 3, 4, or 5) of nucleotides between the target sequence, to which the RNA guide binds, and the PAM. In some embodiments, the PAM sequence as described herein is present in the non-target strand (e.g., the non-spacer-complementary strand). In such a case, the term “adjacent” includes a PAM sequence as described herein as being immediately adjacent to (or within a small number, e.g., 1, 2, 3, 4, or 5 nucleotides of) a sequence in the non-target strand.

As used herein, the term “RNA guide” refers to any RNA molecule that facilitates the targeting of a polypeptide (e.g., a Cas12i polypeptide) described herein to a target sequence (e.g., a sequence of a BCL11A gene). An RNA guide may be designed to include sequences that are complementary to a specific nucleic acid sequence (e.g., a BCL11A nucleic acid sequence). An RNA guide may comprise a DNA targeting sequence (i.e., a spacer sequence) and a direct repeat (DR) sequence. The term “crRNA” is also used herein to refer to an RNA guide.

In some embodiments, a spacer sequence is complementary to a target sequence. As used herein, the term “complementary” refers to the ability of nucleobases of a first nucleic acid molecule, such as an RNA guide, to base pair with nucleobases of a second nucleic acid molecule, such as a target sequence. Two complementary nucleic acid molecules are able to non-covalently bind under appropriate temperature and solution ionic strength conditions. In some embodiments, a first nucleic acid molecule (e.g., a spacer sequence of an RNA guide) comprises 100% complementarity to a second nucleic acid (e.g., a target sequence). In some embodiments, a first nucleic acid molecule (e.g., a spacer sequence of an RNA guide) is complementary to a second nucleic acid molecule (e.g., a target sequence) if the first nucleic acid molecule comprises at least about 80%, 81%, 82%, 83%, 84%, 85%, 86%, 87%, 88%, 89%, 90%, 91%, 92%, 93%, 94%, 95%, 96%, 97%, 98%, or 99% complementarity to the second nucleic acid. As used herein, the term “substantially complementary” refers to a polynucleotide (e.g., a spacer sequence of an RNA guide) that has a certain level of complementarity to a target sequence. In some embodiments, the level of complementarity is such that the polynucleotide can hybridize to the target sequence with sufficient affinity to permit an effector polypeptide (e.g., Cas12i) that is complexed with the polynucleotide to act (e.g., cleave) on the target sequence. In some embodiments, a spacer sequence that is substantially complementary to a target sequence has less than 100% complementarity to the target sequence. In some embodiments, a spacer sequence that is substantially complementary to a target sequence has at least about 80%, 81%, 82%, 83%, 84%, 85%, 86%, 87%, 88%, 89%, 90%, 91%, 92%, 93%, 94%, 95%, 96%, 97%, 98%, or 99% complementarity to the target sequence. In some embodiments, an RNA guide with a spacer sequence that is substantially complementary to a target sequence has 100% complementarity to the target sequence.

As used herein, the terms “target” and “target sequence” refer to a nucleic acid sequence to which an RNA guide specifically binds. In some embodiments, the DNA targeting sequence (e.g., spacer) of an RNA guide binds to a target sequence. In the case of a double-stranded target, the RNA guide binds to a first strand of the target (i.e., the target strand or the spacer-complementary strand), and a PAM sequence as described herein is present in the second, complementary strand (i.e., the non-target strand or the non-spacer-complementary strand). In some embodiments, the target strand (i.e., the spacer-complementary strand) comprises a 5′-NAAN-3′ sequence. In some embodiments, the target sequence is a sequence within a BCL11A gene sequence, including, but not limited, to the sequence set forth in SEQ ID NO: 2635 or the reverse complement thereof.

As used herein, the terms “upstream” and “downstream” refer to relative positions within a single nucleic acid (e.g., DNA) sequence in a nucleic acid molecule. “Upstream” and “downstream” relate to the 5′ to 3′ direction, respectively, in which RNA transcription occurs. A first sequence is upstream of a second sequence when the 3′ end of the first sequence occurs before the 5′ end of the second sequence. A first sequence is downstream of a second sequence when the 5′ end of the first sequence occurs after the 3′ end of the second sequence. In some embodiments, the 5′-NTTN-3′ sequence is upstream of an indel described herein, and a Cas12i-induced indel is downstream of the 5′-NTTN-3′ sequence.

### BRIEF DESCRIPTION OF THE DRAWINGS

FIG. 1 shows indel activity in CD34+ HSPC cells after targeting BCL11A intronic erythroid enhancer with different individual and multiplexed crRNAs in complex with a variant Cas12i2 of SEQ ID NO: 2642 at various RNP concentrations. Error bars represent standard deviation of the mean of two bioreplicates (two individual donors).

FIG. 2 shows viability of modified CD34+ HSPC cells 72 hours following targeting of BCL11A intronic erythroid enhancer in primary CD34+ HSPCs. Different concentrations of BCL11A intronic erythroid enhancer targeting RNPs comprising variant Cas12i2 of SEQ ID NO: 2642 and crRNAs were tested. crRNAs were tested individually and in multiplexed configuration. Error bars represent standard deviation of the mean of two bioreplicates (two individual donors).

### DETAILED DESCRIPTION

The present disclosure relates to an RNA guide capable of binding to BCL11A and methods of use thereof. In some aspects, a composition comprising an RNA guide having one or more characteristics is described herein. In some aspects, a method of producing the RNA guide is described. In some aspects, a method of delivering a composition comprising the RNA guide is described.

### Composition

In some aspects, the invention described herein comprises compositions comprising an RNA guide targeting a BCL11A gene or a portion of the BCL11A gene. In some embodiments, the RNA guide is comprised of a direct repeat component and a spacer component. In some embodiments, the RNA guide binds a Cas12i polypeptide. In some embodiments, the spacer component is substantially complementary to a BCL11A target sequence, wherein the BCL11A target sequence is adjacent to a 5′-NTTN-3′ PAM sequence as described herein. In the case of a double-stranded target, the RNA guide binds to a first strand of the target (i.e., the target strand or the spacer-complementary strand) and a PAM sequence as described herein is present in the second, complementary strand (i.e., the non-target strand or the non-spacer-complementary strand).

In some embodiments, the invention described herein comprises compositions comprising a complex, wherein the complex comprises an RNA guide targeting BCL11A. In some embodiments, the invention comprises a complex comprising an RNA guide and a Cas12i polypeptide. In some embodiments, the RNA guide and the Cas12i polypeptide bind to each other in a molar ratio of about 1:1. In some embodiments, a complex comprising an RNA guide and a Cas12i polypeptide binds to a BCL11A target sequence. In some embodiments, a complex comprising an RNA guide targeting BCL11A and a Cas12i polypeptide binds to a BCL11A target sequence at a molar ratio of about 1:1. In some embodiments, the complex comprises enzymatic activity, such as nuclease activity, that can cleave the BCL11A target sequence. The RNA guide, the Cas12i polypeptide, and the BCL11A target sequence, either alone or together, do not naturally occur.

Use of the compositions disclosed herein has advantages over those of other known nuclease systems. Cas12i polypeptides are smaller than other nucleases. For example, Cas12i2 is 1,054 amino acids in length, whereas S. pyogenes Cas9 (SpCas9) is 1,368 amino acids in length, S. thermophilus Cas9 (StCas9) is 1,128 amino acids in length, FnCpf1 is 1,300 amino acids in length, AsCpf1 is 1,307 amino acids in length, and LbCpf1 is 1,246 amino acids in length. Cas12i RNA guides, which do not require a trans-activating CRISPR RNA (tracrRNA), are also smaller than Cas9 RNA guides. The smaller Cas12i polypeptide and RNA guide sizes are beneficial for delivery. Compositions comprising a Cas12i polypeptide also demonstrate decreased off-target activity compared to compositions comprising an SpCas9 polypeptide. See PCT/US2021/025257, which is incorporated by reference in its entirety. Furthermore, indels induced by compositions comprising a Cas12i polypeptide differ from indels induced by compositions comprising an SpCas9 polypeptide. For example, SpCas9 polypeptides primarily induce insertions and deletions of 1 nucleotide in length. However, Cas12i polypeptides induce larger deletions, which can be beneficial in disrupting a larger portion of a gene such as BCL11A.

### RNA Guide

In some embodiments, the composition described herein comprises an RNA guide targeting BCL11A. In some embodiments, the composition described herein comprises two or more (e.g., 2, 3, 4, 5, 6, 7, 8, 9, or more) RNA guides targeting BCL11A.

The RNA guide may direct the Cas12i polypeptide as described herein to a BCL11A target sequence. Two or more RNA guides may target two or more separate Cas12i polypeptides (e.g., Cas12i polypeptides having the same or different sequence) as described herein to two or more (e.g., 2, 3, 4, 5, 6, 7, 8, 9, or more) BCL11A target sequences.

Those skilled in the art reading the below examples of particular kinds of RNA guides will understand that, in some embodiments, an RNA guide is BCL11A target-specific. That is, in some embodiments, an RNA guide binds specifically to one or more BCL11A target sequences (e.g., within a cell) and not to non-targeted sequences (e.g., non-specific DNA or random sequences within the same cell).

In some embodiments, the RNA guide comprises a spacer sequence followed by a direct repeat sequence, referring to the sequences in the 5′ to 3′ direction. In some embodiments, the RNA guide comprises a first direct repeat sequence followed by a spacer sequence and a second direct repeat sequence, referring to the sequences in the 5′ to 3′ direction. In some embodiments, the first and second direct repeats of such an RNA guide are identical. In some embodiments, the first and second direct repeats of such an RNA guide are different.

In some embodiments, the spacer sequence and the direct repeat sequence(s) of the RNA guide are present within the same RNA molecule. In some embodiments, the spacer and direct repeat sequences are linked directly to one another. In some embodiments, a short linker is present between the spacer and direct repeat sequences, e.g., an RNA linker of 1, 2, or 3 nucleotides in length. In some embodiments, the spacer sequence and the direct repeat sequence(s) of the RNA guide are present in separate molecules, which are joined to one another by base pairing interactions.

Additional information regarding exemplary direct repeat and spacer components of RNA guides is provided as follows.

Direct Repeat

In some embodiments, the RNA guide comprises a direct repeat sequence. In some embodiments, the direct repeat sequence of the RNA guide has a length of between 12-100, 13-75, 14-50, or 15-40 nucleotides (e.g., 15, 16, 17, 18, 19, 20, 21, 22, 23, 24, 25, 26, 27, 28, 29, 30, 31, 32, 33, 34, 35, 36, 37, 38, 39, or 40 nucleotides).

In some embodiments, the direct repeat sequence is or comprises a sequence of Table 1 or a portion of a sequence of Table 1. The direct repeat sequence can comprise nucleotide 1 through nucleotide 36 of any one of SEQ ID NOs: 1, 2, 3, 4, 5, 6, 7, or 8. The direct repeat sequence can comprise nucleotide 2 through nucleotide 36 of any one of SEQ ID NOs: 1, 2, 3, 4, 5, 6, 7, or 8. The direct repeat sequence can comprise nucleotide 3 through nucleotide 36 of any one of SEQ ID NOs: 1, 2, 3, 4, 5, 6, 7, or 8. The direct repeat sequence can comprise nucleotide 4 through nucleotide 36 of any one of SEQ ID NOs: 1, 2, 3, 4, 5, 6, 7, or 8. The direct repeat sequence can comprise nucleotide 5 through nucleotide 36 of any one of SEQ ID NOs: 1, 2, 3, 4, 5, 6, 7, or 8. The direct repeat sequence can comprise nucleotide 6 through nucleotide 36 of any one of SEQ ID NOs: 1, 2, 3, 4, 5, 6, 7, or 8. The direct repeat sequence can comprise nucleotide 7 through nucleotide 36 of any one of SEQ ID NOs: 1, 2, 3, 4, 5, 6, 7, or 8. The direct repeat sequence can comprise nucleotide 8 through nucleotide 36 of any one of SEQ ID NOs: 1, 2, 3, 4, 5, 6, 7, or 8. The direct repeat sequence can comprise nucleotide 9 through nucleotide 36 of any one of SEQ ID NOs: 1, 2, 3, 4, 5, 6, 7, or 8. The direct repeat sequence can comprise nucleotide 10 through nucleotide 36 of any one of SEQ ID NOs: 1, 2, 3, 4, 5, 6, 7, or 8. The direct repeat sequence can comprise nucleotide 11 through nucleotide 36 of any one of SEQ ID NOs: 1, 2, 3, 4, 5, 6, 7, or 8. The direct repeat sequence can comprise nucleotide 12 through nucleotide 36 of any one of SEQ ID NOs: 1, 2, 3, 4, 5, 6, 7, or 8. The direct repeat sequence can comprise nucleotide 13 through nucleotide 36 of any one of SEQ ID NOs: 1, 2, 3, 4, 5, 6, 7, or 8. The direct repeat sequence can comprise nucleotide 14 through nucleotide 36 of any one of SEQ ID NOs: 1, 2, 3, 4, 5, 6, 7, or 8. The direct repeat sequence can comprise nucleotide 1 through nucleotide 34 of SEQ ID NO: 9. The direct repeat sequence can comprise nucleotide 2 through nucleotide 34 of SEQ ID NO: 9. The direct repeat sequence can comprise nucleotide 3 through nucleotide 34 of SEQ ID NO: 9. The direct repeat sequence can comprise nucleotide 4 through nucleotide 34 of SEQ ID NO: 9. The direct repeat sequence can comprise nucleotide 5 through nucleotide 34 of SEQ ID NO: 9. The direct repeat sequence can comprise nucleotide 6 through nucleotide 34 of SEQ ID NO: 9. The direct repeat sequence can comprise nucleotide 7 through nucleotide 34 of SEQ ID NO: 9. The direct repeat sequence can comprise nucleotide 8 through nucleotide 34 of SEQ ID NO: 9. The direct repeat sequence can comprise nucleotide 9 through nucleotide 34 of SEQ ID NO: 9. The direct repeat sequence can comprise nucleotide 10 through nucleotide 34 of SEQ ID NO: 9. The direct repeat sequence can comprise nucleotide 11 through nucleotide 34 of SEQ ID NO: 9. The direct repeat sequence can comprise nucleotide 12 through nucleotide 34 of SEQ ID NO: 9. In some embodiments, the direct repeat sequence is set forth in SEQ ID NO: 10. In some embodiments, the direct repeat sequence comprises a portion of the sequence set forth in SEQ ID NO: 10.

In some embodiments, the direct repeat sequence has or comprises a sequence comprising at least 90% identity (e.g., at least 90%, 91%, 92%, 93%, 94%, 95%, 96%, 97%, 98% or 99% identity) to a sequence of Table 1 or a portion of a sequence of Table 1. The direct repeat sequence can have or comprise a sequence having at least 90% identity to a sequence comprising nucleotide 1 through nucleotide 36 of any one of SEQ ID NOs: 1, 2, 3, 4, 5, 6, 7, or 8. The direct repeat sequence can have or comprise a sequence having at least 90% identity to a sequence comprising 2 through nucleotide 36 of any one of SEQ ID NOs: 1, 2, 3, 4, 5, 6, 7, or 8. The direct repeat sequence can have or comprise a sequence having at least 90% identity to a sequence comprising 3 through nucleotide 36 of any one of SEQ ID NOs: 1, 2, 3, 4, 5, 6, 7, or 8. The direct repeat sequence can have or comprise a sequence having at least 90% identity to a sequence comprising 4 through nucleotide 36 of any one of SEQ ID NOs: 1, 2, 3, 4, 5, 6, 7, or 8. The direct repeat sequence can have or comprise a sequence having at least 90% identity to a sequence comprising 5 through nucleotide 36 of any one of SEQ ID NOs: 1, 2, 3, 4, 5, 6, 7, or 8. The direct repeat sequence can have or comprise a sequence having at least 90% identity to a sequence comprising 6 through nucleotide 36 of any one of SEQ ID NOs: 1, 2, 3, 4, 5, 6, 7, or 8. The direct repeat sequence can have or comprise a sequence having at least 90% identity to a sequence comprising 7 through nucleotide 36 of any one of SEQ ID NOs: 1, 2, 3, 4, 5, 6, 7, or 8. The direct repeat sequence can have or comprise a sequence having at least 90% identity to a sequence comprising 8 through nucleotide 36 of any one of SEQ ID NOs: 1, 2, 3, 4, 5, 6, 7, or 8. The direct repeat sequence can have or comprise a sequence having at least 90% identity to a sequence comprising 9 through nucleotide 36 of any one of SEQ ID NOs: 1, 2, 3, 4, 5, 6, 7, or 8. The direct repeat sequence can have or comprise a sequence having at least 90% identity to a sequence comprising 10 through nucleotide 36 of any one of SEQ ID NOs: 1, 2, 3, 4, 5, 6, 7, or 8. The direct repeat sequence can have or comprise a sequence having at least 90% identity to a sequence comprising 11 through nucleotide 36 of any one of SEQ ID NOs: 1, 2, 3, 4, 5, 6, 7, or 8. The direct repeat sequence can have or comprise a sequence having at least 90% identity to a sequence comprising 12 through nucleotide 36 of any one of SEQ ID NOs: 1, 2, 3, 4, 5, 6, 7, or 8. The direct repeat sequence can have or comprise a sequence having at least 90% identity to a sequence comprising 13 through nucleotide 36 of any one of SEQ ID NOs: 1, 2, 3, 4, 5, 6, 7, or 8. The direct repeat sequence can have or comprise a sequence having at least 90% identity to a sequence comprising 14 through nucleotide 36 of any one of SEQ ID NOs: 1, 2, 3, 4, 5, 6, 7, or 8. The direct repeat sequence can have or comprise a sequence having at least 90% identity to a sequence comprising 1 through nucleotide 34 of SEQ ID NO: 9. The direct repeat sequence can have or comprise a sequence having at least 90% identity to a sequence comprising 2 through nucleotide 34 of SEQ ID NO: 9. The direct repeat sequence can have or comprise a sequence having at least 90% identity to a sequence comprising 3 through nucleotide 34 of SEQ ID NO: 9. The direct repeat sequence can have or comprise a sequence having at least 90% identity to a sequence comprising 4 through nucleotide 34 of SEQ ID NO: 9. The direct repeat sequence can have or comprise a sequence having at least 90% identity to a sequence comprising 5 through nucleotide 34 of SEQ ID NO: 9. The direct repeat sequence can have or comprise a sequence having at least 90% identity to a sequence comprising 6 through nucleotide 34 of SEQ ID NO: 9. The direct repeat sequence can have or comprise a sequence having at least 90% identity to a sequence comprising 7 through nucleotide 34 of SEQ ID NO: 9. The direct repeat sequence can have or comprise a sequence having at least 90% identity to a sequence comprising 8 through nucleotide 34 of SEQ ID NO: 9. The direct repeat sequence can have or comprise a sequence having at least 90% identity to a sequence comprising 9 through nucleotide 34 of SEQ ID NO: 9. The direct repeat sequence can have or comprise a sequence having at least 90% identity to a sequence comprising 10 through nucleotide 34 of SEQ ID NO: 9. The direct repeat sequence can have or comprise a sequence having at least 90% identity to a sequence comprising 11 through nucleotide 34 of SEQ ID NO: 9. The direct repeat sequence can have or comprise a sequence having at least 90% identity to a sequence comprising 12 through nucleotide 34 of SEQ ID NO: 9. In some embodiments, the direct repeat sequence has at least 90% identity (e.g., at least 90%, 91%, 92%, 93%, 94%, 95%, 96%, 97%, 98% or 99% identity) to SEQ ID NO: 10. In some embodiments, the direct repeat sequence has at least 90% identity to a portion of the sequence set forth in SEQ ID NO: 10.

In some embodiments, compositions comprising a Cas12i2 polypeptide and an RNA guide comprising the direct repeat of SEQ ID NO: 10 and a spacer length of 20 nucleotides are capable of introducing indels into a BCL11A target sequence. See Example 1.

In some embodiments, the direct repeat sequence is or comprises a sequence that is at least 90% identical to the reverse complement of any one of SEQ ID NOs: 1-10. In some embodiments, the direct repeat sequence is or comprises the reverse complement of any one of SEQ ID NOs: 1-10.

TABLE 1
Direct repeat sequences

| Sequence | |
|---|---|
| identifier | Direct Repeat Sequence |
| SEQ ID NO: 1 | GUUGCAAAACCCAAGAAA |
| | UCCGUCUUUCAUUGACGG |
| SEQ ID NO: 2 | AAUAGCGGCCCUAAGAAA |
| | UCCGUCUUUCAUUGACGG |
| SEQ ID NO: 3 | AUUGGAACUGGCGAGAAA |
| | UCCGUCUUUCAUUGACGG |
| SEQ ID NO: 4 | CCAGCAACACCUAAGAAA |
| | UCCGUCUUUCAUUGACGG |
| SEQ ID NO: 5 | CGGCGCUCGAAUAGGAAA |
| | UCCGUCUUUCAUUGACGG |
| SEQ ID NO: 6 | GUGGCAACACCUAAGAAA |
| | UCCGUCUUUCAUUGACGG |
| SEQ ID NO: 7 | GUUGCAACACCUAAGAAA |
| | UCCGUCUUUCAUUGACGG |
| SEQ ID NO: 8 | GUUGCAAUGCCUAAGAAA |
| | UCCGUCUUUCAUUGACGG |
| SEQ ID NO: 9 | GCAACACCUAAGAAAUCC |
| | GUCUUUCAUUGACGGG |
| SEQ ID NO: 10 | AGAAAUCCGUCUUUCAUU |
| | GACGG |

In some embodiments, the direct repeat sequence is a sequence of Table 2 or a portion of a sequence of Table 2. The direct repeat sequence can comprise nucleotide 1 through nucleotide 36 of any one of SEQ ID NOs: 2652, 2653, 2654, 2655, 2656, 2657, 2658, 2659, 2660, 2661, 2662, 2663, 2664, 2665, 2666, 2667, 2668, or 2669. The direct repeat sequence can comprise nucleotide 2 through nucleotide 36 of any one of SEQ ID NOs: 2652, 2653, 2654, 2655, 2656, 2657, 2658, 2659, 2660, 2661, 2662, 2663, 2664, 2665, 2666, 2667, 2668, or 2669. The direct repeat sequence can comprise nucleotide 3 through nucleotide 36 of any one of SEQ ID NOs: 2652, 2653, 2654, 2655, 2656, 2657, 2658, 2659, 2660, 2661, 2662, 2663, 2664, 2665, 2666, 2667, 2668, or 2669. The direct repeat sequence can comprise nucleotide 4 through nucleotide 36 of any one of SEQ ID NOs: 2652, 2653, 2654, 2655, 2656, 2657, 2658, 2659, 2660, 2661, 2662, 2663, 2664, 2665, 2666, 2667, 2668, or 2669. The direct repeat sequence can comprise nucleotide 5 through nucleotide 36 of any one of SEQ ID NOs: 2652, 2653, 2654, 2655, 2656, 2657, 2658, 2659, 2660, 2661, 2662, 2663, 2664, 2665, 2666, 2667, 2668, or 2669. The direct repeat sequence can comprise nucleotide 6 through nucleotide 36 of any one of SEQ ID NOs: 2652, 2653, 2654, 2655, 2656, 2657, 2658, 2659, 2660, 2661, 2662, 2663, 2664, 2665, 2666, 2667, 2668, or 2669. The direct repeat sequence can comprise nucleotide 7 through nucleotide 36 of any one of SEQ ID NOs: 2652, 2653, 2654, 2655, 2656, 2657, 2658, 2659, 2660, 2661, 2662, 2663, 2664, 2665, 2666, 2667, 2668, or 2669. The direct repeat sequence can comprise nucleotide 8 through nucleotide 36 of any one of SEQ ID NOs: 2652, 2653, 2654, 2655, 2656, 2657, 2658, 2659, 2660, 2661, 2662, 2663, 2664, 2665, 2666, 2667, 2668, or 2669. The direct repeat sequence can comprise nucleotide 9 through nucleotide 36 of any one of SEQ ID NOs: 2652, 2653, 2654, 2655, 2656, 2657, 2658, 2659, 2660, 2661, 2662, 2663, 2664, 2665, 2666, 2667, 2668, or 2669. The direct repeat sequence can comprise nucleotide 10 through nucleotide 36 of any one of SEQ ID NOs: 2652, 2653, 2654, 2655, 2656, 2657, 2658, 2659, 2660, 2661, 2662, 2663, 2664, 2665, 2666, 2667, 2668, or 2669. The direct repeat sequence can comprise nucleotide 11 through nucleotide 36 of any one of SEQ ID NOs: 2652, 2653, 2654, 2655, 2656, 2657, 2658, 2659, 2660, 2661, 2662, 2663, 2664, 2665, 2666, 2667, 2668, or 2669. The direct repeat sequence can comprise nucleotide 12 through nucleotide 36 of any one of SEQ ID NOs: 2652, 2653, 2654, 2655, 2656, 2657, 2658, 2659, 2660, 2661, 2662, 2663, 2664, 2665, 2666, 2667, 2668, or 2669. The direct repeat sequence can comprise nucleotide 13 through nucleotide 36 of any one of SEQ ID NOs: 2652, 2653, 2654, 2655, 2656, 2657, 2658, 2659, 2660, 2661, 2662, 2663, 2664, 2665, 2666, 2667, 2668, or 2669. The direct repeat sequence can comprise nucleotide 14 through nucleotide 36 of any one of SEQ ID NOs: 2652, 2653, 2654, 2655, 2656, 2657, 2658, 2659, 2660, 2661, 2662, 2663, 2664, 2665, 2666, 2667, 2668, or 2669.

In some embodiments, the direct repeat sequence has at least 95% identity (e.g., at least 95%, 96%, 97%, 98% or 99% identity) to a sequence of Table 2 or a portion of a sequence of Table 2. The direct repeat sequence can have at least 95% identity to a sequence comprising nucleotide 1 through nucleotide 36 of any one of SEQ ID NOs: 2652, 2653, 2654, 2655, 2656, 2657, 2658, 2659, 2660, 2661, 2662, 2663, 2664, 2665, 2666, 2667, 2668, or 2669. The direct repeat sequence can have at least 95% identity to a sequence comprising 2 through nucleotide 36 of any one of SEQ ID NOs: 2652, 2653, 2654, 2655, 2656, 2657, 2658, 2659, 2660, 2661, 2662, 2663, 2664, 2665, 2666, 2667, 2668, or 2669. The direct repeat sequence can have at least 95% identity to a sequence comprising 3 through nucleotide 36 of any one of SEQ ID NOs: 2652, 2653, 2654, 2655, 2656, 2657, 2658, 2659, 2660, 2661, 2662, 2663, 2664, 2665, 2666, 2667, 2668, or 2669. The direct repeat sequence can have at least 95% identity to a sequence comprising 4 through nucleotide 36 of any one of SEQ ID NOs: 2652, 2653, 2654, 2655, 2656, 2657, 2658, 2659, 2660, 2661, 2662, 2663, 2664, 2665, 2666, 2667, 2668, or 2669. The direct repeat sequence can have at least 95% identity to a sequence comprising 5 through nucleotide 36 of any one of SEQ ID NOs: 2652, 2653, 2654, 2655, 2656, 2657, 2658, 2659, 2660, 2661, 2662, 2663, 2664, 2665, 2666, 2667, 2668, or 2669. The direct repeat sequence can have at least 95% identity to a sequence comprising 6 through nucleotide 36 of any one of SEQ ID NOs: 2652, 2653, 2654, 2655, 2656, 2657, 2658, 2659, 2660, 2661, 2662, 2663, 2664, 2665, 2666, 2667, 2668, or 2669. The direct repeat sequence can have at least 95% identity to a sequence comprising 7 through nucleotide 36 of any one of SEQ ID NOs: 2652, 2653, 2654, 2655, 2656, 2657, 2658, 2659, 2660, 2661, 2662, 2663, 2664, 2665, 2666, 2667, 2668, or 2669. The direct repeat sequence can have at least 95% identity to a sequence comprising 8 through nucleotide 36 of any one of SEQ ID NOs: 2652, 2653, 2654, 2655, 2656, 2657, 2658, 2659, 2660, 2661, 2662, 2663, 2664, 2665, 2666, 2667, 2668, or 2669. The direct repeat sequence can have at least 95% identity to a sequence comprising 9 through nucleotide 36 of any one of SEQ ID NOs: 2652, 2653, 2654, 2655, 2656, 2657, 2658, 2659, 2660, 2661, 2662, 2663, 2664, 2665, 2666, 2667, 2668, or 2669. The direct repeat sequence can have at least 95% identity to a sequence comprising 10 through nucleotide 36 of any one of SEQ ID NOs: 2652, 2653, 2654, 2655, 2656, 2657, 2658, 2659, 2660, 2661, 2662, 2663, 2664, 2665, 2666, 2667, 2668, or 2669. The direct repeat sequence can have at least 95% identity to a sequence comprising 11 through nucleotide 36 of any one of SEQ ID NOs: 2652, 2653, 2654, 2655, 2656, 2657, 2658, 2659, 2660, 2661, 2662, 2663, 2664, 2665, 2666, 2667, 2668, or 2669. The direct repeat sequence can have at least 95% identity to a sequence comprising 12 through nucleotide 36 of any one of SEQ ID NOs: 2652, 2653, 2654, 2655, 2656, 2657, 2658, 2659, 2660, 2661, 2662, 2663, 2664, 2665, 2666, 2667, 2668, or 2669. The direct repeat sequence can have at least 95% identity to a sequence comprising 13 through nucleotide 36 of any one of SEQ ID NOs: 2652, 2653, 2654, 2655, 2656, 2657, 2658, 2659, 2660, 2661, 2662, 2663, 2664, 2665, 2666, 2667, 2668, or 2669.

In some embodiments, the direct repeat sequence has at least 90% identity (e.g., at least 90%, 91%, 92%, 93%, 94%, 95%, 96%, 97%, 98% or 99% identity) to a sequence of Table 2 or a portion of a sequence of Table 2. The direct repeat sequence can have at least 90% identity to a sequence comprising nucleotide 1 through nucleotide 36 of any one of SEQ ID NOs: 2652, 2653, 2654, 2655, 2656, 2657, 2658, 2659, 2660, 2661, 2662, 2663, 2664, 2665, 2666, 2667, 2668, or 2669. The direct repeat sequence can have at least 90% identity to a sequence comprising 2 through nucleotide 36 of any one of SEQ ID NOs: 2652, 2653, 2654, 2655, 2656, 2657, 2658, 2659, 2660, 2661, 2662, 2663, 2664, 2665, 2666, 2667, 2668, or 2669. The direct repeat sequence can have at least 90% identity to a sequence comprising 3 through nucleotide 36 of any one of SEQ ID NOs: 2652, 2653, 2654, 2655, 2656, 2657, 2658, 2659, 2660, 2661, 2662, 2663, 2664, 2665, 2666, 2667, 2668, or 2669. The direct repeat sequence can have at least 90% identity to a sequence comprising 4 through nucleotide 36 of any one of SEQ ID NOs: 2652, 2653, 2654, 2655, 2656, 2657, 2658, 2659, 2660, 2661, 2662, 2663, 2664, 2665, 2666, 2667, 2668, or 2669. The direct repeat sequence can have at least 90% identity to a sequence comprising 5 through nucleotide 36 of any one of SEQ ID NOs: 2652, 2653, 2654, 2655, 2656, 2657, 2658, 2659, 2660, 2661, 2662, 2663, 2664, 2665, 2666, 2667, 2668, or 2669. The direct repeat sequence can have at least 90% identity to a sequence comprising 6 through nucleotide 36 of any one of SEQ ID NOs: 2652, 2653, 2654, 2655, 2656, 2657, 2658, 2659, 2660, 2661, 2662, 2663, 2664, 2665, 2666, 2667, 2668, or 2669. The direct repeat sequence can have at least 90% identity to a sequence comprising 7 through nucleotide 36 of any one of SEQ ID NOs: 2652, 2653, 2654, 2655, 2656, 2657, 2658, 2659, 2660, 2661, 2662, 2663, 2664, 2665, 2666, 2667, 2668, or 2669. The direct repeat sequence can have at least 90% identity to a sequence comprising 8 through nucleotide 36 of any one of SEQ ID NOs: 2652, 2653, 2654, 2655, 2656, 2657, 2658, 2659, 2660, 2661, 2662, 2663, 2664, 2665, 2666, 2667, 2668, or 2669. The direct repeat sequence can have at least 90% identity to a sequence comprising 9 through nucleotide 36 of any one of SEQ ID NOs: 2652, 2653, 2654, 2655, 2656, 2657, 2658, 2659, 2660, 2661, 2662, 2663, 2664, 2665, 2666, 2667, 2668, or 2669. The direct repeat sequence can have at least 90% identity to a sequence comprising 10 through nucleotide 36 of any one of SEQ ID NOs: 2652, 2653, 2654, 2655, 2656, 2657, 2658, 2659, 2660, 2661, 2662, 2663, 2664, 2665, 2666, 2667, 2668, or 2669. The direct repeat sequence can have at least 90% identity to a sequence comprising 11 through nucleotide 36 of any one of SEQ ID NOs: 2652, 2653, 2654, 2655, 2656, 2657, 2658, 2659, 2660, 2661, 2662, 2663, 2664, 2665, 2666, 2667, 2668, or 2669. The direct repeat sequence can have at least 90% identity to a sequence comprising 12 through nucleotide 36 of any one of SEQ ID NOs: 2652, 2653, 2654, 2655, 2656, 2657, 2658, 2659, 2660, 2661, 2662, 2663, 2664, 2665, 2666, 2667, 2668, or 2669. The direct repeat sequence can have at least 90% identity to a sequence comprising 13 through nucleotide 36 of any one of SEQ ID NOs: 2652, 2653, 2654, 2655, 2656, 2657, 2658, 2659, 2660, 2661, 2662, 2663, 2664, 2665, 2666, 2667, 2668, or 2669.

In some embodiments, the direct repeat sequence is at least 90% identical to the reverse complement of any one of SEQ ID NOs: 2652, 2653, 2654, 2655, 2656, 2657, 2658, 2659, 2660, 2661, 2662, 2663, 2664, 2665, 2666, 2667, 2668, or 2669. In some embodiments, the direct repeat sequence is at least 95% identical to the reverse complement of any one of SEQ ID NOs: 2652, 2653, 2654, 2655, 2656, 2657, 2658, 2659, 2660, 2661, 2662, 2663, 2664, 2665, 2666, 2667, 2668, or 2669. In some embodiments, the direct repeat sequence is the reverse complement of any one of SEQ ID NOs: 2652, 2653, 2654, 2655, 2656, 2657, 2658, 2659, 2660, 2661, 2662, 2663, 2664, 2665, 2666, 2667, 2668, or 2669.

In some embodiments, the direct repeat sequence is at least 90% identical to SEQ ID NO: 2670 or a portion of SEQ ID NO: 2670. In some embodiments, the direct repeat sequence is at least 95% identical to SEQ ID NO: 2670 or a portion of SEQ ID NO: 2670. In some embodiments, the direct repeat sequence is 100% identical to SEQ ID NO: 2670 or a portion of SEQ ID NO: 2670.

TABLE 2
Cas1214 direct repeat sequences.

| Sequence identifier | Direct Repeat Sequence |
|---|---|
| SEQ ID NO: 2652 | UCUCAACGAUAGUCAGAC |
| | AUGUGUCCUCAGUGACAC |
| SEQ ID NO: 2653 | UUUUAACAACACUCAGGC |
| | AUGUGUCCACAGUGACAC |
| SEQ ID NO: 2654 | UUGAACGGAUACUCAGAC |
| | AUGUGUUUCCAGUGACAC |
| SEQ ID NO: 2655 | UGCCCUCAAUAGUCAGAU |
| | GUGUGUCCACAGUGACAC |
| SEQ ID NO: 2656 | UCUCAAUGAUACUUAGAU |
| | ACGUGUCCUCAGUGACAC |
| SEQ ID NO: 2657 | UCUCAAUGAUACUCAGAC |
| | AUGUGUCCCCAGUGACAC |
| SEQ ID NO: 2658 | UCUCAAUGAUACUAAGAC |
| | AUGUGUCCUCAGUGACAC |
| SEQ ID NO: 2659 | UCUCAACUAUACUCAGAC |
| | AUGUGUCCUCAGUGACAC |
| SEQ ID NO: 2660 | UCUCAACGAUACUCAGAC |
| | AUGUGUCCUCAGUGACAC |
| SEQ ID NO: 2661 | UCUCAACGAUACUAAGAU |
| | AUGUGUCCUCAGCGACAC |
| SEQ ID NO: 2662 | UCUCAACGAUACUAAGAU |
| | AUGUGUCCCCAGUGACAC |
| SEQ ID NO: 2663 | UCUCAACGAUACUAAGAU |
| | AUGUGUCCACAGUGACAC |
| SEQ ID NO: 2664 | UCUCAACAAUACUCAGAC |
| | AUGUGUCCCCAGUGACAC |
| SEQ ID NO: 2665 | UCUCAACAAUACUAAGGC |
| | AUGUGUCCCCAGUGACCC |
| SEQ ID NO: 2666 | UCUCAAAGAUACUCAGAC |
| | ACGUGUCCCCAGUGACAC |
| SEQ ID NO: 2667 | UCUCAAAAAUACUCAGAC |
| | AUGUGUCCUCAGUGACAC |
| SEQ ID NO: 2668 | GCGAAACAACAGUCAGAC |
| | AUGUGUCCCCAGUGACAC |
| SEQ ID NO: 2669 | CCUCAACGAUAUUAAGAC |
| | AUGUGUCCGCAGUGACAC |
| SEQ ID NO: 2670 | AGACAUGUGUCCUCAGUG |
| | ACAC |

In some embodiments, the direct repeat sequence is a sequence of Table 3 or a portion of a sequence of Table 3. In some embodiments, the direct repeat sequence has at least 95% identity (e.g., at least 95%, 96%, 97%, 98% or 99% identity) to a sequence of Table 3 or a portion of a sequence of Table 3. In some embodiments, the direct repeat sequence has at least 90% identity (e.g., at least 90%, 91%, 92%, 93%, 94%, 95%, 96%, 97%, 98% or 99% identity) to a sequence of Table 3 or a portion of a sequence of Table 3. In some embodiments, the direct repeat sequence is at least 90% identical to the reverse complement of any one of SEQ ID NOs: 2671-2673. In some embodiments, the direct repeat sequence is at least 95% identical to the reverse complement of any one of SEQ ID NOs: 2671-2673. In some embodiments, the direct repeat sequence is the reverse complement of any one of SEQ ID NOs: 2671-2673.

TABLE 3
Cas12il direct repeat sequences.

| Sequence identifier | Direct Repeat Sequence |
|---|---|
| SEQ ID NO: 2671 | GUUGGAAUGACUAAUUUU |
| | UGUGCCCACCGUUGGCAC |
| SEQ ID NO: 2672 | AAUUUUUGUGCCCAUCGU |
| | UGGCAC |
| SEQ ID NO: 2673 | AUUUUUGUGCCCAUCGUU |
| | GGCAC |

In some embodiments, the direct repeat sequence is a sequence of Table 4 or a portion of a sequence of Table 4. In some embodiments, the direct repeat sequence has at least 95% identity (e.g., at least 95%, 96%, 97%, 98% or 99% identity) to a sequence of Table 4 or a portion of a sequence of Table 4. In some embodiments, the direct repeat sequence has at least 90% identity (e.g., at least 90%, 91%, 92%, 93%, 94%, 95%, 96%, 97%, 98% or 99% identity) to a sequence of Table 4 or a portion of a sequence of Table 4. In some embodiments, the direct repeat sequence is at least 90% identical to the reverse complement of any one of SEQ ID NOs: 2674-2676. In some embodiments, the direct repeat sequence is at least 95% identical to the reverse complement of any one of SEQ ID NOs: 2674-2676. In some embodiments, the direct repeat sequence is the reverse complement of any one of SEQ ID NOs: 2674-2676.

TABLE 4
Cas12i3 direct repeat sequences.

| Sequence identifier | Direct Repeat Sequence |
|---|---|
| SEQ ID NO: 2674 | CUAGCAAUGACCUAAUAG |
| | UGUGUCCUUAGUUGACAU |
| SEQ ID NO: 2675 | CCUACAAUACCUAAGAAA |
| | UCCGUCCUAAGUUGACGG |
| SEQ ID NO: 2676 | AUAGUGUGUCCUUAGUUG |
| | ACAU |

In some embodiments, a direct repeat sequence described herein comprises a uracil (U). In some embodiments, a direct repeat sequence described herein comprises a thymine (T). In some embodiments, a direct repeat sequence according to Tables 1-4 comprises a sequence comprising a thymine in one or more places indicated as uracil in Tables 1-4.

Spacer

In some embodiments, the RNA guide comprises a DNA targeting or spacer sequence. In some embodiments, the spacer sequence of the RNA guide has a length of between 12-100, 13-75, 14-50, or 15-30 nucleotides (e.g., 15, 16, 17, 18, 19, 20, 21, 22, 23, 24, 25, 26, 27, 28, 29, or 30 nucleotides) and is complementary a specific target sequence. In some embodiments, the spacer sequence is designed to be complementary to a specific DNA strand, e.g., of a genomic locus.

In some embodiments, the RNA guide spacer sequence is substantially identical to a complementary strand of a target sequence. In some embodiments, the RNA guide comprises a sequence having at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to a complementary strand of a reference nucleic acid sequence, e.g., target sequence. The percent identity between two such nucleic acids can be determined manually by inspection of the two optimally aligned nucleic acid sequences or by using software programs or algorithms (e.g., BLAST, ALIGN, CLUSTAL) using standard parameters.

In some embodiments, the RNA guide comprises a spacer sequence that has a length of between 12-100, 13-75, 14-50, or 15-30 nucleotides (e.g., 15, 16, 17, 18, 19, 20, 21, 22, 23, 24, 25, 26, 27, 28, 29, or 30 nucleotides) and at least 80%, at least 90%, at least 95%, at least 96%, at least 97%, at least 98%, at least 99% complementary to a target sequence. In some embodiments, the RNA guide comprises a sequence at least 80%, at least 90%, at least 95%, at least 96%, at least 97%, at least 98%, at least 99% complementary to a target DNA sequence. In some embodiments, the RNA guide comprises a sequence at least 80%, at least 90%, at least 95%, at least 96%, at least 97%, at least 98%, at least 99% complementary to a target genomic sequence.

In some embodiments, the RNA guide comprises a sequence, e.g., RNA sequence, that is a length of up to 50 and at least 80%, at least 90%, at least 95%, at least 96%, at least 97%, at least 98%, at least 99% complementary to a target sequence. In some embodiments, the RNA guide comprises a sequence at least 80%, at least 90%, at least 95%, at least 96%, at least 97%, at least 98%, at least 99% complementary to a target DNA sequence. In some embodiments, the RNA guide comprises a sequence at least 80%, at least 90%, at least 95%, at least 96%, at least 97%, at least 98%, at least 99% complementary to a target genomic sequence.

In some embodiments, the spacer sequence is or comprises a sequence of Table 5 or a portion of a sequence of Table 5. The target sequences listed in Table 5 are on the non-target strand of the BCL11A sequence. It should be understood that an indication of SEQ ID NOs: 1322-2632 should be considered as equivalent to a listing of SEQ ID NOs: 1322-2632, with each of the intervening numbers present in the listing, i.e., 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, 1349, 1350, 1351, 1352, 1353, 1354, 1355, 1356, 1357, 1358, 1359, 1360, 1361, 1362, 1363, 1364, 1365, 1366, 1367, 1368, 1369, 1370, 1371, 1372, 1373, 1374, 1375, 1376, 1377, 1378, 1379, 1380, 1381, 1382, 1383, 1384, 1385, 1386, 1387, 1388, 1389, 1390, 1391, 1392, 1393, 1394, 1395, 1396, 1397, 1398, 1399, 1400, 1401, 1402, 1403, 1404, 1405, 1406, 1407, 1408, 1409, 1410, 1411, 1412, 1413, 1414, 1415, 1416, 1417, 1418, 1419, 1420, 1421, 1422, 1423, 1424, 1425, 1426, 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1451, 1452, 1453, 1454, 1455, 1456, 1457, 1458, 1459, 1460, 1461, 1462, 1463, 1464, 1465, 1466, 1467, 1468, 1469, 1470, 1471, 1472, 1473, 1474, 1475, 1476, 1477, 1478, 1479, 1480, 1481, 1482, 1483, 1484, 1485, 1486, 1487, 1488, 1489, 1490, 1491, 1492, 1493, 1494, 1495, 1496, 1497, 1498, 1499, 1500, 1501, 1502, 1503, 1504, 1505, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, 1603, 1604, 1605, 1606, 1607, 1608, 1609, 1610, 1611, 1612, 1613, 1614, 1615, 1616, 1617, 1618, 1619, 1620, 1621, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1649, 1650, 1651, 1652, 1653, 1654, 1655, 1656, 1657, 1658, 1659, 1660, 1661, 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, 1695, 1696, 1697, 1698, 1699, 1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, 1732, 1733, 1734, 1735, 1736, 1737, 1738, 1739, 1740, 1741, 1742, 1743, 1744, 1745, 1746, 1747, 1748, 1749, 1750, 1751, 1752, 1753, 1754, 1755, 1756, 1757, 1758, 1759, 1760, 1761, 1762, 1763, 1764, 1765, 1766, 1767, 1768, 1769, 1770, 1771, 1772, 1773, 1774, 1775, 1776, 1777, 1778, 1779, 1780, 1781, 1782, 1783, 1784, 1785, 1786, 1787, 1788, 1789, 1790, 1791, 1792, 1793, 1794, 1795, 1796, 1797, 1798, 1799, 1800, 1801, 1802, 1803, 1804, 1805, 1806, 1807, 1808, 1809, 1810, 1811, 1812, 1813, 1814, 1815, 1816, 1817, 1818, 1819, 1820, 1821, 1822, 1823, 1824, 1825, 1826, 1827, 1828, 1829, 1830, 1831, 1832, 1833, 1834, 1835, 1836, 1837, 1838, 1839, 1840, 1841, 1842, 1843, 1844, 1845, 1846, 1847, 1848, 1849, 1850, 1851, 1852, 1853, 1854, 1855, 1856, 1857, 1858, 1859, 1860, 1861, 1862, 1863, 1864, 1865, 1866, 1867, 1868, 1869, 1870, 1871, 1872, 1873, 1874, 1875, 1876, 1877, 1878, 1879, 1880, 1881, 1882, 1883, 1884, 1885, 1886, 1887, 1888, 1889, 1890, 1891, 1892, 1893, 1894, 1895, 1896, 1897, 1898, 1899, 1900, 1901, 1902, 1903, 1904, 1905, 1906, 1907, 1908, 1909, 1910, 1911, 1912, 1913, 1914, 1915, 1916, 1917, 1918, 1919, 1920, 1921, 1922, 1923, 1924, 1925, 1926, 1927, 1928, 1929, 1930, 1931, 1932, 1933, 1934, 1935, 1936, 1937, 1938, 1939, 1940, 1941, 1942, 1943, 1944, 1945, 1946, 1947, 1948, 1949, 1950, 1951, 1952, 1953, 1954, 1955, 1956, 1957, 1958, 1959, 1960, 1961, 1962, 1963, 1964, 1965, 1966, 1967, 1968, 1969, 1970, 1971, 1972, 1973, 1974, 1975, 1976, 1977, 1978, 1979, 1980, 1981, 1982, 1983, 1984, 1985, 1986, 1987, 1988, 1989, 1990, 1991, 1992, 1993, 1994, 1995, 1996, 1997, 1998, 1999, 2000, 2001, 2002, 2003, 2004, 2005, 2006, 2007, 2008, 2009, 2010, 2011, 2012, 2013, 2014, 2015, 2016, 2017, 2018, 2019, 2020, 2021, 2022, 2023, 2024, 2025, 2026, 2027, 2028, 2029, 2030, 2031, 2032, 2033, 2034, 2035, 2036, 2037, 2038, 2039, 2040, 2041, 2042, 2043, 2044, 2045, 2046, 2047, 2048, 2049, 2050, 2051, 2052, 2053, 2054, 2055, 2056, 2057, 2058, 2059, 2060, 2061, 2062, 2063, 2064, 2065, 2066, 2067, 2068, 2069, 2070, 2071, 2072, 2073, 2074, 2075, 2076, 2077, 2078, 2079, 2080, 2081, 2082, 2083, 2084, 2085, 2086, 2087, 2088, 2089, 2090, 2091, 2092, 2093, 2094, 2095, 2096, 2097, 2098, 2099, 2100, 2101, 2102, 2103, 2104, 2105, 2106, 2107, 2108, 2109, 2110, 2111, 2112, 2113, 2114, 2115, 2116, 2117, 2118, 2119, 2120, 2121, 2122, 2123, 2124, 2125, 2126, 2127, 2128, 2129, 2130, 2131, 2132, 2133, 2134, 2135, 2136, 2137, 2138, 2139, 2140, 2141, 2142, 2143, 2144, 2145, 2146, 2147, 2148, 2149, 2150, 2151, 2152, 2153, 2154, 2155, 2156, 2157, 2158, 2159, 2160, 2161, 2162, 2163, 2164, 2165, 2166, 2167, 2168, 2169, 2170, 2171, 2172, 2173, 2174, 2175, 2176, 2177, 2178, 2179, 2180, 2181, 2182, 2183, 2184, 2185, 2186, 2187, 2188, 2189, 2190, 2191, 2192, 2193, 2194, 2195, 2196, 2197, 2198, 2199, 2200, 2201, 2202, 2203, 2204, 2205, 2206, 2207, 2208, 2209, 2210, 2211, 2212, 2213, 2214, 2215, 2216, 2217, 2218, 2219, 2220, 2221, 2222, 2223, 2224, 2225, 2226, 2227, 2228, 2229, 2230, 2231, 2232, 2233, 2234, 2235, 2236, 2237, 2238, 2239, 2240, 2241, 2242, 2243, 2244, 2245, 2246, 2247, 2248, 2249, 2250, 2251, 2252, 2253, 2254, 2255, 2256, 2257, 2258, 2259, 2260, 2261, 2262, 2263, 2264, 2265, 2266, 2267, 2268, 2269, 2270, 2271, 2272, 2273, 2274, 2275, 2276, 2277, 2278, 2279, 2280, 2281, 2282, 2283, 2284, 2285, 2286, 2287, 2288, 2289, 2290, 2291, 2292, 2293, 2294, 2295, 2296, 2297, 2298, 2299, 2300, 2301, 2302, 2303, 2304, 2305, 2306, 2307, 2308, 2309, 2310, 2311, 2312, 2313, 2314, 2315, 2316, 2317, 2318, 2319, 2320, 2321, 2322, 2323, 2324, 2325, 2326, 2327, 2328, 2329, 2330, 2331, 2332, 2333, 2334, 2335, 2336, 2337, 2338, 2339, 2340, 2341, 2342, 2343, 2344, 2345, 2346, 2347, 2348, 2349, 2350, 2351, 2352, 2353, 2354, 2355, 2356, 2357, 2358, 2359, 2360, 2361, 2362, 2363, 2364, 2365, 2366, 2367, 2368, 2369, 2370, 2371, 2372, 2373, 2374, 2375, 2376, 2377, 2378, 2379, 2380, 2381, 2382, 2383, 2384, 2385, 2386, 2387, 2388, 2389, 2390, 2391, 2392, 2393, 2394, 2395, 2396, 2397, 2398, 2399, 2400, 2401, 2402, 2403, 2404, 2405, 2406, 2407, 2408, 2409, 2410, 2411, 2412, 2413, 2414, 2415, 2416, 2417, 2418, 2419, 2420, 2421, 2422, 2423, 2424, 2425, 2426, 2427, 2428, 2429, 2430, 2431, 2432, 2433, 2434, 2435, 2436, 2437, 2438, 2439, 2440, 2441, 2442, 2443, 2444, 2445, 2446, 2447, 2448, 2449, 2450, 2451, 2452, 2453, 2454, 2455, 2456, 2457, 2458, 2459, 2460, 2461, 2462, 2463, 2464, 2465, 2466, 2467, 2468, 2469, 2470, 2471, 2472, 2473, 2474, 2475, 2476, 2477, 2478, 2479, 2480, 2481, 2482, 2483, 2484, 2485, 2486, 2487, 2488, 2489, 2490, 2491, 2492, 2493, 2494, 2495, 2496, 2497, 2498, 2499, 2500, 2501, 2502, 2503, 2504, 2505, 2506, 2507, 2508, 2509, 2510, 2511, 2512, 2513, 2514, 2515, 2516, 2517, 2518, 2519, 2520, 2521, 2522, 2523, 2524, 2525, 2526, 2527, 2528, 2529, 2530, 2531, 2532, 2533, 2534, 2535, 2536, 2537, 2538, 2539, 2540, 2541, 2542, 2543, 2544, 2545, 2546, 2547, 2548, 2549, 2550, 2551, 2552, 2553, 2554, 2555, 2556, 2557, 2558, 2559, 2560, 2561, 2562, 2563, 2564, 2565, 2566, 2567, 2568, 2569, 2570, 2571, 2572, 2573, 2574, 2575, 2576, 2577, 2578, 2579, 2580, 2581, 2582, 2583, 2584, 2585, 2586, 2587, 2588, 2589, 2590, 2591, 2592, 2593, 2594, 2595, 2596, 2597, 2598, 2599, 2600, 2601, 2602, 2603, 2604, 2605, 2606, 2607, 2608, 2609, 2610, 2611, 2612, 2613, 2614, 2615, 2616, 2617, 2618, 2619, 2620, 2621, 2622, 2623, 2624, 2625, 2626, 2627, 2628, 2629, 2630, 2631, and 2632.

The spacer sequence can comprise nucleotide 1 through nucleotide 16 of any one of SEQ ID NOs: 1322-2632. The spacer sequence can comprise nucleotide 1 through nucleotide 17 of any one of SEQ ID NOs: 1322-2632. The spacer sequence can comprise nucleotide 1 through nucleotide 18 of any one of SEQ ID NOs: 1322-2632. The spacer sequence can comprise nucleotide 1 through nucleotide 19 of any one of SEQ ID NOs: 1322-2632. The spacer sequence can comprise nucleotide 1 through nucleotide 20 of any one of SEQ ID NOs: 1322-2632. The spacer sequence can comprise nucleotide 1 through nucleotide 21 of any one of SEQ ID NOs: 1322-2632. The spacer sequence can comprise nucleotide 1 through nucleotide 22 of any one of SEQ ID NOs: 1322-2632. The spacer sequence can comprise nucleotide 1 through nucleotide 23 of any one of SEQ ID NOs: 1322-2632. The spacer sequence can comprise nucleotide 1 through nucleotide 24 of any one of SEQ ID NOs: 1322-2632. The spacer sequence can comprise nucleotide 1 through nucleotide 25 of any one of SEQ ID NOs: 1322-2632. The spacer sequence can comprise nucleotide 1 through nucleotide 26 of any one of SEQ ID NOs: 1322-2632. The spacer sequence can comprise nucleotide 1 through nucleotide 27 of any one of SEQ ID NOs: 1322-2632. The spacer sequence can comprise nucleotide 1 through nucleotide 28 of any one of SEQ ID NOs: 1322-2632. The spacer sequence can comprise nucleotide 1 through nucleotide 29 of any one of SEQ ID NOs: 1322-1425 and 1427-2632. The spacer sequence can comprise nucleotide 1 through nucleotide 30 of any one of SEQ ID NOs: 1322-1425 and 1427-2632.

In some embodiments, the spacer sequence has or comprises a sequence having at least 90% identity (e.g., at least 90%, 91%, 92%, 93%, 94%, 95%, 96%, 97%, 98% or 99% identity) to a sequence of Table 5 or a portion of a sequence of Table 5. The spacer sequence can have or comprise a sequence having at least 90% identity to a sequence comprising nucleotide 1 through nucleotide 16 of any one of SEQ ID NOs: 1322-2632. The spacer sequence can have or comprise a sequence having at least 90% identity to a sequence comprising nucleotide 1 through nucleotide 17 of any one of SEQ ID NOs: 1322-2632. The spacer sequence can have or comprise a sequence having at least 90% identity to a sequence comprising nucleotide 1 through nucleotide 18 of any one of SEQ ID NOs: 1322-2632. The spacer sequence can have or comprise a sequence having at least 90% identity to a sequence comprising nucleotide 1 through nucleotide 19 of any one of SEQ ID NOs: 1322-2632. The spacer sequence can have or comprise a sequence having at least 90% identity to a sequence comprising nucleotide 1 through nucleotide 20 of any one of SEQ ID NOs: 1322-2632. The spacer sequence can have or comprise a sequence having at least 90% identity to a sequence comprising nucleotide 1 through nucleotide 21 of any one of SEQ ID NOs: 1322-2632. The spacer sequence can have or comprise a sequence having at least 90% identity to a sequence comprising nucleotide 1 through nucleotide 22 of any one of SEQ ID NOs: 1322-2632. The spacer sequence can have or comprise a sequence having at least 90% identity to a sequence comprising nucleotide 1 through nucleotide 23 of any one of SEQ ID NOs: 1322-2632. The spacer sequence can have or comprise a sequence having at least 90% identity to a sequence comprising nucleotide 1 through nucleotide 24 of any one of SEQ ID NOs: 1322-2632. The spacer sequence can have or comprise a sequence having at least 90% identity to a sequence comprising nucleotide 1 through nucleotide 25 of any one of SEQ ID NOs: 1322-2632. The spacer sequence can have or comprise a sequence having at least 90% identity to a sequence comprising nucleotide 1 through nucleotide 26 of any one of SEQ ID NOs: 1322-2632. The spacer sequence can have or comprise a sequence having at least 90% identity to a sequence comprising nucleotide 1 through nucleotide 27 of any one of SEQ ID NOs: 1322-2632. The spacer sequence can have or comprise a sequence having at least 90% identity to a sequence comprising nucleotide 1 through nucleotide 28 of any one of SEQ ID NOs: 1322-2632. The spacer sequence can have or comprise a sequence having at least 90% identity to a sequence comprising nucleotide 1 through nucleotide 29 of any one of SEQ ID NOs: 1322-1425 and 1427-2632. The spacer sequence can have or comprise a sequence having at least 90% identity to a sequence comprising nucleotide 1 through nucleotide 30 of any one of SEQ ID NOs: 1322-1425 and 1427-2632.

TABLE 5
Target and spacer sequences

| | | | SEQ | | SEQ | |
|---|---|---|---|---|---|---|
| | | | ID | | ID | |
| BCL11A | strand | PAM | NO | target sequence | NO | spacer sequence |
| BCL11A_ | − | CTTA | 11 | GACATAACACACCAGGG | 1322 | GACAUAACACACCAGGGUC |
| enhancer_ | | | | TCAATACAACTTT | | AAUACAACUUU |
| region | | | | | | |
| BCL11A_ | − | CTTT | 12 | GAAGCTAGTCTAGTGCA | 1323 | GAAGCUAGUCUAGUGCAAG |
| enhancer_ | | | | AGCTAACAGTTGC | | CUAACAGUUGC |
| region | | | | | | |
| BCL11A_ | − | TTTG | 13 | AAGCTAGTCTAGTGCAA | 1324 | AAGCUAGUCUAGUGCAAGC |
| enhancer_ | | | | GCTAACAGTTGCT | | UAACAGUUGCU |
| region | | | | | | |
| BCL11A_ | − | GTTG | 14 | CTTTTATCACAGGCTCC | 1325 | CUUUUAUCACAGGCUCCAG |
| enhancer_ | | | | AGGAAGGGTTTGG | | GAAGGGUUUGG |
| region | | | | | | |
| BCL11A_ | − | CTTT | 15 | TATCACAGGCTCCAGGA | 1326 | UAUCACAGGCUCCAGGAAG |
| enhancer_ | | | | AGGGTTTGGCCTC | | GGUUUGGCCUC |
| region | | | | | | |
| BCL11A_ | − | TTTA | 16 | TCACAGGCTCCAGGAAG | 1327 | UCACAGGCUCCAGGAAGGG |
| enhancer_ | | | | GGTTTGGCCTCTG | | UUUGGCCUCUG |
| region | | | | | | |
| BCL11A_ | − | GTTT | 17 | GGCCTCTGATTAGGGTG | 1328 | GGCCUCUGAUUAGGGUGGG |
| enhancer_ | | | | GGGGCGTGGGTGG | | GGCGUGGGUGG |
| region | | | | | | |
| BCL11A_ | − | TTTG | 18 | GCCTCTGATTAGGGTGG | 1329 | GCCUCUGAUUAGGGUGGGG |
| enhancer_ | | | | GGGCGTGGGTGGG | | GCGUGGGUGGG |
| region | | | | | | |
| BCL11A_ | − | TTTT | 19 | ATCACAGGCTCCAGGAA | 1330 | AUCACAGGCUCCAGGAAGG |
| enhancer_ | | | | GGGTTTGGCCTCT | | GUUUGGCCUCU |
| region | | | | | | |
| BCL11A_ | + | CTTC | 20 | TACCCCACCCACGCCCC | 1331 | UACCCCACCCACGCCCCCA |
| enhancer_ | | | | CACCCTAATCAGA | | CCCUAAUCAGA |
| region | | | | | | |
| BCL11A_ | + | CTTC | 21 | CTGGAGCCTGTGATAAA | 1332 | CUGGAGCCUGUGAUAAAAG |
| enhancer_ | | | | AGCAACTGTTAGC | | CAACUGUUAGC |
| region | | | | | | |
| BCL11A_ | + | GTTA | 22 | GCTTGCACTAGACTAGC | 1333 | GCUUGCACUAGACUAGCUU |
| enhancer_ | | | | TTCAAAGTTGTAT | | CAAAGUUGUAU |
| region | | | | | | |
| BCL11A_ | + | CTTG | 23 | CACTAGACTAGCTTCAA | 1334 | CACUAGACUAGCUUCAAAG |
| enhancer_ | | | | AGTTGTATTGACC | | UUGUAUUGACC |
| region | | | | | | |
| BCL11A_ | + | CTTC | 24 | AAAGTTGTATTGACCCT | 1335 | AAAGUUGUAUUGACCCUGG |
| enhancer_ | | | | GGTGTGTTATGTC | | UGUGUUAUGUC |
| region | | | | | | |
| BCL11A_ | + | GTTG | 25 | TATTGACCCTGGTGTGT | 1336 | UAUUGACCCUGGUGUGUUA |
| enhancer_ | | | | TATGTCTAAGAGT | | UGUCUAAGAGU |
| region | | | | | | |
| BCL11A_ | + | ATTG | 26 | ACCCTGGTGTGTTATGT | 1337 | ACCCUGGUGUGUUAUGUCU |
| enhancer_ | | | | CTAAGAGTAGATG | | AAGAGUAGAUG |
| region | | | | | | |
| BCL11A_ | − | ATTA | 27 | GGGTGGGGGCGTGGGTG | 1338 | GGGUGGGGGCGUGGGUGGG |
| enhancer_ | | | | GGGTAGAAGAGGA | | GUAGAAGAGGA |
| region | | | | | | |
| BCL11A_ | − | TTTT | 28 | TTTGCTTAAAAAAAAGC | 1339 | UUUGCUUAAAAAAAAGCCA |
| exon_1 | | | | CATGACGGCTCTC | | UGACGGCUCUC |
| BCL11A_ | − | TTTT | 29 | TTTTTTTTTGCTTAAAA | 1340 | UUUUUUUUUGCUUAAAAAA |
| exon 1 | | | | AAAAGCCATGACG | | AAGCCAUGACG |
| BCL11A_ | − | TTTT | 30 | TTTTTTTTGCTTAAAAA | 1341 | UUUUUUUUGCUUAAAAAAA |
| exon_1 | | | | AAAGCCATGACGG | | AGCCAUGACGG |
| BCL11A_ | − | TTTT | 31 | TTTTTTTGCTTAAAAAA | 1342 | UUUUUUUGCUUAAAAAAAA |
| exon_1 | | | | AAGCCATGACGGC | | GCCAUGACGGC |
| BCL11A_ | − | TTTT | 32 | TTTTTTGCTTAAAAAAA | 1343 | UUUUUUGCUUAAAAAAAAG |
| exon_1 | | | | AGCCATGACGGCT | | CCAUGACGGCU |
| BCL11A_ | − | TTTT | 33 | TTTTTGCTTAAAAAAAA | 1344 | UUUUUGCUUAAAAAAAAGC |
| exon_1 | | | | GCCATGACGGCTC | | CAUGACGGCUC |
| BCL11A_ | − | TTTT | 34 | TTTTGCTTAAAAAAAAG | 1345 | UUUUGCUUAAAAAAAAGCC |
| exon_1 | | | | CCATGACGGCTCT | | AUGACGGCUCU |
| BCL11A_ | − | TTTT | 35 | TTGCTTAAAAAAAAGCC | 1346 | UUGCUUAAAAAAAAGCCAU |
| exon_1 | | | | ATGACGGCTCTCC | | GACGGCUCUCC |
| BCL11A_ | + | CTTT | 36 | TGACATCCAAAATAAAT | 1347 | UGACAUCCAAAAUAAAUUA |
| exon_1 | | | | TAGAAATAATACA | | GAAAUAAUACA |
| BCL11A_ | − | TTTT | 37 | GCTTAAAAAAAAGCCAT | 1348 | GCUUAAAAAAAAGCCAUGA |
| exon_1 | | | | GACGGCTCTCCCA | | CGGCUCUCCCA |
| BCL11A_ | − | TTTG | 38 | CTTAAAAAAAAGCCATG | 1349 | CUUAAAAAAAAGCCAUGAC |
| exon_1 | | | | ACGGCTCTCCCAC | | GGCUCUCCCAC |
| BCL11A_ | − | CTTA | 39 | AAAAAAAGCCATGACGG | 1350 | AAAAAAAGCCAUGACGGCU |
| exon_1 | | | | CTCTCCCACAATT | | CUCCCACAAUU |
| BCL11A_ | − | ATTC | 40 | ATCTTCCCTGCGCCATC | 135 | AUCUUCCCUGCGCCAUCUU |
| exon_1 | | | | TTTGTATTATTTC | | UGUAUUAUUUC |
| BCL11A_ | − | CTTC | 41 | CCTGCGCCATCTTTGTA | 1352 | CCUGCGCCAUCUUUGUAUU |
| exon_1 | | | | TTATTTCTAATTT | | AUUUCUAAUUU |
| BCL11A_ | − | CTTT | 42 | GTATTATTTCTAATTTA | 1353 | GUAUUAUUUCUAAUUUAUU |
| exon_1 | | | | TTTTGGATGTCAA | | UUGGAUGUCAA |
| BCL11A_ | − | TTTT | 43 | TTTTTTTTTTGCTTAAA | 1354 | UUUUUUUUUUGCUUAAAAA |
| exon_1 | | | | AAAAAGCCATGAC | | AAAGCCAUGAC |
| BCL11A_ | − | TTTT | 44 | TGCTTAAAAAAAAGCCA | 1355 | UGCUUAAAAAAAAGCCAUG |
| exon 1 | | | | TGACGGCTCTCCC | | ACGGCUCUCCC |
| BCL11A_ | − | TTTT | 45 | TTTTTTTTTTTGCTTAA | 1356 | UUUUUUUUUUUGCUUAAAA |
| exon_1 | | | | AAAAAAGCCATGA | | AAAAGCCAUGA |
| BCL11A_ | − | TTTT | 46 | TTTTTTTTTTTTTTTTT | 1357 | UUUUUUUUUUUUUUUUUUU |
| exon 1 | | | | TTTTTGCTTAAAA | | UUUGCUUAAAA |
| BCL11A_ | − | TTTT | 47 | TTTTTTTTTTTTTGCTT | 1358 | UUUUUUUUUUUUUGCUUAA |
| exon_1 | | | | AAAAAAAAGCCAT | | AAAAAAGCCAU |
| BCL11A_ | − | TTTG | 48 | CCATTTTTTTCATCTCT | 1359 | CCAUUUUUUUCAUCUCUCU |
| exon_1 | | | | CTCTCTCTCTCTC | | CUCUCUCUCUC |
| BCL11A_ | − | ATTT | 49 | TTTTCATCTCTCTCTCT | 1360 | UUUUCAUCUCUCUCUCUCU |
| exon_1 | | | | CTCTCTCCCTCTA | | CUCUCCCUCUA |
| BCL11A_ | − | TTTT | 50 | TTTCATCTCTCTCTCTC | 1361 | UUUCAUCUCUCUCUCUCUC |
| exon_1 | | | | TCTCTCCCTCTAT | | UCUCCCUCUAU |
| BCL11A_ | − | TTTT | 51 | TTCATCTCTCTCTCTCT | 1362 | UUCAUCUCUCUCUCUCUCU |
| exon_1 | | | | CTCTCCCTCTATC | | CUCCCUCUAUC |
| BCL11A_ | − | TTTT | 52 | TCATCTCTCTCTCTCTC | 1363 | UCAUCUCUCUCUCUCUCUC |
| exon_1 | | | | TCTCCCTCTATCT | | UCCCUCUAUCU |
| BCL11A_ | − | TTTT | 53 | CATCTCTCTCTCTCTCT | 1364 | CAUCUCUCUCUCUCUCUCU |
| exon_1 | | | | CTCCCTCTATCTC | | CCCUCUAUCUC |
| BCL11A_ | − | TTTC | 54 | ATCTCTCTCTCTCTCTC | 1365 | AUCUCUCUCUCUCUCUCUC |
| exon_1 | | | | TCCCTCTATCTCT | | CCUCUAUCUCU |
| BCL11A_ | − | CTTC | 55 | TCTCTCTCTCCCTCTTT | 1366 | UCUCUCUCUCCCUCUUUUU |
| exon_1 | | | | TTTTTTTTTTTTT | | UUUUUUUUUUU |
| BCL11A_ | − | TTTG | 56 | TATTATTTCTAATTTAT | 1367 | UAUUAUUUCUAAUUUAUUU |
| exon_1 | | | | TTTGGATGTCAAA | | UGGAUGUCAAA |
| BCL11A_ | − | TTTT | 57 | TTTTTTTTTTTTTTTTT | 1368 | UUUUUUUUUUUUUUUUUUU |
| exon_1 | | | | TTGCTTAAAAAAA | | GCUUAAAAAAA |
| BCL11A_ | − | TTTT | 58 | TTTTTTTTTTTTTTTTT | 1369 | UUUUUUUUUUUUUUUUUUG |
| exon 1 | | | | TGCTTAAAAAAAA | | CUUAAAAAAAA |
| BCL11A_ | − | TTTT | 59 | TTTTTTTTTTTTTTTTT | 1370 | UUUUUUUUUUUUUUUUUGC |
| exon_1 | | | | GCTTAAAAAAAAG | | UUAAAAAAAAG |
| BCL11A_ | − | TTTT | 60 | TTTTTTTTTTTTTTTTG | 1371 | UUUUUUUUUUUUUUUUGCU |
| exon_1 | | | | CTTAAAAAAAAGC | | UAAAAAAAAGC |
| BCL11A_ | − | TTTT | 61 | TTTTTTTTTTTTTTTGC | 1372 | UUUUUUUUUUUUUUUGCUU |
| exon_1 | | | | TTAAAAAAAAGCC | | AAAAAAAAGCC |
| BCL11A_ | − | TTTT | 62 | TTTTTTTTTTTTTTGCT | 1373 | UUUUUUUUUUUUUUGCUUA |
| exon_1 | | | | TAAAAAAAAGCCA | | AAAAAAAGCCA |
| BCL11A_ | − | TTTT | 63 | TTTTTTTTTTTTGCTTA | 1374 | UUUUUUUUUUUUGCUUAAA |
| exon_1 | | | | AAAAAAAGCCATG | | AAAAAGCCAUG |
| BCL11A_ | − | ATTA | 64 | TTTCTAATTTATTTTGG | 1375 | UUUCUAAUUUAUUUUGGAU |
| exon_1 | | | | ATGTCAAAAGGCA | | GUCAAAAGGCA |
| BCL11A_ | − | TTTT | 65 | CTCTGGAGTCTCCTTCT | 1376 | CUCUGGAGUCUCCUUCUUU |
| exon_1 | | | | TTCTAACCCGGCT | | CUAACCCGGCU |
| BCL11A_ | − | TTTC | 66 | TAATTTATTTTGGATGT | 1377 | UAAUUUAUUUUGGAUGUCA |
| exon_1 | | | | CAAAAGGCACTGA | | AAAGGCACUGA |
| BCL11A_ | + | GTTA | 67 | CTTACGCGAGAATTCCC | 1378 | CUUACGCGAGAAUUCCCGU |
| exon_1 | | | | GTTTGCTTAAGTG | | UUGCUUAAGUG |
| BCL11A_ | + | CTTA | 68 | CGCGAGAATTCCCGTTT | 1379 | CGCGAGAAUUCCCGUUUGC |
| exon_1 | | | | GCTTAAGTGCTGG | | UUAAGUGCUGG |
| BCL11A_ | + | ATTC | 69 | CCGTTTGCTTAAGTGCT | 1380 | CCGUUUGCUUAAGUGCUGG |
| exon_1 | | | | GGGGTTTGCCTTG | | GGUUUGCCUUG |
| BCL11A_ | + | GTTT | 70 | GCTTAAGTGCTGGGGTT | 1381 | GCUUAAGUGCUGGGGUUUG |
| exon_1 | | | | TGCCTTGCTTGCG | | CCUUGCUUGCG |
| BCL11A_ | + | TTTG | 71 | CTTAAGTGCTGGGGTTT | 1382 | CUUAAGUGCUGGGGUUUGC |
| exon_1 | | | | GCCTTGCTTGCGG | | CUUGCUUGCGG |
| BCL11A_ | + | CTTA | 72 | AGTGCTGGGGTTTGCCT | 1383 | AGUGCUGGGGUUUGCCUUG |
| exon_1 | | | | TGCTTGCGGCGAG | | CUUGCGGCGAG |
| BCL11A_ | + | GTTT | 73 | GCCTTGCTTGCGGCGAG | 1384 | GCCUUGCUUGCGGCGAGAC |
| exon_1 | | | | ACATGGTGGGCTG | | AUGGUGGGCUG |
| BCL11A_ | + | TTTG | 74 | CCTTGCTTGCGGCGAGA | 1385 | CCUUGCUUGCGGCGAGACA |
| exon_1 | | | | CATGGTGGGCTGC | | UGGUGGGCUGC |
| BCL11A_ | + | CTTG | 75 | CTTGCGGCGAGACATGG | 1386 | CUUGCGGCGAGACAUGGUG |
| exon_1 | | | | TGGGCTGCGGGGC | | GGCUGCGGGGC |
| BCL11A_ | + | CTTG | 76 | CGGCGAGACATGGTGGG | 1387 | CGGCGAGACAUGGUGGGCU |
| exon_1 | | | | CTGCGGGGCGGGC | | GCGGGGCGGGC |
| BCL11A_ | + | GTTC | 77 | ACATCGGGAGAGCCGGG | 1388 | ACAUCGGGAGAGCCGGGUU |
| exon_1 | | | | TTAGAAAGAAGGA | | AGAAAGAAGGA |
| BCL11A_ | + | GTTA | 78 | GAAAGAAGGAGACTCCA | 1389 | GAAAGAAGGAGACUCCAGA |
| exon_1 | | | | GAGAAAATATCTT | | GAAAAUAUCUU |
| BCL11A_ | + | CTTC | 79 | ATCAGTGCCTTTTGACA | 1390 | AUCAGUGCCUUUUGACAUC |
| exon_1 | | | | TCCAAAATAAATT | | CAAAAUAAAUU |
| BCL11A_ | + | ATTG | 80 | TGGGAGAGCCGTCATGG | 1391 | UGGGAGAGCCGUCAUGGCU |
| exon_1 | | | | CTTTTTTTTAAGC | | UUUUUUUAAGC |
| BCL11A_ | + | TTTT | 81 | GACATCCAAAATAAATT | 1392 | GACAUCCAAAAUAAAUUAG |
| exon_1 | | | | AGAAATAATACAA | | AAAUAAUACAA |
| BCL11A_ | + | ATTG | 82 | GGTTACTTACGCGAGAA | 1393 | GGUUACUUACGCGAGAAUU |
| exon_1 | | | | TTCCCGTTTGCTT | | CCCGUUUGCUU |
| BCL11A_ | + | ATTA | 83 | TTGGGTTACTTACGCGA | 1394 | UUGGGUUACUUACGCGAGA |
| exon_1 | | | | GAATTCCCGTTTG | | AUUCCCGUUUG |
| BCL11A_ | + | ATTA | 84 | CTATTATTGGGTTACTT | 1395 | CUAUUAUUGGGUUACUUAC |
| exon_1 | | | | ACGCGAGAATTCC | | GCGAGAAUUCC |
| BCL11A_ | + | ATTA | 85 | TTACTATTATTGGGTTA | 1396 | UUACUAUUAUUGGGUUACU |
| exon_1 | | | | CTTACGCGAGAAT | | UACGCGAGAAU |
| BCL11A_ | − | ATTT | 86 | ATTTTGGATGTCAAAAG | 1397 | AUUUUGGAUGUCAAAAGGC |
| exon_1 | | | | GCACTGATGAAGA | | ACUGAUGAAGA |
| BCL11A_ | − | TTTA | 87 | TTTTGGATGTCAAAAGG | 1398 | UUUUGGAUGUCAAAAGGCA |
| exon 1 | | | | CACTGATGAAGAT | | CUGAUGAAGAU |
| BCL11A_ | − | ATTT | 88 | TGGATGTCAAAAGGCAC | 1399 | UGGAUGUCAAAAGGCACUG |
| exon_1 | | | | TGATGAAGATATT | | AUGAAGAUAUU |
| BCL11A_ | − | TTTT | 89 | GGATGTCAAAAGGCACT | 1400 | GGAUGUCAAAAGGCACUGA |
| exon_1 | | | | GATGAAGATATTT | | UGAAGAUAUUU |
| BCL11A_ | − | TTTG | 90 | GATGTCAAAAGGCACTG | 1401 | GAUGUCAAAAGGCACUGAU |
| exon_1 | | | | ATGAAGATATTTT | | GAAGAUAUUUU |
| BCL11A_ | − | ATTT | 91 | TCTCTGGAGTCTCCTTC | 1402 | UCUCUGGAGUCUCCUUCUU |
| exon_1 | | | | TTTCTAACCCGGC | | UCUAACCCGGC |
| BCL11A_ | − | TTTT | 92 | GCCATTTTTTTCATCTC | 1403 | GCCAUUUUUUUCAUCUCUC |
| exon_1 | | | | TCTCTCTCTCTCT | | UCUCUCUCUCU |
| BCL11A_ | − | ATTT | 93 | CTAATTTATTTTGGATG | 1404 | CUAAUUUAUUUUGGAUGUC |
| exon_1 | | | | TCAAAAGGCACTG | | AAAAGGCACUG |
| BCL11A_ | − | TTTC | 94 | TCTGGAGTCTCCTTCTT | 1405 | UCUGGAGUCUCCUUCUUUC |
| exon_1 | | | | TCTAACCCGGCTC | | UAACCCGGCUC |
| BCL11A_ | − | CTTT | 95 | CTAACCCGGCTCTCCCG | 1406 | CUAACCCGGCUCUCCCGAU |
| exon_1 | | | | ATGTGAACCGAGC | | GUGAACCGAGC |
| BCL11A_ | − | TTTC | 96 | TAACCCGGCTCTCCCGA | 1407 | UAACCCGGCUCUCCCGAUG |
| exon_1 | | | | TGTGAACCGAGCC | | UGAACCGAGCC |
| BCL11A_ | − | CTTA | 97 | AGCAAACGGGAATTCTC | 1408 | AGCAAACGGGAAUUCUCGC |
| exon_1 | | | | GCGTAAGTAACCC | | GUAAGUAACCC |
| BCL11A_ | − | ATTC | 98 | TCGCGTAAGTAACCCAA | 1409 | UCGCGUAAGUAACCCAAUA |
| exon_1 | | | | TAATAGTAATAAT | | AUAGUAAUAAU |
| BCL11A_ | + | ATTA | 99 | TTAATAATTATTATTAC | 1410 | UUAAUAAUUAUUAUUACUA |
| exon_1 | | | | TATTATTGGGTTA | | UUAUUGGGUUA |
| BCL11A_ | + | ATTA | 100 | ATAATTATTATTACTAT | 1411 | AUAAUUAUUAUUACUAUUA |
| exon_1 | | | | TATTGGGTTACTT | | UUGGGUUACUU |
| BCL11A_ | + | ATTA | 101 | TTATTACTATTATTGGG | 1412 | UUAUUACUAUUAUUGGGUU |
| exon_1 | | | | TTACTTACGCGAG | | ACUUACGCGAG |
| BCL11A_ | − | CTTC | 102 | TTTCTAACCCGGCTCTC | 1413 | UUUCUAACCCGGCUCUCCC |
| exon_1 | | | | CCGATGTGAACCG | | GAUGUGAACCG |
| BCL11A_ | − | CTTT | 103 | TGCCATTTTTTTCATCT | 1414 | UGCCAUUUUUUUCAUCUCU |
| exon_1 | | | | CTCTCTCTCTCTC | | CUCUCUCUCUC |
| BCL11A_ | + | ATTA | 104 | GAAATAATACAAAGATG | 1415 | GAAAUAAUACAAAGAUGGC |
| exon_1 | | | | GCGCAGGGAAGAT | | GCAGGGAAGAU |
| BCL11A_ | − | CTTG | 105 | AACTTGCAGCTCAGGGG | 1416 | AACUUGCAGCUCAGGGGGG |
| exon_1 | | | | GGCTTTTGCCATT | | CUUUUGCCAUU |
| BCL11A_ | − | CTTG | 106 | CAGCTCAGGGGGGCTTT | 1417 | CAGCUCAGGGGGGCUUUUG |
| exon_1 | | | | TGCCATTTTTTTC | | CCAUUUUUUUC |
| BCL11A_ | + | TTTT | 107 | TTTTAAGCAAAAAAAAA | 1418 | UUUUAAGCAAAAAAAAAAA |
| exon_1 | | | | AAAAAAAAAAAAA | | AAAAAAAAAAA |
| BCL11A_ | + | TTTT | 108 | TTTAAGCAAAAAAAAAA | 1419 | UUUAAGCAAAAAAAAAAAA |
| exon_1 | | | | AAAAAAAAAAAAA | | AAAAAAAAAAA |
| BCL11A_ | + | TTTT | 109 | TTAAGCAAAAAAAAAAA | 1420 | UUAAGCAAAAAAAAAAAAA |
| exon_1 | | | | AAAAAAAAAAAAA | | AAAAAAAAAAA |
| BCL11A_ | + | TTTT | 110 | TAAGCAAAAAAAAAAAA | 1421 | UAAGCAAAAAAAAAAAAAA |
| exon_1 | | | | AAAAAAAAAAAAA | | AAAAAAAAAAA |
| BCL11A_ | + | TTTG | 111 | ACATCCAAAATAAATTA | 1422 | ACAUCCAAAAUAAAUUAGA |
| exon_1 | | | | GAAATAATACAAA | | AAUAAUACAAA |
| BCL11A_ | + | CTTT | 112 | TTTTTAAGCAAAAAAAA | 1423 | UUUUUAAGCAAAAAAAAAA |
| exon_1 | | | | AAAAAAAAAAAAA | | AAAAAAAAAAA |
| BCL11A_ | + | TTTA | 113 | AGCAAAAAAAAAAAAAA | 1424 | AGCAAAAAAAAAAAAAAAA |
| exon_1 | | | | AAAAAAAAAAAAA | | AAAAAAAAAAA |
| BCL11A_ | + | GTTC | 114 | AAGTGCGGACGTGACGT | 1425 | AAGUGCGGACGUGACGUCC |
| exon_1 | | | | CCCTGCGAACTTG | | CUGCGAACUUG |
| BCL11A_ | + | CTTG | 115 | AACGTCAGGAGTCTGGA | 1426 | AACGUCAGGAGUCUGGAUG |
| exon_1 | | | | TGGACAGAGAC | | GACAGAGAC |
| BCL11A_ | − | GTTC | 116 | AAGTTCGCAGGGACGTC | 1427 | AAGUUCGCAGGGACGUCAC |
| exon_1 | | | | ACGTCCGCACTTG | | GUCCGCACUUG |
| BCL11A_ | + | TTTT | 117 | AAGCAAAAAAAAAAAAA | 1428 | AAGCAAAAAAAAAAAAAAA |
| exon_1 | | | | AAAAAAAAAAAAA | | AAAAAAAAAAA |
| BCL11A_ | − | GTTC | 118 | GCAGGGACGTCACGTCC | 1429 | GCAGGGACGUCACGUCCGC |
| exon_1 | | | | GCACTTGAACTTG | | ACUUGAACUUG |
| BCL11A_ | − | TTTT | 119 | TATCGAGCACAAACGGA | 1430 | UAUCGAGCACAAACGGAAA |
| exon_2 | | | | AACAATGCAATGG | | CAAUGCAAUGG |
| BCL11A_ | − | TTTT | 120 | ATCGAGCACAAACGGAA | 1431 | AUCGAGCACAAACGGAAAC |
| exon 2 | | | | ACAATGCAATGGC | | AAUGCAAUGGC |
| BCL11A_ | − | TTTA | 121 | TCGAGCACAAACGGAAA | 1432 | UCGAGCACAAACGGAAACA |
| exon_2 | | | | CAATGCAATGGCA | | AUGCAAUGGCA |
| BCL11A_ | − | CTTA | 122 | GAAAAAGCTGTGGATAA | 1433 | GAAAAAGCUGUGGAUAAGC |
| exon_2 | | | | GCCACCTTCCCCT | | CACCUUCCCCU |
| BCL11A_ | − | GTTG | 123 | GCATCCAGGTCACGCCA | 1434 | GCAUCCAGGUCACGCCAGA |
| exon 2 | | | | GAGGATGACGATT | | GGAUGACGAUU |
| BCL11A_ | − | CTTC | 124 | ACCAATCGAGATGAAAA | 1435 | ACCAAUCGAGAUGAAAAAA |
| exon 2 | | | | AAGCATCCAATCC | | GCAUCCAAUCC |
| BCL11A_ | − | ATTG | 125 | TTTATCAACGTCATCTA | 1436 | UUUAUCAACGUCAUCUAGA |
| exon_2 | | | | GAGGAATTTGCCC | | GGAAUUUGCCC |
| BCL11A_ | − | GTTT | 126 | ATCAACGTCATCTAGAG | 1437 | AUCAACGUCAUCUAGAGGA |
| exon_2 | | | | GAATTTGCCCCAA | | AUUUGCCCCAA |
| BCL11A_ | − | TTTA | 127 | TCAACGTCATCTAGAGG | 1438 | UCAACGUCAUCUAGAGGAA |
| exon_2 | | | | AATTTGCCCCAAA | | UUUGCCCCAAA |
| BCL11A_ | − | ATTT | 128 | TTATCGAGCACAAACGG | 1439 | UUAUCGAGCACAAACGGAA |
| exon_2 | | | | AAACAATGCAATG | | ACAAUGCAAUG |
| BCL11A_ | − | CTTC | 129 | CCCTTCACCAATCGAGA | 1440 | CCCUUCACCAAUCGAGAUG |
| exon_2 | | | | TGAAAAAAGCATC | | AAAAAAGCAUC |
| BCL11A_ | − | CTTA | 130 | TTTTTATCGAGCACAAA | 1441 | UUUUUAUCGAGCACAAACG |
| exon_2 | | | | CGGAAACAATGCA | | GAAACAAUGCA |
| BCL11A_ | − | CTTG | 131 | AAGCCATTCTTACAGAT | 1442 | AAGCCAUUCUUACAGAUGA |
| exon_2 | | | | GATGAACCAGACC | | UGAACCAGACC |
| BCL11A_ | − | ATTG | 132 | GGGGACATTCTTATTTT | 1443 | GGGGACAUUCUUAUUUUUA |
| exon_2 | | | | TATCGAGCACAAA | | UCGAGCACAAA |
| BCL11A_ | − | CTTC | 133 | CCATTGGGGGACATTCT | 1444 | CCAUUGGGGGACAUUCUUA |
| exon 2 | | | | TATTTTTATCGAG | | UUUUUAUCGAG |
| BCL11A_ | − | GTTG | 134 | GGAGCTCCAGAAGGGGA | 1445 | GGAGCUCCAGAAGGGGAUC |
| exon 2 | | | | TCATGACCTCCTC | | AUGACCUCCUC |
| BCL11A_ | − | CTTA | 135 | CAGATGATGAACCAGAC | 1446 | CAGAUGAUGAACCAGACCA |
| exon 2 | | | | CACGGCCCGTTGG | | CGGCCCGUUGG |
| BCL11A_ | − | ATTC | 136 | TTACAGATGATGAACCA | 1447 | UUACAGAUGAUGAACCAGA |
| exon 2 | | | | GACCACGGCCCGT | | CCACGGCCCGU |
| BCL11A_ | − | TTTC | 137 | TCCAACCACAGCCGAGC | 1448 | UCCAACCACAGCCGAGCCU |
| exon 2 | | | | CTCTTGAAGCCAT | | CUUGAAGCCAU |
| BCL11A_ | − | GTTT | 138 | CTCCAACCACAGCCGAG | 1449 | CUCCAACCACAGCCGAGCC |
| exon_2 | | | | CCTCTTGAAGCCA | | UCUUGAAGCCA |
| BCL11A_ | − | TTTG | 139 | TTTCTCCAACCACAGCC | 1450 | UUUCUCCAACCACAGCCGA |
| exon 2 | | | | GAGCCTCTTGAAG | | GCCUCUUGAAG |
| BCL11A_ | − | TTTT | 140 | GTTTCTCCAACCACAGC | 1451 | GUUUCUCCAACCACAGCCG |
| exon_2 | | | | CGAGCCTCTTGAA | | AGCCUCUUGAA |
| BCL11A_ | − | CTTT | 141 | TGTTTCTCCAACCACAG | 1452 | UGUUUCUCCAACCACAGCC |
| exon_2 | | | | CCGAGCCTCTTGA | | GAGCCUCUUGA |
| BCL11A_ | − | ATTG | 142 | TGCTTTTGTTTCTCCAA | 1453 | UGCUUUUGUUUCUCCAACC |
| exon_2 | | | | CCACAGCCGAGCC | | ACAGCCGAGCC |
| BCL11A_ | − | ATTC | 143 | TTATTTTTATCGAGCAC | 1454 | UUAUUUUUAUCGAGCACAA |
| exon_2 | | | | AAACGGAAACAAT | | ACGGAAACAAU |
| BCL11A_ | − | ATTT | 144 | GCCCCAAACAGGAACAC | 1455 | GCCCCAAACAGGAACACAU |
| exon_2 | | | | ATAGCAGGTAAAT | | AGCAGGUAAAU |
| BCL11A_ | + | CTTT | 145 | TCTCCTTGCTTCTCATT | 1456 | UCUCCUUGCUUCUCAUUUA |
| exon 2 | | | | TACCTGCTATGTG | | CCUGCUAUGUG |
| BCL11A_ | + | TTTT | 146 | CTCCTTGCTTCTCATTT | 1457 | CUCCUUGCUUCUCAUUUAC |
| exon_2 | | | | ACCTGCTATGTGT | | CUGCUAUGUGU |
| BCL11A_ | + | TTTT | 147 | TCTAAGCAGAGGCTGCC | 1458 | UCUAAGCAGAGGCUGCCAU |
| exon 2 | | | | ATTGCATTGTTTC | | UGCAUUGUUUC |
| BCL11A_ | + | TTTT | 148 | CTAAGCAGAGGCTGCCA | 1459 | CUAAGCAGAGGCUGCCAUU |
| exon_2 | | | | TTGCATTGTTTCC | | GCAUUGUUUCC |
| BCL11A_ | + | TTTC | 149 | TAAGCAGAGGCTGCCAT | 1460 | UAAGCAGAGGCUGCCAUUG |
| exon 2 | | | | TGCATTGTTTCCG | | CAUUGUUUCCG |
| BCL11A_ | + | ATTG | 150 | CATTGTTTCCGTTTGTG | 1461 | CAUUGUUUCCGUUUGUGCU |
| exon_2 | | | | CTCGATAAAAATA | | CGAUAAAAAUA |
| BCL11A_ | + | ATTG | 151 | TTTCCGTTTGTGCTCGA | 1462 | UUUCCGUUUGUGCUCGAUA |
| exon 2 | | | | TAAAAATAAGAAT | | AAAAUAAGAAU |
| BCL11A_ | + | GTTT | 152 | CCGTTTGTGCTCGATAA | 1463 | CCGUUUGUGCUCGAUAAAA |
| exon_2 | | | | AAATAAGAATGTC | | AUAAGAAUGUC |
| BCL11A_ | + | CTTT | 153 | TTCTAAGCAGAGGCTGC | 1464 | UUCUAAGCAGAGGCUGCCA |
| exon_2 | | | | CATTGCATTGTTT | | UUGCAUUGUUU |
| BCL11A_ | + | TTTC | 154 | CGTTTGTGCTCGATAAA | 1465 | CGUUUGUGCUCGAUAAAAA |
| exon_2 | | | | AATAAGAATGTCC | | UAAGAAUGUCC |
| BCL11A_ | + | TTTG | 155 | TGCTCGATAAAAATAAG | 1466 | UGCUCGAUAAAAAUAAGAA |
| exon 2 | | | | AATGTCCCCCAAT | | UGUCCCCCAAU |
| BCL11A_ | + | GTTC | 156 | ATCTGGCACTGCCCACA | 1467 | AUCUGGCACUGCCCACAGG |
| exon_2 | | | | GGTGAGGAGGTCA | | UGAGGAGGUCA |
| BCL11A_ | + | GTTC | 157 | ATCATCTGTAAGAATGG | 1468 | AUCAUCUGUAAGAAUGGCU |
| exon 2 | | | | CTTCAAGAGGCTC | | UCAAGAGGCUC |
| BCL11A_ | + | CTTC | 158 | AAGAGGCTCGGCTGTGG | 1469 | AAGAGGCUCGGCUGUGGUU |
| exon_2 | | | | TTGGAGAAACAAA | | GGAGAAACAAA |
| BCL11A_ | + | GTTG | 159 | GAGAAACAAAAGCACAA | 1470 | GAGAAACAAAAGCACAAUU |
| exon 2 | | | | TTATTAGAGTGCC | | AUUAGAGUGCC |
| BCL11A_ | + | ATTA | 160 | TTAGAGTGCCAGAGAGG | 1471 | UUAGAGUGCCAGAGAGGAC |
| exon_2 | | | | ACAGAAAGGGGAG | | AGAAAGGGGAG |
| BCL11A_ | + | GTTT | 161 | GTGCTCGATAAAAATAA | 1472 | GUGCUCGAUAAAAAUAAGA |
| exon_2 | | | | GAATGTCCCCCAA | | AUGUCCCCCAA |
| BCL11A_ | + | CTTA | 162 | TCCACAGCTTTTTCTAA | 1473 | UCCACAGCUUUUUCUAAGC |
| exon_2 | | | | GCAGAGGCTGCCA | | AGAGGCUGCCA |
| BCL11A_ | + | ATTG | 163 | GTGAAGGGGAAGGTGGC | 1474 | GUGAAGGGGAAGGUGGCUU |
| exon 2 | | | | TTATCCACAGCTT | | AUCCACAGCUU |
| BCL11A_ | + | TTTC | 164 | ATCTCGATTGGTGAAGG | 1475 | AUCUCGAUUGGUGAAGGGG |
| exon_2 | | | | GGAAGGTGGCTTA | | AAGGUGGCUUA |
| BCL11A_ | + | TTTC | 165 | TCCTTGCTTCTCATTTA | 1476 | UCCUUGCUUCUCAUUUACC |
| exon_2 | | | | CCTGCTATGTGTT | | UGCUAUGUGUU |
| BCL11A_ | + | CTTG | 166 | CTTCTCATTTACCTGCT | 1477 | CUUCUCAUUUACCUGCUAU |
| exon 2 | | | | ATGTGTTCCTGTT | | GUGUUCCUGUU |
| BCL11A_ | + | CTTC | 167 | TCATTTACCTGCTATGT | 1478 | UCAUUUACCUGCUAUGUGU |
| exon 2 | | | | GTTCCTGTTTGGG | | UCCUGUUUGGG |
| BCL11A_ | + | ATTT | 168 | ACCTGCTATGTGTTCCT | 1479 | ACCUGCUAUGUGUUCCUGU |
| exon_2 | | | | GTTTGGGGCAAAT | | UUGGGGCAAAU |
| BCL11A_ | + | TTTA | 169 | CCTGCTATGTGTTCCTG | 1480 | CCUGCUAUGUGUUCCUGUU |
| exon_2 | | | | TTTGGGGCAAATT | | UGGGGCAAAUU |
| BCL11A_ | + | GTTC | 170 | CTGTTTGGGGCAAATTC | 1481 | CUGUUUGGGGCAAAUUCCU |
| exon_2 | | | | CTCTAGATGACGT | | CUAGAUGACGU |
| BCL11A_ | + | GTTT | 171 | GGGGCAAATTCCTCTAG | 1482 | GGGGCAAAUUCCUCUAGAU |
| exon 2 | | | | ATGACGTTGATAA | | GACGUUGAUAA |
| BCL11A_ | + | TTTG | 172 | GGGCAAATTCCTCTAGA | 1483 | GGGCAAAUUCCUCUAGAUG |
| exon_2 | | | | TGACGTTGATAAA | | ACGUUGAUAAA |
| BCL11A_ | + | ATTC | 173 | CTCTAGATGACGTTGAT | 1484 | CUCUAGAUGACGUUGAUAA |
| exon_2 | | | | AAACAATCGTCAT | | ACAAUCGUCAU |
| BCL11A_ | + | GTTG | 174 | ATAAACAATCGTCATCC | 1485 | AUAAACAAUCGUCAUCCUC |
| exon 2 | | | | TCTGGCGTGACCT | | UGGCGUGACCU |
| BCL11A_ | + | ATTG | 175 | GATGCTTTTTTCATCTC | 1486 | GAUGCUUUUUUCAUCUCGA |
| exon 2 | | | | GATTGGTGAAGGG | | UUGGUGAAGGG |
| BCL11A_ | + | CTTT | 176 | TTTCATCTCGATTGGTG | 1487 | UUUCAUCUCGAUUGGUGAA |
| exon_2 | | | | AAGGGGAAGGTGG | | GGGGAAGGUGG |
| BCL11A_ | + | TTTT | 177 | TTCATCTCGATTGGTGA | 1488 | UUCAUCUCGAUUGGUGAAG |
| exon_2 | | | | AGGGGAAGGTGGC | | GGGAAGGUGGC |
| BCL11A_ | + | TTTT | 178 | TCATCTCGATTGGTGAA | 1489 | UCAUCUCGAUUGGUGAAGG |
| exon 2 | | | | GGGGAAGGTGGCT | | GGAAGGUGGCU |
| BCL11A_ | + | TTTT | 179 | CATCTCGATTGGTGAAG | 1490 | CAUCUCGAUUGGUGAAGGG |
| exon_2 | | | | GGGAAGGTGGCTT | | GAAGGUGGCUU |
| BCL11A_ | − | TTTG | 180 | CCCCAAACAGGAACACA | 1491 | CCCCAAACAGGAACACAUA |
| exon 2 | | | | TAGCAGGTAAATG | | GCAGGUAAAUG |
| BCL11A_ | + | CTTC | 181 | TGGAGCTCCCAACGGGC | 1492 | UGGAGCUCCCAACGGGCCG |
| exon_2 | | | | CGTGGTCTGGTTC | | UGGUCUGGUUC |
| BCL11A_ | − | GTTG | 182 | TTTGTAGCTGTAGTGCT | 1493 | UUUGUAGCUGUAGUGCUUG |
| exon_3 | | | | TGATTTTGGGTTT | | AUUUUGGGUUU |
| BCL11A_ | + | TTTA | 183 | TCTGTGAAAGAAACCCA | 1494 | UCUGUGAAAGAAACCCAAA |
| exon_3 | | | | AAATCAAGCACTA | | AUCAAGCACUA |
| BCL11A_ | − | GTTT | 184 | GTAGCTGTAGTGCTTGA | 1495 | GUAGCUGUAGUGCUUGAUU |
| exon_3 | | | | TTTTGGGTTTCTT | | UUGGGUUUCUU |
| BCL11A_ | − | TTTG | 185 | TAGCTGTAGTGCTTGAT | 1496 | UAGCUGUAGUGCUUGAUUU |
| exon_3 | | | | TTTGGGTTTCTTT | | UGGGUUUCUUU |
| BCL11A_ | − | CTTG | 186 | ATTTTGGGTTTCTTTCA | 1497 | AUUUUGGGUUUCUUUCACA |
| exon_3 | | | | CAGATAAACTTCT | | GAUAAACUUCU |
| BCL11A_ | − | ATTT | 187 | TGGGTTTCTTTCACAGA | 1498 | UGGGUUUCUUUCACAGAUA |
| exon_3 | | | | TAAACTTCTGCAC | | AACUUCUGCAC |
| BCL11A_ | − | TTTT | 188 | GGGTTTCTTTCACAGAT | 1499 | GGGUUUCUUUCACAGAUAA |
| exon_3 | | | | AAACTTCTGCACT | | ACUUCUGCACU |
| BCL11A_ | − | GTTT | 189 | CTTTCACAGATAAACTT | 1500 | CUUUCACAGAUAAACUUCU |
| exon_3 | | | | CTGCACTGGAGGG | | GCACUGGAGGG |
| BCL11A_ | − | TTTC | 190 | TTTCACAGATAAACTTC | 1501 | UUUCACAGAUAAACUUCUG |
| exon_3 | | | | TGCACTGGAGGGG | | CACUGGAGGGG |
| BCL11A_ | − | CTTT | 191 | CACAGATAAACTTCTGC | 1502 | CACAGAUAAACUUCUGCAC |
| exon_3 | | | | ACTGGAGGGGCCT | | UGGAGGGGCCU |
| BCL11A_ | − | TTTC | 192 | ACAGATAAACTTCTGCA | 1503 | ACAGAUAAACUUCUGCACU |
| exon_3 | | | | CTGGAGGGGCCTC | | GGAGGGGCCUC |
| BCL11A_ | − | CTTC | 193 | TGCACTGGAGGGGCCTC | 1504 | UGCACUGGAGGGGCCUCUC |
| exon_3 | | | | TCCTCCCCTCGTT | | CUCCCCUCGUU |
| BCL11A_ | − | GTTC | 194 | TGCACATGGAGCTCTAA | 1505 | UGCACAUGGAGCUCUAAUC |
| exon_3 | | | | TCCCCACGCCTGG | | CCCACGCCUGG |
| BCL11A_ | − | ATTT | 195 | GTAAGTTGAGCCTTATT | 1506 | GUAAGUUGAGCCUUAUUUC |
| exon_3 | | | | TCTTCTACAAATG | | UUCUACAAAUG |
| BCL11A_ | − | TTTG | 196 | GGTTTCTTTCACAGATA | 1507 | GGUUUCUUUCACAGAUAAA |
| exon_3 | | | | AACTTCTGCACTG | | CUUCUGCACUG |
| BCL11A_ | − | GTTG | 197 | AGCCTTATTTCTTCTAC | 1508 | AGCCUUAUUUCUUCUACAA |
| exon_3 | | | | AAATGTCCATGTG | | AUGUCCAUGUG |
| BCL11A_ | − | TTTG | 198 | TAAGTTGAGCCTTATTT | 1509 | UAAGUUGAGCCUUAUUUCU |
| exon_3 | | | | CTTCTACAAATGT | | UCUACAAAUGU |
| BCL11A_ | + | GTTT | 199 | ATCTGTGAAAGAAACCC | 1510 | AUCUGUGAAAGAAACCCAA |
| exon_3 | | | | AAAATCAAGCACT | | AAUCAAGCACU |
| BCL11A_ | + | ATTA | 200 | GAGCTCCATGTGCAGAA | 1511 | GAGCUCCAUGUGCAGAACG |
| exon_3 | | | | CGAGGGGAGGAGA | | AGGGGAGGAGA |
| BCL11A_ | + | ATTC | 201 | TGCACTCATCCCAGGCG | 1512 | UGCACUCAUCCCAGGCGUG |
| exon_3 | | | | TGGGGATTAGAGC | | GGGAUUAGAGC |
| BCL11A_ | + | TTTG | 202 | TAGAAGAAATAAGGCTC | 1513 | UAGAAGAAAUAAGGCUCAA |
| exon_3 | | | | AACTTACAAATAC | | CUUACAAAUAC |
| BCL11A_ | + | CTTA | 203 | CAAATACCCTGCGGGGC | 1514 | CAAAUACCCUGCGGGGCAU |
| exon 3 | | | | ATATTCTGCACTC | | AUUCUGCACUC |
| BCL11A_ | + | ATTT | 204 | GTAGAAGAAATAAGGCT | 1515 | GUAGAAGAAAUAAGGCUCA |
| exon_3 | | | | CAACTTACAAATA | | ACUUACAAAUA |
| BCL11A_ | − | CTTC | 205 | TACAAATGTCCATGTGT | 1516 | UACAAAUGUCCAUGUGUAU |
| exon_3 | | | | ATAGAGATGAGAA | | AGAGAUGAGAA |
| BCL11A_ | − | TTTC | 206 | TTCTACAAATGTCCATG | 1517 | UUCUACAAAUGUCCAUGUG |
| exon_3 | | | | TGTATAGAGATGA | | UAUAGAGAUGA |
| BCL11A_ | − | ATTT | 207 | CTTCTACAAATGTCCAT | 1518 | CUUCUACAAAUGUCCAUGU |
| exon_3 | | | | GTGTATAGAGATG | | GUAUAGAGAUG |
| BCL11A_ | − | CTTA | 208 | TTTCTTCTACAAATGTC | 1519 | UUUCUUCUACAAAUGUCCA |
| exon_3 | | | | CATGTGTATAGAG | | UGUGUAUAGAG |
| BCL11A_ | + | GTTT | 209 | TTTAAAAAAAATTTTTC | 1520 | UUUAAAAAAAAUUUUUCUU |
| exon_4 | | | | TTAACATTTATAT | | AACAUUUAUAU |
| BCL11A_ | + | TTTT | 210 | AAAAAAAATTTTTCTTA | 1521 | AAAAAAAAUUUUUCUUAAC |
| exon_4 | | | | ACATTTATATTTA | | AUUUAUAUUUA |
| BCL11A_ | + | TTTT | 211 | TAAAAAAAATTTTTCTT | 1522 | UAAAAAAAAUUUUUCUUAA |
| exon_4 | | | | AACATTTATATTT | | CAUUUAUAUUU |
| BCL11A_ | + | TTTT | 212 | TTAAAAAAAATTTTTCT | 1523 | UUAAAAAAAAUUUUUCUUA |
| exon_4 | | | | TAACATTTATATT | | ACAUUUAUAUU |
| BCL11A_ | + | TTTA | 213 | AAAAAAATTTTTCTTAA | 1524 | AAAAAAAUUUUUCUUAACA |
| exon_4 | | | | CATTTATATTTAA | | UUUAUAUUUAA |
| BCL11A_ | + | GTTC | 214 | CCCCCTAAACATAATGA | 1525 | CCCCCUAAACAUAAUGAAG |
| exon_4 | | | | AGTGTTTTTTAAA | | UGUUUUUUAAA |
| BCL11A_ | + | TTTC | 215 | CACTACCATTTTTAAAT | 1526 | CACUACCAUUUUUAAAUGG |
| exon_4 | | | | GGATAACAAGTCT | | AUAACAAGUCU |
| BCL11A_ | + | TTTA | 216 | AATGGATAACAAGTCTT | 1527 | AAUGGAUAACAAGUCUUGU |
| exon_4 | | | | GTAACACCACCAA | | AACACCACCAA |
| BCL11A_ | + | TTTT | 217 | AAATGGATAACAAGTCT | 1528 | AAAUGGAUAACAAGUCUUG |
| exon_4 | | | | TGTAACACCACCA | | UAACACCACCA |
| BCL11A_ | + | TTTT | 218 | TAAATGGATAACAAGTC | 1529 | UAAAUGGAUAACAAGUCUU |
| exon_4 | | | | TTGTAACACCACC | | GUAACACCACC |
| BCL11A_ | + | ATTT | 219 | TTAAATGGATAACAAGT | 1530 | UUAAAUGGAUAACAAGUCU |
| exon_4 | | | | CTTGTAACACCAC | | UGUAACACCAC |
| BCL11A_ | + | ATTT | 220 | CCACTACCATTTTTAAA | 1531 | CCACUACCAUUUUUAAAUG |
| exon_4 | | | | TGGATAACAAGTC | | GAUAACAAGUC |
| BCL11A_ | + | ATTT | 221 | TTCTTAACATTTATATT | 1532 | UUCUUAACAUUUAUAUUUA |
| exon_4 | | | | TAAAAAAGTTTTG | | AAAAAGUUUUG |
| BCL11A_ | + | CTTG | 222 | TAACACCACCAAGACAA | 1533 | UAACACCACCAAGACAAUG |
| exon 4 | | | | TGGAACCCTAAAA | | GAACCCUAAAA |
| BCL11A_ | + | TTTT | 223 | TCTTAACATTTATATTT | 1534 | UCUUAACAUUUAUAUUUAA |
| exon_4 | | | | AAAAAAGTTTTGT | | AAAAGUUUUGU |
| BCL11A_ | + | ATTT | 224 | CTATGTTAAGTGTATTC | 1535 | CUAUGUUAAGUGUAUUCUG |
| exon_4 | | | | TGTTTCCATTCAC | | UUUCCAUUCAC |
| BCL11A_ | + | TTTC | 225 | TTAACATTTATATTTAA | 1536 | UUAACAUUUAUAUUUAAAA |
| exon_4 | | | | AAAAGTTTTGTAC | | AAGUUUUGUAC |
| BCL11A_ | + | CTTA | 226 | ACATTTATATTTAAAAA | 1537 | ACAUUUAUAUUUAAAAAAG |
| exon_4 | | | | AGTTTTGTACAAA | | UUUUGUACAAA |
| BCL11A_ | + | ATTT | 227 | ATATTTAAAAAAGTTTT | 1538 | AUAUUUAAAAAAGUUUUGU |
| exon_4 | | | | GTACAAAAAAATC | | ACAAAAAAAUC |
| BCL11A_ | + | TTTA | 228 | TATTTAAAAAAGTTTTG | 1539 | UAUUUAAAAAAGUUUUGUA |
| exon_4 | | | | TACAAAAAAATCC | | CAAAAAAAUCC |
| BCL11A_ | + | ATTT | 229 | AAAAAAGTTTTGTACAA | 1540 | AAAAAAGUUUUGUACAAAA |
| exon_4 | | | | AAAAATCCTTGCA | | AAAUCCUUGCA |
| BCL11A_ | + | TTTA | 230 | AAAAAGTTTTGTACAAA | 1541 | AAAAAGUUUUGUACAAAAA |
| exon 4 | | | | AAAATCCTTGCAC | | AAUCCUUGCAC |
| BCL11A_ | + | GTTT | 231 | TGTACAAAAAAATCCTT | 1542 | UGUACAAAAAAAUCCUUGC |
| exon 4 | | | | GCACTGTAGAAGC | | ACUGUAGAAGC |
| BCL11A_ | + | TTTT | 232 | GTACAAAAAAATCCTTG | 1543 | GUACAAAAAAAUCCUUGCA |
| exon 4 | | | | CACTGTAGAAGCG | | CUGUAGAAGCG |
| BCL11A_ | + | TTTG | 233 | TACAAAAAAATCCTTGC | 1544 | UACAAAAAAAUCCUUGCAC |
| exon 4 | | | | ACTGTAGAAGCGA | | UGUAGAAGCGA |
| BCL11A_ | + | CTTG | 234 | CACTGTAGAAGCGAAAG | 1545 | CACUGUAGAAGCGAAAGCA |
| exon_4 | | | | CAATCATTCATTT | | AUCAUUCAUUU |
| BCL11A_ | + | ATTC | 235 | ATTTCTATGTTAAGTGT | 1546 | AUUUCUAUGUUAAGUGUAU |
| exon_4 | | | | ATTCTGTTTCCAT | | UCUGUUUCCAU |
| BCL11A_ | + | TTTA | 236 | CAACCTGAAGAGCGGTG | 1547 | CAACCUGAAGAGCGGUGUG |
| exon_4 | | | | TGTATCCAAGGCA | | UAUCCAAGGCA |
| BCL11A_ | + | TTTC | 237 | TATGTTAAGTGTATTCT | 1548 | UAUGUUAAGUGUAUUCUGU |
| exon_4 | | | | GTTTCCATTCACA | | UUCCAUUCACA |
| BCL11A_ | + | GTTA | 238 | AGTGTATTCTGTTTCCA | 1549 | AGUGUAUUCUGUUUCCAUU |
| exon_4 | | | | TTCACAGCGCTTG | | CACAGCGCUUG |
| BCL11A_ | + | TTTT | 239 | CTTAACATTTATATTTA | 1550 | CUUAACAUUUAUAUUUAAA |
| exon_4 | | | | AAAAAGTTTTGTA | | AAAGUUUUGUA |
| BCL11A_ | + | TTTT | 240 | ACAACCTGAAGAGCGGT | 1551 | ACAACCUGAAGAGCGGUGU |
| exon_4 | | | | GTGTATCCAAGGC | | GUAUCCAAGGC |
| BCL11A_ | + | TTTA | 241 | AGTACTATATAATCTTA | 1552 | AGUACUAUAUAAUCUUAAA |
| exon_4 | | | | AACCTTTCCCCAA | | CCUUUCCCCAA |
| BCL11A_ | + | TTTT | 242 | TTACAACCTGAAGAGCG | 1553 | UUACAACCUGAAGAGCGGU |
| exon_4 | | | | GTGTGTATCCAAG | | GUGUAUCCAAG |
| BCL11A_ | + | TTTT | 243 | TCCACTACCAAAAAAGG | 1554 | UCCACUACCAAAAAAGGUA |
| exon_4 | | | | TACATTGATACCT | | CAUUGAUACCU |
| BCL11A_ | + | TTTT | 244 | CCACTACCAAAAAAGGT | 1555 | CCACUACCAAAAAAGGUAC |
| exon_4 | | | | ACATTGATACCTT | | AUUGAUACCUU |
| BCL11A_ | + | TTTC | 245 | CACTACCAAAAAAGGTA | 1556 | CACUACCAAAAAAGGUACA |
| exon_4 | | | | CATTGATACCTTT | | UUGAUACCUUU |
| BCL11A_ | + | ATTG | 246 | ATACCTTTTAAGAGAAC | 1557 | AUACCUUUUAAGAGAACAA |
| exon_4 | | | | AAGCAACAGTTAA | | GCAACAGUUAA |
| BCL11A_ | + | CTTT | 247 | TAAGAGAACAAGCAACA | 1558 | UAAGAGAACAAGCAACAGU |
| exon_4 | | | | GTTAAAAATACAA | | UAAAAAUACAA |
| BCL11A_ | + | TTTT | 248 | AAGAGAACAAGCAACAG | 1559 | AAGAGAACAAGCAACAGUU |
| exon_4 | | | | TTAAAAATACAAG | | AAAAAUACAAG |
| BCL11A_ | + | TTTA | 249 | AGAGAACAAGCAACAGT | 1560 | AGAGAACAAGCAACAGUUA |
| exon_4 | | | | TAAAAATACAAGC | | AAAAUACAAGC |
| BCL11A_ | + | GTTA | 250 | AAAATACAAGCTTCAAT | 1561 | AAAAUACAAGCUUCAAUAU |
| exon_4 | | | | ATAAATACTATAG | | AAAUACUAUAG |
| BCL11A_ | + | CTTC | 251 | AATATAAATACTATAGT | 1562 | AAUAUAAAUACUAUAGUGC |
| exon_4 | | | | GCCTAACACTAGA | | CUAACACUAGA |
| BCL11A_ | + | ATTT | 252 | AATTCAAATACCATTCT | 1563 | AAUUCAAAUACCAUUCUAG |
| exon_4 | | | | AGAAATACAGAAA | | AAAUACAGAAA |
| BCL11A_ | + | TTTA | 253 | ATTCAAATACCATTCTA | 1564 | AUUCAAAUACCAUUCUAGA |
| exon 4 | | | | GAAATACAGAAAA | | AAUACAGAAAA |
| BCL11A_ | + | ATTC | 254 | AAATACCATTCTAGAAA | 1565 | AAAUACCAUUCUAGAAAUA |
| exon_4 | | | | TACAGAAAAAAGA | | CAGAAAAAAGA |
| BCL11A_ | + | ATTC | 255 | TAGAAATACAGAAAAAA | 1566 | UAGAAAUACAGAAAAAAGA |
| exon_4 | | | | GACCATAAATGTA | | CCAUAAAUGUA |
| BCL11A_ | + | ATTT | 256 | TAGCATAGGAATCAACA | 1567 | UAGCAUAGGAAUCAACAUG |
| exon_4 | | | | TGAGTGTGCATTT | | AGUGUGCAUUU |
| BCL11A_ | + | TTTT | 257 | AGCATAGGAATCAACAT | 1568 | AGCAUAGGAAUCAACAUGA |
| exon_4 | | | | GAGTGTGCATTTT | | GUGUGCAUUUU |
| BCL11A_ | + | TTTA | 258 | GCATAGGAATCAACATG | 1569 | GCAUAGGAAUCAACAUGAG |
| exon_4 | | | | AGTGTGCATTTTC | | UGUGCAUUUUC |
| BCL11A_ | + | ATTT | 259 | TCCTATATTTAAGTACT | 1570 | UCCUAUAUUUAAGUACUAU |
| exon_4 | | | | ATATAATCTTAAA | | AUAAUCUUAAA |
| BCL11A_ | + | TTTT | 260 | TTTACAACCTGAAGAGC | 1571 | UUUACAACCUGAAGAGCGG |
| exon_4 | | | | GGTGTGTATCCAA | | UGUGUAUCCAA |
| BCL11A_ | + | TTTT | 261 | TTTTACAACCTGAAGAG | 1572 | UUUUACAACCUGAAGAGCG |
| exon 4 | | | | CGGTGTGTATCCA | | GUGUGUAUCCA |
| BCL11A_ | + | TTTT | 262 | TTTTTACAACCTGAAGA | 1573 | UUUUUACAACCUGAAGAGC |
| exon_4 | | | | GCGGTGTGTATCC | | GGUGUGUAUCC |
| BCL11A_ | + | TTTT | 263 | TTTTTTACAACCTGAAG | 1574 | UUUUUUACAACCUGAAGAG |
| exon_4 | | | | AGCGGTGTGTATC | | CGGUGUGUAUC |
| BCL11A_ | + | TTTT | 264 | TTTTTTTACAACCTGAA | 1575 | UUUUUUUACAACCUGAAGA |
| exon_4 | | | | GAGCGGTGTGTAT | | GCGGUGUGUAU |
| BCL11A_ | + | TTTT | 265 | TTTTTTTTACAACCTGA | 1576 | UUUUUUUUACAACCUGAAG |
| exon_4 | | | | AGAGCGGTGTGTA | | AGCGGUGUGUA |
| BCL11A_ | + | TTTT | 266 | TACAACCTGAAGAGCGG | 1577 | UACAACCUGAAGAGCGGUG |
| exon_4 | | | | TGTGTATCCAAGG | | UGUAUCCAAGG |
| BCL11A_ | + | GTTT | 267 | TTTTTTTTTACAACCTG | 1578 | UUUUUUUUUACAACCUGAA |
| exon_4 | | | | AAGAGCGGTGTGT | | GAGCGGUGUGU |
| BCL11A_ | + | CTTT | 268 | CCCCAATGTATGTTTTT | 1579 | CCCCAAUGUAUGUUUUUUU |
| exon_4 | | | | TTTTTTTACAACC | | UUUUUACAACC |
| BCL11A_ | + | CTTA | 269 | AACCTTTCCCCAATGTA | 1580 | AACCUUUCCCCAAUGUAUG |
| exon_4 | | | | TGTTTTTTTTTTT | | UUUUUUUUUUU |
| BCL11A_ | + | ATTC | 270 | TGTTTCCATTCACAGCG | 1581 | UGUUUCCAUUCACAGCGCU |
| exon_4 | | | | CTTGCAATGTTGC | | UGCAAUGUUGC |
| BCL11A_ | + | ATTT | 271 | AAGTACTATATAATCTT | 1582 | AAGUACUAUAUAAUCUUAA |
| exon_4 | | | | AAACCTTTCCCCA | | ACCUUUCCCCA |
| BCL11A_ | + | TTTC | 272 | CTATATTTAAGTACTAT | 1583 | CUAUAUUUAAGUACUAUAU |
| exon_4 | | | | ATAATCTTAAACC | | AAUCUUAAACC |
| BCL11A_ | + | TTTT | 273 | CCTATATTTAAGTACTA | 1584 | CCUAUAUUUAAGUACUAUA |
| exon_4 | | | | TATAATCTTAAAC | | UAAUCUUAAAC |
| BCL11A_ | + | TTTC | 274 | CCCAATGTATGTTTTTT | 1585 | CCCAAUGUAUGUUUUUUUU |
| exon_4 | | | | TTTTTTACAACCT | | UUUUACAACCU |
| BCL11A_ | + | GTTT | 275 | CCATTCACAGCGCTTGC | 1586 | CCAUUCACAGCGCUUGCAA |
| exon_4 | | | | AATGTTGCGTCCA | | UGUUGCGUCCA |
| BCL11A_ | + | TTTT | 276 | TTAGTTTTTAAAAAATG | 1587 | UUAGUUUUUAAAAAAUGCU |
| exon_4 | | | | CTCCTCAATGAGA | | CCUCAAUGAGA |
| BCL11A_ | + | ATTC | 277 | ACAGCGCTTGCAATGTT | 1588 | ACAGCGCUUGCAAUGUUGC |
| exon_4 | | | | GCGTCCAAGTAAG | | GUCCAAGUAAG |
| BCL11A_ | + | ATTG | 278 | TCCTATCTGAGCAGGTT | 1589 | UCCUAUCUGAGCAGGUUUA |
| exon_4 | | | | TATTTTATACTCA | | UUUUAUACUCA |
| BCL11A_ | + | GTTT | 279 | ATTTTATACTCAACCTC | 1590 | AUUUUAUACUCAACCUCUG |
| exon_4 | | | | TGTATCTCTGATT | | UAUCUCUGAUU |
| BCL11A_ | + | TTTA | 280 | TTTTATACTCAACCTCT | 1591 | UUUUAUACUCAACCUCUGU |
| exon 4 | | | | GTATCTCTGATTA | | AUCUCUGAUUA |
| BCL11A_ | + | ATTT | 281 | TATACTCAACCTCTGTA | 1592 | UAUACUCAACCUCUGUAUC |
| exon_4 | | | | TCTCTGATTAGAG | | UCUGAUUAGAG |
| BCL11A_ | + | TTTT | 282 | ATACTCAACCTCTGTAT | 1593 | AUACUCAACCUCUGUAUCU |
| exon 4 | | | | CTCTGATTAGAGA | | CUGAUUAGAGA |
| BCL11A_ | + | TTTA | 283 | TACTCAACCTCTGTATC | 1594 | UACUCAACCUCUGUAUCUC |
| exon_4 | | | | TCTGATTAGAGAA | | UGAUUAGAGAA |
| BCL11A_ | + | ATTA | 284 | GAGAAAAGATACAGATA | 1595 | GAGAAAAGAUACAGAUAUC |
| exon_4 | | | | TCACAGGCAGAGT | | ACAGGCAGAGU |
| BCL11A_ | + | ATTT | 285 | GAACACCAACTGGGGCA | 1596 | GAACACCAACUGGGGCAGA |
| exon 4 | | | | GATGCTAGCTTAA | | UGCUAGCUUAA |
| BCL11A_ | + | TTTG | 286 | AACACCAACTGGGGCAG | 1597 | AACACCAACUGGGGCAGAU |
| exon_4 | | | | ATGCTAGCTTAAT | | GCUAGCUUAAU |
| BCL11A_ | + | CTTA | 287 | ATAAAAAAGAAAAAATT | 1598 | AUAAAAAAGAAAAAAUUAA |
| exon_4 | | | | AAAAAAATAAAAA | | AAAAAUAAAAA |
| BCL11A_ | + | ATTA | 288 | AAAAAATAAAAATAAAA | 1599 | AAAAAAUAAAAAUAAAAAC |
| exon 4 | | | | ACAATGAATCCTC | | AAUGAAUCCUC |
| BCL11A_ | + | CTTC | 289 | CATGTTAACACAAATAG | 1600 | CAUGUUAACACAAAUAGCA |
| exon 4 | | | | CACACAGTGTATG | | CACAGUGUAUG |
| BCL11A_ | + | GTTA | 290 | ACACAAATAGCACACAG | 1601 | ACACAAAUAGCACACAGUG |
| exon 4 | | | | TGTATGGAAAAGA | | UAUGGAAAAGA |
| BCL11A_ | + | CTTT | 291 | TAGGGAGCACAGACATA | 1602 | UAGGGAGCACAGACAUAUA |
| exon_4 | | | | TATACTGCTACTC | | UACUGCUACUC |
| BCL11A_ | + | TTTT | 292 | AGGGAGCACAGACATAT | 1603 | AGGGAGCACAGACAUAUAU |
| exon_4 | | | | ATACTGCTACTCT | | ACUGCUACUCU |
| BCL11A_ | + | TTTA | 293 | GGGAGCACAGACATATA | 1604 | GGGAGCACAGACAUAUAUA |
| exon_4 | | | | TACTGCTACTCTT | | CUGCUACUCUU |
| BCL11A_ | + | CTTA | 294 | AAATTCTTTCTCTTCTT | 1605 | AAAUUCUUUCUCUUCUUUU |
| exon_4 | | | | TTTTTAAGAATGT | | UUUAAGAAUGU |
| BCL11A_ | + | ATTC | 295 | ATAGTTAATCATCATTG | 1606 | AUAGUUAAUCAUCAUUGUA |
| exon_4 | | | | TATCAATATTAGC | | UCAAUAUUAGC |
| BCL11A_ | + | CTTA | 296 | AGAATTCATAGTTAATC | 1607 | AGAAUUCAUAGUUAAUCAU |
| exon_4 | | | | ATCATTGTATCAA | | CAUUGUAUCAA |
| BCL11A_ | + | TTTA | 297 | AATGCAAGTCTTAAGAA | 1608 | AAUGCAAGUCUUAAGAAUU |
| exon 4 | | | | TTCATAGTTAATC | | CAUAGUUAAUC |
| BCL11A_ | + | ATTT | 298 | AAATGCAAGTCTTAAGA | 1609 | AAAUGCAAGUCUUAAGAAU |
| exon 4 | | | | ATTCATAGTTAAT | | UCAUAGUUAAU |
| BCL11A_ | + | TTTA | 299 | AGAATGTCACATTTAAA | 1610 | AGAAUGUCACAUUUAAAUG |
| exon 4 | | | | TGCAAGTCTTAAG | | CAAGUCUUAAG |
| BCL11A_ | + | TTTT | 300 | AAGAATGTCACATTTAA | 1611 | AAGAAUGUCACAUUUAAAU |
| exon 4 | | | | ATGCAAGTCTTAA | | GCAAGUCUUAA |
| BCL11A_ | + | CTTA | 301 | ATTGTCCTATCTGAGCA | 1612 | AUUGUCCUAUCUGAGCAGG |
| exon 4 | | | | GGTTTATTTTATA | | UUUAUUUUAUA |
| BCL11A_ | + | TTTT | 302 | TAAGAATGTCACATTTA | 1613 | UAAGAAUGUCACAUUUAAA |
| exon_4 | | | | AATGCAAGTCTTA | | UGCAAGUCUUA |
| BCL11A_ | + | TTTT | 303 | TTTAAGAATGTCACATT | 1614 | UUUAAGAAUGUCACAUUUA |
| exon_4 | | | | TAAATGCAAGTCT | | AAUGCAAGUCU |
| BCL11A_ | + | CTTT | 304 | TTTTAAGAATGTCACAT | 1615 | UUUUAAGAAUGUCACAUUU |
| exon 4 | | | | TTAAATGCAAGTC | | AAAUGCAAGUC |
| BCL11A_ | + | CTTC | 305 | TTTTTTTAAGAATGTCA | 1616 | UUUUUUUAAGAAUGUCACA |
| exon 4 | | | | CATTTAAATGCAA | | UUUAAAUGCAA |
| BCL11A_ | + | TTTC | 306 | TCTTCTTTTTTTAAGAA | 1617 | UCUUCUUUUUUUAAGAAUG |
| exon_4 | | | | TGTCACATTTAAA | | UCACAUUUAAA |
| BCL11A_ | + | CTTT | 307 | CTCTTCTTTTTTTAAGA | 1618 | CUCUUCUUUUUUUAAGAAU |
| exon_4 | | | | ATGTCACATTTAA | | GUCACAUUUAA |
| BCL11A_ | + | ATTC | 308 | TTTCTCTTCTTTTTTTA | 1619 | UUUCUCUUCUUUUUUUAAG |
| exon_4 | | | | AGAATGTCACATT | | AAUGUCACAUU |
| BCL11A_ | + | TTTT | 309 | TTAAGAATGTCACATTT | 1620 | UUAAGAAUGUCACAUUUAA |
| exon_4 | | | | AAATGCAAGTCTT | | AUGCAAGUCUU |
| BCL11A_ | + | TTTC | 310 | CATTCACAGCGCTTGCA | 1621 | CAUUCACAGCGCUUGCAAU |
| exon_4 | | | | ATGTTGCGTCCAA | | GUUGCGUCCAA |
| BCL11A_ | + | ATTG | 311 | TACAGTGCACTTAATTG | 1622 | UACAGUGCACUUAAUUGUC |
| exon_4 | | | | TCCTATCTGAGCA | | CUAUCUGAGCA |
| BCL11A_ | + | TTTC | 312 | CCTTAAGTATAGACCTG | 1623 | CCUUAAGUAUAGACCUGUA |
| exon_4 | | | | TAAACTGGGAAAA | | AACUGGGAAAA |
| BCL11A_ | + | CTTG | 313 | CAATGTTGCGTCCAAGT | 1624 | CAAUGUUGCGUCCAAGUAA |
| exon_4 | | | | AAGTAAGCTCAAT | | GUAAGCUCAAU |
| BCL11A_ | + | GTTG | 314 | CGTCCAAGTAAGTAAGC | 1625 | CGUCCAAGUAAGUAAGCUC |
| exon_4 | | | | TCAATAGTCAAGT | | AAUAGUCAAGU |
| BCL11A_ | + | GTTT | 315 | TTTTTTTTTTAGTTTTT | 1626 | UUUUUUUUUUAGUUUUUAA |
| exon_4 | | | | AAAAAATGCTCCT | | AAAAUGCUCCU |
| BCL11A_ | + | TTTT | 316 | TTTTTTTTTAGTTTTTA | 1627 | UUUUUUUUUAGUUUUUAAA |
| exon_4 | | | | AAAAATGCTCCTC | | AAAUGCUCCUC |
| BCL11A_ | + | TTTT | 317 | TTTTTTTTAGTTTTTAA | 1628 | UUUUUUUUAGUUUUUAAAA |
| exon_4 | | | | AAAATGCTCCTCA | | AAUGCUCCUCA |
| BCL11A_ | + | TTTT | 318 | TTTTTTTAGTTTTTAAA | 1629 | UUUUUUUAGUUUUUAAAAA |
| exon_4 | | | | AAATGCTCCTCAA | | AUGCUCCUCAA |
| BCL11A_ | + | TTTT | 319 | TTTTTTAGTTTTTAAAA | 1630 | UUUUUUAGUUUUUAAAAAA |
| exon 4 | | | | AATGCTCCTCAAT | | UGCUCCUCAAU |
| BCL11A_ | + | TTTT | 320 | TTTTTAGTTTTTAAAAA | 1631 | UUUUUAGUUUUUAAAAAAU |
| exon_4 | | | | ATGCTCCTCAATG | | GCUCCUCAAUG |
| BCL11A_ | + | TTTT | 321 | TTTTAGTTTTTAAAAAA | 1632 | UUUUAGUUUUUAAAAAAUG |
| exon_4 | | | | TGCTCCTCAATGA | | CUCCUCAAUGA |
| BCL11A_ | + | TTTT | 322 | TTTAGTTTTTAAAAAAT | 1633 | UUUAGUUUUUAAAAAAUGC |
| exon_4 | | | | GCTCCTCAATGAG | | UCCUCAAUGAG |
| BCL11A_ | + | TTTT | 323 | TTCCACTACCAAAAAAG | 1634 | UUCCACUACCAAAAAAGGU |
| exon 4 | | | | GTACATTGATACC | | ACAUUGAUACC |
| BCL11A_ | + | TTTT | 324 | TAGTTTTTAAAAAATGC | 1635 | UAGUUUUUAAAAAAUGCUC |
| exon_4 | | | | TCCTCAATGAGAT | | CUCAAUGAGAU |
| BCL11A_ | + | TTTT | 325 | AGTTTTTAAAAAATGCT | 1636 | AGUUUUUAAAAAAUGCUCC |
| exon 4 | | | | CCTCAATGAGATT | | UCAAUGAGAUU |
| BCL11A_ | + | TTTA | 326 | GTTTTTAAAAAATGCTC | 1637 | GUUUUUAAAAAAUGCUCCU |
| exon_4 | | | | CTCAATGAGATTG | | CAAUGAGAUUG |
| BCL11A_ | + | GTTT | 327 | TTAAAAAATGCTCCTCA | 1638 | UUAAAAAAUGCUCCUCAAU |
| exon_4 | | | | ATGAGATTGTGTT | | GAGAUUGUGUU |
| BCL11A_ | + | TTTT | 328 | TAAAAAATGCTCCTCAA | 1639 | UAAAAAAUGCUCCUCAAUG |
| exon 4 | | | | TGAGATTGTGTTC | | AGAUUGUGUUC |
| BCL11A_ | + | TTTT | 329 | AAAAAATGCTCCTCAAT | 1640 | AAAAAAUGCUCCUCAAUGA |
| exon 4 | | | | GAGATTGTGTTCA | | GAUUGUGUUCA |
| BCL11A_ | + | TTTT | 330 | CCCTTAAGTATAGACCT | 1641 | CCCUUAAGUAUAGACCUGU |
| exon 4 | | | | GTAAACTGGGAAA | | AAACUGGGAAA |
| BCL11A_ | + | CTTT | 331 | TCCCTTAAGTATAGACC | 1642 | UCCCUUAAGUAUAGACCUG |
| exon 4 | | | | TGTAAACTGGGAA | | UAAACUGGGAA |
| BCL11A_ | + | CTTG | 332 | CAACTTTTCCCTTAAGT | 1643 | CAACUUUUCCCUUAAGUAU |
| exon 4 | | | | ATAGACCTGTAAA | | AGACCUGUAAA |
| BCL11A_ | + | ATTC | 333 | TTGCAACTTTTCCCTTA | 1644 | UUGCAACUUUUCCCUUAAG |
| exon 4 | | | | AGTATAGACCTGT | | UAUAGACCUGU |
| BCL11A_ | + | TTTC | 334 | AGCATTCTTGCAACTTT | 1645 | AGCAUUCUUGCAACUUUUC |
| exon_4 | | | | TCCCTTAAGTATA | | CCUUAAGUAUA |
| BCL11A_ | + | TTTT | 335 | CAGCATTCTTGCAACTT | 1646 | CAGCAUUCUUGCAACUUUU |
| exon_4 | | | | TTCCCTTAAGTAT | | CCCUUAAGUAU |
| BCL11A_ | + | CTTA | 336 | AGTATAGACCTGTAAAC | 1647 | AGUAUAGACCUGUAAACUG |
| exon_4 | | | | TGGGAAAATTGTA | | GGAAAAUUGUA |
| BCL11A_ | + | TTTT | 337 | TCAGCATTCTTGCAACT | 1648 | UCAGCAUUCUUGCAACUUU |
| exon_4 | | | | TTTCCCTTAAGTA | | UCCCUUAAGUA |
| BCL11A_ | + | TTTT | 338 | TTTCAGCATTCTTGCAA | 1649 | UUUCAGCAUUCUUGCAACU |
| exon_4 | | | | CTTTTCCCTTAAG | | UUUCCCUUAAG |
| BCL11A_ | + | TTTT | 339 | TTTTCAGCATTCTTGCA | 1650 | UUUUCAGCAUUCUUGCAAC |
| exon 4 | | | | ACTTTTCCCTTAA | | UUUUCCCUUAA |
| BCL11A_ | + | ATTT | 340 | TTTTTCAGCATTCTTGC | 1651 | UUUUUCAGCAUUCUUGCAA |
| exon_4 | | | | AACTTTTCCCTTA | | CUUUUCCCUUA |
| BCL11A_ | + | GTTC | 341 | AATTTTTTTTCAGCATT | 1652 | AAUUUUUUUUCAGCAUUCU |
| exon_4 | | | | CTTGCAACTTTTC | | UGCAACUUUUC |
| BCL11A_ | + | ATTG | 342 | TGTTCAATTTTTTTTCA | 1653 | UGUUCAAUUUUUUUUCAGC |
| exon_4 | | | | GCATTCTTGCAAC | | AUUCUUGCAAC |
| BCL11A_ | + | TTTA | 343 | AAAAATGCTCCTCAATG | 1654 | AAAAAUGCUCCUCAAUGAG |
| exon_4 | | | | AGATTGTGTTCAA | | AUUGUGUUCAA |
| BCL11A_ | + | TTTT | 344 | TTCAGCATTCTTGCAAC | 1655 | UUCAGCAUUCUUGCAACUU |
| exon 4 | | | | TTTTCCCTTAAGT | | UUCCCUUAAGU |
| BCL11A_ | + | TTTT | 345 | TTTCCACTACCAAAAAA | 1656 | UUUCCACUACCAAAAAAGG |
| exon 4 | | | | GGTACATTGATAC | | UACAUUGAUAC |
| BCL11A_ | + | TTTC | 346 | CAATAGAACTTAACAAA | 1657 | CAAUAGAACUUAACAAAGA |
| exon 4 | | | | GACCAGAAACAAA | | CCAGAAACAAA |
| BCL11A_ | + | TTTT | 347 | TTTTTCCACTACCAAAA | 1658 | UUUUUCCACUACCAAAAAA |
| exon 4 | | | | AAGGTACATTGAT | | GGUACAUUGAU |
| BCL11A_ | + | GTTT | 348 | TTCCAATAGAACTTAAC | 1659 | UUCCAAUAGAACUUAACAA |
| exon 4 | | | | AAAGACCAGAAAC | | AGACCAGAAAC |
| BCL11A_ | + | TTTT | 349 | TCCAATAGAACTTAACA | 1660 | UCCAAUAGAACUUAACAAA |
| exon_4 | | | | AAGACCAGAAACA | | GACCAGAAACA |
| BCL11A_ | + | TTTT | 350 | CCAATAGAACTTAACAA | 1661 | CCAAUAGAACUUAACAAAG |
| exon_4 | | | | AGACCAGAAACAA | | ACCAGAAACAA |
| BCL11A_ | + | GTTA | 351 | ATCATCATTGTATCAAT | 1662 | AUCAUCAUUGUAUCAAUAU |
| exon_4 | | | | ATTAGCTTATATA | | UAGCUUAUAUA |
| BCL11A_ | + | CTTA | 352 | ACAAAGACCAGAAACAA | 1663 | ACAAAGACCAGAAACAAAU |
| exon_4 | | | | ATACAATAAAAAG | | ACAAUAAAAAG |
| BCL11A_ | + | GTTG | 353 | TAATGACCTTTGGTCAT | 1664 | UAAUGACCUUUGGUCAUCU |
| exon_4 | | | | CTAAATAAAAAAA | | AAAUAAAAAAA |
| BCL11A_ | + | CTTT | 354 | GGTCATCTAAATAAAAA | 1665 | GGUCAUCUAAAUAAAAAAA |
| exon_4 | | | | AAAAAATAAAAAC | | AAAAUAAAAAC |
| BCL11A_ | + | TTTG | 355 | GTCATCTAAATAAAAAA | 1666 | GUCAUCUAAAUAAAAAAAA |
| exon_4 | | | | AAAAATAAAAACA | | AAAAAAAACA |
| BCL11A_ | + | ATTA | 356 | AGTGCCTCTGTTTTGAA | 1667 | AGUGCCUCUGUUUUGAACA |
| exon_4 | | | | CAGGGCACATAAG | | GGGCACAUAAG |
| BCL11A_ | + | GTTT | 357 | TGAACAGGGCACATAAG | 1668 | UGAACAGGGCACAUAAGCA |
| exon_4 | | | | CAATAATAAATAG | | AUAAUAAAUAG |
| BCL11A_ | + | TTTT | 358 | GAACAGGGCACATAAGC | 1669 | GAACAGGGCACAUAAGCAA |
| exon 4 | | | | AATAATAAATAGT | | UAAUAAAUAGU |
| BCL11A_ | + | TTTG | 359 | AACAGGGCACATAAGCA | 1670 | AACAGGGCACAUAAGCAAU |
| exon_4 | | | | ATAATAAATAGTG | | AAUAAAUAGUG |
| BCL11A_ | + | ATTT | 360 | CAAGTTACGACAAACAG | 1671 | CAAGUUACGACAAACAGCU |
| exon_4 | | | | CTTTCATTACAGG | | UUCAUUACAGG |
| BCL11A_ | + | TTTC | 361 | AAGTTACGACAAACAGC | 1672 | AAGUUACGACAAACAGCUU |
| exon_4 | | | | TTTCATTACAGGA | | UCAUUACAGGA |
| BCL11A_ | + | GTTA | 362 | ATGCAGACAACTGCCAA | 1673 | AUGCAGACAACUGCCAAAA |
| exon_4 | | | | AAAAACACAGACA | | AAACACAGACA |
| BCL11A_ | + | GTTA | 363 | CGACAAACAGCTTTCAT | 1674 | CGACAAACAGCUUUCAUUA |
| exon_4 | | | | TACAGGAATAGAA | | CAGGAAUAGAA |
| BCL11A_ | + | TTTC | 364 | ATTACAGGAATAGAAAA | 1675 | AUUACAGGAAUAGAAAAGG |
| exon_4 | | | | GGCCAATAACAAA | | CCAAUAACAAA |
| BCL11A_ | + | ATTA | 365 | CAGGAATAGAAAAGGCC | 1676 | CAGGAAUAGAAAAGGCCAA |
| exon_4 | | | | AATAACAAAATAT | | UAACAAAAUAU |
| BCL11A_ | + | ATTC | 366 | TGCATTGCCATTTACAA | 1677 | UGCAUUGCCAUUUACAAAA |
| exon 4 | | | | AAAAGTATTGACT | | AAGUAUUGACU |
| BCL11A_ | + | ATTG | 367 | CCATTTACAAAAAAGTA | 1678 | CCAUUUACAAAAAAGUAUU |
| exon 4 | | | | TTGACTAAAGCGG | | GACUAAAGCGG |
| BCL11A_ | + | ATTT | 368 | ACAAAAAAGTATTGACT | 1679 | ACAAAAAAGUAUUGACUAA |
| exon_4 | | | | AAAGCGGGCTTTC | | AGCGGGCUUUC |
| BCL11A_ | + | TTTA | 369 | CAAAAAAGTATTGACTA | 1680 | CAAAAAAGUAUUGACUAAA |
| exon_4 | | | | AAGCGGGCTTTCT | | GCGGGCUUUCU |
| BCL11A_ | + | ATTG | 370 | ACTAAAGCGGGCTTTCT | 1681 | ACUAAAGCGGGCUUUCUCU |
| exon 4 | | | | CTTTAATATGCTT | | UUAAUAUGCUU |
| BCL11A_ | + | CTTT | 371 | CTCTTTAATATGCTTTG | 1682 | CUCUUUAAUAUGCUUUGCA |
| exon_4 | | | | CATATGAAATTCT | | UAUGAAAUUCU |
| BCL11A_ | + | TTTC | 372 | TCTTTAATATGCTTTGC | 1683 | UCUUUAAUAUGCUUUGCAU |
| exon_4 | | | | ATATGAAATTCTT | | AUGAAAUUCUU |
| BCL11A_ | + | CTTT | 373 | AATATGCTTTGCATATG | 1684 | AAUAUGCUUUGCAUAUGAA |
| exon_4 | | | | AAATTCTTTCCAA | | AUUCUUUCCAA |
| BCL11A_ | + | TTTA | 374 | ATATGCTTTGCATATGA | 1685 | AUAUGCUUUGCAUAUGAAA |
| exon 4 | | | | AATTCTTTCCAAT | | UUCUUUCCAAU |
| BCL11A_ | + | CTTT | 375 | GCATATGAAATTCTTTC | 1686 | GCAUAUGAAAUUCUUUCCA |
| exon_4 | | | | CAATCTAAATATA | | AUCUAAAUAUA |
| BCL11A_ | + | TTTG | 376 | CATATGAAATTCTTTCC | 1687 | CAUAUGAAAUUCUUUCCAA |
| exon_4 | | | | AATCTAAATATAA | | UCUAAAUAUAA |
| BCL11A_ | + | ATTC | 377 | TTTCCAATCTAAATATA | 1688 | UUUCCAAUCUAAAUAUAAA |
| exon 4 | | | | AAGCACCATTTAG | | GCACCAUUUAG |
| BCL11A_ | + | CTTT | 378 | CATTACAGGAATAGAAA | 1689 | CAUUACAGGAAUAGAAAAG |
| exon_4 | | | | AGGCCAATAACAA | | GCCAAUAACAA |
| BCL11A_ | + | TTTC | 379 | AATAAAGGGACAAAATG | 1690 | AAUAAAGGGACAAAAUGGG |
| exon_4 | | | | GGTGTATGAACAG | | UGUAUGAACAG |
| BCL11A_ | + | TTTT | 380 | CAATAAAGGGACAAAAT | 1691 | CAAUAAAGGGACAAAAUGG |
| exon_4 | | | | GGGTGTATGAACA | | GUGUAUGAACA |
| BCL11A_ | + | TTTT | 381 | TCAATAAAGGGACAAAA | 1692 | UCAAUAAAGGGACAAAAUG |
| exon_4 | | | | TGGGTGTATGAAC | | GGUGUAUGAAC |
| BCL11A_ | − | TTTT | 382 | GGCAGTTGTCTGCATTA | 1693 | GGCAGUUGUCUGCAUUAAC |
| exon_4 | | | | ACCTGTTCATACA | | CUGUUCAUACA |
| BCL11A_ | − | TTTG | 383 | GCAGTTGTCTGCATTAA | 1694 | GCAGUUGUCUGCAUUAACC |
| exon 4 | | | | CCTGTTCATACAC | | UGUUCAUACAC |
| BCL11A_ | − | GTTG | 384 | TCTGCATTAACCTGTTC | 1695 | UCUGCAUUAACCUGUUCAU |
| exon 4 | | | | ATACACCCATTTT | | ACACCCAUUUU |
| BCL11A_ | − | ATTA | 385 | ACCTGTTCATACACCCA | 1696 | ACCUGUUCAUACACCCAUU |
| exon 4 | | | | TTTTGTCCCTTTA | | UUGUCCCUUUA |
| BCL11A_ | − | GTTC | 386 | ATACACCCATTTTGTCC | 1697 | AUACACCCAUUUUGUCCCU |
| exon 4 | | | | CTTTATTGAAAAA | | UUAUUGAAAAA |
| BCL11A_ | − | ATTT | 387 | TGTCCCTTTATTGAAAA | 1698 | UGUCCCUUUAUUGAAAAAA |
| exon 4 | | | | AATAAAAAAAATT | | UAAAAAAAAUU |
| BCL11A_ | − | TTTT | 388 | GTCCCTTTATTGAAAAA | 1699 | GUCCCUUUAUUGAAAAAAU |
| exon 4 | | | | ATAAAAAAAATTA | | AAAAAAAAUUA |
| BCL11A_ | − | TTTG | 389 | TCCCTTTATTGAAAAAA | 1700 | UCCCUUUAUUGAAAAAAUA |
| exon_4 | | | | TAAAAAAAATTAA | | AAAAAAAUUAA |
| BCL11A_ | − | CTTT | 390 | ATTGAAAAAATAAAAAA | 1701 | AUUGAAAAAAUAAAAAAAA |
| exon_4 | | | | AATTAAAGTACAC | | UUAAAGUACAC |
| BCL11A_ | − | TTTA | 391 | TTGAAAAAATAAAAAAA | 1702 | UUGAAAAAAUAAAAAAAAU |
| exon_4 | | | | ATTAAAGTACACA | | UAAAGUACACA |
| BCL11A_ | − | ATTG | 392 | AAAAAATAAAAAAAATT | 1703 | AAAAAAUAAAAAAAAUUAA |
| exon_4 | | | | AAAGTACACATTG | | AGUACACAUUG |
| BCL11A_ | − | ATTA | 393 | AAGTACACATTGTAAGC | 1704 | AAGUACACAUUGUAAGCUU |
| exon_4 | | | | TTCTTGTGTCCTC | | CUUGUGUCCUC |
| BCL11A_ | − | ATTG | 394 | TAAGCTTCTTGTGTCCT | 1705 | UAAGCUUCUUGUGUCCUCA |
| exon_4 | | | | CATTTGACACACT | | UUUGACACACU |
| BCL11A_ | − | CTTC | 395 | TTGTGTCCTCATTTGAC | 1706 | UUGUGUCCUCAUUUGACAC |
| exon_4 | | | | ACACTCTGTAAAT | | ACUCUGUAAAU |
| BCL11A_ | − | CTTG | 396 | TGTCCTCATTTGACACA | 1707 | UGUCCUCAUUUGACACACU |
| exon_4 | | | | CTCTGTAAATTAC | | CUGUAAAUUAC |
| BCL11A_ | − | ATTT | 397 | GACACACTCTGTAAATT | 1708 | GACACACUCUGUAAAUUAC |
| exon_4 | | | | ACTTGCAAGAAAA | | UUGCAAGAAAA |
| BCL11A_ | − | TTTG | 398 | ACACACTCTGTAAATTA | 1709 | ACACACUCUGUAAAUUACU |
| exon_4 | | | | CTTGCAAGAAAAT | | UGCAAGAAAAU |
| BCL11A_ | + | TTTT | 399 | TTCAATAAAGGGACAAA | 1710 | UUCAAUAAAGGGACAAAAU |
| exon_4 | | | | ATGGGTGTATGAA | | GGGUGUAUGAA |
| BCL11A_ | + | ATTT | 400 | TTTCAATAAAGGGACAA | 1711 | UUUCAAUAAAGGGACAAAA |
| exon_4 | | | | AATGGGTGTATGA | | UGGGUGUAUGA |
| BCL11A_ | + | TTTA | 401 | TTTTTTCAATAAAGGGA | 1712 | UUUUUUCAAUAAAGGGACA |
| exon_4 | | | | CAAAATGGGTGTA | | AAAUGGGUGUA |
| BCL11A_ | + | TTTT | 402 | ATTTTTTCAATAAAGGG | 1713 | AUUUUUUCAAUAAAGGGAC |
| exon_4 | | | | ACAAAATGGGTGT | | AAAAUGGGUGU |
| BCL11A_ | + | TTTT | 403 | TATTTTTTCAATAAAGG | 1714 | UAUUUUUUCAAUAAAGGGA |
| exon_4 | | | | GACAAAATGGGTG | | CAAAAUGGGUG |
| BCL11A_ | + | TTTT | 404 | TTATTTTTTCAATAAAG | 1715 | UUAUUUUUUCAAUAAAGGG |
| exon 4 | | | | GGACAAAATGGGT | | ACAAAAUGGGU |
| BCL11A_ | + | CTTT | 405 | CCAATCTAAATATAAAG | 1716 | CCAAUCUAAAUAUAAAGCA |
| exon 4 | | | | CACCATTTAGTTT | | CCAUUUAGUUU |
| BCL11A_ | + | TTTT | 406 | TTTATTTTTTCAATAAA | 1717 | UUUAUUUUUUCAAUAAAGG |
| exon 4 | | | | GGGACAAAATGGG | | GACAAAAUGGG |
| BCL11A_ | + | ATTT | 407 | TTTTTATTTTTTCAATA | 1718 | UUUUUAUUUUUUCAAUAAA |
| exon_4 | | | | AAGGGACAAAATG | | GGGACAAAAUG |
| BCL11A_ | + | TTTA | 408 | ATTTTTTTTATTTTTTC | 1719 | AUUUUUUUUAUUUUUUCAA |
| exon_4 | | | | AATAAAGGGACAA | | UAAAGGGACAA |
| BCL11A_ | + | CTTT | 409 | AATTTTTTTTATTTTTT | 1720 | AAUUUUUUUUAUUUUUUCA |
| exon_4 | | | | CAATAAAGGGACA | | AUAAAGGGACA |
| BCL11A_ | + | CTTA | 410 | CAATGTGTACTTTAATT | 1721 | CAAUGUGUACUUUAAUUUU |
| exon_4 | | | | TTTTTTATTTTTT | | UUUUAUUUUUU |
| BCL11A_ | + | TTTA | 411 | CAGAGTGTGTCAAATGA | 1722 | CAGAGUGUGUCAAAUGAGG |
| exon_4 | | | | GGACACAAGAAGC | | ACACAAGAAGC |
| BCL11A_ | + | ATTT | 412 | ACAGAGTGTGTCAAATG | 1723 | ACAGAGUGUGUCAAAUGAG |
| exon_4 | | | | AGGACACAAGAAG | | GACACAAGAAG |
| BCL11A_ | + | TTTT | 413 | TTTTATTTTTTCAATAA | 1724 | UUUUAUUUUUUCAAUAAAG |
| exon_4 | | | | AGGGACAAAATGG | | GGACAAAAUGG |
| BCL11A_ | + | TTTC | 414 | CAATCTAAATATAAAGC | 1725 | CAAUCUAAAUAUAAAGCAC |
| exon_4 | | | | ACCATTTAGTTTT | | CAUUUAGUUUU |
| BCL11A_ | + | ATTT | 415 | AGTTTTTGGCAATGAAA | 1726 | AGUUUUUGGCAAUGAAAAA |
| exon_4 | | | | AAAACTGCAAAAC | | AACUGCAAAAC |
| BCL11A_ | + | TTTA | 416 | GTTTTTGGCAATGAAAA | 1727 | GUUUUUGGCAAUGAAAAAA |
| exon 4 | | | | AAACTGCAAAACA | | ACUGCAAAACA |
| BCL11A_ | + | ATTA | 417 | GCTTGCAGTACTGCATA | 1728 | GCUUGCAGUACUGCAUACA |
| exon_4 | | | | CAGTATGGCAGCA | | GUAUGGCAGCA |
| BCL11A_ | + | CTTG | 418 | CAGTACTGCATACAGTA | 1729 | CAGUACUGCAUACAGUAUG |
| exon_4 | | | | TGGCAGCAGGAAA | | GCAGCAGGAAA |
| BCL11A_ | + | ATTC | 419 | TAGCAGGCTCCCCCAAA | 1730 | UAGCAGGCUCCCCCAAACC |
| exon_4 | | | | CCGCCATTATATG | | GCCAUUAUAUG |
| BCL11A_ | + | ATTA | 420 | TATGGCTTCTCATCTGT | 1731 | UAUGGCUUCUCAUCUGUAA |
| exon_4 | | | | AATGTCACACTTT | | UGUCACACUUU |
| BCL11A_ | + | CTTC | 421 | TCATCTGTAATGTCACA | 1732 | UCAUCUGUAAUGUCACACU |
| exon_4 | | | | CTTTTTTGTTTCT | | UUUUUGUUUCU |
| BCL11A_ | + | CTTT | 422 | TTTGTTTCTCTCTTTTT | 1733 | UUUGUUUCUCUCUUUUUUU |
| exon_4 | | | | TTTTTTTTTGAAG | | UUUUUUUGAAG |
| BCL11A_ | + | TTTT | 423 | TTGTTTCTCTCTTTTTT | 1734 | UUGUUUCUCUCUUUUUUUU |
| exon_4 | | | | TTTTTTTTGAAGC | | UUUUUUGAAGC |
| BCL11A_ | + | TTTT | 424 | TGTTTCTCTCTTTTTTT | 1735 | UGUUUCUCUCUUUUUUUUU |
| exon 4 | | | | TTTTTTTGAAGCA | | UUUUUGAAGCA |
| BCL11A_ | + | TTTT | 425 | GTTTCTCTCTTTTTTTT | 1736 | GUUUCUCUCUUUUUUUUUU |
| exon_4 | | | | TTTTTTGAAGCAT | | UUUUGAAGCAU |
| BCL11A_ | + | TTTG | 426 | TTTCTCTCTTTTTTTTT | 1737 | UUUCUCUCUUUUUUUUUUU |
| exon_4 | | | | TTTTTGAAGCATA | | UUUGAAGCAUA |
| BCL11A_ | + | GTTT | 427 | CTCTCTTTTTTTTTTTT | 1738 | CUCUCUUUUUUUUUUUUUU |
| exon_4 | | | | TTGAAGCATACAA | | GAAGCAUACAA |
| BCL11A_ | + | TTTC | 428 | TCTCTTTTTTTTTTTTT | 1739 | UCUCUUUUUUUUUUUUUUG |
| exon_4 | | | | TGAAGCATACAAA | | AAGCAUACAAA |
| BCL11A_ | + | CTTT | 429 | TTTTTTTTTTTGAAGCA | 1740 | UUUUUUUUUUUGAAGCAUA |
| exon_4 | | | | TACAAATAATTTG | | CAAAUAAUUUG |
| BCL11A_ | + | TTTT | 430 | TTTTTTTTTTGAAGCAT | 1741 | UUUUUUUUUUGAAGCAUAC |
| exon_4 | | | | ACAAATAATTTGC | | AAAUAAUUUGC |
| BCL11A_ | + | TTTT | 431 | TTTTTTTTTGAAGCATA | 1742 | UUUUUUUUUGAAGCAUACA |
| exon_4 | | | | CAAATAATTTGCA | | AAUAAUUUGCA |
| BCL11A_ | + | TTTT | 432 | TTTTTTTTGAAGCATAC | 1743 | UUUUUUUUGAAGCAUACAA |
| exon_4 | | | | AAATAATTTGCAC | | AUAAUUUGCAC |
| BCL11A_ | + | TTTT | 433 | TTTTTTTGAAGCATACA | 1744 | UUUUUUUGAAGCAUACAAA |
| exon_4 | | | | AATAATTTGCACT | | UAAUUUGCACU |
| BCL11A_ | + | TTTT | 434 | TTTTTTCCACTACCAAA | 1745 | UUUUUUCCACUACCAAAAA |
| exon_4 | | | | AAAGGTACATTGA | | AGGUACAUUGA |
| BCL11A_ | + | CTTT | 435 | TTTTTTTCCACTACCAA | 1746 | UUUUUUUCCACUACCAAAA |
| exon 4 | | | | AAAAGGTACATTG | | AAGGUACAUUG |
| BCL11A_ | + | ATTA | 436 | AAAAAATATACTGTGGC | 1747 | AAAAAAUAUACUGUGGCAG |
| exon_4 | | | | AGCCTGTCTTTTT | | CCUGUCUUUUU |
| BCL11A_ | + | ATTA | 437 | TCCTGCCAAATTAAAAA | 1748 | UCCUGCCAAAUUAAAAAAA |
| exon 4 | | | | AATATACTGTGGC | | UAUACUGUGGC |
| BCL11A_ | + | TTTG | 438 | CACTATATTATCCTGCC | 1749 | CACUAUAUUAUCCUGCCAA |
| exon 4 | | | | AAATTAAAAAAAT | | AUUAAAAAAAU |
| BCL11A_ | + | ATTT | 439 | GCACTATATTATCCTGC | 1750 | GCACUAUAUUAUCCUGCCA |
| exon 4 | | | | CAAATTAAAAAAA | | AAUUAAAAAAA |
| BCL11A_ | + | GTTA | 440 | TTAGCTTGCAGTACTGC | 1751 | UUAGCUUGCAGUACUGCAU |
| exon_4 | | | | ATACAGTATGGCA | | ACAGUAUGGCA |
| BCL11A_ | + | TTTG | 441 | AAGCATACAAATAATTT | 1752 | AAGCAUACAAAUAAUUUGC |
| exon 4 | | | | GCACTATATTATC | | ACUAUAUUAUC |
| BCL11A_ | + | TTTT | 442 | TGAAGCATACAAATAAT | 1753 | UGAAGCAUACAAAUAAUUU |
| exon_4 | | | | TTGCACTATATTA | | GCACUAUAUUA |
| BCL11A_ | + | TTTT | 443 | TTGAAGCATACAAATAA | 1754 | UUGAAGCAUACAAAUAAUU |
| exon_4 | | | | TTTGCACTATATT | | UGCACUAUAUU |
| BCL11A_ | + | TTTT | 444 | TTTGAAGCATACAAATA | 1755 | UUUGAAGCAUACAAAUAAU |
| exon_4 | | | | ATTTGCACTATAT | | UUGCACUAUAU |
| BCL11A_ | + | TTTT | 445 | TTTTGAAGCATACAAAT | 1756 | UUUUGAAGCAUACAAAUAA |
| exon_4 | | | | AATTTGCACTATA | | UUUGCACUAUA |
| BCL11A_ | + | TTTT | 446 | TTTTTGAAGCATACAAA | 1757 | UUUUUGAAGCAUACAAAUA |
| exon_4 | | | | TAATTTGCACTAT | | AUUUGCACUAU |
| BCL11A_ | + | TTTT | 447 | TTTTTTGAAGCATACAA | 1758 | UUUUUUGAAGCAUACAAAU |
| exon_4 | | | | ATAATTTGCACTA | | AAUUUGCACUA |
| BCL11A_ | + | TTTT | 448 | GAAGCATACAAATAATT | 1759 | GAAGCAUACAAAUAAUUUG |
| exon_4 | | | | TGCACTATATTAT | | CACUAUAUUAU |
| BCL11A_ | + | TTTT | 449 | TTTTCCACTACCAAAAA | 1760 | UUUUCCACUACCAAAAAAG |
| exon_4 | | | | AGGTACATTGATA | | GUACAUUGAUA |
| BCL11A_ | + | TTTA | 450 | CTGCATATGAAGGTAAG | 1761 | CUGCAUAUGAAGGUAAGAU |
| exon_4 | | | | ATGCTGGAATGTA | | GCUGGAAUGUA |
| BCL11A_ | + | CTTT | 451 | TACTGCATATGAAGGTA | 1762 | UACUGCAUAUGAAGGUAAG |
| exon_4 | | | | AGATGCTGGAATG | | AUGCUGGAAUG |
| BCL11A_ | + | GTTT | 452 | TTGGCAATGAAAAAAAC | 1763 | UUGGCAAUGAAAAAAACUG |
| exon_4 | | | | TGCAAAACATTGG | | CAAAACAUUGG |
| BCL11A_ | + | TTTT | 453 | TGGCAATGAAAAAAACT | 1764 | UGGCAAUGAAAAAAACUGC |
| exon_4 | | | | GCAAAACATTGGT | | AAAACAUUGGU |
| BCL11A_ | + | TTTT | 454 | GGCAATGAAAAAAACTG | 1765 | GGCAAUGAAAAAAACUGCA |
| exon_4 | | | | CAAAACATTGGTT | | AAACAUUGGUU |
| BCL11A_ | + | TTTG | 455 | GCAATGAAAAAAACTGC | 1766 | GCAAUGAAAAAAACUGCAA |
| exon_4 | | | | AAAACATTGGTTT | | AACAUUGGUUU |
| BCL11A_ | + | ATTG | 456 | GTTTTTTTTTTTTTTTC | 1767 | GUUUUUUUUUUUCCUUUUU |
| exon_4 | | | | CTTTTTTTTTCTT | | UUUUUUUUCUU |
| BCL11A_ | + | GTTT | 457 | TTTTTTTTTTTTCCTTT | 1768 | UUUUUUUUUUUUCCUUUUU |
| exon_4 | | | | TTTTTTCTTTCTT | | UUUUCUUUCUU |
| BCL11A_ | + | TTTT | 458 | TTTTTTTTTTTCCTTTT | 1769 | UUUUUUUUUUUCCUUUUUU |
| exon_4 | | | | TTTTTCTTTCTTT | | UUUCUUUCUUU |
| BCL11A_ | + | TTTT | 459 | TTTTTTTTTTCCTTTTT | 1770 | UUUUUUUUUUCCUUUUUUU |
| exon_4 | | | | TTTTCTTTCTTTC | | UUCUUUCUUUC |
| BCL11A_ | + | TTTT | 460 | TTTTTTTTTCCTTTTTT | 1771 | UUUUUUUUUCCUUUUUUUU |
| exon_4 | | | | TTTCTTTCTTTCT | | UCUUUCUUUCU |
| BCL11A_ | + | TTTT | 461 | TTTTTTTTCCTTTTTTT | 1772 | UUUUUUUUUUUUCCUUUUU |
| exon_4 | | | | TTCTTTCTTTCTT | | CUUUCUUUCUU |
| BCL11A_ | + | TTTT | 462 | TTTTTTTCCTTTTTTTT | 1773 | UUUUUUUCCUUUUUUUUUC |
| exon_4 | | | | TCTTTCTTTCTTT | | UUUCUUUCUUU |
| BCL11A_ | + | TTTT | 463 | TTTTTTCCTTTTTTTTT | 1774 | UUUUUUCCUUUUUUUUUCU |
| exon_4 | | | | CTTTCTTTCTTTT | | UUCUUUCUUUU |
| BCL11A_ | + | TTTT | 464 | TTTTTCCTTTTTTTTTC | 1775 | UUUUUCCUUUUUUUUUCUU |
| exon 4 | | | | TTTCTTTCTTTTA | | UCUUUCUUUUA |
| BCL11A_ | + | TTTT | 465 | TTTTCCTTTTTTTTTCT | 1776 | UUUUCCUUUUUUUUUCUUU |
| exon_4 | | | | TTCTTTCTTTTAC | | CUUUCUUUUAC |
| BCL11A_ | + | TTTT | 466 | TTTCCTTTTTTTTTCTT | 1777 | UUUCCUUUUUUUUUCUUUC |
| exon_4 | | | | TCTTTCTTTTACT | | UUUCUUUUACU |
| BCL11A_ | + | TTTT | 467 | TTCCTTTTTTTTTCTTT | 1778 | UUCCUUUUUUUUUCUUUCU |
| exon_4 | | | | CTTTCTTTTACTG | | UUCUUUUACUG |
| BCL11A_ | + | TTTT | 468 | TCCTTTTTTTTTCTTTC | 1779 | UCCUUUUUUUUUCUUUCUU |
| exon_4 | | | | TTTCTTTTACTGC | | UCUUUUACUGC |
| BCL11A_ | + | TTTC | 469 | TTTTACTGCATATGAAG | 1780 | UUUUACUGCAUAUGAAGGU |
| exon_4 | | | | GTAAGATGCTGGA | | AAGAUGCUGGA |
| BCL11A_ | + | CTTT | 470 | CTTTTACTGCATATGAA | 1781 | CUUUUACUGCAUAUGAAGG |
| exon 4 | | | | GGTAAGATGCTGG | | UAAGAUGCUGG |
| BCL11A_ | + | TTTC | 471 | TTTCTTTTACTGCATAT | 1782 | UUUCUUUUACUGCAUAUGA |
| exon_4 | | | | GAAGGTAAGATGC | | AGGUAAGAUGC |
| BCL11A_ | + | CTTT | 472 | CTTTCTTTTACTGCATA | 1783 | CUUUCUUUUACUGCAUAUG |
| exon_4 | | | | TGAAGGTAAGATG | | AAGGUAAGAUG |
| BCL11A_ | + | TTTC | 473 | TTTCTTTCTTTTACTGC | 1784 | UUUCUUUCUUUUACUGCAU |
| exon_4 | | | | ATATGAAGGTAAG | | AUGAAGGUAAG |
| BCL11A_ | + | TTTT | 474 | CTTTCTTTCTTTTACTG | 1785 | CUUUCUUUCUUUUACUGCA |
| exon 4 | | | | CATATGAAGGTAA | | UAUGAAGGUAA |
| BCL11A_ | + | TTTT | 475 | ACTGCATATGAAGGTAA | 1786 | ACUGCAUAUGAAGGUAAGA |
| exon_4 | | | | GATGCTGGAATGT | | UGCUGGAAUGU |
| BCL11A_ | + | TTTT | 476 | TCTTTCTTTCTTTTACT | 1787 | UCUUUCUUUCUUUUACUGC |
| exon_4 | | | | GCATATGAAGGTA | | AUAUGAAGGUA |
| BCL11A_ | + | TTTT | 477 | TTTCTTTCTTTCTTTTA | 1788 | UUUCUUUCUUUCUUUUACU |
| exon_4 | | | | CTGCATATGAAGG | | GCAUAUGAAGG |
| BCL11A_ | + | TTTT | 478 | TTTTCTTTCTTTCTTTT | 1789 | UUUUCUUUCUUUCUUUUAC |
| exon_4 | | | | ACTGCATATGAAG | | UGCAUAUGAAG |
| BCL11A_ | + | TTTT | 479 | TTTTTCTTTCTTTCTTT | 1790 | UUUUUCUUUCUUUCUUUUA |
| exon_4 | | | | TACTGCATATGAA | | CUGCAUAUGAA |
| BCL11A_ | + | CTTT | 480 | TTTTTTCTTTCTTTCTT | 1791 | UUUUUUUUUCUUUCUUUU |
| exon_4 | | | | TTACTGCATATGA | | ACUGCAUAUGA |
| BCL11A_ | + | TTTC | 481 | CTTTTTTTTTCTTTCTT | 1792 | CUUUUUUUUUCUUUCUUUC |
| exon_4 | | | | TCTTTTACTGCAT | | UUUUACUGCAU |
| BCL11A_ | + | TTTT | 482 | CCTTTTTTTTTCTTTCT | 1793 | CCUUUUUUUUUCUUUCUUU |
| exon 4 | | | | TTCTTTTACTGCA | | CUUUUACUGCA |
| BCL11A_ | + | TTTT | 483 | TTCTTTCTTTCTTTTAC | 1794 | UUCUUUCUUUCUUUUACUG |
| exon 4 | | | | TGCATATGAAGGT | | CAUAUGAAGGU |
| BCL11A_ | + | ATTG | 484 | TATCAATATTAGCTTAT | 1795 | UAUCAAUAUUAGCUUAUAU |
| exon 4 | | | | ATACCTGTTCTAG | | ACCUGUUCUAG |
| BCL11A_ | + | ATTC | 485 | AAGGCCTTTTTTCTTCC | 1796 | AAGGCCUUUUUUCUUCCUU |
| exon 4 | | | | TTTCCAATTGATA | | UCCAAUUGAUA |
| BCL11A_ | + | CTTA | 486 | TATACCTGTTCTAGTTT | 1797 | UAUACCUGUUCUAGUUUUA |
| exon_4 | | | | TAAATGGCAAATA | | AAUGGCAAAUA |
| BCL11A_ | + | TTTC | 487 | ATGTGTTTCTCCAGGGT | 1798 | AUGUGUUUCUCCAGGGUAC |
| exon_4 | | | | ACTGTACACGCTA | | UGUACACGCUA |
| BCL11A_ | + | GTTT | 488 | CTCCAGGGTACTGTACA | 1799 | CUCCAGGGUACUGUACACG |
| exon_4 | | | | CGCTAAAAGGCAT | | CUAAAAGGCAU |
| BCL11A_ | + | TTTC | 489 | TCCAGGGTACTGTACAC | 1800 | UCCAGGGUACUGUACACGC |
| exon_4 | | | | GCTAAAAGGCATC | | UAAAAGGCAUC |
| BCL11A_ | + | CTTA | 490 | CAAATTTCACATTTGTA | 1801 | CAAAUUUCACAUUUGUAAA |
| exon_4 | | | | AACGTCCTTCCCC | | CGUCCUUCCCC |
| BCL11A_ | + | ATTT | 491 | CACATTTGTAAACGTCC | 1802 | CACAUUUGUAAACGUCCUU |
| exon_4 | | | | TTCCCCACCTGGC | | CCCCACCUGGC |
| BCL11A_ | + | TTTC | 492 | ACATTTGTAAACGTCCT | 1803 | ACAUUUGUAAACGUCCUUC |
| exon_4 | | | | TCCCCACCTGGCC | | CCCACCUGGCC |
| BCL11A_ | + | ATTT | 493 | GTAAACGTCCTTCCCCA | 1804 | GUAAACGUCCUUCCCCACC |
| exon 4 | | | | CCTGGCCATGCGT | | UGGCCAUGCGU |
| BCL11A_ | + | TTTG | 494 | TAAACGTCCTTCCCCAC | 1805 | UAAACGUCCUUCCCCACCU |
| exon_4 | | | | CTGGCCATGCGTT | | GGCCAUGCGUU |
| BCL11A_ | + | CTTC | 495 | CCCACCTGGCCATGCGT | 1806 | CCCACCUGGCCAUGCGUUU |
| exon 4 | | | | TTTCATGTGCCTG | | UCAUGUGCCUG |
| BCL11A_ | + | GTTT | 496 | TCATGTGCCTGGTGAGC | 1807 | UCAUGUGCCUGGUGAGCUU |
| exon_4 | | | | TTGCTACTCTGGG | | GCUACUCUGGG |
| BCL11A_ | + | TTTT | 497 | CATGTGCCTGGTGAGCT | 1808 | CAUGUGCCUGGUGAGCUUG |
| exon_4 | | | | TGCTACTCTGGGC | | CUACUCUGGGC |
| BCL11A_ | + | TTTC | 498 | ATGTGCCTGGTGAGCTT | 1809 | AUGUGCCUGGUGAGCUUGC |
| exon_4 | | | | GCTACTCTGGGCA | | UACUCUGGGCA |
| BCL11A_ | + | CTTG | 499 | CTACTCTGGGCACAGGC | 1810 | CUACUCUGGGCACAGGCAU |
| exon 4 | | | | ATAGTTGCACAGC | | AGUUGCACAGC |
| BCL11A_ | + | GTTG | 500 | CACAGCTCGCATTTATA | 1811 | CACAGCUCGCAUUUAUAAG |
| exon_4 | | | | AGGCCTTTCGCCC | | GCCUUUCGCCC |
| BCL11A_ | + | TTTT | 501 | CATGTGTTTCTCCAGGG | 1812 | CAUGUGUUUCUCCAGGGUA |
| exon_4 | | | | TACTGTACACGCT | | CUGUACACGCU |
| BCL11A_ | + | ATTT | 502 | ATAAGGCCTTTCGCCCG | 1813 | AUAAGGCCUUUCGCCCGUG |
| exon_4 | | | | TGTGGCTTCTCCT | | UGGCUUCUCCU |
| BCL11A_ | + | CTTT | 503 | CGCCCGTGTGGCTTCTC | 1814 | CGCCCGUGUGGCUUCUCCU |
| exon_4 | | | | CTGTGGACAGTGA | | GUGGACAGUGA |
| BCL11A_ | + | TTTC | 504 | GCCCGTGTGGCTTCTCC | 1815 | GCCCGUGUGGCUUCUCCUG |
| exon 4 | | | | TGTGGACAGTGAG | | UGGACAGUGAG |
| BCL11A_ | + | CTTC | 505 | TCCTGTGGACAGTGAGA | 1816 | UCCUGUGGACAGUGAGAUU |
| exon_4 | | | | TTGCTACAGTTCT | | GCUACAGUUCU |
| BCL11A_ | + | ATTG | 506 | CTACAGTTCTTGAAGAC | 1817 | CUACAGUUCUUGAAGACUU |
| exon_4 | | | | TTTCCCACAGTAC | | UCCCACAGUAC |
| BCL11A_ | + | GTTC | 507 | TTGAAGACTTTCCCACA | 1818 | UUGAAGACUUUCCCACAGU |
| exon_4 | | | | GTACTCACAAGTG | | ACUCACAAGUG |
| BCL11A_ | + | CTTG | 508 | AAGACTTTCCCACAGTA | 1819 | AAGACUUUCCCACAGUACU |
| exon 4 | | | | CTCACAAGTGTCG | | CACAAGUGUCG |
| BCL11A_ | + | CTTT | 509 | CCCACAGTACTCACAAG | 1820 | CCCACAGUACUCACAAGUG |
| exon_4 | | | | TGTCGCTGCGTCT | | UCGCUGCGUCU |
| BCL11A_ | + | TTTC | 510 | CCACAGTACTCACAAGT | 1821 | CCACAGUACUCACAAGUGU |
| exon_4 | | | | GTCGCTGCGTCTG | | CGCUGCGUCUG |
| BCL11A_ | + | CTTT | 511 | TGAGCTGGGCCTGCCCG | 1822 | UGAGCUGGGCCUGCCCGGG |
| exon_4 | | | | GGCCCGGACCACT | | CCCGGACCACU |
| BCL11A_ | + | TTTT | 512 | GAGCTGGGCCTGCCCGG | 1823 | GAGCUGGGCCUGCCCGGGC |
| exon_4 | | | | GCCCGGACCACTA | | CCGGACCACUA |
| BCL11A_ | + | TTTG | 513 | AGCTGGGCCTGCCCGGG | 1824 | AGCUGGGCCUGCCCGGGCC |
| exon 4 | | | | CCCGGACCACTAA | | CGGACCACUAA |
| BCL11A_ | + | CTTC | 514 | CCGTGCCGCTGCGCCCC | 1825 | CCGUGCCGCUGCGCCCCGA |
| exon 4 | | | | GAGATCCCTCCGT | | GAUCCCUCCGU |
| BCL11A_ | + | GTTC | 515 | TCCGAGGAGTGCTCCGA | 1826 | UCCGAGGAGUGCUCCGACG |
| exon_4 | | | | CGAGGAGGCAAAA | | AGGAGGCAAAA |
| BCL11A_ | + | ATTG | 516 | TCTGGAGTCTCCGAAGC | 1827 | UCUGGAGUCUCCGAAGCUA |
| exon_4 | | | | TAAGGAAGGGATC | | AGGAAGGGAUC |
| BCL11A_ | + | TTTA | 517 | TAAGGCCTTTCGCCCGT | 1828 | UAAGGCCUUUCGCCCGUGU |
| exon_4 | | | | GTGGCTTCTCCTG | | GGCUUCUCCUG |
| BCL11A_ | + | TTTT | 518 | TCATGTGTTTCTCCAGG | 1829 | UCAUGUGUUUCUCCAGGGU |
| exon_4 | | | | GTACTGTACACGC | | ACUGUACACGC |
| BCL11A_ | + | TTTT | 519 | TTCATGTGTTTCTCCAG | 1830 | UUCAUGUGUUUCUCCAGGG |
| exon_4 | | | | GGTACTGTACACG | | UACUGUACACG |
| BCL11A_ | + | ATTT | 520 | TTTCATGTGTTTCTCCA | 1831 | UUUCAUGUGUUUCUCCAGG |
| exon_4 | | | | GGGTACTGTACAC | | GUACUGUACAC |
| BCL11A_ | + | GTTT | 521 | AAAAAAAAACATACACA | 1832 | AAAAAAAAACAUACACAAC |
| exon_4 | | | | ACATGTAAATTAT | | AUGUAAAUUAU |
| BCL11A_ | + | TTTA | 522 | AAAAAAAACATACACAA | 1833 | AAAAAAAACAUACACAACA |
| exon 4 | | | | CATGTAAATTATT | | UGUAAAUUAUU |
| BCL11A_ | + | ATTA | 523 | TTGCACAAGAGAAAGGC | 1834 | UUGCACAAGAGAAAGGCUC |
| exon_4 | | | | TCAAAGTTTGCGT | | AAAGUUUGCGU |
| BCL11A_ | + | ATTG | 524 | CACAAGAGAAAGGCTCA | 1835 | CACAAGAGAAAGGCUCAAA |
| exon_4 | | | | AAGTTTGCGTAAA | | GUUUGCGUAAA |
| BCL11A_ | + | GTTT | 525 | GCGTAAAATGCAATAGT | 1836 | GCGUAAAAUGCAAUAGUAU |
| exon_4 | | | | ATTGCCCCATACA | | UGCCCCAUACA |
| BCL11A_ | + | TTTG | 526 | CGTAAAATGCAATAGTA | 1837 | CGUAAAAUGCAAUAGUAUU |
| exon_4 | | | | TTGCCCCATACAG | | GCCCCAUACAG |
| BCL11A_ | + | ATTG | 527 | CCCCATACAGATCATGC | 1838 | CCCCAUACAGAUCAUGCAU |
| exon 4 | | | | ATTCAAACGGTGA | | UCAAACGGUGA |
| BCL11A_ | + | ATTC | 528 | AAACGGTGAGAACATAA | 1839 | AAACGGUGAGAACAUAAAG |
| exon_4 | | | | AGGAAAAAAAAAA | | GAAAAAAAAAA |
| BCL11A_ | + | ATTC | 529 | TTAGCTTCGTTACTTCT | 1840 | UUAGCUUCGUUACUUCUGU |
| exon_4 | | | | GTTTGTTTGTTTG | | UUGUUUGUUUG |
| BCL11A_ | + | CTTA | 530 | GCTTCGTTACTTCTGTT | 1841 | GCUUCGUUACUUCUGUUUG |
| exon_4 | | | | TGTTTGTTTGTTT | | UUUGUUUGUUU |
| BCL11A_ | + | CTTC | 531 | GTTACTTCTGTTTGTTT | 1842 | GUUACUUCUGUUUGUUUGU |
| exon_4 | | | | GTTTGTTTGTTTA | | UUGUUUGUUUA |
| BCL11A_ | + | GTTA | 532 | CTTCTGTTTGTTTGTTT | 1843 | CUUCUGUUUGUUUGUUUGU |
| exon_4 | | | | GTTTGTTTAAATC | | UUGUUUAAAUC |
| BCL11A_ | + | CTTC | 533 | TGTTTGTTTGTTTGTTT | 1844 | UGUUUGUUUGUUUGUUUGU |
| exon_4 | | | | GTTTAAATCACAT | | UUAAAUCACAU |
| BCL11A_ | + | GTTT | 534 | GTTTGTTTGTTTGTTTA | 1845 | GUUUGUUUGUUUGUUUAAA |
| exon_4 | | | | AATCACATGGGAC | | UCACAUGGGAC |
| BCL11A_ | + | TTTG | 535 | TTTGTTTGTTTGTTTAA | 1846 | UUUGUUUGUUUGUUUAAAU |
| exon_4 | | | | ATCACATGGGACT | | CACAUGGGACU |
| BCL11A_ | + | GTTT | 536 | GTTTGTTTGTTTAAATC | 1847 | GUUUGUUUGUUUAAAUCAC |
| exon_4 | | | | ACATGGGACTAGA | | AUGGGACUAGA |
| BCL11A_ | + | TTTG | 537 | TTTGTTTGTTTAAATCA | 1848 | UUUGUUUGUUUAAAUCACA |
| exon_4 | | | | CATGGGACTAGAA | | UGGGACUAGAA |
| BCL11A_ | + | ATTC | 538 | AACACTCGATCACTGTG | 1849 | AACACUCGAUCACUGUGCC |
| exon_4 | | | | CCATTTTTTCATG | | AUUUUUUCAUG |
| BCL11A_ | + | ATTA | 539 | TTCAACACTCGATCACT | 1850 | UUCAACACUCGAUCACUGU |
| exon_4 | | | | GTGCCATTTTTTC | | GCCAUUUUUUC |
| BCL11A_ | + | TTTA | 540 | TATCATTATTCAACACT | 1851 | UAUCAUUAUUCAACACUCG |
| exon_4 | | | | CGATCACTGTGCC | | AUCACUGUGCC |
| BCL11A_ | + | TTTT | 541 | ATATCATTATTCAACAC | 1852 | AUAUCAUUAUUCAACACUC |
| exon 4 | | | | TCGATCACTGTGC | | GAUCACUGUGC |
| BCL11A_ | + | TTTT | 542 | TATATCATTATTCAACA | 1853 | UAUAUCAUUAUUCAACACU |
| exon_4 | | | | CTCGATCACTGTG | | CGAUCACUGUG |
| BCL11A_ | + | GTTT | 543 | TTATATCATTATTCAAC | 1854 | UUAUAUCAUUAUUCAACAC |
| exon 4 | | | | ACTCGATCACTGT | | UCGAUCACUGU |
| BCL11A_ | + | CTTT | 544 | GAGCTGCCTGGAGGCCG | 1855 | GAGCUGCCUGGAGGCCGCG |
| exon_4 | | | | CGTAGCCGGCGAG | | UAGCCGGCGAG |
| BCL11A_ | + | ATTC | 545 | AGTTTTTATATCATTAT | 1856 | AGUUUUUAUAUCAUUAUUC |
| exon_4 | | | | TCAACACTCGATC | | AACACUCGAUC |
| BCL11A_ | + | TTTA | 546 | AATCACATGGGACTAGA | 1857 | AAUCACAUGGGACUAGAAA |
| exon_4 | | | | AAAAAATCCTACA | | AAAAUCCUACA |
| BCL11A_ | + | GTTT | 547 | AAATCACATGGGACTAG | 1858 | AAAUCACAUGGGACUAGAA |
| exon_4 | | | | AAAAAAATCCTAC | | AAAAAUCCUAC |
| BCL11A_ | + | TTTG | 548 | TTTAAATCACATGGGAC | 1859 | UUUAAAUCACAUGGGACUA |
| exon_4 | | | | TAGAAAAAAATCC | | GAAAAAAAUCC |
| BCL11A_ | − | GTTT | 549 | GTTTAAATCACATGGGA | 1860 | GUUUAAAUCACAUGGGACU |
| exon_4 | | | | CTAGAAAAAAATC | | AGAAAAAAAUC |
| BCL11A_ | + | TTTG | 550 | TTTGTTTAAATCACATG | 1861 | UUUGUUUAAAUCACAUGGG |
| exon 4 | | | | GGACTAGAAAAAA | | ACUAGAAAAAA |
| BCL11A_ | + | GTTT | 551 | GTTTGTTTAAATCACAT | 1862 | GUUUGUUUAAAUCACAUGG |
| exon 4 | | | | GGGACTAGAAAAA | | GACUAGAAAAA |
| BCL11A_ | + | ATTA | 552 | ATATACCTCTATTCAGT | 1863 | AUAUACCUCUAUUCAGUUU |
| exon_4 | | | | TTTTATATCATTA | | UUAUAUCAUUA |
| BCL11A_ | + | TTTG | 553 | AGCTGCCTGGAGGCCGC | 1864 | AGCUGCCUGGAGGCCGCGU |
| exon 4 | | | | GTAGCCGGCGAGC | | AGCCGGCGAGC |
| BCL11A_ | + | GTTC | 554 | TCCGTGTTGGGCATCGC | 1865 | UCCGUGUUGGGCAUCGCGG |
| exon_4 | | | | GGCCGGGGGCAGG | | CCGGGGGCAGG |
| BCL11A_ | + | GTTG | 555 | GGCATCGCGGCCGGGGG | 1866 | GGCAUCGCGGCCGGGGGCA |
| exon_4 | | | | CAGGTCGAACTCC | | GGUCGAACUCC |
| BCL11A_ | + | TTTG | 556 | AACGTCTTGCCGCAGAA | 1867 | AACGUCUUGCCGCAGAACU |
| exon_4 | | | | CTCGCATGACTTG | | CGCAUGACUUG |
| BCL11A_ | + | CTTG | 557 | CCGCAGAACTCGCATGA | 1868 | CCGCAGAACUCGCAUGACU |
| exon_4 | | | | CTTGGACTTGACC | | UGGACUUGACC |
| BCL11A_ | + | CTTG | 558 | GACTTGACCGGGGGCTG | 1869 | GACUUGACCGGGGGCUGGG |
| exon_4 | | | | GGAGGGAGGAGGG | | AGGGAGGAGGG |
| BCL11A_ | + | CTTG | 559 | ACCGGGGGCTGGGAGGG | 1870 | ACCGGGGGCUGGGAGGGAG |
| exon 4 | | | | AGGAGGGGCGGAT | | GAGGGGCGGAU |
| BCL11A_ | + | ATTG | 560 | CAGAGGAGGGAGGGGGG | 1871 | CAGAGGAGGGAGGGGGGGC |
| exon 4 | | | | GCGTCGCCAGGAA | | GUCGCCAGGAA |
| BCL11A_ | + | CTTG | 561 | CTACCTGGCTGGAATGG | 1872 | CUACCUGGCUGGAAUGGUU |
| exon 4 | | | | TTGCAGTAACCTT | | GCAGUAACCUU |
| BCL11A_ | + | GTTG | 562 | CAGTAACCTTTGCATAG | 1873 | CAGUAACCUUUGCAUAGGG |
| exon_4 | | | | GGCTGGGCCGGCC | | CUGGGCCGGCC |
| BCL11A_ | + | CTTT | 563 | GCATAGGGCTGGGCCGG | 1874 | GCAUAGGGCUGGGCCGGCC |
| exon_4 | | | | CCTGGGGACAGCG | | UGGGGACAGCG |
| BCL11A_ | + | TTTG | 564 | CATAGGGCTGGGCCGGC | 1875 | CAUAGGGCUGGGCCGGCCU |
| exon_4 | | | | CTGGGGACAGCGG | | GGGGACAGCGG |
| BCL11A_ | + | GTTC | 565 | CCTGCCAGCTCTCTAAG | 1876 | CCUGCCAGCUCUCUAAGUC |
| exon_4 | | | | TCTCCTAGAGAAA | | UCCUAGAGAAA |
| BCL11A_ | + | ATTG | 566 | GATTCAACCGCAGCACC | 1877 | GAUUCAACCGCAGCACCCU |
| exon_4 | | | | CTGTCAAAGGCAC | | GUCAAAGGCAC |
| BCL11A_ | + | ATTC | 567 | AACCGCAGCACCCTGTC | 1878 | AACCGCAGCACCCUGUCAA |
| exon_4 | | | | AAAGGCACTCGGG | | AGGCACUCGGG |
| BCL11A_ | + | CTTC | 568 | CGCCCCCAGGCGCTCTA | 1879 | CGCCCCCAGGCGCUCUAUG |
| exon_4 | | | | TGCGGTGGGGGTC | | CGGUGGGGGUC |
| BCL11A_ | + | CTTC | 569 | TGCCAGGCCGGAAGCCT | 1880 | UGCCAGGCCGGAAGCCUCU |
| exon_4 | | | | CTCTCGATACTGA | | CUCGAUACUGA |
| BCL11A_ | + | ATTC | 570 | TTAGCAGGTTAAAGGGG | 1881 | UUAGCAGGUUAAAGGGGUU |
| exon_4 | | | | TTATTGTCTGCAA | | AUUGUCUGCAA |
| BCL11A_ | + | CTTA | 571 | GCAGGTTAAAGGGGTTA | 1882 | GCAGGUUAAAGGGGUUAUU |
| exon_4 | | | | TTGTCTGCAATAT | | GUCUGCAAUAU |
| BCL11A_ | + | GTTA | 572 | AAGGGGTTATTGTCTGC | 1883 | AAGGGGUUAUUGUCUGCAA |
| exon 4 | | | | AATATGAATCCCA | | UAUGAAUCCCA |
| BCL11A_ | + | GTTG | 573 | TACATGTGTAGCTGCTG | 1884 | UACAUGUGUAGCUGCUGGG |
| exon_4 | | | | GGCTCATCTTTAC | | CUCAUCUUUAC |
| BCL11A_ | + | TTTG | 574 | CAAGTTGTACATGTGTA | 1885 | CAAGUUGUACAUGUGUAGC |
| exon_4 | | | | GCTGCTGGGCTCA | | UGCUGGGCUCA |
| BCL11A_ | + | GTTT | 575 | GCAAGTTGTACATGTGT | 1886 | GCAAGUUGUACAUGUGUAG |
| exon_4 | | | | AGCTGCTGGGCTC | | CUGCUGGGCUC |
| BCL11A_ | + | GTTG | 576 | CAAGAGAAACCATGCAC | 1887 | CAAGAGAAACCAUGCACUG |
| exon 4 | | | | TGGTGAATGGCTG | | GUGAAUGGCUG |
| BCL11A_ | + | GTTC | 577 | TGTGCGTGTTGCAAGAG | 1888 | UGUGCGUGUUGCAAGAGAA |
| exon_4 | | | | AAACCATGCACTG | | ACCAUGCACUG |
| BCL11A_ | + | CTTA | 578 | ATCCATGAGTGTTCTGT | 1889 | AUCCAUGAGUGUUCUGUGC |
| exon_4 | | | | GCGTGTTGCAAGA | | GUGUUGCAAGA |
| BCL11A_ | + | ATTT | 579 | GAACGTCTTGCCGCAGA | 1890 | GAACGUCUUGCCGCAGAAC |
| exon_4 | | | | ACTCGCATGACTT | | UCGCAUGACUU |
| BCL11A_ | + | ATTC | 580 | TTAATCCATGAGTGTTC | 1891 | UUAAUCCAUGAGUGUUCUG |
| exon 4 | | | | TGTGCGTGTTGCA | | UGCGUGUUGCA |
| BCL11A_ | + | CTTT | 581 | CTAAGTAGATTCTTAAT | 1892 | CUAAGUAGAUUCUUAAUCC |
| exon 4 | | | | CCATGAGTGTTCT | | AUGAGUGUUCU |
| BCL11A_ | + | GTTC | 582 | GCTTTCTAAGTAGATTC | 1893 | GCUUUCUAAGUAGAUUCUU |
| exon_4 | | | | TTAATCCATGAGT | | AAUCCAUGAGU |
| BCL11A_ | + | CTTC | 583 | CGTGTTCGCTTTCTAAG | 1894 | CGUGUUCGCUUUCUAAGUA |
| exon_4 | | | | TAGATTCTTAATC | | GAUUCUUAAUC |
| BCL11A_ | + | ATTC | 584 | TGCACCTAGTCCTGAAG | 1895 | UGCACCUAGUCCUGAAGGG |
| exon_4 | | | | GGATACCAACCCG | | AUACCAACCCG |
| BCL11A_ | + | ATTG | 585 | TCTGCAATATGAATCCC | 1896 | UCUGCAAUAUGAAUCCCAU |
| exon_4 | | | | ATGGAGAGGTGGC | | GGAGAGGUGGC |
| BCL11A_ | + | GTTA | 586 | TTGTCTGCAATATGAAT | 1897 | UUGUCUGCAAUAUGAAUCC |
| exon_4 | | | | CCCATGGAGAGGT | | CAUGGAGAGGU |
| BCL11A_ | + | TTTC | 587 | TAAGTAGATTCTTAATC | 1898 | UAAGUAGAUUCUUAAUCCA |
| exon_4 | | | | CATGAGTGTTCTG | | UGAGUGUUCUG |
| BCL11A_ | + | TTTA | 588 | AAATAGCCATAACATAC | 1899 | AAAUAGCCAUAACAUACCA |
| exon_4 | | | | CATACATGCTGTC | | UACAUGCUGUC |
| BCL11A_ | + | GTTG | 589 | CTCTGAAATTTGAACGT | 1900 | CUCUGAAAUUUGAACGUCU |
| exon_4 | | | | CTTGCCGCAGAAC | | UGCCGCAGAAC |
| BCL11A_ | + | CTTG | 590 | TAGGGCTTCTCGCCCGT | 1901 | UAGGGCUUCUCGCCCGUGU |
| exon_4 | | | | GTGGCTGCGCCGG | | GGCUGCGCCGG |
| BCL11A_ | + | CTTC | 591 | TCGAGCTTGATGCGCTT | 1902 | UCGAGCUUGAUGCGCUUAG |
| exon_4 | | | | AGAGAAGGGGCTC | | AGAAGGGGCUC |
| BCL11A_ | + | CTTG | 592 | ATGCGCTTAGAGAAGGG | 1903 | AUGCGCUUAGAGAAGGGGC |
| exon_4 | | | | GCTCAGCGAGCTG | | UCAGCGAGCUG |
| BCL11A_ | + | CTTA | 593 | GAGAAGGGGCTCAGCGA | 1904 | GAGAAGGGGCUCAGCGAGC |
| exon_4 | | | | GCTGGGGCTGCCC | | UGGGGCUGCCC |
| BCL11A_ | + | CTTT | 594 | TTGGACAGGCCCCCCGA | 1905 | UUGGACAGGCCCCCCGAGG |
| exon_4 | | | | GGCCGACTCGCCC | | CCGACUCGCCC |
| BCL11A_ | + | TTTT | 595 | TGGACAGGCCCCCCGAG | 1906 | UGGACAGGCCCCCCGAGGC |
| exon_4 | | | | GCCGACTCGCCCG | | CGACUCGCCCG |
| BCL11A_ | + | TTTT | 596 | GGACAGGCCCCCCGAGG | 1907 | GGACAGGCCCCCCGAGGCC |
| exon 4 | | | | CCGACTCGCCCGG | | GACUCGCCCGG |
| BCL11A_ | + | TTTG | 597 | GACAGGCCCCCCGAGGC | 1908 | GACAGGCCCCCCGAGGCCG |
| exon_4 | | | | CGACTCGCCCGGG | | ACUCGCCCGGG |
| BCL11A_ | + | ATTA | 598 | ACAGTGCCATCGTCTAT | 1909 | ACAGUGCCAUCGUCUAUGC |
| exon 4 | | | | GCGGTCCGACTCG | | GGUCCGACUCG |
| BCL11A_ | + | CTTC | 599 | GTCGCAAGTGTCCCTGT | 1910 | GUCGCAAGUGUCCCUGUGG |
| exon_4 | | | | GGCCCTCGGCCTC | | CCCUCGGCCUC |
| BCL11A_ | + | CTTA | 600 | TGCTTCTCGCCCAGGAC | 1911 | UGCUUCUCGCCCAGGACCU |
| exon_4 | | | | CTGGTGGAAGGCC | | GGUGGAAGGCC |
| BCL11A_ | + | CTTC | 601 | TCGCCCAGGACCTGGTG | 1912 | UCGCCCAGGACCUGGUGGA |
| exon_4 | | | | GAAGGCCTCGCTG | | AGGCCUCGCUG |
| BCL11A_ | + | GTTC | 602 | TCGTGGTGGCGCGCCGC | 1913 | UCGUGGUGGCGCGCCGCCU |
| exon_4 | | | | CTCCAGGCTCAGC | | CCAGGCUCAGC |
| BCL11A_ | + | CTTC | 603 | CTCCTCTTCTTCCTCTT | 1914 | CUCCUCUUCUUCCUCUUCC |
| exon_4 | | | | CCTCGTCGTCCTC | | UCGUCGUCCUC |
| BCL11A_ | + | CTTC | 604 | TTCCTCTTCCTCGTCGT | 1915 | UUCCUCUUCCUCGUCGUCC |
| exon_4 | | | | CCTCCTCTTCCTC | | UCCUCUUCCUC |
| BCL11A_ | + | CTTC | 605 | CTCTTCCTCGTCGTCCT | 1916 | CUCUUCCUCGUCGUCCUCC |
| exon_4 | | | | CCTCTTCCTCCTC | | UCUUCCUCCUC |
| BCL11A_ | + | CTTC | 606 | CTCGTCGTCCTCCTCTT | 1917 | CUCGUCGUCCUCCUCUUCC |
| exon_4 | | | | CCTCCTCGTCCCC | | UCCUCGUCCCC |
| BCL11A_ | + | CTTC | 607 | CTCCTCGTCCCCGTTCT | 1918 | CUCCUCGUCCCCGUUCUCC |
| exon_4 | | | | CCGGGATCAGGTT | | GGGAUCAGGUU |
| BCL11A_ | + | GTTG | 608 | CACTTGTAGGGCTTCTC | 1919 | CACUUGUAGGGCUUCUCGC |
| exon_4 | | | | GCCCGTGTGGCTG | | CCGUGUGGCUG |
| BCL11A_ | + | CTTG | 609 | CTGGCCTGGGTGCACGC | 1920 | CUGGCCUGGGUGCACGCGU |
| exon 4 | | | | GTGGTCGCACAGG | | GGUCGCACAGG |
| BCL11A_ | + | CTTC | 610 | AGCTTGCTGGCCTGGGT | 1921 | AGCUUGCUGGCCUGGGUGC |
| exon_4 | | | | GCACGCGTGGTCG | | ACGCGUGGUCG |
| BCL11A_ | + | CTTC | 611 | ATGTGGCGCTTCAGCTT | 1922 | AUGUGGCGCUUCAGCUUGC |
| exon_4 | | | | GCTGGCCTGGGTG | | UGGCCUGGGUG |
| BCL11A_ | + | TTTG | 612 | TGCATGTGCGTCTTCAT | 1923 | UGCAUGUGCGUCUUCAUGU |
| exon_4 | | | | GTGGCGCTTCAGC | | GGCGCUUCAGC |
| BCL11A_ | + | ATTT | 613 | GTGCATGTGCGTCTTCA | 1924 | GUGCAUGUGCGUCUUCAUG |
| exon_4 | | | | TGTGGCGCTTCAG | | UGGCGCUUCAG |
| BCL11A_ | + | CTTC | 614 | TCGCCCGTGTGGCTGCG | 1925 | UCGCCCGUGUGGCUGCGCC |
| exon_4 | | | | CCGGTGCACCACC | | GGUGCACCACC |
| BCL11A_ | + | CTTG | 615 | ACCGTCATGGGGGACGA | 1926 | ACCGUCAUGGGGGACGAUU |
| exon_4 | | | | TTTGTGCATGTGC | | UGUGCAUGUGC |
| BCL11A_ | + | CTTG | 616 | AGCGCGCTGCTGGCGCT | 1927 | AGCGCGCUGCUGGCGCUGC |
| exon_4 | | | | GCCCACCAAGTCG | | CCACCAAGUCG |
| BCL11A_ | + | CTTG | 617 | GCCACCACGGACTTGAG | 1928 | GCCACCACGGACUUGAGCG |
| exon_4 | | | | CGCGCTGCTGGCG | | CGCUGCUGGCG |
| BCL11A_ | + | CTTG | 618 | AACTTGGCCACCACGGA | 1929 | AACUUGGCCACCACGGACU |
| exon_4 | | | | CTTGAGCGCGCTG | | UGAGCGCGCUG |
| BCL11A_ | + | GTTC | 619 | TCGCTCTTGAACTTGGC | 1930 | UCGCUCUUGAACUUGGCCA |
| exon_4 | | | | CACCACGGACTTG | | CCACGGACUUG |
| BCL11A_ | + | GTTG | 620 | GGGTCGTTCTCGCTCTT | 1931 | GGGUCGUUCUCGCUCUUGA |
| exon_4 | | | | GAACTTGGCCACC | | ACUUGGCCACC |
| BCL11A_ | + | GTTC | 621 | TCCGGGATCAGGTTGGG | 1932 | UCCGGGAUCAGGUUGGGGU |
| exon_4 | | | | GTCGTTCTCGCTC | | CGUUCUCGCUC |
| BCL11A_ | + | GTTC | 622 | CGGGGAGCTGGCGGTGG | 1933 | CGGGGAGCUGGCGGUGGAG |
| exon_4 | | | | AGAGACCGTCGTC | | AGACCGUCGUC |
| BCL11A_ | + | ATTT | 623 | AAAATAGCCATAACATA | 1934 | AAAAUAGCCAUAACAUACC |
| exon_4 | | | | CCATACATGCTGT | | AUACAUGCUGU |
| BCL11A_ | + | ATTA | 624 | GGGACAATTTAAAATAG | 1935 | GGGACAAUUUAAAAUAGCC |
| exon_4 | | | | CCATAACATACCA | | AUAACAUACCA |
| BCL11A_ | + | TTTG | 625 | CTCAGCAACGAATTAGG | 1936 | CUCAGCAACGAAUUAGGGA |
| exon_4 | | | | GACAATTTAAAAT | | CAAUUUAAAAU |
| BCL11A_ | + | CTTA | 626 | CTAGTGTATTTAATTGC | 1937 | CUAGUGUAUUUAAUUGCGU |
| exon_4 | | | | GTTCCAGGGCTTT | | UCCAGGGCUUU |
| BCL11A_ | + | ATTT | 627 | AATTGCGTTCCAGGGCT | 1938 | AAUUGCGUUCCAGGGCUUU |
| exon_4 | | | | TTTGCACATTACA | | UGCACAUUACA |
| BCL11A_ | + | TTTA | 628 | ATTGCGTTCCAGGGCTT | 1939 | AUUGCGUUCCAGGGCUUUU |
| exon_4 | | | | TTGCACATTACAC | | GCACAUUACAC |
| BCL11A_ | + | ATTG | 629 | CGTTCCAGGGCTTTTGC | 1940 | CGUUCCAGGGCUUUUGCAC |
| exon_4 | | | | ACATTACACATTC | | AUUACACAUUC |
| BCL11A_ | + | GTTC | 630 | CAGGGCTTTTGCACATT | 1941 | CAGGGCUUUUGCACAUUAC |
| exon_4 | | | | ACACATTCAATTT | | ACAUUCAAUUU |
| BCL11A_ | + | CTTT | 631 | TGCACATTACACATTCA | 1942 | UGCACAUUACACAUUCAAU |
| exon_4 | | | | ATTTAATCATTGT | | UUAAUCAUUGU |
| BCL11A_ | + | TTTT | 632 | GCACATTACACATTCAA | 1943 | GCACAUUACACAUUCAAUU |
| exon_4 | | | | TTTAATCATTGTT | | UAAUCAUUGUU |
| BCL11A_ | + | TTTG | 633 | CACATTACACATTCAAT | 1944 | CACAUUACACAUUCAAUUU |
| exon_4 | | | | TTAATCATTGTTT | | AAUCAUUGUUU |
| BCL11A_ | + | ATTA | 634 | CACATTCAATTTAATCA | 1945 | CACAUUCAAUUUAAUCAUU |
| exon_4 | | | | TTGTTTAAAAAAA | | GUUUAAAAAAA |
| BCL11A_ | + | ATTC | 635 | AATTTAATCATTGTTTA | 1946 | AAUUUAAUCAUUGUUUAAA |
| exon_4 | | | | AAAAAAATAAAAC | | AAAAAAAAAC |
| BCL11A_ | + | ATTT | 636 | AATCATTGTTTAAAAAA | 1947 | AAUCAUUGUUUAAAAAAAA |
| exon_4 | | | | AATAAAACTTTGG | | UAAAACUUUGG |
| BCL11A_ | + | TTTA | 637 | ATCATTGTTTAAAAAAA | 1948 | AUCAUUGUUUAAAAAAAAU |
| exon_4 | | | | ATAAAACTTTGGG | | AAAACUUUGGG |
| BCL11A_ | + | ATTG | 638 | TTTAAAAAAAATAAAAC | 1949 | UUUAAAAAAAAUAAAACUU |
| exon 4 | | | | TTTGGGCAAAACA | | UGGGCAAAACA |
| BCL11A_ | + | GTTT | 639 | AAAAAAAATAAAACTTT | 1950 | AAAAAAAAUAAAACUUUGG |
| exon_4 | | | | GGGCAAAACAGCC | | GCAAAACAGCC |
| BCL11A_ | + | TTTA | 640 | AAAAAAATAAAACTTTG | 1951 | AAAAAAAUAAAACUUUGGG |
| exon_4 | | | | GGCAAAACAGCCC | | CAAAACAGCCC |
| BCL11A_ | + | CTTT | 641 | GGGCAAAACAGCCCATT | 1952 | GGGCAAAACAGCCCAUUUC |
| exon_4 | | | | TCTTTTAAGCTCT | | UUUUAAGCUCU |
| BCL11A_ | + | TTTG | 642 | GGCAAAACAGCCCATTT | 1953 | GGCAAAACAGCCCAUUUCU |
| exon_4 | | | | CTTTTAAGCTCTC | | UUUAAGCUCUC |
| BCL11A_ | + | ATTA | 643 | AACTAAAGGAAAAATGA | 1954 | AACUAAAGGAAAAAUGAUG |
| exon_4 | | | | TGATTAACTAGGA | | AUUAACUAGGA |
| BCL11A_ | + | TTTA | 644 | TAAAATTAAACTAAAGG | 1955 | UAAAAUUAAACUAAAGGAA |
| exon_4 | | | | AAAAATGATGATT | | AAAUGAUGAUU |
| BCL11A_ | + | GTTT | 645 | ATAAAATTAAACTAAAG | 1956 | AUAAAAUUAAACUAAAGGA |
| exon_4 | | | | GAAAAATGATGAT | | AAAAUGAUGAU |
| BCL11A_ | + | TTTG | 646 | TTTATAAAATTAAACTA | 1957 | UUUAUAAAAUUAAACUAAA |
| exon_4 | | | | AAGGAAAAATGAT | | GGAAAAAUGAU |
| BCL11A_ | + | TTTT | 647 | GTTTATAAAATTAAACT | 1958 | GUUUAUAAAAUUAAACUAA |
| exon_4 | | | | AAAGGAAAAATGA | | AGGAAAAAUGA |
| BCL11A_ | + | GTTT | 648 | TGTTTATAAAATTAAAC | 1959 | UGUUUAUAAAAUUAAACUA |
| exon_4 | | | | TAAAGGAAAAATG | | AAGGAAAAAUG |
| BCL11A_ | + | CTTC | 649 | ATAAAATGAACTCCTTA | 1960 | AUAAAAUGAACUCCUUACU |
| exon_4 | | | | CTAGTGTATTTAA | | AGUGUAUUUAA |
| BCL11A_ | + | TTTA | 650 | TACTGGTATAATCAGTT | 1961 | UACUGGUAUAAUCAGUUUU |
| exon_4 | | | | TTGTTTATAAAAT | | GUUUAUAAAAU |
| BCL11A_ | + | CTTT | 651 | TATACTGGTATAATCAG | 1962 | UAUACUGGUAUAAUCAGUU |
| exon_4 | | | | TTTTGTTTATAAA | | UUGUUUAUAAA |
| BCL11A_ | + | TTTA | 652 | AGCTCTCACCAGGAGCA | 1963 | AGCUCUCACCAGGAGCAAA |
| exon_4 | | | | AAGTAGCTTTTAT | | GUAGCUUUUAU |
| BCL11A_ | + | TTTT | 653 | AAGCTCTCACCAGGAGC | 1964 | AAGCUCUCACCAGGAGCAA |
| exon_4 | | | | AAAGTAGCTTTTA | | AGUAGCUUUUA |
| BCL11A_ | + | CTTT | 654 | TAAGCTCTCACCAGGAG | 1965 | UAAGCUCUCACCAGGAGCA |
| exon_4 | | | | CAAAGTAGCTTTT | | AAGUAGCUUUU |
| BCL11A_ | + | TTTC | 655 | TTTTAAGCTCTCACCAG | 1966 | UUUUAAGCUCUCACCAGGA |
| exon_4 | | | | GAGCAAAGTAGCT | | GCAAAGUAGCU |
| BCL11A_ | + | ATTT | 656 | CTTTTAAGCTCTCACCA | 1967 | CUUUUAAGCUCUCACCAGG |
| exon_4 | | | | GGAGCAAAGTAGC | | AGCAAAGUAGC |
| BCL11A_ | + | TTTT | 657 | ATACTGGTATAATCAGT | 1968 | AUACUGGUAUAAUCAGUUU |
| exon 4 | | | | TTTGTTTATAAAA | | UGUUUAUAAAA |
| BCL11A_ | + | ATTA | 658 | ACTAGGACATAATGGGT | 1969 | ACUAGGACAUAAUGGGUCA |
| exon_4 | | | | CATCTTTTTAGGT | | UCUUUUUAGGU |
| BCL11A_ | + | ATTA | 659 | AAGCAAATATCTTCATA | 1970 | AAGCAAAUAUCUUCAUAAA |
| exon_4 | | | | AAATGAACTCCTT | | AUGAACUCCUU |
| BCL11A_ | + | TTTA | 660 | AAAAGACATTATTAAAG | 1971 | AAAAGACAUUAUUAAAGCA |
| exon_4 | | | | CAAATATCTTCAT | | AAUAUCUUCAU |
| BCL11A_ | + | GTTC | 661 | TAGTTTTAAATGGCAAA | 1972 | UAGUUUUAAAUGGCAAAUA |
| exon_4 | | | | TAGTACCACGTTG | | GUACCACGUUG |
| BCL11A_ | + | GTTT | 662 | TAAATGGCAAATAGTAC | 1973 | UAAAUGGCAAAUAGUACCA |
| exon_4 | | | | CACGTTGTGCTAA | | CGUUGUGCUAA |
| BCL11A_ | + | TTTT | 663 | AAATGGCAAATAGTACC | 1974 | AAAUGGCAAAUAGUACCAC |
| exon_4 | | | | ACGTTGTGCTAAT | | GUUGUGCUAAU |
| BCL11A_ | + | TTTA | 664 | AATGGCAAATAGTACCA | 1975 | AAUGGCAAAUAGUACCACG |
| exon_4 | | | | CGTTGTGCTAATA | | UUGUGCUAAUA |
| BCL11A_ | + | GTTG | 665 | TGCTAATAAATCATATT | 1976 | UGCUAAUAAAUCAUAUUAU |
| exon 4 | | | | ATTTTCTTCTGTT | | UUUCUUCUGUU |
| BCL11A_ | + | ATTA | 666 | TTTTCTTCTGTTCCCCT | 1977 | UUUUCUUCUGUUCCCCUCU |
| exon 4 | | | | CTGTCAAACCTTA | | GUCAAACCUUA |
| BCL11A_ | + | ATTT | 667 | TCTTCTGTTCCCCTCTG | 1978 | UCUUCUGUUCCCCUCUGUC |
| exon 4 | | | | TCAAACCTTATTG | | AAACCUUAUUG |
| BCL11A_ | + | TTTT | 668 | CTTCTGTTCCCCTCTGT | 1979 | CUUCUGUUCCCCUCUGUCA |
| exon_4 | | | | CAAACCTTATTGT | | AACCUUAUUGU |
| BCL11A_ | + | TTTC | 669 | TTCTGTTCCCCTCTGTC | 1980 | UUCUGUUCCCCUCUGUCAA |
| exon_4 | | | | AAACCTTATTGTC | | ACCUUAUUGUC |
| BCL11A_ | + | CTTC | 670 | TGTTCCCCTCTGTCAAA | 1981 | UGUUCCCCUCUGUCAAACC |
| exon_4 | | | | CCTTATTGTCAGC | | UUAUUGUCAGC |
| BCL11A_ | + | GTTC | 671 | CCCTCTGTCAAACCTTA | 1982 | CCCUCUGUCAAACCUUAUU |
| exon_4 | | | | TTGTCAGCCTCTT | | GUCAGCCUCUU |
| BCL11A_ | + | CTTA | 672 | TTGTCAGCCTCTTCCTT | 1983 | UUGUCAGCCUCUUCCUUUC |
| exon 4 | | | | TCAATATGGTATA | | AAUAUGGUAUA |
| BCL11A_ | + | ATTG | 673 | TCAGCCTCTTCCTTTCA | 1984 | UCAGCCUCUUCCUUUCAAU |
| exon 4 | | | | ATATGGTATACAA | | AUGGUAUACAA |
| BCL11A_ | + | CTTC | 674 | CTTTCAATATGGTATAC | 1985 | CUUUCAAUAUGGUAUACAA |
| exon 4 | | | | AAGGTCTTAAAGT | | GGUCUUAAAGU |
| BCL11A_ | + | CTTT | 675 | CAATATGGTATACAAGG | 1986 | CAAUAUGGUAUACAAGGUC |
| exon 4 | | | | TCTTAAAGTTTAT | | UUAAAGUUUAU |
| BCL11A_ | − | TTTC | 676 | AATATGGTATACAAGGT | 1987 | AAUAUGGUAUACAAGGUCU |
| exon_4 | | | | CTTAAAGTTTATC | | UAAAGUUUAUC |
| BCL11A_ | + | CTTA | 677 | AAGTTTATCATTTGATT | 1988 | AAGUUUAUCAUUUGAUUGU |
| exon_4 | | | | GTCCACTTGACAA | | CCACUUGACAA |
| BCL11A_ | + | TTTT | 678 | AAAAAGACATTATTAAA | 1989 | AAAAAGACAUUAUUAAAGC |
| exon_4 | | | | GCAAATATCTTCA | | AAAUAUCUUCA |
| BCL11A_ | + | TTTT | 679 | TAAAAAGACATTATTAA | 1990 | UAAAAAGACAUUAUUAAAG |
| exon_4 | | | | AGCAAATATCTTC | | CAAAUAUCUUC |
| BCL11A_ | + | ATTT | 680 | TTAAAAAGACATTATTA | 199 | UUAAAAAGACAUUAUUAAA |
| exon 4 | | | | AAGCAAATATCTT | | GCAAAUAUCUU |
| BCL11A_ | + | TTTG | 681 | GTGCCAGTATTTTTAAA | 1992 | GUGCCAGUAUUUUUAAAAA |
| exon_4 | | | | AAGACATTATTAA | | GACAUUAUUAA |
| BCL11A_ | + | TTTT | 682 | GGTGCCAGTATTTTTAA | 1993 | GGUGCCAGUAUUUUUAAAA |
| exon 4 | | | | AAAGACATTATTA | | AGACAUUAUUA |
| BCL11A_ | + | CTTT | 683 | TGGTGCCAGTATTTTTA | 1994 | UGGUGCCAGUAUUUUUAAA |
| exon_4 | | | | AAAAGACATTATT | | AAGACAUUAUU |
| BCL11A_ | + | ATTA | 684 | TTAAAGCAAATATCTTC | 1995 | UUAAAGCAAAUAUCUUCAU |
| exon 4 | | | | ATAAAATGAACTC | | AAAAUGAACUC |
| BCL11A_ | + | TTTC | 685 | TTTTGGTGCCAGTATTT | 1996 | UUUUGGUGCCAGUAUUUUU |
| exon_4 | | | | TTAAAAAGACATT | | AAAAAGACAUU |
| BCL11A_ | + | CTTG | 686 | ACAACCAAGTAGATCTG | 1997 | ACAACCAAGUAGAUCUGGA |
| exon_4 | | | | GATCTATTTCTTT | | UCUAUUUCUUU |
| BCL11A_ | + | ATTG | 687 | TCCACTTGACAACCAAG | 1998 | UCCACUUGACAACCAAGUA |
| exon_4 | | | | TAGATCTGGATCT | | GAUCUGGAUCU |
| BCL11A_ | + | TTTG | 688 | ATTGTCCACTTGACAAC | 1999 | AUUGUCCACUUGACAACCA |
| exon_4 | | | | CAAGTAGATCTGG | | AGUAGAUCUGG |
| BCL11A_ | + | ATTT | 689 | GATTGTCCACTTGACAA | 2000 | GAUUGUCCACUUGACAACC |
| exon 4 | | | | CCAAGTAGATCTG | | AAGUAGAUCUG |
| BCL11A_ | + | TTTA | 690 | TCATTTGATTGTCCACT | 2001 | UCAUUUGAUUGUCCACUUG |
| exon 4 | | | | TGACAACCAAGTA | | ACAACCAAGUA |
| BCL11A_ | + | GTTT | 691 | ATCATTTGATTGTCCAC | 2002 | AUCAUUUGAUUGUCCACUU |
| exon_4 | | | | TTGACAACCAAGT | | GACAACCAAGU |
| BCL11A_ | + | ATTT | 692 | CTTTTGGTGCCAGTATT | 2003 | CUUUUGGUGCCAGUAUUUU |
| exon_4 | | | | TTTAAAAAGACAT | | UAAAAAGACAU |
| BCL11A_ | + | ATTA | 693 | GCTTATATACCTGTTCT | 2004 | GCUUAUAUACCUGUUCUAG |
| exon_4 | | | | AGTTTTAAATGGC | | UUUUAAAUGGC |
| BCL11A_ | + | CTTT | 694 | TTAGGTAGCCATTGTTG | 2005 | UUAGGUAGCCAUUGUUGUG |
| exon_4 | | | | TGAGAAATACAAT | | AGAAAUACAAU |
| BCL11A_ | + | TTTT | 695 | AGGTAGCCATTGTTGTG | 2006 | AGGUAGCCAUUGUUGUGAG |
| exon_4 | | | | AGAAATACAATAT | | AAAUACAAUAU |
| BCL11A_ | + | ATTG | 696 | ATACATTTAACCCTTTA | 2007 | AUACAUUUAACCCUUUAGA |
| exon 4 | | | | GAGACAGACATTT | | GACAGACAUUU |
| BCL11A_ | + | ATTT | 697 | AACCCTTTAGAGACAGA | 2008 | AACCCUUUAGAGACAGACA |
| exon_4 | | | | CATTTAGCTCATA | | UUUAGCUCAUA |
| BCL11A_ | + | TTTA | 698 | ACCCTTTAGAGACAGAC | 2009 | ACCCUUUAGAGACAGACAU |
| exon_4 | | | | ATTTAGCTCATAG | | UUAGCUCAUAG |
| BCL11A_ | + | CTTT | 699 | AGAGACAGACATTTAGC | 2010 | AGAGACAGACAUUUAGCUC |
| exon_4 | | | | TCATAGAGATTTT | | AUAGAGAUUUU |
| BCL11A_ | + | TTTA | 700 | GAGACAGACATTTAGCT | 2011 | GAGACAGACAUUUAGCUCA |
| exon_4 | | | | CATAGAGATTTTT | | UAGAGAUUUUU |
| BCL11A_ | + | ATTT | 701 | AGCTCATAGAGATTTTT | 2012 | AGCUCAUAGAGAUUUUUUU |
| exon_4 | | | | TTTCAGTGCTATC | | UCAGUGCUAUC |
| BCL11A_ | + | TTTA | 702 | GCTCATAGAGATTTTTT | 2013 | GCUCAUAGAGAUUUUUUUU |
| exon 4 | | | | TTCAGTGCTATCT | | CAGUGCUAUCU |
| BCL11A_ | + | ATTT | 703 | TTTTTCAGTGCTATCTA | 2014 | UUUUUCAGUGCUAUCUAUU |
| exon 4 | | | | TTCTGTCTATAGA | | CUGUCUAUAGA |
| BCL11A_ | + | TTTT | 704 | TTTTCAGTGCTATCTAT | 2015 | UUUUCAGUGCUAUCUAUUC |
| exon 4 | | | | TCTGTCTATAGAG | | UGUCUAUAGAG |
| BCL11A_ | + | TTTT | 705 | TTTCAGTGCTATCTATT | 2016 | UUUCAGUGCUAUCUAUUCU |
| exon_4 | | | | CTGTCTATAGAGG | | GUCUAUAGAGG |
| BCL11A_ | + | TTTT | 706 | TTCAGTGCTATCTATTC | 2017 | UUCAGUGCUAUCUAUUCUG |
| exon 4 | | | | TGTCTATAGAGGG | | UCUAUAGAGGG |
| BCL11A_ | + | TTTT | 707 | TCAGTGCTATCTATTCT | 2018 | UCAGUGCUAUCUAUUCUGU |
| exon_4 | | | | GTCTATAGAGGGT | | CUAUAGAGGGU |
| BCL11A_ | + | TTTT | 708 | CAGTGCTATCTATTCTG | 2019 | CAGUGCUAUCUAUUCUGUC |
| exon 4 | | | | TCTATAGAGGGTT | | UAUAGAGGGUU |
| BCL11A_ | + | TTTC | 709 | AGTGCTATCTATTCTGT | 2020 | AGUGCUAUCUAUUCUGUCU |
| exon_4 | | | | CTATAGAGGGTTA | | AUAGAGGGUUA |
| BCL11A_ | + | ATTC | 710 | TGTCTATAGAGGGTTAA | 2021 | UGUCUAUAGAGGGUUAAUC |
| exon 4 | | | | TCCAAAGACTGTT | | CAAAGACUGUU |
| BCL11A_ | + | GTTA | 711 | ATCCAAAGACTGTTTTT | 2022 | AUCCAAAGACUGUUUUUCC |
| exon 4 | | | | CCTCCTCACGTTA | | UCCUCACGUUA |
| BCL11A_ | + | GTTT | 712 | TTCCTCCTCACGTTATA | 2023 | UUCCUCCUCACGUUAUAAA |
| exon_4 | | | | AAATAAAACTGTA | | AUAAAACUGUA |
| BCL11A_ | + | GTTT | 713 | GCTCAGCAACGAATTAG | 2024 | GCUCAGCAACGAAUUAGGG |
| exon_4 | | | | GGACAATTTAAAA | | ACAAUUUAAAA |
| BCL11A_ | + | TTTC | 714 | TCTCAGAACGGAACTGG | 2025 | UCUCAGAACGGAACUGGAA |
| exon 4 | | | | AAACAGCAACATG | | ACAGCAACAUG |
| BCL11A_ | + | TTTT | 715 | CTCTCAGAACGGAACTG | 2026 | CUCUCAGAACGGAACUGGA |
| exon 4 | | | | GAAACAGCAACAT | | AACAGCAACAU |
| BCL11A_ | + | TTTT | 716 | TCTCTCAGAACGGAACT | 2027 | UCUCUCAGAACGGAACUGG |
| exon 4 | | | | GGAAACAGCAACA | | AAACAGCAACA |
| BCL11A_ | + | CTTT | 717 | TTCTCTCAGAACGGAAC | 2028 | UUCUCUCAGAACGGAACUG |
| exon 4 | | | | TGGAAACAGCAAC | | GAAACAGCAAC |
| BCL11A_ | + | TTTC | 718 | TCTCTCTCTCTCTTTTT | 2029 | UCUCUCUCUCUCUUUUUCU |
| exon_4 | | | | CTCTCAGAACGGA | | CUCAGAACGGA |
| BCL11A_ | + | TTTC | 719 | CAATTGATACATTTAAC | 2030 | CAAUUGAUACAUUUAACCC |
| exon_4 | | | | CCTTTAGAGACAG | | UUUAGAGACAG |
| BCL11A_ | + | TTTT | 720 | CTCTCTCTCTCTCTTTT | 2031 | CUCUCUCUCUCUCUUUUUC |
| exon_4 | | | | TCTCTCAGAACGG | | UCUCAGAACGG |
| BCL11A_ | + | CTTT | 721 | TTCTCTCTCTCTCTCTT | 2032 | UUCUCUCUCUCUCUCUUUU |
| exon_4 | | | | TTTCTCTCAGAAC | | UCUCUCAGAAC |
| BCL11A_ | + | ATTA | 722 | CAGAATGTATGCAGCAT | 2033 | CAGAAUGUAUGCAGCAUGG |
| exon_4 | | | | GGTCTTTTTCTCT | | UCUUUUUCUCU |
| BCL11A_ | + | GTTA | 723 | TAAAATAAAACTGTACA | 2034 | UAAAAUAAAACUGUACAUG |
| exon_4 | | | | TGATATGTATTAC | | AUAUGUAUUAC |
| BCL11A_ | + | TTTC | 724 | CTCCTCACGTTATAAAA | 2035 | CUCCUCACGUUAUAAAAUA |
| exon_4 | | | | TAAAACTGTACAT | | AAACUGUACAU |
| BCL11A_ | + | TTTT | 725 | CCTCCTCACGTTATAAA | 2036 | CCUCCUCACGUUAUAAAAU |
| exon 4 | | | | ATAAAACTGTACA | | AAAACUGUACA |
| BCL11A_ | + | TTTT | 726 | TCCTCCTCACGTTATAA | 2037 | UCCUCCUCACGUUAUAAAA |
| exon_4 | | | | AATAAAACTGTAC | | UAAAACUGUAC |
| BCL11A_ | + | TTTT | 727 | TCTCTCTCTCTCTCTTT | 2038 | UCUCUCUCUCUCUCUUUUU |
| exon_4 | | | | TTCTCTCAGAACG | | CUCUCAGAACG |
| BCL11A_ | + | TTTT | 728 | TAGGTAGCCATTGTTGT | 2039 | UAGGUAGCCAUUGUUGUGA |
| exon_4 | | | | GAGAAATACAATA | | GAAAUACAAUA |
| BCL11A_ | + | CTTT | 729 | CCAATTGATACATTTAA | 2040 | CCAAUUGAUACAUUUAACC |
| exon_4 | | | | CCCTTTAGAGACA | | CUUUAGAGACA |
| BCL11A_ | + | TTTC | 730 | TTCCTTTCCAATTGATA | 2041 | UUCCUUUCCAAUUGAUACA |
| exon_4 | | | | CATTTAACCCTTT | | UUUAACCCUUU |
| BCL11A_ | + | TTTA | 731 | GGTAGCCATTGTTGTGA | 2042 | GGUAGCCAUUGUUGUGAGA |
| exon 4 | | | | GAAATACAATATA | | AAUACAAUAUA |
| BCL11A_ | + | ATTG | 732 | TTGTGAGAAATACAATA | 2043 | UUGUGAGAAAUACAAUAUA |
| exon 4 | | | | TAGAATTATATGC | | GAAUUAUAUGC |
| BCL11A_ | + | GTTG | 733 | TGAGAAATACAATATAG | 2044 | UGAGAAAUACAAUAUAGAA |
| exon 4 | | | | AATTATATGCTAG | | UUAUAUGCUAG |
| BCL11A_ | + | ATTA | 734 | TATGCTAGTTCCTAAGG | 2045 | UAUGCUAGUUCCUAAGGUU |
| exon 4 | | | | TTTATTACCTCAC | | UAUUACCUCAC |
| BCL11A_ | + | GTTC | 735 | CTAAGGTTTATTACCTC | 2046 | CUAAGGUUUAUUACCUCAC |
| exon 4 | | | | ACCCAATGCTGAA | | CCAAUGCUGAA |
| BCL11A_ | + | GTTT | 736 | ATTACCTCACCCAATGC | 2047 | AUUACCUCACCCAAUGCUG |
| exon 4 | | | | TGAATTAAGCTAC | | AAUUAAGCUAC |
| BCL11A_ | + | TTTA | 737 | TTACCTCACCCAATGCT | 2048 | UUACCUCACCCAAUGCUGA |
| exon_4 | | | | GAATTAAGCTACA | | AUUAAGCUACA |
| BCL11A_ | + | ATTA | 738 | CCTCACCCAATGCTGAA | 2049 | CCUCACCCAAUGCUGAAUU |
| exon_4 | | | | TTAAGCTACAAGT | | AAGCUACAAGU |
| BCL11A_ | + | ATTA | 739 | AGCTACAAGTTTATAAC | 2050 | AGCUACAAGUUUAUAACAA |
| exon_4 | | | | AAGTAGAAAGAAC | | GUAGAAAGAAC |
| BCL11A_ | + | GTTT | 740 | ATAACAAGTAGAAAGAA | 2051 | AUAACAAGUAGAAAGAACC |
| exon_4 | | | | CCATCGATGTGGT | | AUCGAUGUGGU |
| BCL11A_ | + | TTTA | 741 | TAACAAGTAGAAAGAAC | 2052 | UAACAAGUAGAAAGAACCA |
| exon_4 | | | | CATCGATGTGGTT | | UCGAUGUGGUU |
| BCL11A_ | + | GTTT | 742 | TAATAGATCCAAGGCAC | 2053 | UAAUAGAUCCAAGGCACUC |
| exon_4 | | | | TCATATTTTAAAA | | AUAUUUUAAAA |
| BCL11A_ | + | TTTT | 743 | AATAGATCCAAGGCACT | 2054 | AAUAGAUCCAAGGCACUCA |
| exon 4 | | | | CATATTTTAAAAC | | UAUUUUAAAAC |
| BCL11A_ | + | TTTA | 744 | ATAGATCCAAGGCACTC | 2055 | AUAGAUCCAAGGCACUCAU |
| exon 4 | | | | ATATTTTAAAACC | | AUUUUAAAACC |
| BCL11A_ | + | ATTT | 745 | TAAAACCAAATGATAGA | 2056 | UAAAACCAAAUGAUAGAAU |
| exon 4 | | | | ATAAACTTGTTCT | | AAACUUGUUCU |
| BCL11A_ | + | TTTT | 746 | AAAACCAAATGATAGAA | 2057 | AAAACCAAAUGAUAGAAUA |
| exon 4 | | | | TAAACTTGTTCTG | | AACUUGUUCUG |
| BCL11A_ | + | TTTA | 747 | AAACCAAATGATAGAAT | 2058 | AAACCAAAUGAUAGAAUAA |
| exon_4 | | | | AAACTTGTTCTGT | | ACUUGUUCUGU |
| BCL11A_ | + | TTTT | 748 | CTTCCTTTCCAATTGAT | 2059 | CUUCCUUUCCAAUUGAUAC |
| exon_4 | | | | ACATTTAACCCTT | | AUUUAACCCUU |
| BCL11A_ | + | TTTT | 749 | TCTTCCTTTCCAATTGA | 2060 | UCUUCCUUUCCAAUUGAUA |
| exon_4 | | | | TACATTTAACCCT | | CAUUUAACCCU |
| BCL11A_ | + | TTTT | 750 | TTCTTCCTTTCCAATTG | 2061 | UUCUUCCUUUCCAAUUGAU |
| exon_4 | | | | ATACATTTAACCC | | ACAUUUAACCC |
| BCL11A_ | + | CTTT | 751 | TTTCTTCCTTTCCAATT | 2062 | UUUCUUCCUUUCCAAUUGA |
| exon_4 | | | | GATACATTTAACC | | UACAUUUAACC |
| BCL11A_ | − | TTTT | 752 | TGGCAGTTGTCTGCATT | 2063 | UGGCAGUUGUCUGCAUUAA |
| exon_4 | | | | AACCTGTTCATAC | | CCUGUUCAUAC |
| BCL11A_ | + | TTTG | 753 | TCAATTCAAGGCCTTTT | 2064 | UCAAUUCAAGGCCUUUUUU |
| exon 4 | | | | TTCTTCCTTTCCA | | CUUCCUUUCCA |
| BCL11A_ | + | CTTC | 754 | CTTTCCAATTGATACAT | 2065 | CUUUCCAAUUGAUACAUUU |
| exon 4 | | | | TTAACCCTTTAGA | | AACCCUUUAGA |
| BCL11A_ | + | ATTT | 755 | GTCAATTCAAGGCCTTT | 2066 | GUCAAUUCAAGGCCUUUUU |
| exon 4 | | | | TTTCTTCCTTTCC | | UCUUCCUUUCC |
| BCL11A_ | + | TTTC | 756 | TGTTAATTTGTCAATTC | 2067 | UGUUAAUUUGUCAAUUCAA |
| exon_4 | | | | AAGGCCTTTTTTC | | GGCCUUUUUUC |
| BCL11A_ | + | TTTT | 757 | CTGTTAATTTGTCAATT | 2068 | CUGUUAAUUUGUCAAUUCA |
| exon_4 | | | | CAAGGCCTTTTTT | | AGGCCUUUUUU |
| BCL11A_ | + | TTTT | 758 | TCTGTTAATTTGTCAAT | 2069 | UCUGUUAAUUUGUCAAUUC |
| exon_4 | | | | TCAAGGCCTTTTT | | AAGGCCUUUUU |
| BCL11A_ | + | GTTT | 759 | TTCTGTTAATTTGTCAA | 2070 | UUCUGUUAAUUUGUCAAUU |
| exon_4 | | | | TTCAAGGCCTTTT | | CAAGGCCUUUU |
| BCL11A_ | + | GTTC | 760 | TGTTTTTCTGTTAATTT | 2071 | UGUUUUUCUGUUAAUUUGU |
| exon_4 | | | | GTCAATTCAAGGC | | CAAUUCAAGGC |
| BCL11A_ | + | CTTG | 761 | TTCTGTTTTTCTGTTAA | 2072 | UUCUGUUUUUCUGUUAAUU |
| exon_4 | | | | TTTGTCAATTCAA | | UGUCAAUUCAA |
| BCL11A_ | + | GTTA | 762 | ATTTGTCAATTCAAGGC | 2073 | AUUUGUCAAUUCAAGGCCU |
| exon 4 | | | | CTTTTTTCTTCCT | | UUUUUCUUCCU |
| BCL11A_ | − | TTTT | 763 | TTGGCAGTTGTCTGCAT | 2074 | UUGGCAGUUGUCUGCAUUA |
| exon 4 | | | | TAACCTGTTCATA | | ACCUGUUCAUA |
| BCL11A_ | − | TTTC | 764 | CTTCTATCACCCTACAT | 2075 | CUUCUAUCACCCUACAUUC |
| exon 4 | | | | TCCAGCATCTTAC | | CAGCAUCUUAC |
| BCL11A_ | − | GTTT | 765 | TTTTGGCAGTTGTCTGC | 2076 | UUUUGGCAGUUGUCUGCAU |
| exon_4 | | | | ATTAACCTGTTCA | | UAACCUGUUCA |
| BCL11A_ | − | ATTA | 766 | ACAGAAAAACAGAACAA | 2077 | ACAGAAAAACAGAACAAGU |
| exon_4 | | | | GTTTATTCTATCA | | UUAUUCUAUCA |
| BCL11A_ | − | GTTT | 767 | ATTCTATCATTTGGTTT | 2078 | AUUCUAUCAUUUGGUUUUA |
| exon_4 | | | | TAAAATATGAGTG | | AAAUAUGAGUG |
| BCL11A_ | − | TTTA | 768 | TTCTATCATTTGGTTTT | 2079 | UUCUAUCAUUUGGUUUUAA |
| exon_4 | | | | AAAATATGAGTGC | | AAUAUGAGUGC |
| BCL11A_ | − | ATTC | 769 | TATCATTTGGTTTTAAA | 2080 | UAUCAUUUGGUUUUAAAAU |
| exon_4 | | | | ATATGAGTGCCTT | | AUGAGUGCCUU |
| BCL11A_ | − | ATTT | 770 | GGTTTTAAAATATGAGT | 2081 | GGUUUUAAAAUAUGAGUGC |
| exon_4 | | | | GCCTTGGATCTAT | | CUUGGAUCUAU |
| BCL11A_ | − | TTTG | 771 | GTTTTAAAATATGAGTG | 2082 | GUUUUAAAAUAUGAGUGCC |
| exon_4 | | | | CCTTGGATCTATT | | UUGGAUCUAUU |
| BCL11A_ | − | GTTT | 772 | TAAAATATGAGTGCCTT | 2083 | UAAAAUAUGAGUGCCUUGG |
| exon 4 | | | | GGATCTATTAAAA | | AUCUAUUAAAA |
| BCL11A_ | − | TTTT | 773 | AAAATATGAGTGCCTTG | 2084 | AAAAUAUGAGUGCCUUGGA |
| exon 4 | | | | GATCTATTAAAAC | | UCUAUUAAAAC |
| BCL11A_ | − | TTTA | 774 | AAATATGAGTGCCTTGG | 2085 | AAAUAUGAGUGCCUUGGAU |
| exon_4 | | | | ATCTATTAAAACC | | CUAUUAAAACC |
| BCL11A_ | − | CTTG | 775 | GATCTATTAAAACCACA | 2086 | GAUCUAUUAAAACCACAUC |
| exon 4 | | | | TCGATGGTTCTTT | | GAUGGUUCUUU |
| BCL11A_ | − | ATTA | 776 | AAACCACATCGATGGTT | 2087 | AAACCACAUCGAUGGUUCU |
| exon_4 | | | | CTTTCTACTTGTT | | UUCUACUUGUU |
| BCL11A_ | − | GTTC | 777 | TTTCTACTTGTTATAAA | 2088 | UUUCUACUUGUUAUAAACU |
| exon_4 | | | | CTTGTAGCTTAAT | | UGUAGCUUAAU |
| BCL11A_ | − | CTTT | 778 | CTACTTGTTATAAACTT | 2089 | CUACUUGUUAUAAACUUGU |
| exon_4 | | | | GTAGCTTAATTCA | | AGCUUAAUUCA |
| BCL11A_ | − | TTTC | 779 | TACTTGTTATAAACTTG | 2090 | UACUUGUUAUAAACUUGUA |
| exon_4 | | | | TAGCTTAATTCAG | | GCUUAAUUCAG |
| BCL11A_ | − | ATTG | 780 | ACAAATTAACAGAAAAA | 2091 | ACAAAUUAACAGAAAAACA |
| exon_4 | | | | CAGAACAAGTTTA | | GAACAAGUUUA |
| BCL11A_ | − | CTTG | 781 | TTATAAACTTGTAGCTT | 2092 | UUAUAAACUUGUAGCUUAA |
| exon_4 | | | | AATTCAGCATTGG | | UUCAGCAUUGG |
| BCL11A_ | − | CTTG | 782 | TAGCTTAATTCAGCATT | 2093 | UAGCUUAAUUCAGCAUUGG |
| exon 4 | | | | GGGTGAGGTAATA | | GUGAGGUAAUA |
| BCL11A_ | − | CTTA | 783 | ATTCAGCATTGGGTGAG | 2094 | AUUCAGCAUUGGGUGAGGU |
| exon 4 | | | | GTAATAAACCTTA | | AAUAAACCUUA |
| BCL11A_ | − | ATTC | 784 | AGCATTGGGTGAGGTAA | 2095 | AGCAUUGGGUGAGGUAAUA |
| exon_4 | | | | TAAACCTTAGGAA | | AACCUUAGGAA |
| BCL11A_ | − | ATTG | 785 | GGTGAGGTAATAAACCT | 2096 | GGUGAGGUAAUAAACCUUA |
| exon_4 | | | | TAGGAACTAGCAT | | GGAACUAGCAU |
| BCL11A_ | − | CTTA | 786 | GGAACTAGCATATAATT | 2097 | GGAACUAGCAUAUAAUUCU |
| exon_4 | | | | CTATATTGTATTT | | AUAUUGUAUUU |
| BCL11A_ | − | ATTC | 787 | TATATTGTATTTCTCAC | 2098 | UAUAUUGUAUUUCUCACAA |
| exon_4 | | | | AACAATGGCTACC | | CAAUGGCUACC |
| BCL11A_ | − | ATTG | 788 | TATTTCTCACAACAATG | 2099 | UAUUUCUCACAACAAUGGC |
| exon_4 | | | | GCTACCTAAAAAG | | UACCUAAAAAG |
| BCL11A_ | − | ATTT | 789 | CTCACAACAATGGCTAC | 2100 | CUCACAACAAUGGCUACCU |
| exon_4 | | | | CTAAAAAGATGAC | | AAAAAGAUGAC |
| BCL11A_ | − | TTTC | 790 | TCACAACAATGGCTACC | 2101 | UCACAACAAUGGCUACCUA |
| exon_4 | | | | TAAAAAGATGACC | | AAAAGAUGACC |
| BCL11A_ | − | ATTA | 791 | TGTCCTAGTTAATCATC | 2102 | UGUCCUAGUUAAUCAUCAU |
| exon_4 | | | | ATTTTTCCTTTAG | | UUUUCCUUUAG |
| BCL11A_ | − | GTTA | 792 | ATCATCATTTTTCCTTT | 2103 | AUCAUCAUUUUUCCUUUAG |
| exon_4 | | | | AGTTTAATTTTAT | | UUUAAUUUUAU |
| BCL11A_ | − | ATTT | 793 | TTCCTTTAGTTTAATTT | 2104 | UUCCUUUAGUUUAAUUUUA |
| exon_4 | | | | TATAAACAAAACT | | UAAACAAAACU |
| BCL11A_ | − | TTTT | 794 | TCCTTTAGTTTAATTTT | 2105 | UCCUUUAGUUUAAUUUUAU |
| exon_4 | | | | ATAAACAAAACTG | | AAACAAAACUG |
| BCL11A_ | − | TTTT | 795 | CCTTTAGTTTAATTTTA | 2106 | CCUUUAGUUUAAUUUUAUA |
| exon_4 | | | | TAAACAAAACTGA | | AACAAAACUGA |
| BCL11A_ | − | GTTA | 796 | TAAACTTGTAGCTTAAT | 2107 | UAAACUUGUAGCUUAAUUC |
| exon_4 | | | | TCAGCATTGGGTG | | AGCAUUGGGUG |
| BCL11A_ | − | CTTG | 797 | AATTGACAAATTAACAG | 2108 | AAUUGACAAAUUAACAGAA |
| exon_4 | | | | AAAAACAGAACAA | | AAACAGAACAA |
| BCL11A_ | − | ATTG | 798 | GAAAGGAAGAAAAAAGG | 2109 | GAAAGGAAGAAAAAAGGCC |
| exon_4 | | | | CCTTGAATTGACA | | UUGAAUUGACA |
| BCL11A_ | − | GTTA | 799 | AATGTATCAATTGGAAA | 2110 | AAUGUAUCAAUUGGAAAGG |
| exon_4 | | | | GGAAGAAAAAAGG | | AAGAAAAAAGG |
| BCL11A_ | − | GTTT | 800 | TTTTTTAAACTTAGACA | 2111 | UUUUUUAAACUUAGACAGC |
| exon_4 | | | | GCATGTATGGTAT | | AUGUAUGGUAU |
| BCL11A_ | − | TTTT | 801 | TTTTTAAACTTAGACAG | 2112 | UUUUUAAACUUAGACAGCA |
| exon_4 | | | | CATGTATGGTATG | | UGUAUGGUAUG |
| BCL11A_ | − | TTTT | 802 | TTTTAAACTTAGACAGC | 2113 | UUUUAAACUUAGACAGCAU |
| exon_4 | | | | ATGTATGGTATGT | | GUAUGGUAUGU |
| BCL11A_ | − | TTTT | 803 | TTTAAACTTAGACAGCA | 2114 | UUUAAACUUAGACAGCAUG |
| exon_4 | | | | TGTATGGTATGTT | | UAUGGUAUGUU |
| BCL11A_ | − | TTTT | 804 | TTAAACTTAGACAGCAT | 2115 | UUAAACUUAGACAGCAUGU |
| exon_4 | | | | GTATGGTATGTTA | | AUGGUAUGUUA |
| BCL11A_ | − | TTTT | 805 | TAAACTTAGACAGCATG | 2116 | UAAACUUAGACAGCAUGUA |
| exon_4 | | | | TATGGTATGTTAT | | UGGUAUGUUAU |
| BCL11A_ | − | TTTT | 806 | AAACTTAGACAGCATGT | 2117 | AAACUUAGACAGCAUGUAU |
| exon_4 | | | | ATGGTATGTTATG | | GGUAUGUUAUG |
| BCL11A_ | − | TTTA | 807 | AACTTAGACAGCATGTA | 2118 | AACUUAGACAGCAUGUAUG |
| exon 4 | | | | TGGTATGTTATGG | | GUAUGUUAUGG |
| BCL11A_ | − | CTTA | 808 | GACAGCATGTATGGTAT | 2119 | GACAGCAUGUAUGGUAUGU |
| exon_4 | | | | GTTATGGCTATTT | | UAUGGCUAUUU |
| BCL11A_ | − | GTTA | 809 | TGGCTATTTTAAATTGT | 2120 | UGGCUAUUUUAAAUUGUCC |
| exon_4 | | | | CCCTAATTCGTTG | | CUAAUUCGUUG |
| BCL11A_ | − | ATTT | 810 | TAAATTGTCCCTAATTC | 2121 | UAAAUUGUCCCUAAUUCGU |
| exon_4 | | | | GTTGCTGAGCAAA | | UGCUGAGCAAA |
| BCL11A_ | − | TTTT | 811 | AAATTGTCCCTAATTCG | 2122 | AAAUUGUCCCUAAUUCGUU |
| exon 4 | | | | TTGCTGAGCAAAC | | GCUGAGCAAAC |
| BCL11A_ | − | TTTA | 812 | AATTGTCCCTAATTCGT | 2123 | AAUUGUCCCUAAUUCGUUG |
| exon 4 | | | | TGCTGAGCAAACA | | CUGAGCAAACA |
| BCL11A_ | − | ATTG | 813 | TCCCTAATTCGTTGCTG | 2124 | UCCCUAAUUCGUUGCUGAG |
| exon_4 | | | | AGCAAACATGTTG | | CAAACAUGUUG |
| BCL11A_ | − | ATTC | 814 | GTTGCTGAGCAAACATG | 2125 | GUUGCUGAGCAAACAUGUU |
| exon_4 | | | | TTGCTGTTTCCAG | | GCUGUUUCCAG |
| BCL11A_ | − | GTTG | 815 | CTGAGCAAACATGTTGC | 2126 | CUGAGCAAACAUGUUGCUG |
| exon_4 | | | | TGTTTCCAGTTCC | | UUUCCAGUUCC |
| BCL11A_ | − | GTTG | 816 | CTGTTTCCAGTTCCGTT | 2127 | CUGUUUCCAGUUCCGUUCU |
| exon_4 | | | | CTGAGAGAAAAAG | | GAGAGAAAAAG |
| BCL11A_ | − | ATTA | 817 | ACCCTCTATAGACAGAA | 2128 | ACCCUCUAUAGACAGAAUA |
| exon_4 | | | | TAGATAGCACTGA | | GAUAGCACUGA |
| BCL11A_ | − | TTTG | 818 | GATTAACCCTCTATAGA | 2129 | GAUUAACCCUCUAUAGACA |
| exon 4 | | | | CAGAATAGATAGC | | GAAUAGAUAGC |
| BCL11A_ | − | CTTT | 819 | GGATTAACCCTCTATAG | 2130 | GGAUUAACCCUCUAUAGAC |
| exon 4 | | | | ACAGAATAGATAG | | AGAAUAGAUAG |
| BCL11A_ | − | TTTA | 820 | TAACGTGAGGAGGAAAA | 2131 | UAACGUGAGGAGGAAAAAC |
| exon 4 | | | | ACAGTCTTTGGAT | | AGUCUUUGGAU |
| BCL11A_ | − | TTTT | 821 | ATAACGTGAGGAGGAAA | 2132 | AUAACGUGAGGAGGAAAAA |
| exon_4 | | | | AACAGTCTTTGGA | | CAGUCUUUGGA |
| BCL11A_ | − | ATTT | 822 | TATAACGTGAGGAGGAA | 2133 | UAUAACGUGAGGAGGAAAA |
| exon_4 | | | | AAACAGTCTTTGG | | ACAGUCUUUGG |
| BCL11A_ | − | TTTC | 823 | CTTTAGTTTAATTTTAT | 2134 | CUUUAGUUUAAUUUUAUAA |
| exon 4 | | | | AAACAAAACTGAT | | ACAAAACUGAU |
| BCL11A_ | − | TTTA | 824 | TTTTATAACGTGAGGAG | 2135 | UUUUAUAACGUGAGGAGGA |
| exon 4 | | | | GAAAAACAGTCTT | | AAAACAGUCUU |
| BCL11A_ | − | GTTT | 825 | TATTTTATAACGTGAGG | 2136 | UAUUUUAUAACGUGAGGAG |
| exon_4 | | | | AGGAAAAACAGTC | | GAAAAACAGUC |
| BCL11A_ | − | ATTC | 826 | TGTAATACATATCATGT | 2137 | UGUAAUACAUAUCAUGUAC |
| exon_4 | | | | ACAGTTTTATTTT | | AGUUUUAUUUU |
| BCL11A_ | − | GTTC | 827 | TGAGAGAAAAAGAGAGA | 2138 | UGAGAGAAAAAGAGAGAGA |
| exon_4 | | | | GAGAGAGAAAAAG | | GAGAGAAAAAG |
| BCL11A_ | − | GTTC | 828 | CGTTCTGAGAGAAAAAG | 2139 | CGUUCUGAGAGAAAAAGAG |
| exon_4 | | | | AGAGAGAGAGAGA | | AGAGAGAGAGA |
| BCL11A_ | − | TTTC | 829 | CAGTTCCGTTCTGAGAG | 2140 | CAGUUCCGUUCUGAGAGAA |
| exon_4 | | | | AAAAAGAGAGAGA | | AAAGAGAGAGA |
| BCL11A_ | − | GTTT | 830 | CCAGTTCCGTTCTGAGA | 2141 | CCAGUUCCGUUCUGAGAGA |
| exon_4 | | | | GAAAAAGAGAGAG | | AAAAGAGAGAG |
| BCL11A_ | − | TTTT | 831 | ATTTTATAACGTGAGGA | 2142 | AUUUUAUAACGUGAGGAGG |
| exon_4 | | | | GGAAAAACAGTCT | | AAAAACAGUCU |
| BCL11A_ | − | CTTT | 832 | AGTTTAATTTTATAAAC | 2143 | AGUUUAAUUUUAUAAACAA |
| exon_4 | | | | AAAACTGATTATA | | AACUGAUUAUA |
| BCL11A_ | − | TTTA | 833 | GTTTAATTTTATAAACA | 2144 | GUUUAAUUUUAUAAACAAA |
| exon_4 | | | | AAACTGATTATAC | | ACUGAUUAUAC |
| BCL11A_ | − | GTTT | 834 | AATTTTATAAACAAAAC | 2145 | AAUUUUAUAAACAAAACUG |
| exon_4 | | | | TGATTATACCAGT | | AUUAUACCAGU |
| BCL11A_ | − | TTTA | 835 | AAAATACTGGCACCAAA | 2146 | AAAAUACUGGCACCAAAAG |
| exon_4 | | | | AGAAATAGATCCA | | AAAUAGAUCCA |
| BCL11A_ | − | CTTG | 836 | GTTGTCAAGTGGACAAT | 2147 | GUUGUCAAGUGGACAAUCA |
| exon_4 | | | | CAAATGATAAACT | | AAUGAUAAACU |
| BCL11A_ | − | GTTG | 837 | TCAAGTGGACAATCAAA | 2148 | UCAAGUGGACAAUCAAAUG |
| exon_4 | | | | TGATAAACTTTAA | | AUAAACUUUAA |
| BCL11A_ | − | CTTT | 838 | AAGACCTTGTATACCAT | 2149 | AAGACCUUGUAUACCAUAU |
| exon_4 | | | | ATTGAAAGGAAGA | | UGAAAGGAAGA |
| BCL11A_ | − | TTTA | 839 | AGACCTTGTATACCATA | 2150 | AGACCUUGUAUACCAUAUU |
| exon_4 | | | | TTGAAAGGAAGAG | | GAAAGGAAGAG |
| BCL11A_ | − | CTTG | 840 | TATACCATATTGAAAGG | 2151 | UAUACCAUAUUGAAAGGAA |
| exon_4 | | | | AAGAGGCTGACAA | | GAGGCUGACAA |
| BCL11A_ | − | ATTG | 841 | AAAGGAAGAGGCTGACA | 2152 | AAAGGAAGAGGCUGACAAU |
| exon 4 | | | | ATAAGGTTTGACA | | AAGGUUUGACA |
| BCL11A_ | − | GTTT | 842 | GACAGAGGGGAACAGAA | 2153 | GACAGAGGGGAACAGAAGA |
| exon_4 | | | | GAAAATAATATGA | | AAAUAAUAUGA |
| BCL11A_ | − | TTTG | 843 | ACAGAGGGGAACAGAAG | 2154 | ACAGAGGGGAACAGAAGAA |
| exon_4 | | | | AAAATAATATGAT | | AAUAAUAUGAU |
| BCL11A_ | − | ATTT | 844 | ATTAGCACAACGTGGTA | 2155 | AUUAGCACAACGUGGUACU |
| exon 4 | | | | CTATTTGCCATTT | | AUUUGCCAUUU |
| BCL11A_ | − | TTTA | 845 | TTAGCACAACGTGGTAC | 2156 | UUAGCACAACGUGGUACUA |
| exon 4 | | | | TATTTGCCATTTA | | UUUGCCAUUUA |
| BCL11A_ | − | ATTA | 846 | GCACAACGTGGTACTAT | 2157 | GCACAACGUGGUACUAUUU |
| exon 4 | | | | TTGCCATTTAAAA | | GCCAUUUAAAA |
| BCL11A_ | − | ATTT | 847 | GCCATTTAAAACTAGAA | 2158 | GCCAUUUAAAACUAGAACA |
| exon 4 | | | | CAGGTATATAAGC | | GGUAUAUAAGC |
| BCL11A_ | − | TTTG | 848 | CCATTTAAAACTAGAAC | 2159 | CCAUUUAAAACUAGAACAG |
| exon 4 | | | | AGGTATATAAGCT | | GUAUAUAAGCU |
| BCL11A_ | − | ATTT | 849 | AAAACTAGAACAGGTAT | 2160 | AAAACUAGAACAGGUAUAU |
| exon 4 | | | | ATAAGCTAATATT | | AAGCUAAUAUU |
| BCL11A_ | − | TTTA | 850 | AAACTAGAACAGGTATA | 2161 | AAACUAGAACAGGUAUAUA |
| exon 4 | | | | TAAGCTAATATTG | | AGCUAAUAUUG |
| BCL11A_ | − | ATTG | 851 | ATACAATGATGATTAAC | 2162 | AUACAAUGAUGAUUAACUA |
| exon 4 | | | | TATGAATTCTTAA | | UGAAUUCUUAA |
| BCL11A_ | − | ATTT | 852 | CTTTTCCATACACTGTG | 2163 | CUUUUCCAUACACUGUGUG |
| exon_4 | | | | TGCTATTTGTGTT | | CUAUUUGUGUU |
| BCL11A_ | − | CTTC | 853 | ATTTCTTTTCCATACAC | 2164 | AUUUCUUUUCCAUACACUG |
| exon_4 | | | | TGTGTGCTATTTG | | UGUGCUAUUUG |
| BCL11A_ | − | GTTG | 854 | TACTTCATTTCTTTTCC | 2165 | UACUUCAUUUCUUUUCCAU |
| exon_4 | | | | ATACACTGTGTGC | | ACACUGUGUGC |
| BCL11A_ | − | TTTA | 855 | AGAGTAGCAGTATATAT | 2166 | AGAGUAGCAGUAUAUAUGU |
| exon_4 | | | | GTCTGTGCTCCCT | | CUGUGCUCCCU |
| BCL11A_ | − | TTTT | 856 | AAGAGTAGCAGTATATA | 2167 | AAGAGUAGCAGUAUAUAUG |
| exon_4 | | | | TGTCTGTGCTCCC | | UCUGUGCUCCC |
| BCL11A_ | − | ATTT | 857 | TAAGAGTAGCAGTATAT | 2168 | UAAGAGUAGCAGUAUAUAU |
| exon_4 | | | | ATGTCTGTGCTCC | | GUCUGUGCUCC |
| BCL11A_ | − | TTTT | 858 | AAAAATACTGGCACCAA | 2169 | AAAAAUACUGGCACCAAAA |
| exon_4 | | | | AAGAAATAGATCC | | GAAAUAGAUCC |
| BCL11A_ | − | CTTA | 859 | AAAAAAGAAGAGAAAGA | 2170 | AAAAAAGAAGAGAAAGAAU |
| exon_4 | | | | ATTTTAAGAGTAG | | UUUAAGAGUAG |
| BCL11A_ | − | TTTA | 860 | AATGTGACATTCTTAAA | 2171 | AAUGUGACAUUCUUAAAAA |
| exon 4 | | | | AAAAGAAGAGAAA | | AAGAAGAGAAA |
| BCL11A_ | − | ATTT | 861 | AAATGTGACATTCTTAA | 2172 | AAAUGUGACAUUCUUAAAA |
| exon_4 | | | | AAAAAGAAGAGAA | | AAAGAAGAGAA |
| BCL11A_ | − | CTTG | 862 | CATTTAAATGTGACATT | 2173 | CAUUUAAAUGUGACAUUCU |
| exon 4 | | | | CTTAAAAAAAGAA | | UAAAAAAAGAA |
| BCL11A_ | − | CTTA | 863 | AGACTTGCATTTAAATG | 2174 | AGACUUGCAUUUAAAUGUG |
| exon_4 | | | | TGACATTCTTAAA | | ACAUUCUUAAA |
| BCL11A_ | − | ATTC | 864 | TTAAGACTTGCATTTAA | 2175 | UUAAGACUUGCAUUUAAAU |
| exon_4 | | | | ATGTGACATTCTT | | GUGACAUUCUU |
| BCL11A_ | − | ATTA | 865 | ACTATGAATTCTTAAGA | 2176 | ACUAUGAAUUCUUAAGACU |
| exon_4 | | | | CTTGCATTTAAAT | | UGCAUUUAAAU |
| BCL11A_ | − | ATTC | 866 | TTAAAAAAAGAAGAGAA | 2177 | UUAAAAAAAGAAGAGAAAG |
| exon_4 | | | | AGAATTTTAAGAG | | AAUUUUAAGAG |
| BCL11A_ | − | GTTG | 867 | TGTATGTTTTTTTTTAA | 2178 | UGUAUGUUUUUUUUUAAAC |
| exon_4 | | | | ACTTAGACAGCAT | | UUAGACAGCAU |
| BCL11A_ | − | TTTT | 868 | TAAAAATACTGGCACCA | 2179 | UAAAAAUACUGGCACCAAA |
| exon_4 | | | | AAAGAAATAGATC | | AGAAAUAGAUC |
| BCL11A_ | − | TTTA | 869 | ATAATGTCTTTTTAAAA | 2180 | AUAAUGUCUUUUUAAAAAU |
| exon_4 | | | | ATACTGGCACCAA | | ACUGGCACCAA |
| BCL11A_ | − | TTTA | 870 | ATTTTATAAACAAAACT | 2181 | AUUUUAUAAACAAAACUGA |
| exon 4 | | | | GATTATACCAGTA | | UUAUACCAGUA |
| BCL11A_ | − | ATTT | 871 | TATAAACAAAACTGATT | 2182 | UAUAAACAAAACUGAUUAU |
| exon_4 | | | | ATACCAGTATAAA | | ACCAGUAUAAA |
| BCL11A_ | − | TTTT | 872 | ATAAACAAAACTGATTA | 2183 | AUAAACAAAACUGAUUAUA |
| exon_4 | | | | TACCAGTATAAAA | | CCAGUAUAAAA |
| BCL11A_ | − | TTTA | 873 | TAAACAAAACTGATTAT | 2184 | UAAACAAAACUGAUUAUAC |
| exon_4 | | | | ACCAGTATAAAAG | | CAGUAUAAAAG |
| BCL11A_ | − | ATTA | 874 | TACCAGTATAAAAGCTA | 2185 | UACCAGUAUAAAAGCUACU |
| exon_4 | | | | CTTTGCTCCTGGT | | UUGCUCCUGGU |
| BCL11A_ | − | CTTT | 875 | GCTCCTGGTGAGAGCTT | 2186 | GCUCCUGGUGAGAGCUUAA |
| exon_4 | | | | AAAAGAAATGGGC | | AAGAAAUGGGC |
| BCL11A_ | − | TTTG | 876 | CTCCTGGTGAGAGCTTA | 2187 | CUCCUGGUGAGAGCUUAAA |
| exon_4 | | | | AAAGAAATGGGCT | | AGAAAUGGGCU |
| BCL11A_ | − | CTTA | 877 | AAAGAAATGGGCTGTTT | 2188 | AAAGAAAUGGGCUGUUUUG |
| exon_4 | | | | TGCCCAAAGTTTT | | CCCAAAGUUUU |
| BCL11A_ | − | GTTT | 878 | TGCCCAAAGTTTTATTT | 2189 | UGCCCAAAGUUUUAUUUUU |
| exon_4 | | | | TTTTTAAACAATG | | UUUAAACAAUG |
| BCL11A_ | − | TTTT | 879 | GCCCAAAGTTTTATTTT | 2190 | GCCCAAAGUUUUAUUUUUU |
| exon_4 | | | | TTTTAAACAATGA | | UUAAACAAUGA |
| BCL11A_ | − | TTTG | 880 | CCCAAAGTTTTATTTTT | 2191 | CCCAAAGUUUUAUUUUUUU |
| exon_4 | | | | TTTAAACAATGAT | | UAAACAAUGAU |
| BCL11A_ | − | GTTT | 881 | TATTTTTTTTAAACAAT | 2192 | UAUUUUUUUUAAACAAUGA |
| exon_4 | | | | GATTAAATTGAAT | | UUAAAUUGAAU |
| BCL11A_ | − | TTTT | 882 | ATTTTTTTTAAACAATG | 2193 | AUUUUUUUUAAACAAUGAU |
| exon_4 | | | | ATTAAATTGAATG | | UAAAUUGAAUG |
| BCL11A_ | − | TTTA | 883 | TTTTTTTTAAACAATGA | 2194 | UUUUUUUUAAACAAUGAUU |
| exon_4 | | | | TTAAATTGAATGT | | AAAUUGAAUGU |
| BCL11A_ | − | ATTT | 884 | TTTTTAAACAATGATTA | 2195 | UUUUUAAACAAUGAUUAAA |
| exon 4 | | | | AATTGAATGTGTA | | UUGAAUGUGUA |
| BCL11A_ | − | TTTT | 885 | TTTTAAACAATGATTAA | 2196 | UUUUAAACAAUGAUUAAAU |
| exon_4 | | | | ATTGAATGTGTAA | | UGAAUGUGUAA |
| BCL11A_ | − | TTTT | 886 | TTTAAACAATGATTAAA | 2197 | UUUAAACAAUGAUUAAAUU |
| exon_4 | | | | TTGAATGTGTAAT | | GAAUGUGUAAU |
| BCL11A_ | − | CTTT | 887 | AATAATGTCTTTTTAAA | 2198 | AAUAAUGUCUUUUUAAAAA |
| exon_4 | | | | AATACTGGCACCA | | UACUGGCACCA |
| BCL11A_ | − | TTTG | 888 | CTTTAATAATGTCTTTT | 2199 | CUUUAAUAAUGUCUUUUUA |
| exon 4 | | | | TAAAAATACTGGC | | AAAAUACUGGC |
| BCL11A_ | − | ATTT | 889 | GCTTTAATAATGTCTTT | 2200 | GCUUUAAUAAUGUCUUUUU |
| exon 4 | | | | TTAAAAATACTGG | | AAAAAUACUGG |
| BCL11A_ | − | TTTA | 890 | TGAAGATATTTGCTTTA | 2201 | UGAAGAUAUUUGCUUUAAU |
| exon_4 | | | | ATAATGTCTTTTT | | AAUGUCUUUUU |
| BCL11A_ | − | TTTT | 891 | ATGAAGATATTTGCTTT | 2202 | AUGAAGAUAUUUGCUUUAA |
| exon_4 | | | | AATAATGTCTTTT | | UAAUGUCUUUU |
| BCL11A_ | − | ATTT | 892 | TATGAAGATATTTGCTT | 2203 | UAUGAAGAUAUUUGCUUUA |
| exon_4 | | | | TAATAATGTCTTT | | AUAAUGUCUUU |
| BCL11A_ | − | CTTT | 893 | TTAAAAATACTGGCACC | 2204 | UUAAAAAUACUGGCACCAA |
| exon_4 | | | | AAAAGAAATAGAT | | AAGAAAUAGAU |
| BCL11A_ | − | GTTC | 894 | ATTTTATGAAGATATTT | 2205 | AUUUUAUGAAGAUAUUUGC |
| exon_4 | | | | GCTTTAATAATGT | | UUUAAUAAUGU |
| BCL11A_ | − | ATTG | 895 | AATGTGTAATGTGCAAA | 2206 | AAUGUGUAAUGUGCAAAAG |
| exon_4 | | | | AGCCCTGGAACGC | | CCCUGGAACGC |
| BCL11A_ | − | ATTA | 896 | AATTGAATGTGTAATGT | 2207 | AAUUGAAUGUGUAAUGUGC |
| exon_4 | | | | GCAAAAGCCCTGG | | AAAAGCCCUGG |
| BCL11A_ | − | TTTA | 897 | AACAATGATTAAATTGA | 2208 | AACAAUGAUUAAAUUGAAU |
| exon_4 | | | | ATGTGTAATGTGC | | GUGUAAUGUGC |
| BCL11A_ | − | TTTT | 898 | AAACAATGATTAAATTG | 2209 | AAACAAUGAUUAAAUUGAA |
| exon_4 | | | | AATGTGTAATGTG | | UGUGUAAUGUG |
| BCL11A_ | − | TTTT | 899 | TAAACAATGATTAAATT | 2210 | UAAACAAUGAUUAAAUUGA |
| exon 4 | | | | GAATGTGTAATGT | | AUGUGUAAUGU |
| BCL11A_ | − | TTTT | 900 | TTAAACAATGATTAAAT | 2211 | UUAAACAAUGAUUAAAUUG |
| exon 4 | | | | TGAATGTGTAATG | | AAUGUGUAAUG |
| BCL11A_ | − | ATTA | 901 | AATACACTAGTAAGGAG | 2212 | AAUACACUAGUAAGGAGUU |
| exon_4 | | | | TTCATTTTATGAA | | CAUUUUAUGAA |
| BCL11A_ | − | TTTA | 902 | CATGTTGTGTATGTTTT | 2213 | CAUGUUGUGUAUGUUUUUU |
| exon_4 | | | | TTTTTAAACTTAG | | UUUAAACUUAG |
| BCL11A_ | − | ATTT | 903 | ACATGTTGTGTATGTTT | 2214 | ACAUGUUGUGUAUGUUUUU |
| exon_4 | | | | TTTTTTAAACTTA | | UUUUAAACUUA |
| BCL11A_ | − | CTTG | 904 | TGCAATAATTTACATGT | 2215 | UGCAAUAAUUUACAUGUUG |
| exon_4 | | | | TGTGTATGTTTTT | | UGUAUGUUUUU |
| BCL11A_ | − | ATTC | 905 | CAGCCAGGTAGCAAGCC | 2216 | CAGCCAGGUAGCAAGCCGC |
| exon_4 | | | | GCCCTTCCTGGCG | | CCUUCCUGGCG |
| BCL11A_ | − | CTTC | 906 | CTGGCGACGCCCCCCCT | 2217 | CUGGCGACGCCCCCCCUCC |
| exon_4 | | | | CCCTCCTCTGCAA | | CUCCUCUGCAA |
| BCL11A_ | − | GTTC | 907 | TGCGGCAAGACGTTCAA | 2218 | UGCGGCAAGACGUUCAAAU |
| exon_4 | | | | ATTTCAGAGCAAC | | UUCAGAGCAAC |
| BCL11A_ | − | GTTC | 908 | AAATTTCAGAGCAACCT | 2219 | AAAUUUCAGAGCAACCUGG |
| exon_4 | | | | GGTGGTGCACCGG | | UGGUGCACCGG |
| BCL11A_ | − | ATTT | 909 | CAGAGCAACCTGGTGGT | 2220 | CAGAGCAACCUGGUGGUGC |
| exon_4 | | | | GCACCGGCGCAGC | | ACCGGCGCAGC |
| BCL11A_ | − | TTTC | 910 | AGAGCAACCTGGTGGTG | 2221 | AGAGCAACCUGGUGGUGCA |
| exon_4 | | | | CACCGGCGCAGCC | | CCGGCGCAGCC |
| BCL11A_ | − | CTTG | 911 | GTGGGCAGCGCCAGCAG | 2222 | GUGGGCAGCGCCAGCAGCG |
| exon_4 | | | | CGCGCTCAAGTCC | | CGCUCAAGUCC |
| BCL11A_ | − | GTTC | 912 | AAGAGCGAGAACGACCC | 2223 | AAGAGCGAGAACGACCCCA |
| exon_4 | | | | CAACCTGATCCCG | | ACCUGAUCCCG |
| BCL11A_ | − | CTTC | 913 | GGGCTGAGCCTGGAGGC | 2224 | GGGCUGAGCCUGGAGGCGG |
| exon_4 | | | | GGCGCGCCACCAC | | CGCGCCACCAC |
| BCL11A_ | − | CTTC | 914 | AGCGAGGCCTTCCACCA | 2225 | AGCGAGGCCUUCCACCAGG |
| exon_4 | | | | GGTCCTGGGCGAG | | UCCUGGGCGAG |
| BCL11A_ | − | CTTC | 915 | CACCAGGTCCTGGGCGA | 2226 | CACCAGGUCCUGGGCGAGA |
| exon_4 | | | | GAAGCATAAGCGC | | AGCAUAAGCGC |
| BCL11A_ | − | CTTG | 916 | CGACGAAGACTCGGTGG | 2227 | CGACGAAGACUCGGUGGCC |
| exon_4 | | | | CCGGCGAGTCGGA | | GGCGAGUCGGA |
| BCL11A_ | − | GTTA | 917 | ATGGCCGCGGCTGCTCC | 2228 | AUGGCCGCGGCUGCUCCCC |
| exon_4 | | | | CCGGGCGAGTCGG | | GGGCGAGUCGG |
| BCL11A_ | − | CTTC | 918 | TCTAAGCGCATCAAGCT | 2229 | UCUAAGCGCAUCAAGCUCG |
| exon_4 | | | | CGAGAAGGAGTTC | | AGAAGGAGUUC |
| BCL11A_ | − | GTTC | 919 | GACCTGCCCCCGGCCGC | 2230 | GACCUGCCCCCGGCCGCGA |
| exon_4 | | | | GATGCCCAACACG | | UGCCCAACACG |
| BCL11A_ | − | CTTC | 920 | CTTAGCTTCGGAGACTC | 2231 | CUUAGCUUCGGAGACUCCA |
| exon_4 | | | | CAGACAATCGCCT | | GACAAUCGCCU |
| BCL11A_ | − | CTTA | 921 | GCTTCGGAGACTCCAGA | 2232 | GCUUCGGAGACUCCAGACA |
| exon 4 | | | | CAATCGCCTTTTG | | AUCGCCUUUUG |
| BCL11A_ | − | ATTT | 922 | GTAAGATGCCTTTTAGC | 2233 | GUAAGAUGCCUUUUAGCGU |
| exon_4 | | | | GTGTACAGTACCC | | GUACAGUACCC |
| BCL11A_ | − | TTTA | 923 | CAAATGTGAAATTTGTA | 2234 | CAAAUGUGAAAUUUGUAAG |
| exon 4 | | | | AGATGCCTTTTAG | | AUGCCUUUUAG |
| BCL11A_ | − | GTTT | 924 | ACAAATGTGAAATTTGT | 2235 | ACAAAUGUGAAAUUUGUAA |
| exon_4 | | | | AAGATGCCTTTTA | | GAUGCCUUUUA |
| BCL11A_ | − | CTTA | 925 | TAAATGCGAGCTGTGCA | 2236 | UAAAUGCGAGCUGUGCAAC |
| exon 4 | | | | ACTATGCCTGTGC | | UAUGCCUGUGC |
| BCL11A_ | − | CTTC | 926 | AAGAACTGTAGCAATCT | 2237 | AAGAACUGUAGCAAUCUCA |
| exon_4 | | | | CACTGTCCACAGG | | CUGUCCACAGG |
| BCL11A_ | − | CTTG | 927 | TGAGTACTGTGGGAAAG | 2238 | UGAGUACUGUGGGAAAGUC |
| exon 4 | | | | TCTTCAAGAACTG | | UUCAAGAACUG |
| BCL11A_ | − | GTTA | 928 | CTGCAACCATTCCAGCC | 2239 | CUGCAACCAUUCCAGCCAG |
| exon 4 | | | | AGGTAGCAAGCCG | | GUAGCAAGCCG |
| BCL11A_ | − | ATTA | 929 | GTGGTCCGGGCCCGGGC | 2240 | GUGGUCCGGGCCCGGGCAG |
| exon_4 | | | | AGGCCCAGCTCAA | | GCCCAGCUCAA |
| BCL11A_ | − | TTTG | 930 | CGCTTCTCCACACCGCC | 2241 | CGCUUCUCCACACCGCCCG |
| exon_4 | | | | CGGGGAGCTGGAC | | GGGAGCUGGAC |
| BCL11A_ | − | GTTT | 931 | GCGCTTCTCCACACCGC | 2242 | GCGCUUCUCCACACCGCCC |
| exon_4 | | | | CCGGGGAGCTGGA | | GGGGAGCUGGA |
| BCL11A_ | − | TTTG | 932 | CCTCCTCGTCGGAGCAC | 2243 | CCUCCUCGUCGGAGCACUC |
| exon_4 | | | | TCCTCGGAGAACG | | CUCGGAGAACG |
| BCL11A_ | − | TTTT | 933 | GCCTCCTCGTCGGAGCA | 2244 | GCCUCCUCGUCGGAGCACU |
| exon_4 | | | | CTCCTCGGAGAAC | | CCUCGGAGAAC |
| BCL11A_ | − | CTTT | 934 | TGCCTCCTCGTCGGAGC | 2245 | UGCCUCCUCGUCGGAGCAC |
| exon_4 | | | | ACTCCTCGGAGAA | | UCCUCGGAGAA |
| BCL11A_ | − | CTTC | 935 | GGAGACTCCAGACAATC | 2246 | GGAGACUCCAGACAAUCGC |
| exon_4 | | | | GCCTTTTGCCTCC | | CUUUUGCCUCC |
| BCL11A_ | − | CTTC | 936 | TCCACACCGCCCGGGGA | 2247 | UCCACACCGCCCGGGGAGC |
| exon 4 | | | | GCTGGACGGAGGG | | UGGACGGAGGG |
| BCL11A_ | − | TTTG | 937 | TAAGATGCCTTTTAGCG | 2248 | UAAGAUGCCUUUUAGCGUG |
| exon_4 | | | | TGTACAGTACCCT | | UACAGUACCCU |
| BCL11A_ | − | CTTA | 938 | GAGAGCTGGCAGGGAAC | 2249 | GAGAGCUGGCAGGGAACAC |
| exon_4 | | | | ACGTCTAGCCCAC | | GUCUAGCCCAC |
| BCL11A_ | − | ATTT | 939 | CTCTAGGAGACTTAGAG | 2250 | CUCUAGGAGACUUAGAGAG |
| exon_4 | | | | AGCTGGCAGGGAA | | CUGGCAGGGAA |
| BCL11A_ | − | ATTA | 940 | AACATTGATGTTGGTGT | 2251 | AACAUUGAUGUUGGUGUUG |
| exon 4 | | | | TGTATTATTTTGC | | UAUUAUUUUGC |
| BCL11A_ | − | ATTG | 941 | ATGTTGGTGTTGTATTA | 2252 | AUGUUGGUGUUGUAUUAUU |
| exon 4 | | | | TTTTGCAGGTAAA | | UUGCAGGUAAA |
| BCL11A_ | − | GTTG | 942 | GTGTTGTATTATTTTGC | 2253 | GUGUUGUAUUAUUUUGCAG |
| exon_4 | | | | AGGTAAAGATGAG | | GUAAAGAUGAG |
| BCL11A_ | − | GTTG | 943 | TATTATTTTGCAGGTAA | 2254 | UAUUAUUUUGCAGGUAAAG |
| exon_4 | | | | AGATGAGCCCAGC | | AUGAGCCCAGC |
| BCL11A_ | − | ATTA | 944 | TTTTGCAGGTAAAGATG | 2255 | UUUUGCAGGUAAAGAUGAG |
| exon_4 | | | | AGCCCAGCAGCTA | | CCCAGCAGCUA |
| BCL11A_ | − | ATTT | 945 | TGCAGGTAAAGATGAGC | 2256 | UGCAGGUAAAGAUGAGCCC |
| exon_4 | | | | CCAGCAGCTACAC | | AGCAGCUACAC |
| BCL11A_ | − | TTTT | 946 | GCAGGTAAAGATGAGCC | 2257 | GCAGGUAAAGAUGAGCCCA |
| exon_4 | | | | CAGCAGCTACACA | | GCAGCUACACA |
| BCL11A_ | − | TTTG | 947 | CAGGTAAAGATGAGCCC | 2258 | CAGGUAAAGAUGAGCCCAG |
| exon_4 | | | | AGCAGCTACACAT | | CAGCUACACAU |
| BCL11A_ | − | CTTG | 948 | CAAACAGCCATTCACCA | 2259 | CAAACAGCCAUUCACCAGU |
| exon 4 | | | | GTGCATGGTTTCT | | GCAUGGUUUCU |
| BCL11A_ | − | ATTC | 949 | ACCAGTGCATGGTTTCT | 2260 | ACCAGUGCAUGGUUUCUCU |
| exon_4 | | | | CTTGCAACACGCA | | UGCAACACGCA |
| BCL11A_ | − | GTTT | 950 | CTCTTGCAACACGCACA | 2261 | CUCUUGCAACACGCACAGA |
| exon_4 | | | | GAACACTCATGGA | | ACACUCAUGGA |
| BCL11A_ | − | TTTC | 951 | TCTTGCAACACGCACAG | 2262 | UCUUGCAACACGCACAGAA |
| exon_4 | | | | AACACTCATGGAT | | CACUCAUGGAU |
| BCL11A_ | − | CTTG | 952 | CAACACGCACAGAACAC | 2263 | CAACACGCACAGAACACUC |
| exon_4 | | | | TCATGGATTAAGA | | AUGGAUUAAGA |
| BCL11A_ | − | ATTA | 953 | AGAATCTACTTAGAAAG | 2264 | AGAAUCUACUUAGAAAGCG |
| exon_4 | | | | CGAACACGGAAGT | | AACACGGAAGU |
| BCL11A_ | − | CTTA | 954 | GAAAGCGAACACGGAAG | 2265 | GAAAGCGAACACGGAAGUC |
| exon 4 | | | | TCCCCTGACCCCG | | CCCUGACCCCG |
| BCL11A_ | − | GTTG | 955 | GTATCCCTTCAGGACTA | 2266 | GUAUCCCUUCAGGACUAGG |
| exon 4 | | | | GGTGCAGAATGTC | | UGCAGAAUGUC |
| BCL11A_ | − | CTTC | 956 | AGGACTAGGTGCAGAAT | 2267 | AGGACUAGGUGCAGAAUGU |
| exon_4 | | | | GTCCTTCCCAGCC | | CCUUCCCAGCC |
| BCL11A_ | − | GTTG | 957 | AATCCAATGGCTATGGA | 2268 | AAUCCAAUGGCUAUGGAGC |
| exon_4 | | | | GCCTCCCGCCATG | | CUCCCGCCAUG |
| BCL11A_ | − | TTTG | 958 | ACAGGGTGCTGCGGTTG | 2269 | ACAGGGUGCUGCGGUUGAA |
| exon_4 | | | | AATCCAATGGCTA | | UCCAAUGGCUA |
| BCL11A_ | − | CTTT | 959 | GACAGGGTGCTGCGGTT | 2270 | GACAGGGUGCUGCGGUUGA |
| exon_4 | | | | GAATCCAATGGCT | | AUCCAAUGGCU |
| BCL11A_ | − | CTTG | 960 | GACCCCCACCGCATAGA | 2271 | GACCCCCACCGCAUAGAGC |
| exon_4 | | | | GCGCCTGGGGGCG | | GCCUGGGGGCG |
| BCL11A_ | − | TTTA | 961 | GTCCACCACCGAGACAT | 2272 | GUCCACCACCGAGACAUCA |
| exon_4 | | | | CACTTGGACCCCC | | CUUGGACCCCC |
| BCL11A_ | − | GTTT | 962 | AGTCCACCACCGAGACA | 2273 | AGUCCACCACCGAGACAUC |
| exon_4 | | | | TCACTTGGACCCC | | ACUUGGACCCC |
| BCL11A_ | − | TTTC | 963 | TCTAGGAGACTTAGAGA | 2274 | UCUAGGAGACUUAGAGAGC |
| exon_4 | | | | GCTGGCAGGGAAC | | UGGCAGGGAAC |
| BCL11A_ | − | TTTC | 964 | CACCCACTCCCCCCCTG | 2275 | CACCCACUCCCCCCCUGUU |
| exon_4 | | | | TTTAGTCCACCAC | | UAGUCCACCAC |
| BCL11A_ | − | CTTC | 965 | CGGCCTGGCAGAAGGGC | 2276 | CGGCCUGGCAGAAGGGCGC |
| exon_4 | | | | GCTTTCCACCCAC | | UUUCCACCCAC |
| BCL11A_ | − | TTTA | 966 | ACCTGCTAAGAATACCA | 2277 | ACCUGCUAAGAAUACCAGG |
| exon 4 | | | | GGATCAGTATCGA | | AUCAGUAUCGA |
| BCL11A_ | − | CTTT | 967 | AACCTGCTAAGAATACC | 2278 | AACCUGCUAAGAAUACCAG |
| exon 4 | | | | AGGATCAGTATCG | | GAUCAGUAUCG |
| BCL11A_ | − | ATTG | 968 | CAGACAATAACCCCTTT | 2279 | CAGACAAUAACCCCUUUAA |
| exon 4 | | | | AACCTGCTAAGAA | | CCUGCUAAGAA |
| BCL11A_ | − | ATTC | 969 | ATATTGCAGACAATAAC | 2280 | AUAUUGCAGACAAUAACCC |
| exon 4 | | | | CCCTTTAACCTGC | | CUUUAACCUGC |
| BCL11A_ | − | CTTC | 970 | CCAGCCACCTCTCCATG | 2281 | CCAGCCACCUCUCCAUGGG |
| exon 4 | | | | GGATTCATATTGC | | AUUCAUAUUGC |
| BCL11A_ | − | CTTT | 971 | CCACCCACTCCCCCCCT | 2282 | CCACCCACUCCCCCCCUGU |
| exon_4 | | | | GTTTAGTCCACCA | | UUAGUCCACCA |
| BCL11A_ | − | TTTC | 972 | TTTTCCATACACTGTGT | 2283 | UUUUCCAUACACUGUGUGC |
| exon_4 | | | | GCTATTTGTGTTA | | UAUUUGUGUUA |
| BCL11A_ | − | CTTT | 973 | TAGCGTGTACAGTACCC | 2284 | UAGCGUGUACAGUACCCUG |
| exon_4 | | | | TGGAGAAACACAT | | GAGAAACACAU |
| BCL11A_ | − | TTTA | 974 | GCGTGTACAGTACCCTG | 2285 | GCGUGUACAGUACCCUGGA |
| exon_4 | | | | GAGAAACACATGA | | GAAACACAUGA |
| BCL11A_ | − | TTTC | 975 | TTTTTCCTTTTTTTTTT | 2286 | UUUUUCCUUUUUUUUUUUU |
| exon_4 | | | | TTTTCCTTTATGT | | UUCCUUUAUGU |
| BCL11A_ | − | CTTT | 976 | TTCCTTTTTTTTTTTTT | 2287 | UUCCUUUUUUUUUUUUUUC |
| exon_4 | | | | TCCTTTATGTTCT | | CUUUAUGUUCU |
| BCL11A_ | − | TTTT | 977 | TCCTTTTTTTTTTTTTT | 2288 | UCCUUUUUUUUUUUUUUCC |
| exon_4 | | | | CCTTTATGTTCTC | | UUUAUGUUCUC |
| BCL11A_ | − | TTTT | 978 | CCTTTTTTTTTTTTTTC | 2289 | CCUUUUUUUUUUUUUUCCU |
| exon_4 | | | | CTTTATGTTCTCA | | UUAUGUUCUCA |
| BCL11A_ | − | TTTC | 979 | CTTTTTTTTTTTTTTCC | 2290 | CUUUUUUUUUUUUUUCCUU |
| exon_4 | | | | TTTATGTTCTCAC | | UAUGUUCUCAC |
| BCL11A_ | − | CTTT | 980 | TTTTTTTTTTTCCTTTA | 2291 | UUUUUUUUUUUCCUUUAUG |
| exon 4 | | | | TGTTCTCACCGTT | | UUCUCACCGUU |
| BCL11A_ | − | TTTT | 981 | TTTTTTTTTTCCTTTAT | 2292 | UUUUUUUUUUCCUUUAUGU |
| exon 4 | | | | GTTCTCACCGTTT | | UCUCACCGUUU |
| BCL11A_ | − | TTTT | 982 | TTTTTTTTTCCTTTATG | 2293 | UUUUUUUUUCCUUUAUGUU |
| exon_4 | | | | TTCTCACCGTTTG | | CUCACCGUUUG |
| BCL11A_ | − | TTTT | 983 | TTTTTTTTCCTTTATGT | 2294 | UUUUUUUUCCUUUAUGUUC |
| exon 4 | | | | TCTCACCGTTTGA | | UCACCGUUUGA |
| BCL11A_ | − | TTTT | 984 | TTTTTTTCCTTTATGTT | 2295 | UUUUUUUCCUUUAUGUUCU |
| exon 4 | | | | CTCACCGTTTGAA | | CACCGUUUGAA |
| BCL11A_ | − | TTTT | 985 | TTTTTTCCTTTATGTTC | 2296 | UUUUUUCCUUUAUGUUCUC |
| exon_4 | | | | TCACCGTTTGAAT | | ACCGUUUGAAU |
| BCL11A_ | − | TTTT | 986 | TTTTTCCTTTATGTTCT | 2297 | UUUUUCCUUUAUGUUCUCA |
| exon 4 | | | | CACCGTTTGAATG | | CCGUUUGAAUG |
| BCL11A_ | − | TTTT | 987 | TTTTCCTTTATGTTCTC | 2298 | UUUUCCUUUAUGUUCUCAC |
| exon_4 | | | | ACCGTTTGAATGC | | CGUUUGAAUGC |
| BCL11A_ | − | TTTT | 988 | TTTCCTTTATGTTCTCA | 2299 | UUUCCUUUAUGUUCUCACC |
| exon_4 | | | | CCGTTTGAATGCA | | GUUUGAAUGCA |
| BCL11A_ | − | TTTT | 989 | TTCCTTTATGTTCTCAC | 2300 | UUCCUUUAUGUUCUCACCG |
| exon 4 | | | | CGTTTGAATGCAT | | UUUGAAUGCAU |
| BCL11A_ | − | TTTT | 990 | TCCTTTATGTTCTCACC | 2301 | UCCUUUAUGUUCUCACCGU |
| exon 4 | | | | GTTTGAATGCATG | | UUGAAUGCAUG |
| BCL11A_ | − | TTTT | 991 | CCTTTATGTTCTCACCG | 2302 | CCUUUAUGUUCUCACCGUU |
| exon_4 | | | | TTTGAATGCATGA | | UGAAUGCAUGA |
| BCL11A_ | − | TTTC | 992 | TCTTGTGCAATAATTTA | 2303 | UCUUGUGCAAUAAUUUACA |
| exon_4 | | | | CATGTTGTGTATG | | UGUUGUGUAUG |
| BCL11A_ | − | CTTT | 993 | CTCTTGTGCAATAATTT | 2304 | CUCUUGUGCAAUAAUUUAC |
| exon 4 | | | | ACATGTTGTGTAT | | AUGUUGUGUAU |
| BCL11A_ | − | TTTG | 994 | AGCCTTTCTCTTGTGCA | 2305 | AGCCUUUCUCUUGUGCAAU |
| exon_4 | | | | ATAATTTACATGT | | AAUUUACAUGU |
| BCL11A_ | − | CTTT | 995 | GAGCCTTTCTCTTGTGC | 2306 | GAGCCUUUCUCUUGUGCAA |
| exon_4 | | | | AATAATTTACATG | | UAAUUUACAUG |
| BCL11A_ | − | TTTA | 996 | CGCAAACTTTGAGCCTT | 2307 | CGCAAACUUUGAGCCUUUC |
| exon_4 | | | | TCTCTTGTGCAAT | | UCUUGUGCAAU |
| BCL11A_ | − | TTTT | 997 | ACGCAAACTTTGAGCCT | 2308 | ACGCAAACUUUGAGCCUUU |
| exon_4 | | | | TTCTCTTGTGCAA | | CUCUUGUGCAA |
| BCL11A_ | − | TTTT | 998 | CTTTTTCCTTTTTTTTT | 2309 | CUUUUUCCUUUUUUUUUUU |
| exon_4 | | | | TTTTTCCTTTATG | | UUUCCUUUAUG |
| BCL11A_ | − | ATTT | 999 | TACGCAAACTTTGAGCC | 2310 | UACGCAAACUUUGAGCCUU |
| exon_4 | | | | TTTCTCTTGTGCA | | UCUCUUGUGCA |
| BCL11A_ | − | TTTG | 1000 | AATGCATGATCTGTATG | 2311 | AAUGCAUGAUCUGUAUGGG |
| exon_4 | | | | GGGCAATACTATT | | GCAAUACUAUU |
| BCL11A_ | − | GTTT | 1001 | GAATGCATGATCTGTAT | 2312 | GAAUGCAUGAUCUGUAUGG |
| exon_4 | | | | GGGGCAATACTAT | | GGCAAUACUAU |
| BCL11A_ | − | GTTC | 1002 | TCACCGTTTGAATGCAT | 2313 | UCACCGUUUGAAUGCAUGA |
| exon_4 | | | | GATCTGTATGGGG | | UCUGUAUGGGG |
| BCL11A_ | − | TTTA | 1003 | TGTTCTCACCGTTTGAA | 2314 | UGUUCUCACCGUUUGAAUG |
| exon_4 | | | | TGCATGATCTGTA | | CAUGAUCUGUA |
| BCL11A_ | − | CTTT | 1004 | ATGTTCTCACCGTTTGA | 2315 | AUGUUCUCACCGUUUGAAU |
| exon_4 | | | | ATGCATGATCTGT | | GCAUGAUCUGU |
| BCL11A_ | − | TTTC | 1005 | CTTTATGTTCTCACCGT | 2316 | CUUUAUGUUCUCACCGUUU |
| exon_4 | | | | TTGAATGCATGAT | | GAAUGCAUGAU |
| BCL11A_ | − | ATTG | 1006 | CATTTTACGCAAACTTT | 2317 | CAUUUUACGCAAACUUUGA |
| exon_4 | | | | GAGCCTTTCTCTT | | GCCUUUCUCUU |
| BCL11A_ | − | TTTT | 1007 | AGCGTGTACAGTACCCT | 2318 | AGCGUGUACAGUACCCUGG |
| exon_4 | | | | GGAGAAACACATG | | AGAAACACAUG |
| BCL11A_ | − | TTTT | 1008 | TCTTTTTCCTTTTTTTT | 2319 | UCUUUUUCCUUUUUUUUUU |
| exon_4 | | | | TTTTTTCCTTTAT | | UUUUCCUUUAU |
| BCL11A_ | − | CTTT | 1009 | TTTCTTTTTCCTTTTTT | 2320 | UUUCUUUUUCCUUUUUUUU |
| exon_4 | | | | TTTTTTTTCCTTT | | UUUUUUCCUUU |
| BCL11A_ | − | GTTG | 1010 | AATAATGATATAAAAAC | 2321 | AAUAAUGAUAUAAAAACUG |
| exon_4 | | | | TGAATAGAGGTAT | | AAUAGAGGUAU |
| BCL11A_ | − | ATTA | 1011 | ATACCCCTCCCTCACTC | 2322 | AUACCCCUCCCUCACUCCC |
| exon_4 | | | | CCACCTGACACCC | | ACCUGACACCC |
| BCL11A_ | − | CTTT | 1012 | TTCACCACTCCCCTTCC | 2323 | UUCACCACUCCCCUUCCCC |
| exon_4 | | | | CCATCGCCCTCCA | | AUCGCCCUCCA |
| BCL11A_ | − | TTTT | 1013 | TCACCACTCCCCTTCCC | 2324 | UCACCACUCCCCUUCCCCA |
| exon 4 | | | | CATCGCCCTCCAG | | UCGCCCUCCAG |
| BCL11A_ | − | TTTT | 1014 | CACCACTCCCCTTCCCC | 2325 | CACCACUCCCCUUCCCCAU |
| exon_4 | | | | ATCGCCCTCCAGC | | CGCCCUCCAGC |
| BCL11A_ | − | TTTC | 1015 | ACCACTCCCCTTCCCCA | 2326 | ACCACUCCCCUUCCCCAUC |
| exon 4 | | | | TCGCCCTCCAGCC | | GCCCUCCAGCC |
| BCL11A_ | − | CTTC | 1016 | CCCATCGCCCTCCAGCC | 2327 | CCCAUCGCCCUCCAGCCCC |
| exon_4 | | | | CCACTCCCTGTAG | | ACUCCCUGUAG |
| BCL11A_ | − | ATTT | 1017 | TTTTCTAGTCCCATGTG | 2328 | UUUUCUAGUCCCAUGUGAU |
| exon_4 | | | | ATTTAAACAAACA | | UUAAACAAACA |
| BCL11A_ | − | TTTT | 1018 | TTTCTAGTCCCATGTGA | 2329 | UUUCUAGUCCCAUGUGAUU |
| exon_4 | | | | TTTAAACAAACAA | | UAAACAAACAA |
| BCL11A_ | − | TTTT | 1019 | TTCTAGTCCCATGTGAT | 2330 | UUCUAGUCCCAUGUGAUUU |
| exon_4 | | | | TTAAACAAACAAA | | AAACAAACAAA |
| BCL11A_ | − | TTTT | 1020 | TCTAGTCCCATGTGATT | 2331 | UCUAGUCCCAUGUGAUUUA |
| exon_4 | | | | TAAACAAACAAAC | | AACAAACAAAC |
| BCL11A_ | − | TTTT | 1021 | CTAGTCCCATGTGATTT | 2332 | CUAGUCCCAUGUGAUUUAA |
| exon_4 | | | | AAACAAACAAACA | | ACAAACAAACA |
| BCL11A_ | − | TTTC | 1022 | TAGTCCCATGTGATTTA | 2333 | UAGUCCCAUGUGAUUUAAA |
| exon_4 | | | | AACAAACAAACAA | | CAAACAAACAA |
| BCL11A_ | − | ATTT | 1023 | AAACAAACAAACAAACA | 2334 | AAACAAACAAACAAACAAA |
| exon_4 | | | | AACAGAAGTAACG | | CAGAAGUAACG |
| BCL11A_ | − | TTTA | 1024 | AACAAACAAACAAACAA | 2335 | AACAAACAAACAAACAAAC |
| exon 4 | | | | ACAGAAGTAACGA | | AGAAGUAACGA |
| BCL11A_ | − | CTTG | 1025 | TCACCAGCACACCTGTT | 2336 | UCACCAGCACACCUGUUUU |
| exon 4 | | | | TTTTTTCTTTTTC | | UUUUCUUUUUC |
| BCL11A_ | − | GTTT | 1026 | TTTTTCTTTTTCTTTTT | 2337 | UUUUUCUUUUUCUUUUUCU |
| exon 4 | | | | CTTTTTTCTTTTT | | UUUUUCUUUUU |
| BCL11A_ | − | TTTC | 1027 | TTTTTTCTTTTTCCTTT | 2338 | UUUUUUCUUUUUCCUUUUU |
| exon_4 | | | | TTTTTTTTTTTCC | | UUUUUUUUUCC |
| BCL11A_ | − | TTTT | 1028 | CTTTTTTCTTTTTCCTT | 2339 | CUUUUUUCUUUUUCCUUUU |
| exon_4 | | | | TTTTTTTTTTTTC | | UUUUUUUUUUC |
| BCL11A_ | − | TTTT | 1029 | TCTTTTTTCTTTTTCCT | 2340 | UCUUUUUUCUUUUUCCUUU |
| exon_4 | | | | TTTTTTTTTTTTT | | UUUUUUUUUUU |
| BCL11A_ | − | CTTT | 1030 | TTCTTTTTTCTTTTTCC | 2341 | UUCUUUUUUCUUUUUCCUU |
| exon_4 | | | | TTTTTTTTTTTTT | | UUUUUUUUUUU |
| BCL11A_ | − | TTTC | 1031 | TTTTTCTTTTTTCTTTT | 2342 | UUUUUCUUUUUUUUUUUC |
| exon_4 | | | | TCCTTTTTTTTTT | | CUUUUUUUUUU |
| BCL11A_ | − | TTTT | 1032 | CTTTTTCTTTTTTCTTT | 2343 | CUUUUUCUUUUUUCUUUUU |
| exon_4 | | | | TTCCTTTTTTTTT | | CCUUUUUUUUU |
| BCL11A_ | − | TTTT | 1033 | TTCTTTTTCCTTTTTTT | 2344 | UUCUUUUUCCUUUUUUUUU |
| exon_4 | | | | TTTTTTTCCTTTA | | UUUUUCCUUUA |
| BCL11A_ | − | TTTT | 1034 | TCTTTTTCTTTTTTCTT | 2345 | UCUUUUUCUUUUUUUUUU |
| exon_4 | | | | TTTCCTTTTTTTT | | UCCUUUUUUUU |
| BCL11A_ | − | TTTC | 1035 | TTTTTCTTTTTCTTTTT | 2346 | UUUUUCUUUUUCUUUUUUC |
| exon_4 | | | | TCTTTTTCCTTTT | | UUUUUCCUUUU |
| BCL11A_ | − | TTTT | 1036 | CTTTTTCTTTTTCTTTT | 2347 | CUUUUUCUUUUUCUUUUUU |
| exon_4 | | | | TTCTTTTTCCTTT | | CUUUUUCCUUU |
| BCL11A_ | − | TTTT | 1037 | TCTTTTTCTTTTTCTTT | 2348 | UCUUUUUCUUUUUCUUUUU |
| exon_4 | | | | TTTCTTTTTCCTT | | UCUUUUUCCUU |
| BCL11A_ | − | TTTT | 1038 | TTCTTTTTCTTTTTCTT | 2349 | UUCUUUUUCUUUUUCUUUU |
| exon_4 | | | | TTTTCTTTTTCCT | | UUCUUUUUCCU |
| BCL11A_ | − | TTTT | 1039 | TTTCTTTTTCTTTTTCT | 2350 | UUUCUUUUUCUUUUUCUUU |
| exon_4 | | | | TTTTTCTTTTTCC | | UUUCUUUUUCC |
| BCL11A_ | − | TTTT | 1040 | TTTTCTTTTTCTTTTTC | 2351 | UUUUCUUUUUCUUUUUCUU |
| exon 4 | | | | TTTTTTCTTTTTC | | UUUUCUUUUUC |
| BCL11A_ | − | CTTT | 1041 | TTCTTTTTCTTTTTTCT | 2352 | UUCUUUUUCUUUUUUCUUU |
| exon_4 | | | | TTTTCCTTTTTTT | | UUCCUUUUUUU |
| BCL11A_ | − | TTTT | 1042 | TTTGGCAGTTGTCTGCA | 2353 | UUUGGCAGUUGUCUGCAUU |
| exon 4 | | | | TTAACCTGTTCAT | | AACCUGUUCAU |
| BCL11A_ | − | CTTT | 1043 | TCCATACACTGTGTGCT | 2354 | UCCAUACACUGUGUGCUAU |
| exon_4 | | | | ATTTGTGTTAACA | | UUGUGUUAACA |
| BCL11A_ | − | TTTC | 1044 | CATACACTGTGTGCTAT | 2355 | CAUACACUGUGUGCUAUUU |
| exon 4 | | | | TTGTGTTAACATG | | GUGUUAACAUG |
| BCL11A_ | − | TTTT | 1045 | GTCCCTTTCCTTCTATC | 2356 | GUCCCUUUCCUUCUAUCAC |
| exon_4 | | | | ACCCTACATTCCA | | CCUACAUUCCA |
| BCL11A_ | − | TTTG | 1046 | TCCCTTTCCTTCTATCA | 2357 | UCCCUUUCCUUCUAUCACC |
| exon_4 | | | | CCCTACATTCCAG | | CUACAUUCCAG |
| BCL11A_ | − | CTTT | 1047 | CCTTCTATCACCCTACA | 2358 | CCUUCUAUCACCCUACAUU |
| exon_4 | | | | TTCCAGCATCTTA | | CCAGCAUCUUA |
| BCL11A_ | + | CTTT | 1048 | ACCTGCAAAATAATACA | 2359 | ACCUGCAAAAUAAUACAAC |
| exon_4 | | | | ACACCAACATCAA | | ACCAACAUCAA |
| BCL11A_ | − | CTTC | 1049 | TATCACCCTACATTCCA | 2360 | UAUCACCCUACAUUCCAGC |
| exon_4 | | | | GCATCTTACCTTC | | AUCUUACCUUC |
| BCL11A_ | − | ATTC | 1050 | CAGCATCTTACCTTCAT | 2361 | CAGCAUCUUACCUUCAUAU |
| exon_4 | | | | ATGCAGTAAAAGA | | GCAGUAAAAGA |
| BCL11A_ | − | CTTA | 1051 | CCTTCATATGCAGTAAA | 2362 | CCUUCAUAUGCAGUAAAAG |
| exon_4 | | | | AGAAAGAAAGAAA | | AAAGAAAGAAA |
| BCL11A_ | − | CTTC | 1052 | ATATGCAGTAAAAGAAA | 2363 | AUAUGCAGUAAAAGAAAGA |
| exon_4 | | | | GAAAGAAAAAAAA | | AAGAAAAAAAA |
| BCL11A_ | − | GTTT | 1053 | TGCAGTTTTTTTCATTG | 2364 | UGCAGUUUUUUUCAUUGCC |
| exon_4 | | | | CCAAAAACTAAAT | | AAAAACUAAAU |
| BCL11A_ | − | TTTT | 1054 | GCAGTTTTTTTCATTGC | 2365 | GCAGUUUUUUUCAUUGCCA |
| exon_4 | | | | CAAAAACTAAATG | | AAAACUAAAUG |
| BCL11A_ | − | TTTG | 1055 | CAGTTTTTTTCATTGCC | 2366 | CAGUUUUUUUCAUUGCCAA |
| exon_4 | | | | AAAAACTAAATGG | | AAACUAAAUGG |
| BCL11A_ | − | GTTT | 1056 | TTTTCATTGCCAAAAAC | 2367 | UUUUCAUUGCCAAAAACUA |
| exon_4 | | | | TAAATGGTGCTTT | | AAUGGUGCUUU |
| BCL11A_ | − | TTTT | 1057 | TTTCATTGCCAAAAACT | 2368 | UUUCAUUGCCAAAAACUAA |
| exon_4 | | | | AAATGGTGCTTTA | | AUGGUGCUUUA |
| BCL11A_ | − | TTTT | 1058 | TTCATTGCCAAAAACTA | 2369 | UUCAUUGCCAAAAACUAAA |
| exon_4 | | | | AATGGTGCTTTAT | | UGGUGCUUUAU |
| BCL11A_ | − | TTTT | 1059 | TGTCCCTTTCCTTCTAT | 2370 | UGUCCCUUUCCUUCUAUCA |
| exon_4 | | | | CACCCTACATTCC | | CCCUACAUUCC |
| BCL11A_ | − | TTTT | 1060 | TCATTGCCAAAAACTAA | 2371 | UCAUUGCCAAAAACUAAAU |
| exon_4 | | | | ATGGTGCTTTATA | | GGUGCUUUAUA |
| BCL11A_ | − | TTTC | 1061 | ATTGCCAAAAACTAAAT | 2372 | AUUGCCAAAAACUAAAUGG |
| exon_4 | | | | GGTGCTTTATATT | | UGCUUUAUAUU |
| BCL11A_ | − | ATTG | 1062 | CCAAAAACTAAATGGTG | 2373 | CCAAAAACUAAAUGGUGCU |
| exon_4 | | | | CTTTATATTTAGA | | UUAUAUUUAGA |
| BCL11A_ | − | CTTT | 1063 | ATATTTAGATTGGAAAG | 2374 | AUAUUUAGAUUGGAAAGAA |
| exon_4 | | | | AATTTCATATGCA | | UUUCAUAUGCA |
| BCL11A_ | − | TTTA | 1064 | TATTTAGATTGGAAAGA | 2375 | UAUUUAGAUUGGAAAGAAU |
| exon_4 | | | | ATTTCATATGCAA | | UUCAUAUGCAA |
| BCL11A_ | − | ATTT | 1065 | AGATTGGAAAGAATTTC | 2376 | AGAUUGGAAAGAAUUUCAU |
| exon_4 | | | | ATATGCAAAGCAT | | AUGCAAAGCAU |
| BCL11A_ | − | TTTA | 1066 | GATTGGAAAGAATTTCA | 2377 | GAUUGGAAAGAAUUUCAUA |
| exon_4 | | | | TATGCAAAGCATA | | UGCAAAGCAUA |
| BCL11A_ | − | ATTG | 1067 | GAAAGAATTTCATATGC | 2378 | GAAAGAAUUUCAUAUGCAA |
| exon_4 | | | | AAAGCATATTAAA | | AGCAUAUUAAA |
| BCL11A_ | − | ATTT | 1068 | CATATGCAAAGCATATT | 2379 | CAUAUGCAAAGCAUAUUAA |
| exon_4 | | | | AAAGAGAAAGCCC | | AGAGAAAGCCC |
| BCL11A_ | − | TTTC | 1069 | ATATGCAAAGCATATTA | 2380 | AUAUGCAAAGCAUAUUAAA |
| exon_4 | | | | AAGAGAAAGCCCG | | GAGAAAGCCCG |
| BCL11A_ | − | ATTA | 1070 | AAGAGAAAGCCCGCTTT | 2381 | AAGAGAAAGCCCGCUUUAG |
| exon_4 | | | | AGTCAATACTTTT | | UCAAUACUUUU |
| BCL11A_ | − | CTTT | 1071 | AGTCAATACTTTTTTGT | 2382 | AGUCAAUACUUUUUUGUAA |
| exon_4 | | | | AAATGGCAATGCA | | AUGGCAAUGCA |
| BCL11A_ | − | TTTA | 1072 | GTCAATACTTTTTTGTA | 2383 | GUCAAUACUUUUUUGUAAA |
| exon_4 | | | | AATGGCAATGCAG | | UGGCAAUGCAG |
| BCL11A_ | − | CTTT | 1073 | TTTGTAAATGGCAATGC | 2384 | UUUGUAAAUGGCAAUGCAG |
| exon 4 | | | | AGAATATTTTGTT | | AAUAUUUUGUU |
| BCL11A_ | − | TTTT | 1074 | TTGTAAATGGCAATGCA | 2385 | UUGUAAAUGGCAAUGCAGA |
| exon_4 | | | | GAATATTTTGTTA | | AUAUUUUGUUA |
| BCL11A_ | − | TTTT | 1075 | CATTGCCAAAAACTAAA | 2386 | CAUUGCCAAAAACUAAAUG |
| exon_4 | | | | TGGTGCTTTATAT | | GUGCUUUAUAU |
| BCL11A_ | − | CTTT | 1076 | TTGTCCCTTTCCTTCTA | 2387 | UUGUCCCUUUCCUUCUAUC |
| exon_4 | | | | TCACCCTACATTC | | ACCCUACAUUC |
| BCL11A_ | − | GTTA | 1077 | TGTAGTGTGCTTTTTGT | 2388 | UGUAGUGUGCUUUUUGUCC |
| exon_4 | | | | CCCTTTCCTTCTA | | CUUUCCUUCUA |
| BCL11A_ | − | TTTG | 1078 | TTATGTAGTGTGCTTTT | 2389 | UUAUGUAGUGUGCUUUUUG |
| exon_4 | | | | TGTCCCTTTCCTT | | UCCCUUUCCUU |
| BCL11A_ | − | TTTT | 1079 | TGGTAGTGGAAAAAAAA | 2390 | UGGUAGUGGAAAAAAAAAA |
| exon_4 | | | | AAGACAGGCTGCC | | GACAGGCUGCC |
| BCL11A_ | − | TTTT | 1080 | GGTAGTGGAAAAAAAAA | 2391 | GGUAGUGGAAAAAAAAAAG |
| exon_4 | | | | AGACAGGCTGCCA | | ACAGGCUGCCA |
| BCL11A_ | − | TTTG | 1081 | GTAGTGGAAAAAAAAAA | 2392 | GUAGUGGAAAAAAAAAAGA |
| exon_4 | | | | GACAGGCTGCCAC | | CAGGCUGCCAC |
| BCL11A_ | − | ATTT | 1082 | TTTTAATTTGGCAGGAT | 2393 | UUUUAAUUUGGCAGGAUAA |
| exon_4 | | | | AATATAGTGCAAA | | UAUAGUGCAAA |
| BCL11A_ | − | TTTT | 1083 | TTTAATTTGGCAGGATA | 2394 | UUUAAUUUGGCAGGAUAAU |
| exon_4 | | | | ATATAGTGCAAAT | | AUAGUGCAAAU |
| BCL11A_ | − | TTTT | 1084 | TTAATTTGGCAGGATAA | 2395 | UUAAUUUGGCAGGAUAAUA |
| exon_4 | | | | TATAGTGCAAATT | | UAGUGCAAAUU |
| BCL11A_ | − | TTTT | 1085 | TAATTTGGCAGGATAAT | 2396 | UAAUUUGGCAGGAUAAUAU |
| exon_4 | | | | ATAGTGCAAATTA | | AGUGCAAAUUA |
| BCL11A_ | − | TTTT | 1086 | AATTTGGCAGGATAATA | 2397 | AAUUUGGCAGGAUAAUAUA |
| exon_4 | | | | TAGTGCAAATTAT | | GUGCAAAUUAU |
| BCL11A_ | − | TTTA | 1087 | ATTTGGCAGGATAATAT | 2398 | AUUUGGCAGGAUAAUAUAG |
| exon_4 | | | | AGTGCAAATTATT | | UGCAAAUUAUU |
| BCL11A_ | − | ATTT | 1088 | GGCAGGATAATATAGTG | 2399 | GGCAGGAUAAUAUAGUGCA |
| exon_4 | | | | CAAATTATTTGTA | | AAUUAUUUGUA |
| BCL11A_ | − | TTTG | 1089 | GCAGGATAATATAGTGC | 2400 | GCAGGAUAAUAUAGUGCAA |
| exon_4 | | | | AAATTATTTGTAT | | AUUAUUUGUAU |
| BCL11A_ | − | ATTA | 1090 | TTTGTATGCTTCAAAAA | 2401 | UUUGUAUGCUUCAAAAAAA |
| exon 4 | | | | AAAAAAAAAGAGA | | AAAAAAAGAGA |
| BCL11A_ | − | ATTT | 1091 | GTATGCTTCAAAAAAAA | 2402 | GUAUGCUUCAAAAAAAAAA |
| exon 4 | | | | AAAAAAGAGAGAA | | AAAAGAGAGAA |
| BCL11A_ | − | TTTG | 1092 | TATGCTTCAAAAAAAAA | 2403 | UAUGCUUCAAAAAAAAAAA |
| exon_4 | | | | AAAAAGAGAGAAA | | AAAGAGAGAAA |
| BCL11A_ | − | CTTC | 1093 | AAAAAAAAAAAAAAGAG | 2404 | AAAAAAAAAAAAAAGAGAG |
| exon_4 | | | | AGAAACAAAAAAG | | AAACAAAAAAG |
| BCL11A_ | − | ATTA | 1094 | CAGATGAGAAGCCATAT | 2405 | CAGAUGAGAAGCCAUAUAA |
| exon_4 | | | | AATGGCGGTTTGG | | UGGCGGUUUGG |
| BCL11A_ | − | GTTT | 1095 | GGGGGAGCCTGCTAGAA | 2406 | GGGGGAGCCUGCUAGAAUG |
| exon_4 | | | | TGTCACATGGATG | | UCACAUGGAUG |
| BCL11A_ | − | GTTT | 1096 | GTTATGTAGTGTGCTTT | 2407 | GUUAUGUAGUGUGCUUUUU |
| exon_4 | | | | TTGTCCCTTTCCT | | GUCCCUUUCCU |
| BCL11A_ | − | GTTG | 1097 | GTTTGTTATGTAGTGTG | 2408 | GUUUGUUAUGUAGUGUGCU |
| exon_4 | | | | CTTTTTGTCCCTT | | UUUUGUCCCUU |
| BCL11A_ | − | TTTC | 1098 | CTGCTGCCATACTGTAT | 2409 | CUGCUGCCAUACUGUAUGC |
| exon_4 | | | | GCAGTACTGCAAG | | AGUACUGCAAG |
| BCL11A_ | − | TTTT | 1099 | CCTGCTGCCATACTGTA | 2410 | CCUGCUGCCAUACUGUAUG |
| exon_4 | | | | TGCAGTACTGCAA | | CAGUACUGCAA |
| BCL11A_ | − | TTTT | 1100 | TCCTGCTGCCATACTGT | 2411 | UCCUGCUGCCAUACUGUAU |
| exon_4 | | | | ATGCAGTACTGCA | | GCAGUACUGCA |
| BCL11A_ | − | CTTT | 1101 | TTCCTGCTGCCATACTG | 2412 | UUCCUGCUGCCAUACUGUA |
| exon_4 | | | | TATGCAGTACTGC | | UGCAGUACUGC |
| BCL11A_ | − | TTTT | 1102 | TGTAAATGGCAATGCAG | 2413 | UGUAAAUGGCAAUGCAGAA |
| exon_4 | | | | AATATTTTGTTAT | | UAUUUUGUUAU |
| BCL11A_ | − | GTTC | 1103 | CTTTTTCCTGCTGCCAT | 2414 | CUUUUUCCUGCUGCCAUAC |
| exon_4 | | | | ACTGTATGCAGTA | | UGUAUGCAGUA |
| BCL11A_ | − | TTTT | 1104 | GTTCCTTTTTCCTGCTG | 2415 | GUUCCUUUUUCCUGCUGCC |
| exon_4 | | | | CCATACTGTATGC | | AUACUGUAUGC |
| BCL11A_ | − | TTTT | 1105 | TGTTCCTTTTTCCTGCT | 2416 | UGUUCCUUUUUCCUGCUGC |
| exon_4 | | | | GCCATACTGTATG | | CAUACUGUAUG |
| BCL11A_ | − | TTTT | 1106 | TTGTTCCTTTTTCCTGC | 2417 | UUGUUCCUUUUUCCUGCUG |
| exon_4 | | | | TGCCATACTGTAT | | CCAUACUGUAU |
| BCL11A_ | − | CTTT | 1107 | TTTGTTCCTTTTTCCTG | 2418 | UUUGUUCCUUUUUCCUGCU |
| exon_4 | | | | CTGCCATACTGTA | | GCCAUACUGUA |
| BCL11A_ | − | GTTG | 1108 | TACATATCCTTTTTTGT | 2419 | UACAUAUCCUUUUUUGUUC |
| exon_4 | | | | TCCTTTTTCCTGC | | CUUUUUCCUGC |
| BCL11A_ | − | TTTG | 1109 | GGGGAGCCTGCTAGAAT | 2420 | GGGGAGCCUGCUAGAAUGU |
| exon_4 | | | | GTCACATGGATGG | | CACAUGGAUGG |
| BCL11A_ | − | TTTG | 1110 | TTCCTTTTTCCTGCTGC | 2421 | UUCCUUUUUCCUGCUGCCA |
| exon 4 | | | | CATACTGTATGCA | | UACUGUAUGCA |
| BCL11A_ | − | TTTT | 1111 | GTAAATGGCAATGCAGA | 2422 | GUAAAUGGCAAUGCAGAAU |
| exon 4 | | | | ATATTTTGTTATT | | AUUUUGUUAUU |
| BCL11A_ | − | TTTG | 1112 | TAAATGGCAATGCAGAA | 2423 | UAAAUGGCAAUGCAGAAUA |
| exon_4 | | | | TATTTTGTTATTG | | UUUUGUUAUUG |
| BCL11A_ | − | ATTT | 1113 | TGTTATTGGCCTTTTCT | 2424 | UGUUAUUGGCCUUUUCUAU |
| exon_4 | | | | ATTCCTGTAATGA | | UCCUGUAAUGA |
| BCL11A_ | − | GTTT | 1114 | TTATTTTTTTTTTTATT | 2425 | UUAUUUUUUUUUUUAUUUA |
| exon_4 | | | | TAGATGACCAAAG | | GAUGACCAAAG |
| BCL11A_ | − | TTTT | 1115 | TATTTTTTTTTTTATTT | 2426 | UAUUUUUUUUUUUAUUUAG |
| exon_4 | | | | AGATGACCAAAGG | | AUGACCAAAGG |
| BCL11A_ | − | TTTT | 1116 | ATTTTTTTTTTTATTTA | 2427 | AUUUUUUUUUUUAUUUAGA |
| exon_4 | | | | GATGACCAAAGGT | | UGACCAAAGGU |
| BCL11A_ | − | TTTA | 1117 | TTTTTTTTTTTATTTAG | 2428 | UUUUUUUUUUUAUUUAGAU |
| exon_4 | | | | ATGACCAAAGGTC | | GACCAAAGGUC |
| BCL11A_ | − | ATTT | 1118 | TTTTTTTTATTTAGATG | 2429 | UUUUUUUUAUUUAGAUGAC |
| exon_4 | | | | ACCAAAGGTCATT | | CAAAGGUCAUU |
| BCL11A_ | − | TTTT | 1119 | TTTTTTTATTTAGATGA | 2430 | UUUUUUUAUUUAGAUGACC |
| exon_4 | | | | CCAAAGGTCATTA | | AAAGGUCAUUA |
| BCL11A_ | − | TTTT | 1120 | TTTTTTATTTAGATGAC | 2431 | UUUUUUAUUUAGAUGACCA |
| exon_4 | | | | CAAAGGTCATTAC | | AAGGUCAUUAC |
| BCL11A_ | − | TTTT | 1121 | TTTTTATTTAGATGACC | 2432 | UUUUUAUUUAGAUGACCAA |
| exon_4 | | | | AAAGGTCATTACA | | AGGUCAUUACA |
| BCL11A_ | − | TTTT | 1122 | TTTTATTTAGATGACCA | 2433 | UUUUAUUUAGAUGACCAAA |
| exon 4 | | | | AAGGTCATTACAA | | GGUCAUUACAA |
| BCL11A_ | − | TTTT | 1123 | TTTATTTAGATGACCAA | 2434 | UUUAUUUAGAUGACCAAAG |
| exon_4 | | | | AGGTCATTACAAC | | GUCAUUACAAC |
| BCL11A_ | − | TTTT | 1124 | TTATTTAGATGACCAAA | 2435 | UUAUUUAGAUGACCAAAGG |
| exon_4 | | | | GGTCATTACAACC | | UCAUUACAACC |
| BCL11A_ | − | TTTT | 1125 | TATTTAGATGACCAAAG | 2436 | UAUUUAGAUGACCAAAGGU |
| exon_4 | | | | GTCATTACAACCT | | CAUUACAACCU |
| BCL11A_ | − | TTTT | 1126 | ATTTAGATGACCAAAGG | 2437 | AUUUAGAUGACCAAAGGUC |
| exon_4 | | | | TCATTACAACCTG | | AUUACAACCUG |
| BCL11A_ | − | TTTA | 1127 | TTTAGATGACCAAAGGT | 2438 | UUUAGAUGACCAAAGGUCA |
| exon_4 | | | | CATTACAACCTGG | | UUACAACCUGG |
| BCL11A_ | − | ATTT | 1128 | AGATGACCAAAGGTCAT | 2439 | AGAUGACCAAAGGUCAUUA |
| exon_4 | | | | TACAACCTGGCTT | | CAACCUGGCUU |
| BCL11A_ | − | TTTA | 1129 | GATGACCAAAGGTCATT | 2440 | GAUGACCAAAGGUCAUUAC |
| exon_4 | | | | ACAACCTGGCTTT | | AACCUGGCUUU |
| BCL11A_ | − | ATTA | 1130 | CAACCTGGCTTTTTATT | 2441 | CAACCUGGCUUUUUAUUGU |
| exon 4 | | | | GTATTTGTTTCTG | | AUUUGUUUCUG |
| BCL11A_ | − | ATTG | 1131 | GAAAAACCACTGTCTGT | 2442 | GAAAAACCACUGUCUGUGU |
| exon 4 | | | | GTTTTTTTGGCAG | | UUUUUUGGCAG |
| BCL11A_ | − | GTTC | 1132 | TATTGGAAAAACCACTG | 2443 | UAUUGGAAAAACCACUGUC |
| exon_4 | | | | TCTGTGTTTTTTT | | UGUGUUUUUUU |
| BCL11A_ | − | GTTA | 1133 | AGTTCTATTGGAAAAAC | 2444 | AGUUCUAUUGGAAAAACCA |
| exon_4 | | | | CACTGTCTGTGTT | | CUGUCUGUGUU |
| BCL11A_ | − | TTTG | 1134 | TTAAGTTCTATTGGAAA | 2445 | UUAAGUUCUAUUGGAAAAA |
| exon_4 | | | | AACCACTGTCTGT | | CCACUGUCUGU |
| BCL11A_ | − | CTTT | 1135 | GTTAAGTTCTATTGGAA | 2446 | GUUAAGUUCUAUUGGAAAA |
| exon_4 | | | | AAACCACTGTCTG | | ACCACUGUCUG |
| BCL11A_ | − | TTTC | 1136 | TGGTCTTTGTTAAGTTC | 2447 | UGGUCUUUGUUAAGUUCUA |
| exon_4 | | | | TATTGGAAAAACC | | UUGGAAAAACC |
| BCL11A_ | − | TTTG | 1137 | TTTTTATTTTTTTTTTT | 2448 | UUUUUAUUUUUUUUUUUAU |
| exon_4 | | | | ATTTAGATGACCA | | UUAGAUGACCA |
| BCL11A_ | − | GTTT | 1138 | CTGGTCTTTGTTAAGTT | 2449 | CUGGUCUUUGUUAAGUUCU |
| exon 4 | | | | CTATTGGAAAAAC | | AUUGGAAAAAC |
| BCL11A_ | − | ATTT | 1139 | GTTTCTGGTCTTTGTTA | 2450 | GUUUCUGGUCUUUGUUAAG |
| exon_4 | | | | AGTTCTATTGGAA | | UUCUAUUGGAA |
| BCL11A_ | − | ATTG | 1140 | TATTTGTTTCTGGTCTT | 2451 | UAUUUGUUUCUGGUCUUUG |
| exon 4 | | | | TGTTAAGTTCTAT | | UUAAGUUCUAU |
| BCL11A_ | − | TTTA | 1141 | TTGTATTTGTTTCTGGT | 2452 | UUGUAUUUGUUUCUGGUCU |
| exon 4 | | | | CTTTGTTAAGTTC | | UUGUUAAGUUC |
| BCL11A_ | − | TTTT | 1142 | ATTGTATTTGTTTCTGG | 2453 | AUUGUAUUUGUUUCUGGUC |
| exon 4 | | | | TCTTTGTTAAGTT | | UUUGUUAAGUU |
| BCL11A_ | − | TTTT | 1143 | TATTGTATTTGTTTCTG | 2454 | UAUUGUAUUUGUUUCUGGU |
| exon_4 | | | | GTCTTTGTTAAGT | | CUUUGUUAAGU |
| BCL11A_ | − | CTTT | 1144 | TTATTGTATTTGTTTCT | 2455 | UUAUUGUAUUUGUUUCUGG |
| exon_4 | | | | GGTCTTTGTTAAG | | UCUUUGUUAAG |
| BCL11A_ | − | TTTG | 1145 | TTTCTGGTCTTTGTTAA | 2456 | UUUCUGGUCUUUGUUAAGU |
| exon_4 | | | | GTTCTATTGGAAA | | UCUAUUGGAAA |
| BCL11A_ | − | TTTT | 1146 | TTGGTAGTGGAAAAAAA | 2457 | UUGGUAGUGGAAAAAAAAA |
| exon_4 | | | | AAAGACAGGCTGC | | AGACAGGCUGC |
| BCL11A_ | − | CTTT | 1147 | GTTTTTATTTTTTTTTT | 2458 | GUUUUUAUUUUUUUUUUUA |
| exon_4 | | | | TATTTAGATGACC | | UUUAGAUGACC |
| BCL11A_ | − | TTTT | 1148 | CTTTGTTTTTATTTTTT | 2459 | CUUUGUUUUUAUUUUUUUU |
| exon 4 | | | | TTTTTATTTAGAT | | UUUAUUUAGAU |
| BCL11A_ | − | TTTT | 1149 | GTTATTGGCCTTTTCTA | 2460 | GUUAUUGGCCUUUUCUAUU |
| exon 4 | | | | TTCCTGTAATGAA | | CCUGUAAUGAA |
| BCL11A_ | − | TTTG | 1150 | TTATTGGCCTTTTCTAT | 2461 | UUAUUGGCCUUUUCUAUUC |
| exon 4 | | | | TCCTGTAATGAAA | | CUGUAAUGAAA |
| BCL11A_ | − | GTTA | 1151 | TTGGCCTTTTCTATTCC | 2462 | UUGGCCUUUUCUAUUCCUG |
| exon_4 | | | | TGTAATGAAAGCT | | UAAUGAAAGCU |
| BCL11A_ | − | ATTG | 1152 | GCCTTTTCTATTCCTGT | 2463 | GCCUUUUCUAUUCCUGUAA |
| exon 4 | | | | AATGAAAGCTGTT | | UGAAAGCUGUU |
| BCL11A_ | − | CTTT | 1153 | TCTATTCCTGTAATGAA | 2464 | UCUAUUCCUGUAAUGAAAG |
| exon 4 | | | | AGCTGTTTGTCGT | | CUGUUUGUCGU |
| BCL11A_ | − | TTTT | 1154 | CTATTCCTGTAATGAAA | 2465 | CUAUUCCUGUAAUGAAAGC |
| exon_4 | | | | GCTGTTTGTCGTA | | UGUUUGUCGUA |
| BCL11A_ | − | TTTC | 1155 | TATTCCTGTAATGAAAG | 2466 | UAUUCCUGUAAUGAAAGCU |
| exon_4 | | | | CTGTTTGTCGTAA | | GUUUGUCGUAA |
| BCL11A_ | − | ATTC | 1156 | CTGTAATGAAAGCTGTT | 2467 | CUGUAAUGAAAGCUGUUUG |
| exon 4 | | | | TGTCGTAACTTGA | | UCGUAACUUGA |
| BCL11A_ | − | GTTT | 1157 | GTCGTAACTTGAAATTT | 2468 | GUCGUAACUUGAAAUUUUA |
| exon_4 | | | | TATCTTTTACTAT | | UCUUUUACUAU |
| BCL11A_ | − | TTTG | 1158 | TCGTAACTTGAAATTTT | 2469 | UCGUAACUUGAAAUUUUAU |
| exon_4 | | | | ATCTTTTACTATG | | CUUUUACUAUG |
| BCL11A_ | − | CTTG | 1159 | AAATTTTATCTTTTACT | 2470 | AAAUUUUAUCUUUUACUAU |
| exon_4 | | | | ATGGGAGTCACTA | | GGGAGUCACUA |
| BCL11A_ | − | ATTT | 1160 | TATCTTTTACTATGGGA | 2471 | UAUCUUUUACUAUGGGAGU |
| exon 4 | | | | GTCACTATTTATT | | CACUAUUUAUU |
| BCL11A_ | − | TTTT | 1161 | ATCTTTTACTATGGGAG | 2472 | AUCUUUUACUAUGGGAGUC |
| exon_4 | | | | TCACTATTTATTA | | ACUAUUUAUUA |
| BCL11A_ | − | TTTA | 1162 | TCTTTTACTATGGGAGT | 2473 | UCUUUUACUAUGGGAGUCA |
| exon_4 | | | | CACTATTTATTAT | | CUAUUUAUUAU |
| BCL11A_ | − | CTTT | 1163 | TACTATGGGAGTCACTA | 2474 | UACUAUGGGAGUCACUAUU |
| exon 4 | | | | TTTATTATTGCTT | | UAUUAUUGCUU |
| BCL11A_ | − | TTTT | 1164 | ACTATGGGAGTCACTAT | 2475 | ACUAUGGGAGUCACUAUUU |
| exon 4 | | | | TTATTATTGCTTA | | AUUAUUGCUUA |
| BCL11A_ | − | TTTA | 1165 | CTATGGGAGTCACTATT | 2476 | CUAUGGGAGUCACUAUUUA |
| exon_4 | | | | TATTATTGCTTAT | | UUAUUGCUUAU |
| BCL11A_ | − | TTTT | 1166 | TCTTTGTTTTTATTTTT | 2477 | UCUUUGUUUUUAUUUUUUU |
| exon 4 | | | | TTTTTTATTTAGA | | UUUUAUUUAGA |
| BCL11A_ | − | ATTT | 1167 | TTCTTTGTTTTTATTTT | 2478 | UUCUUUGUUUUUAUUUUUU |
| exon_4 | | | | TTTTTTTATTTAG | | UUUUUAUUUAG |
| BCL11A_ | − | TTTA | 1168 | TTTTTCTTTGTTTTTAT | 2479 | UUUUUCUUUGUUUUUAUUU |
| exon_4 | | | | TTTTTTTTTTATT | | UUUUUUUUAUU |
| BCL11A_ | − | TTTT | 1169 | ATTTTTCTTTGTTTTTA | 2480 | AUUUUUCUUUGUUUUUAUU |
| exon_4 | | | | TTTTTTTTTTTAT | | UUUUUUUUUAU |
| BCL11A_ | − | CTTT | 1170 | TATTTTTCTTTGTTTTT | 2481 | UAUUUUUCUUUGUUUUUAU |
| exon_4 | | | | ATTTTTTTTTTTA | | UUUUUUUUUUA |
| BCL11A_ | − | TTTG | 1171 | ATCTTTTATTTTTCTTT | 2482 | AUCUUUUAUUUUUCUUUGU |
| exon 4 | | | | GTTTTTATTTTTT | | UUUUAUUUUUU |
| BCL11A_ | − | TTTC | 1172 | TTTGTTTTTATTTTTTT | 2483 | UUUGUUUUUAUUUUUUUUU |
| exon_4 | | | | TTTTATTTAGATG | | UUAUUUAGAUG |
| BCL11A_ | − | ATTT | 1173 | GATCTTTTATTTTTCTT | 2484 | GAUCUUUUAUUUUUCUUUG |
| exon_4 | | | | TGTTTTTATTTTT | | UUUUUAUUUUU |
| BCL11A_ | − | GTTC | 1174 | AAAACAGAGGCACTTAA | 2485 | AAAACAGAGGCACUUAAUU |
| exon 4 | | | | TTTGATCTTTTAT | | UGAUCUUUUAU |
| BCL11A_ | − | CTTA | 1175 | TGTGCCCTGTTCAAAAC | 2486 | UGUGCCCUGUUCAAAACAG |
| exon_4 | | | | AGAGGCACTTAAT | | AGGCACUUAAU |
| BCL11A_ | − | ATTG | 1176 | CTTATGTGCCCTGTTCA | 2487 | CUUAUGUGCCCUGUUCAAA |
| exon_4 | | | | AAACAGAGGCACT | | ACAGAGGCACU |
| BCL11A_ | − | ATTA | 1177 | TTGCTTATGTGCCCTGT | 2488 | UUGCUUAUGUGCCCUGUUC |
| exon_4 | | | | TCAAAACAGAGGC | | AAAACAGAGGC |
| BCL11A_ | − | TTTA | 1178 | TTATTGCTTATGTGCCC | 2489 | UUAUUGCUUAUGUGCCCUG |
| exon 4 | | | | TGTTCAAAACAGA | | UUCAAAACAGA |
| BCL11A_ | − | ATTT | 1179 | ATTATTGCTTATGTGCC | 2490 | AUUAUUGCUUAUGUGCCCU |
| exon 4 | | | | CTGTTCAAAACAG | | GUUCAAAACAG |
| BCL11A_ | − | CTTA | 1180 | ATTTGATCTTTTATTTT | 2491 | AUUUGAUCUUUUAUUUUUC |
| exon 4 | | | | TCTTTGTTTTTAT | | UUUGUUUUUAU |
| BCL11A_ | − | CTTT | 1181 | TTTGGTAGTGGAAAAAA | 2492 | UUUGGUAGUGGAAAAAAAA |
| exon_4 | | | | AAAAGACAGGCTG | | AAGACAGGCUG |
| BCL11A_ | − | CTTA | 1182 | AAAGGTATCAATGTACC | 2493 | AAAGGUAUCAAUGUACCUU |
| exon_4 | | | | TTTTTTGGTAGTG | | UUUUGGUAGUG |
| BCL11A_ | − | GTTC | 1183 | TCTTAAAAGGTATCAAT | 2494 | UCUUAAAAGGUAUCAAUGU |
| exon_4 | | | | GTACCTTTTTTGG | | ACCUUUUUUGG |
| BCL11A_ | − | TTTC | 1184 | TCTAATCAGAGATACAG | 2495 | UCUAAUCAGAGAUACAGAG |
| exon_4 | | | | AGGTTGAGTATAA | | GUUGAGUAUAA |
| BCL11A_ | − | GTTG | 1185 | AGTATAAAATAAACCTG | 2496 | AGUAUAAAAUAAACCUGCU |
| exon_4 | | | | CTCAGATAGGACA | | CAGAUAGGACA |
| BCL11A_ | − | ATTA | 1186 | AGTGCACTGTACAATTT | 2497 | AGUGCACUGUACAAUUUUC |
| exon 4 | | | | TCCCAGTTTACAG | | CCAGUUUACAG |
| BCL11A_ | − | ATTT | 1187 | TCCCAGTTTACAGGTCT | 2498 | UCCCAGUUUACAGGUCUAU |
| exon 4 | | | | ATACTTAAGGGAA | | ACUUAAGGGAA |
| BCL11A_ | − | TTTT | 1188 | CCCAGTTTACAGGTCTA | 2499 | CCCAGUUUACAGGUCUAUA |
| exon_4 | | | | TACTTAAGGGAAA | | CUUAAGGGAAA |
| BCL11A_ | − | TTTC | 1189 | CCAGTTTACAGGTCTAT | 2500 | CCAGUUUACAGGUCUAUAC |
| exon 4 | | | | ACTTAAGGGAAAA | | UUAAGGGAAAA |
| BCL11A_ | − | GTTT | 1190 | ACAGGTCTATACTTAAG | 2501 | ACAGGUCUAUACUUAAGGG |
| exon_4 | | | | GGAAAAGTTGCAA | | AAAAGUUGCAA |
| BCL11A_ | − | TTTA | 1191 | CAGGTCTATACTTAAGG | 2502 | CAGGUCUAUACUUAAGGGA |
| exon_4 | | | | GAAAAGTTGCAAG | | AAAGUUGCAAG |
| BCL11A_ | − | CTTA | 1192 | AGGGAAAAGTTGCAAGA | 2503 | AGGGAAAAGUUGCAAGAAU |
| exon_4 | | | | ATGCTGAAAAAAA | | GCUGAAAAAAA |
| BCL11A_ | − | GTTG | 1193 | CAAGAATGCTGAAAAAA | 2504 | CAAGAAUGCUGAAAAAAAA |
| exon 4 | | | | AATTGAACACAAT | | UUGAACACAAU |
| BCL11A_ | − | ATTG | 1194 | AACACAATCTCATTGAG | 2505 | AACACAAUCUCAUUGAGGA |
| exon 4 | | | | GAGCATTTTTTAA | | GCAUUUUUUAA |
| BCL11A_ | − | ATTG | 1195 | AGGAGCATTTTTTAAAA | 2506 | AGGAGCAUUUUUUAAAAAC |
| exon_4 | | | | ACTAAAAAAAAAA | | UAAAAAAAAAA |
| BCL11A_ | − | ATTT | 1196 | TTTAAAAACTAAAAAAA | 2507 | UUUAAAAACUAAAAAAAAA |
| exon 4 | | | | AAAAAACTTTGCC | | AAAACUUUGCC |
| BCL11A_ | − | TTTT | 1197 | TTAAAAACTAAAAAAAA | 2508 | UUAAAAACUAAAAAAAAAA |
| exon_4 | | | | AAAAACTTTGCCA | | AAACUUUGCCA |
| BCL11A_ | − | TTTT | 1198 | TAAAAACTAAAAAAAAA | 2509 | UAAAAACUAAAAAAAAAAA |
| exon_4 | | | | AAAACTTTGCCAG | | AACUUUGCCAG |
| BCL11A_ | − | TTTT | 1199 | AAAAACTAAAAAAAAAA | 2510 | AAAAACUAAAAAAAAAAAA |
| exon_4 | | | | AAACTTTGCCAGC | | ACUUUGCCAGC |
| BCL11A_ | − | TTTA | 1200 | AAAACTAAAAAAAAAAA | 2511 | AAAACUAAAAAAAAAAAAA |
| exon_4 | | | | AACTTTGCCAGCC | | CUUUGCCAGCC |
| BCL11A_ | − | TTTC | 1201 | GCTTCTACAGTGCAAGG | 2512 | GCUUCUACAGUGCAAGGAU |
| exon_4 | | | | ATTTTTTTGTACA | | UUUUUUGUACA |
| BCL11A_ | − | CTTT | 1202 | CGCTTCTACAGTGCAAG | 2513 | CGCUUCUACAGUGCAAGGA |
| exon_4 | | | | GATTTTTTTGTAC | | UUUUUUUGUAC |
| BCL11A_ | − | ATTG | 1203 | CTTTCGCTTCTACAGTG | 2514 | CUUUCGCUUCUACAGUGCA |
| exon_4 | | | | CAAGGATTTTTTT | | AGGAUUUUUUU |
| BCL11A_ | − | CTTA | 1204 | ACATAGAAATGAATGAT | 2515 | ACAUAGAAAUGAAUGAUUG |
| exon_4 | | | | TGCTTTCGCTTCT | | CUUUCGCUUCU |
| BCL11A_ | − | ATTG | 1205 | CAAGCGCTGTGAATGGA | 2516 | CAAGCGCUGUGAAUGGAAA |
| exon_4 | | | | AACAGAATACACT | | CAGAAUACACU |
| BCL11A_ | − | CTTG | 1206 | GACGCAACATTGCAAGC | 2517 | GACGCAACAUUGCAAGCGC |
| exon_4 | | | | GCTGTGAATGGAA | | UGUGAAUGGAA |
| BCL11A_ | − | TTTT | 1207 | CTCTAATCAGAGATACA | 2518 | CUCUAAUCAGAGAUACAGA |
| exon 4 | | | | GAGGTTGAGTATA | | GGUUGAGUAUA |
| BCL11A_ | − | CTTA | 1208 | CTTGGACGCAACATTGC | 2519 | CUUGGACGCAACAUUGCAA |
| exon 4 | | | | AAGCGCTGTGAAT | | GCGCUGUGAAU |
| BCL11A_ | − | ATTG | 1209 | AGCTTACTTACTTGGAC | 2520 | AGCUUACUUACUUGGACGC |
| exon 4 | | | | GCAACATTGCAAG | | AACAUUGCAAG |
| BCL11A_ | − | CTTG | 1210 | ACTATTGAGCTTACTTA | 2521 | ACUAUUGAGCUUACUUACU |
| exon_4 | | | | CTTGGACGCAACA | | UGGACGCAACA |
| BCL11A_ | − | TTTA | 1211 | CTTGACTATTGAGCTTA | 2522 | CUUGACUAUUGAGCUUACU |
| exon_4 | | | | CTTACTTGGACGC | | UACUUGGACGC |
| BCL11A_ | − | ATTT | 1212 | ACTTGACTATTGAGCTT | 2523 | ACUUGACUAUUGAGCUUAC |
| exon_4 | | | | ACTTACTTGGACG | | UUACUUGGACG |
| BCL11A_ | − | TTTG | 1213 | CCAGCCATTTACTTGAC | 2524 | CCAGCCAUUUACUUGACUA |
| exon_4 | | | | TATTGAGCTTACT | | UUGAGCUUACU |
| BCL11A_ | − | CTTT | 1214 | GCCAGCCATTTACTTGA | 2525 | GCCAGCCAUUUACUUGACU |
| exon_4 | | | | CTATTGAGCTTAC | | AUUGAGCUUAC |
| BCL11A_ | − | CTTA | 1215 | CTTACTTGGACGCAACA | 2526 | CUUACUUGGACGCAACAUU |
| exon_4 | | | | TTGCAAGCGCTGT | | GCAAGCGCUGU |
| BCL11A_ | − | CTTC | 1216 | TACAGTGCAAGGATTTT | 2527 | UACAGUGCAAGGAUUUUUU |
| exon_4 | | | | TTTGTACAAAACT | | UGUACAAAACU |
| BCL11A_ | − | CTTT | 1217 | TCTCTAATCAGAGATAC | 2528 | UCUCUAAUCAGAGAUACAG |
| exon_4 | | | | AGAGGTTGAGTAT | | AGGUUGAGUAU |
| BCL11A_ | − | GTTC | 1218 | AAATAGCACTTGACTCT | 2529 | AAAUAGCACUUGACUCUGC |
| exon 4 | | | | GCCTGTGATATCT | | CUGUGAUAUCU |
| BCL11A_ | − | ATTT | 1219 | GTGTTAACATGGAAGAG | 2530 | GUGUUAACAUGGAAGAGGA |
| exon_4 | | | | GATTCATTGTTTT | | UUCAUUGUUUU |
| BCL11A_ | − | TTTG | 1220 | TGTTAACATGGAAGAGG | 2531 | UGUUAACAUGGAAGAGGAU |
| exon_4 | | | | ATTCATTGTTTTT | | UCAUUGUUUUU |
| BCL11A_ | − | GTTA | 1221 | ACATGGAAGAGGATTCA | 2532 | ACAUGGAAGAGGAUUCAUU |
| exon_4 | | | | TTGTTTTTATTTT | | GUUUUUAUUUU |
| BCL11A_ | − | ATTC | 1222 | ATTGTTTTTATTTTTAT | 2533 | AUUGUUUUUAUUUUUAUUU |
| exon_4 | | | | TTTTTTAATTTTT | | UUUUAAUUUUU |
| BCL11A_ | − | ATTG | 1223 | TTTTTATTTTTATTTTT | 2534 | UUUUUAUUUUUAUUUUUUU |
| exon_4 | | | | TTAATTTTTTCTT | | AAUUUUUUCUU |
| BCL11A_ | − | GTTT | 1224 | TTATTTTTATTTTTTTA | 2535 | UUAUUUUUAUUUUUUUAAU |
| exon_4 | | | | ATTTTTTCTTTTT | | UUUUUUUUUU |
| BCL11A_ | − | TTTT | 1225 | TATTTTTATTTTTTTAA | 2536 | UAUUUUUAUUUUUUUAAUU |
| exon 4 | | | | TTTTTTCTTTTTT | | UUUUCUUUUUU |
| BCL11A_ | − | TTTT | 1226 | ATTTTTATTTTTTTAAT | 2537 | AUUUUUAUUUUUUUAAUUU |
| exon 4 | | | | TTTTTCTTTTTTA | | UUUCUUUUUUA |
| BCL11A_ | − | TTTA | 1227 | TTTTTATTTTTTTAATT | 2538 | UUUUUAUUUUUUUAAUUUU |
| exon_4 | | | | TTTTCTTTTTTAT | | UUCUUUUUUAU |
| BCL11A_ | − | ATTT | 1228 | TTATTTTTTTAATTTTT | 2539 | UUAUUUUUUUAAUUUUUUC |
| exon 4 | | | | TCTTTTTTATTAA | | UUUUUUAUUAA |
| BCL11A_ | − | TTTT | 1229 | TATTTTTTTAATTTTTT | 2540 | UAUUUUUUUAAUUUUUUCU |
| exon 4 | | | | CTTTTTTATTAAG | | UUUUUAUUAAG |
| BCL11A_ | − | TTTT | 1230 | ATTTTTTTAATTTTTTC | 2541 | AUUUUUUUAAUUUUUUCUU |
| exon 4 | | | | TTTTTTATTAAGC | | UUUUAUUAAGC |
| BCL11A_ | − | TTTA | 1231 | TTTTTTTAATTTTTTCT | 2542 | UUUUUUUAAUUUUUUCUUU |
| exon_4 | | | | TTTTTATTAAGCT | | UUUAUUAAGCU |
| BCL11A_ | − | ATTT | 1232 | TTTTAATTTTTTCTTTT | 2543 | UUUUAAUUUUUUCUUUUUU |
| exon 4 | | | | TTATTAAGCTAGC | | AUUAAGCUAGC |
| BCL11A_ | − | TTTT | 1233 | TTTAATTTTTTCTTTTT | 2544 | UUUAAUUUUUUCUUUUUUA |
| exon 4 | | | | TATTAAGCTAGCA | | UUAAGCUAGCA |
| BCL11A_ | − | TTTT | 1234 | TTAATTTTTTCTTTTTT | 2545 | UUAAUUUUUUCUUUUUUAU |
| exon_4 | | | | ATTAAGCTAGCAT | | UAAGCUAGCAU |
| BCL11A_ | − | TTTT | 1235 | TAATTTTTTCTTTTTTA | 2546 | UAAUUUUUUCUUUUUUAUU |
| exon_4 | | | | TTAAGCTAGCATC | | AAGCUAGCAUC |
| BCL11A_ | − | GTTG | 1236 | GTGTTCAAATAGCACTT | 2547 | GUGUUCAAAUAGCACUUGA |
| exon 4 | | | | GACTCTGCCTGTG | | CUCUGCCUGUG |
| BCL11A_ | − | ATTA | 1237 | AGCTAGCATCTGCCCCA | 2548 | AGCUAGCAUCUGCCCCAGU |
| exon 4 | | | | GTTGGTGTTCAAA | | UGGUGUUCAAA |
| BCL11A_ | − | TTTA | 1238 | TTAAGCTAGCATCTGCC | 2549 | UUAAGCUAGCAUCUGCCCC |
| exon 4 | | | | CCAGTTGGTGTTC | | AGUUGGUGUUC |
| BCL11A_ | − | TTTT | 1239 | ATTAAGCTAGCATCTGC | 2550 | AUUAAGCUAGCAUCUGCCC |
| exon 4 | | | | CCCAGTTGGTGTT | | CAGUUGGUGUU |
| BCL11A_ | − | TTTT | 1240 | TATTAAGCTAGCATCTG | 2551 | UAUUAAGCUAGCAUCUGCC |
| exon_4 | | | | CCCCAGTTGGTGT | | CCAGUUGGUGU |
| BCL11A_ | − | TTTT | 1241 | TTATTAAGCTAGCATCT | 2552 | UUAUUAAGCUAGCAUCUGC |
| exon_4 | | | | GCCCCAGTTGGTG | | CCCAGUUGGUG |
| BCL11A_ | − | CTTG | 1242 | ACTCTGCCTGTGATATC | 2553 | ACUCUGCCUGUGAUAUCUG |
| exon_4 | | | | TGTATCTTTTCTC | | UAUCUUUUCUC |
| BCL11A_ | − | CTTT | 1243 | TTTATTAAGCTAGCATC | 2554 | UUUAUUAAGCUAGCAUCUG |
| exon_4 | | | | TGCCCCAGTTGGT | | CCCCAGUUGGU |
| BCL11A_ | − | TTTT | 1244 | CTTTTTTATTAAGCTAG | 2555 | CUUUUUUAUUAAGCUAGCA |
| exon_4 | | | | CATCTGCCCCAGT | | UCUGCCCCAGU |
| BCL11A_ | − | TTTT | 1245 | TCTTTTTTATTAAGCTA | 2556 | UCUUUUUUAUUAAGCUAGC |
| exon_4 | | | | GCATCTGCCCCAG | | AUCUGCCCCAG |
| BCL11A_ | − | TTTT | 1246 | TTCTTTTTTATTAAGCT | 2557 | UUCUUUUUUAUUAAGCUAG |
| exon_4 | | | | AGCATCTGCCCCA | | CAUCUGCCCCA |
| BCL11A_ | − | ATTT | 1247 | TTTCTTTTTTATTAAGC | 2558 | UUUUUUUUUAUUAAGCUA |
| exon 4 | | | | TAGCATCTGCCCC | | GCAUCUGCCCC |
| BCL11A_ | − | TTTA | 1248 | ATTTTTTCTTTTTTATT | 2559 | AUUUUUUCUUUUUUAUUAA |
| exon_4 | | | | AAGCTAGCATCTG | | GCUAGCAUCUG |
| BCL11A_ | − | TTTT | 1249 | AATTTTTTCTTTTTTAT | 2560 | AAUUUUUUCUUUUUUAUUA |
| exon 4 | | | | TAAGCTAGCATCT | | AGCUAGCAUCU |
| BCL11A_ | − | TTTC | 1250 | TTTTTTATTAAGCTAGC | 2561 | UUUUUUAUUAAGCUAGCAU |
| exon 4 | | | | ATCTGCCCCAGTT | | CUGCCCCAGUU |
| BCL11A_ | − | TTTT | 1251 | CCATACACTGTGTGCTA | 2562 | CCAUACACUGUGUGCUAUU |
| exon 4 | | | | TTTGTGTTAACAT | | UGUGUUAACAU |
| BCL11A_ | − | ATTT | 1252 | TTTTGTACAAAACTTTT | 2563 | UUUUGUACAAAACUUUUUU |
| exon_4 | | | | TTAAATATAAATG | | AAAUAUAAAUG |
| BCL11A_ | − | TTTT | 1253 | TTGTACAAAACTTTTTT | 2564 | UUGUACAAAACUUUUUUAA |
| exon 4 | | | | AAATATAAATGTT | | AUAUAAAUGUU |
| BCL11A_ | − | ATTG | 1254 | GGGAAAGGTTTAAGATT | 2565 | GGGAAAGGUUUAAGAUUAU |
| exon 4 | | | | ATATAGTACTTAA | | AUAGUACUUAA |
| BCL11A_ | − | GTTT | 1255 | AAGATTATATAGTACTT | 2566 | AAGAUUAUAUAGUACUUAA |
| exon 4 | | | | AAATATAGGAAAA | | AUAUAGGAAAA |
| BCL11A_ | − | TTTA | 1256 | AGATTATATAGTACTTA | 2567 | AGAUUAUAUAGUACUUAAA |
| exon 4 | | | | AATATAGGAAAAT | | UAUAGGAAAAU |
| BCL11A_ | − | ATTA | 1257 | TATAGTACTTAAATATA | 2568 | UAUAGUACUUAAAUAUAGG |
| exon 4 | | | | GGAAAATGCACAC | | AAAAUGCACAC |
| BCL11A_ | − | CTTA | 1258 | AATATAGGAAAATGCAC | 2569 | AAUAUAGGAAAAUGCACAC |
| exon 4 | | | | ACTCATGTTGATT | | UCAUGUUGAUU |
| BCL11A_ | − | GTTG | 1259 | ATTCCTATGCTAAAATA | 2570 | AUUCCUAUGCUAAAAUACA |
| exon_4 | | | | CATTTATGGTCTT | | UUUAUGGUCUU |
| BCL11A_ | − | ATTC | 1260 | CTATGCTAAAATACATT | 2571 | CUAUGCUAAAAUACAUUUA |
| exon_4 | | | | TATGGTCTTTTTT | | UGGUCUUUUUU |
| BCL11A_ | − | ATTT | 1261 | ATGGTCTTTTTTCTGTA | 2572 | AUGGUCUUUUUUCUGUAUU |
| exon_4 | | | | TTTCTAGAATGGT | | UCUAGAAUGGU |
| BCL11A_ | − | TTTA | 1262 | TGGTCTTTTTTCTGTAT | 2573 | UGGUCUUUUUUCUGUAUUU |
| exon_4 | | | | TTCTAGAATGGTA | | CUAGAAUGGUA |
| BCL11A_ | − | CTTT | 1263 | TTTCTGTATTTCTAGAA | 2574 | UUUCUGUAUUUCUAGAAUG |
| exon_4 | | | | TGGTATTTGAATT | | GUAUUUGAAUU |
| BCL11A_ | − | TTTT | 1264 | TTCTGTATTTCTAGAAT | 2575 | UUCUGUAUUUCUAGAAUGG |
| exon_4 | | | | GGTATTTGAATTA | | UAUUUGAAUUA |
| BCL11A_ | − | TTTT | 1265 | TCTGTATTTCTAGAATG | 2576 | UCUGUAUUUCUAGAAUGGU |
| exon_4 | | | | GTATTTGAATTAA | | AUUUGAAUUAA |
| BCL11A_ | − | TTTT | 1266 | CTGTATTTCTAGAATGG | 2577 | CUGUAUUUCUAGAAUGGUA |
| exon_4 | | | | TATTTGAATTAAA | | UUUGAAUUAAA |
| BCL11A_ | − | TTTC | 1267 | TGTATTTCTAGAATGGT | 2578 | UGUAUUUCUAGAAUGGUAU |
| exon_4 | | | | ATTTGAATTAAAT | | UUGAAUUAAAU |
| BCL11A_ | − | ATTT | 1268 | CTAGAATGGTATTTGAA | 2579 | CUAGAAUGGUAUUUGAAUU |
| exon_4 | | | | TTAAATGTTCATC | | AAAUGUUCAUC |
| BCL11A_ | − | TTTC | 1269 | TAGAATGGTATTTGAAT | 2580 | UAGAAUGGUAUUUGAAUUA |
| exon_4 | | | | TAAATGTTCATCT | | AAUGUUCAUCU |
| BCL11A_ | − | ATTT | 1270 | GAATTAAATGTTCATCT | 2581 | GAAUUAAAUGUUCAUCUAG |
| exon_4 | | | | AGTGTTAGGCACT | | UGUUAGGCACU |
| BCL11A_ | − | CTTG | 1271 | TTCTCTTAAAAGGTATC | 2582 | UUCUCUUAAAAGGUAUCAA |
| exon_4 | | | | AATGTACCTTTTT | | UGUACCUUUUU |
| BCL11A_ | − | GTTG | 1272 | CTTGTTCTCTTAAAAGG | 2583 | CUUGUUCUCUUAAAAGGUA |
| exon_4 | | | | TATCAATGTACCT | | UCAAUGUACCU |
| BCL11A_ | − | TTTA | 1273 | ACTGTTGCTTGTTCTCT | 2584 | ACUGUUGCUUGUUCUCUUA |
| exon_4 | | | | TAAAAGGTATCAA | | AAAGGUAUCAA |
| BCL11A_ | − | TTTT | 1274 | AACTGTTGCTTGTTCTC | 2585 | AACUGUUGCUUGUUCUCUU |
| exon 4 | | | | TTAAAAGGTATCA | | AAAAGGUAUCA |
| BCL11A_ | − | TTTT | 1275 | TAACTGTTGCTTGTTCT | 2586 | UAACUGUUGCUUGUUCUCU |
| exon 4 | | | | CTTAAAAGGTATC | | UAAAAGGUAUC |
| BCL11A_ | − | ATTT | 1276 | TTAACTGTTGCTTGTTC | 2587 | UUAACUGUUGCUUGUUCUC |
| exon 4 | | | | TCTTAAAAGGTAT | | UUAAAAGGUAU |
| BCL11A_ | − | GTTG | 1277 | TAAAAAAAAAAAACATA | 2588 | UAAAAAAAAAAAACAUACA |
| exon_4 | | | | CATTGGGGAAAGG | | UUGGGGAAAGG |
| BCL11A_ | − | CTTG | 1278 | TATTTTTAACTGTTGCT | 2589 | UAUUUUUAACUGUUGCUUG |
| exon 4 | | | | TGTTCTCTTAAAA | | UUCUCUUAAAA |
| BCL11A_ | − | TTTA | 1279 | TATTGAAGCTTGTATTT | 2590 | UAUUGAAGCUUGUAUUUUU |
| exon_4 | | | | TTAACTGTTGCTT | | AACUGUUGCUU |
| BCL11A_ | − | ATTT | 1280 | ATATTGAAGCTTGTATT | 2591 | AUAUUGAAGCUUGUAUUUU |
| exon_4 | | | | TTTAACTGTTGCT | | UAACUGUUGCU |
| BCL11A_ | − | GTTA | 1281 | GGCACTATAGTATTTAT | 2592 | GGCACUAUAGUAUUUAUAU |
| exon_4 | | | | ATTGAAGCTTGTA | | UGAAGCUUGUA |
| BCL11A_ | − | GTTC | 1282 | ATCTAGTGTTAGGCACT | 2593 | AUCUAGUGUUAGGCACUAU |
| exon_4 | | | | ATAGTATTTATAT | | AGUAUUUAUAU |
| BCL11A_ | − | ATTA | 1283 | AATGTTCATCTAGTGTT | 2594 | AAUGUUCAUCUAGUGUUAG |
| exon_4 | | | | AGGCACTATAGTA | | GCACUAUAGUA |
| BCL11A_ | − | TTTG | 1284 | AATTAAATGTTCATCTA | 2595 | AAUUAAAUGUUCAUCUAGU |
| exon_4 | | | | GTGTTAGGCACTA | | GUUAGGCACUA |
| BCL11A_ | − | ATTG | 1285 | AAGCTTGTATTTTTAAC | 2596 | AAGCUUGUAUUUUUAACUG |
| exon 4 | | | | TGTTGCTTGTTCT | | UUGCUUGUUCU |
| BCL11A_ | − | TTTT | 1286 | TTTGTACAAAACTTTTT | 2597 | UUUGUACAAAACUUUUUUA |
| exon 4 | | | | TAAATATAAATGT | | AAUAUAAAUGU |
| BCL11A_ | − | CTTC | 1287 | AGGTTGTAAAAAAAAAA | 2598 | AGGUUGUAAAAAAAAAAAA |
| exon 4 | | | | AACATACATTGGG | | CAUACAUUGGG |
| BCL11A_ | − | ATTC | 1288 | TATGCCTTGGATACACA | 2599 | UAUGCCUUGGAUACACACC |
| exon 4 | | | | CCGCTCTTCAGGT | | GCUCUUCAGGU |
| BCL11A_ | − | TTTT | 1289 | TGTACAAAACTTTTTTA | 2600 | UGUACAAAACUUUUUUAAA |
| exon_4 | | | | AATATAAATGTTA | | UAUAAAUGUUA |
| BCL11A_ | − | TTTT | 1290 | GTACAAAACTTTTTTAA | 2601 | GUACAAAACUUUUUUAAAU |
| exon_4 | | | | ATATAAATGTTAA | | AUAAAUGUUAA |
| BCL11A_ | − | TTTG | 1291 | TACAAAACTTTTTTAAA | 2602 | UACAAAACUUUUUUAAAUA |
| exon_4 | | | | TATAAATGTTAAG | | UAAAUGUUAAG |
| BCL11A_ | − | CTTT | 1292 | TTTAAATATAAATGTTA | 2603 | UUUAAAUAUAAAUGUUAAG |
| exon_4 | | | | AGAAAAATTTTTT | | AAAAAUUUUUU |
| BCL11A_ | − | TTTT | 1293 | TTAAATATAAATGTTAA | 2604 | UUAAAUAUAAAUGUUAAGA |
| exon_4 | | | | GAAAAATTTTTTT | | AAAAUUUUUUU |
| BCL11A_ | − | TTTT | 1294 | TAAATATAAATGTTAAG | 2605 | UAAAUAUAAAUGUUAAGAA |
| exon_4 | | | | AAAAATTTTTTTT | | AAAUUUUUUUU |
| BCL11A_ | − | TTTT | 1295 | AAATATAAATGTTAAGA | 2606 | AAAUAUAAAUGUUAAGAAA |
| exon 4 | | | | AAAATTTTTTTTA | | AAUUUUUUUUA |
| BCL11A_ | − | TTTA | 1296 | AATATAAATGTTAAGAA | 2607 | AAUAUAAAUGUUAAGAAAA |
| exon_4 | | | | AAATTTTTTTTAA | | AUUUUUUUUAA |
| BCL11A_ | − | GTTA | 1297 | AGAAAAATTTTTTTTAA | 2608 | AGAAAAAUUUUUUUUAAAA |
| exon_4 | | | | AAAACACTTCATT | | AACACUUCAUU |
| BCL11A_ | − | ATTT | 1298 | TTTTTAAAAAACACTTC | 2609 | UUUUUAAAAAACACUUCAU |
| exon_4 | | | | ATTATGTTTAGGG | | UAUGUUUAGGG |
| BCL11A_ | − | TTTT | 1299 | TTTTAAAAAACACTTCA | 2610 | UUUUAAAAAACACUUCAUU |
| exon_4 | | | | TTATGTTTAGGGG | | AUGUUUAGGGG |
| BCL11A_ | − | TTTT | 1300 | TTTAAAAAACACTTCAT | 2611 | UUUAAAAAACACUUCAUUA |
| exon 4 | | | | TATGTTTAGGGGG | | UGUUUAGGGGG |
| BCL11A_ | − | TTTT | 1301 | TTAAAAAACACTTCATT | 2612 | UUAAAAAACACUUCAUUAU |
| exon_4 | | | | ATGTTTAGGGGGG | | GUUUAGGGGGG |
| BCL11A_ | − | TTTT | 1302 | TAAAAAACACTTCATTA | 2613 | UAAAAAACACUUCAUUAUG |
| exon 4 | | | | TGTTTAGGGGGGA | | UUUAGGGGGGA |
| BCL11A_ | − | TTTT | 1303 | AAAAAACACTTCATTAT | 2614 | AAAAAACACUUCAUUAUGU |
| exon_4 | | | | GTTTAGGGGGGAA | | UUAGGGGGGAA |
| BCL11A_ | − | TTTA | 1304 | AAAAACACTTCATTATG | 2615 | AAAAACACUUCAUUAUGUU |
| exon 4 | | | | TTTAGGGGGGAAC | | UAGGGGGGAAC |
| BCL11A_ | − | CTTC | 1305 | ATTATGTTTAGGGGGGA | 2616 | AUUAUGUUUAGGGGGGAAC |
| exon 4 | | | | ACTGCATTTTAGG | | UGCAUUUUAGG |
| BCL11A_ | − | TTTA | 1306 | AAAATGGTAGTGGAAAT | 2617 | AAAAUGGUAGUGGAAAUUC |
| exon_4 | | | | TCTATGCCTTGGA | | UAUGCCUUGGA |
| BCL11A_ | − | ATTT | 1307 | AAAAATGGTAGTGGAAA | 2618 | AAAAAUGGUAGUGGAAAUU |
| exon_4 | | | | TTCTATGCCTTGG | | CUAUGCCUUGG |
| BCL11A_ | − | GTTA | 1308 | TCCATTTAAAAATGGTA | 2619 | UCCAUUUAAAAAUGGUAGU |
| exon_4 | | | | GTGGAAATTCTAT | | GGAAAUUCUAU |
| BCL11A_ | − | CTTG | 1309 | TTATCCATTTAAAAATG | 2620 | UUAUCCAUUUAAAAAUGGU |
| exon_4 | | | | GTAGTGGAAATTC | | AGUGGAAAUUC |
| BCL11A_ | − | GTTA | 1310 | CAAGACTTGTTATCCAT | 2621 | CAAGACUUGUUAUCCAUUU |
| exon_4 | | | | TTAAAAATGGTAG | | AAAAAUGGUAG |
| BCL11A_ | − | CTTG | 1311 | GTGGTGTTACAAGACTT | 2622 | GUGGUGUUACAAGACUUGU |
| exon_4 | | | | GTTATCCATTTAA | | UAUCCAUUUAA |
| BCL11A_ | − | CTTG | 1312 | GATACACACCGCTCTTC | 2623 | GAUACACACCGCUCUUCAG |
| exon_4 | | | | AGGTTGTAAAAAA | | GUUGUAAAAAA |
| BCL11A_ | − | ATTG | 1313 | TCTTGGTGGTGTTACAA | 2624 | UCUUGGUGGUGUUACAAGA |
| exon_4 | | | | GACTTGTTATCCA | | CUUGUUAUCCA |
| BCL11A_ | − | TTTA | 1314 | GGGTTCCATTGTCTTGG | 2625 | GGGUUCCAUUGUCUUGGUG |
| exon_4 | | | | TGGTGTTACAAGA | | GUGUUACAAGA |
| BCL11A_ | − | TTTT | 1315 | AGGGTTCCATTGTCTTG | 2626 | AGGGUUCCAUUGUCUUGGU |
| exon_4 | | | | GTGGTGTTACAAG | | GGUGUUACAAG |
| BCL11A_ | − | ATTT | 1316 | TAGGGTTCCATTGTCTT | 2627 | UAGGGUUCCAUUGUCUUGG |
| exon_4 | | | | GGTGGTGTTACAA | | UGGUGUUACAA |
| BCL11A_ | − | TTTA | 1317 | GGGGGGAACTGCATTTT | 2628 | GGGGGGAACUGCAUUUUAG |
| exon_4 | | | | AGGGTTCCATTGT | | GGUUCCAUUGU |
| BCL11A_ | − | GTTT | 1318 | AGGGGGGAACTGCATTT | 2629 | AGGGGGGAACUGCAUUUUA |
| exon_4 | | | | TAGGGTTCCATTG | | GGGUUCCAUUG |
| BCL11A_ | − | ATTA | 1319 | TGTTTAGGGGGGAACTG | 2630 | UGUUUAGGGGGGAACUGCA |
| exon_4 | | | | CATTTTAGGGTTC | | UUUUAGGGUUC |
| BCL11A_ | − | GTTC | 1320 | CATTGTCTTGGTGGTGT | 2631 | CAUUGUCUUGGUGGUGUUA |
| exon_4 | | | | TACAAGACTTGTT | | CAAGACUUGUU |
| BCL11A_ | + | TTTA | 1321 | CCTGCAAAATAATACAA | 2632 | CCUGCAAAAUAAUACAACA |
| exon_4 | | | | CACCAACATCAAT | | CCAACAUCAAU |

The invention includes all combinations of the direct repeats and spacers listed above, consistent with the disclosure herein.

In some embodiments, one or more RNA guides disrupt the GATAA motif of the enhancer region of the BCL11A gene. In some embodiments, two RNA guides disrupt the GATAA motif of the enhancer region of the BCL11A gene. For example, in some embodiments, the RNA guide of SEQ ID NO: 2677 (or an RNA guide with at least 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 2677) and the RNA guide of SEQ ID NO: 2678 (or an RNA guide with at least 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 2678) disrupt the GATAA motif. In other embodiments, the RNA guide of SEQ ID NO: 2677 (or an RNA guide with at least 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 2677) and the RNA guide of SEQ ID NO: 2679 (or an RNA guide with at least 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 2679) disrupt the GATAA motif. In yet other embodiments, the RNA guide of SEQ ID NO: 2678 (or an RNA guide with at least 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 2678) and the RNA guide of SEQ ID NO: 2679 (or an RNA guide with at least 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO: 2679) disrupt the GATAA motif.

In embodiments, the RNA guide does not consist of the sequence of

(SEQ ID NO: 2677)
AGAAAUCCGUCUUUCAUUGACGGGAAGCUAGUCUAGUGCAAGC;
(SEQ ID NO: 2678)
AGAAAUCCGUCUUUCAUUGACGGCUGGAGCCUGUGAUAAAAGC;
or
(SEQ ID NO: 2679)
AGAAAUCCGUCUUUCAUUGACGGUACCCCACCCACGCCCCCAC.

In some embodiments, a spacer sequence described herein comprises a uracil (U). In some embodiments, a spacer sequence described herein comprises a thymine (T). In some embodiments, a spacer sequence according to Table 5 comprises a sequence comprising a thymine in one or more places indicated as uracil in Table 5.

Modifications

The RNA guide may include one or more covalent modifications with respect to a reference sequence, in particular the parent polyribonucleotide, which are included within the scope of this invention.

Exemplary modifications can include any modification to the sugar, the nucleobase, the internucleoside linkage (e.g. to a linking phosphate/to a phosphodiester linkage/to the phosphodiester backbone), and any combination thereof. Some of the exemplary modifications provided herein are described in detail below.

The RNA guide may include any useful modification, such as to the sugar, the nucleobase, or the internucleoside linkage (e.g. to a linking phosphate/to a phosphodiester linkage/to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof). Additional modifications are described herein.

In some embodiments, the modification may include a chemical or cellular induced modification. For example, some nonlimiting examples of intracellular RNA modifications are described by Lewis and Pan in “RNA modifications and structures cooperate to guide RNA-protein interactions” from Nat Reviews Mol Cell Biol, 2017, 18:202-210.

Different sugar modifications, nucleotide modifications, and/or internucleoside linkages (e.g., backbone structures) may exist at various positions in the sequence. One of ordinary skill in the art will appreciate that the nucleotide analogs or other modification(s) may be located at any position(s) of the sequence, such that the function of the sequence is not substantially decreased. The sequence may include from about 1% to about 100% modified nucleotides (either in relation to overall nucleotide content, or in relation to one or more types of nucleotide, i.e. any one or more of A, G, U or C) or any intervening percentage (e.g., from 1% to 20%>, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%).

In some embodiments, sugar modifications (e.g., at the 2′ position or 4′ position) or replacement of the sugar at one or more ribonucleotides of the sequence may, as well as backbone modifications, include modification or replacement of the phosphodiester linkages. Specific examples of a sequence include, but are not limited to, sequences including modified backbones or no natural internucleoside linkages such as internucleoside modifications, including modification or replacement of the phosphodiester linkages. Sequences having modified backbones include, among others, those that do not have a phosphorus atom in the backbone. For the purposes of this application, and as sometimes referenced in the art, modified RNAs that do not have a phosphorus atom in their internucleoside backbone can also be considered to be oligonucleosides. In particular embodiments, a sequence will include ribonucleotides with a phosphorus atom in its internucleoside backbone.

Modified sequence backbones may include, for example, phosphorothioates, chiral phosphorothioates, phosphorodithioates, phosphotriesters, aminoalkylphosphotriesters, methyl and other alkyl phosphonates such as 3′-alkylene phosphonates and chiral phosphonates, phosphinates, phosphoramidates such as 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. In some embodiments, the sequence may be negatively or positively charged.

The modified nucleotides, which may be incorporated into the sequence, can be modified on the internucleoside linkage (e.g., phosphate backbone). Herein, in the context of the polynucleotide backbone, the phrases “phosphate” and “phosphodiester” are used interchangeably. Backbone phosphate groups can be modified by replacing one or more of the oxygen atoms with a different substituent. Further, the modified nucleosides and nucleotides can include the wholesale replacement of an unmodified phosphate moiety with another internucleoside linkage as described herein. Examples of modified phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be modified by the replacement of a linking oxygen with nitrogen (bridged phosphoramidates), sulfur (bridged phosphorothioates), and carbon (bridged methylene-phosphonates).

The α-thio substituted phosphate moiety is provided to confer stability to RNA and DNA polymers through the unnatural phosphorothioate backbone linkages. Phosphorothioate DNA and RNA have increased nuclease resistance and subsequently a longer half-life in a cellular environment.

In specific embodiments, a modified nucleoside includes an alpha-thio-nucleoside (e.g., 5′-O-(1-thiophosphate)-adenosine, 5′-O-(1-thiophosphate)-cytidine (α-thio-cytidine), 5′-O-(1-thiophosphate)-guanosine, 5′-O-(1-thiophosphate)-uridine, or 5′-O-(1-thiophosphate)-pseudouridine).

Other internucleoside linkages that may be employed according to the present invention, including internucleoside linkages which do not contain a phosphorous atom, are described herein.

In some embodiments, the sequence may include one or more cytotoxic nucleosides. For example, cytotoxic nucleosides may be incorporated into sequence, such as bifunctional modification. Cytotoxic nucleoside may include, but are not limited to, adenosine arabinoside, 5-azacytidine, 4′-thio-aracytidine, cyclopentenylcytosine, cladribine, clofarabine, cytarabine, cytosine arabinoside, 1-(2-C-cyano-2-deoxy-beta-D-arabino-pentofuranosyl)-cytosine, decitabine, 5-fluorouracil, fludarabine, floxuridine, gemcitabine, a combination of tegafur and uracil, tegafur ((RS)-5-fluoro-1-(tetrahydrofuran-2-yl)pyrimidine-2,4(1H,3H)-dione), troxacitabine, tezacitabine, 2′-deoxy-2′-methylidenecytidine (DMDC), and 6-mercaptopurine. Additional examples include fludarabine phosphate, N4-behenoyl-1-beta-D-arabinofuranosylcytosine, N4-octadecyl-1-beta-D-arabinofuranosylcytosine, N4-palmitoyl-1-(2-C-cyano-2-deoxy-beta-D-arabino-pentofuranosyl) cytosine, and P-4055 (cytarabine 5′-elaidic acid ester).

In some embodiments, the sequence includes one or more post-transcriptional modifications (e.g., capping, cleavage, polyadenylation, splicing, poly-A sequence, methylation, acylation, phosphorylation, methylation of lysine and arginine residues, acetylation, and nitrosylation of thiol groups and tyrosine residues, etc). The one or more post-transcriptional modifications can be any post-transcriptional modification, such as any of the more than one hundred different nucleoside modifications that have been identified in RNA (Rozenski, J, Crain, P, and McCloskey, J. (1999). The RNA Modification Database: 1999 update. Nucl Acids Res 27: 196-197) In some embodiments, the first isolated nucleic acid comprises messenger RNA (mRNA). In some embodiments, the mRNA comprises at least one nucleoside selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some embodiments, the mRNA comprises at least one nucleoside selected from the group consisting of 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In some embodiments, the mRNA comprises at least one nucleoside selected from the group consisting of 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. In some embodiments, mRNA comprises at least one nucleoside selected from the group consisting of inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

The sequence may or may not be uniformly modified along the entire length of the molecule. For example, one or more or all types of nucleotide (e.g., naturally-occurring nucleotides, purine or pyrimidine, or any one or more or all of A, G, U, C, I, pU) may or may not be uniformly modified in the sequence, or in a given predetermined sequence region thereof. In some embodiments, the sequence includes a pseudouridine. In some embodiments, the sequence includes an inosine, which may aid in the immune system characterizing the sequence as endogenous versus viral RNAs. The incorporation of inosine may also mediate improved RNA stability/reduced degradation. See for example, Yu, Z. et al. (2015) RNA editing by ADAR1 marks dsRNA as “self”. Cell Res. 25, 1283-1284, which is incorporated by reference in its entirety.

### Cas12i Polypeptide

In some embodiments, the composition of the present invention includes a Cas12i polypeptide as described in PCT/US2019/022375.

In some embodiments, the composition of the present invention includes a Cas12i2 polypeptide described herein (e.g., a polypeptide comprising SEQ ID NO: 2634 and/or encoded by SEQ ID NO: 2633). In some embodiments, the Cas12i2 polypeptide comprises at least one RuvC domain.

A nucleic acid sequence encoding the Cas12i2 polypeptide described herein may be substantially identical to a reference nucleic acid sequence, e.g., SEQ ID NO: 2633. In some embodiments, the Cas12i2 polypeptide is encoded by a nucleic acid comprising a sequence having least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the reference nucleic acid sequence, e.g., SEQ ID NO: 2633. The percent identity between two such nucleic acids can be determined manually by inspection of the two optimally aligned nucleic acid sequences or by using software programs or algorithms (e.g., BLAST, ALIGN, CLUSTAL) using standard parameters. One indication that two nucleic acid sequences are substantially identical is that the nucleic acid molecules hybridize to the complementary sequence of the other under stringent conditions of temperature and ionic strength (e.g., within a range of medium to high stringency). See, e.g., Tijssen, “Hybridization with Nucleic Acid Probes. Part I. Theory and Nucleic Acid Preparation” (Laboratory Techniques in Biochemistry and Molecular Biology, Vol 24).

In some embodiments, the Cas12i2 polypeptide is encoded by a nucleic acid sequence having at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more sequence identity, but not 100% sequence identity, to a reference nucleic acid sequence, e.g., SEQ ID NO: 2633.

In some embodiments, the Cas12i2 polypeptide of the present invention comprises a polypeptide sequence having at least 50%, 60%, 65%, 70%, 75%, 80%, 85%, 90%, 91%, 92%, 93%, 94%, 95%, 96%, 97%, 98%, 99%, or 100% identity to SEQ ID NO: 2634.

In some embodiments, the present invention describes a Cas12i2 polypeptide having a specified degree of amino acid sequence identity to one or more reference polypeptides, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99%, but not 100%, sequence identity to the amino acid sequence of SEQ ID NO: 2634. Homology or identity can be determined by amino acid sequence alignment, e.g., using a program such as BLAST, ALIGN, or CLUSTAL, as described herein.

Also provided is a Cas12i2 polypeptide of the present invention having enzymatic activity, e.g., nuclease or endonuclease activity, and comprising an amino acid sequence which differs from the amino acid sequences of SEQ ID NO: 2634 by 50, 40, 35, 30, 25, 20, 19, 18, 17, 16, 15, 14, 13, 12, 11, 10, 9, 8, 7, 6, 5, 4, 3, 2, 1, or 0 amino acid residue(s), when aligned using any of the previously described alignment methods.

In some embodiments, the Cas12i2 polypeptide comprises a polypeptide having a sequence of SEQ ID NO: 2641, SEQ ID NO: 2642, SEQ ID NO: 2643, SEQ ID NO: 2644, or SEQ ID NO: 2645.

In some embodiments, the Cas12i2 polypeptide of the present invention comprises a polypeptide sequence having at least 50%, 60%, 65%, 70%, 75%, 80%, 85%, 90%, 91%, 92%, 93%, 94%, 95%, 96%, 97%, 98%, 99%, or 100% identity to SEQ ID NO: 2641, SEQ ID NO: 2642, SEQ ID NO: 2643, SEQ ID NO: 2644, or SEQ ID NO: 2645. In some embodiments, a Cas12i2 polypeptide having at least 50%, 60%, 65%, 70%, 75%, 80%, 85%, 90%, 91%, 92%, 93%, 94%, 95%, 96%, 97%, 98%, 99%, or 100% identity to SEQ ID NO: 2641, SEQ ID NO: 2642, SEQ ID NO: 2643, SEQ ID NO: 2644, or SEQ ID NO: 2645 maintains the amino acid changes (or at least 1, 2, 3 etc. of these changes) that differentiate it from its respective parent/reference sequence.

In some embodiments, the present invention describes a Cas12i2 polypeptide having a specified degree of amino acid sequence identity to one or more reference polypeptides, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99%, but not 100%, sequence identity to the amino acid sequence of SEQ ID NO: 2641, SEQ ID NO: 2642, SEQ ID NO: 2643, SEQ ID NO: 2644, or SEQ ID NO: 2645. Homology or identity can be determined by amino acid sequence alignment, e.g., using a program such as BLAST, ALIGN, or CLUSTAL, as described herein.

Also provided is a Cas12i2 polypeptide of the present invention having enzymatic activity, e.g., nuclease or endonuclease activity, and comprising an amino acid sequence which differs from the amino acid sequences of SEQ ID NO: 2641, SEQ ID NO: 2642, SEQ ID NO: 2643, SEQ ID NO: 2644, or SEQ ID NO: 2645 by 50, 40, 35, 30, 25, 20, 19, 18, 17, 16, 15, 14, 13, 12, 11, 10, 9, 8, 7, 6, 5, 4, 3, 2, 1, or 0 amino acid residue(s), when aligned using any of the previously described alignment methods.

In some embodiments, the composition of the present invention includes a Cas12i4 polypeptide described herein (e.g., a polypeptide comprising SEQ ID NO: 2647 and/or encoded by SEQ ID NO: 2646). In some embodiments, the Cas12i4 polypeptide comprises at least one RuvC domain.

A nucleic acid sequence encoding the Cas12i4 polypeptide described herein may be substantially identical to a reference nucleic acid sequence, e.g., SEQ ID NO: 2646. In some embodiments, the Cas12i4 polypeptide is encoded by a nucleic acid comprising a sequence having least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the reference nucleic acid sequence, e.g., SEQ ID NO: 2646. The percent identity between two such nucleic acids can be determined manually by inspection of the two optimally aligned nucleic acid sequences or by using software programs or algorithms (e.g., BLAST, ALIGN, CLUSTAL) using standard parameters. One indication that two nucleic acid sequences are substantially identical is that the nucleic acid molecules hybridize to the complementary sequence of the other under stringent conditions of temperature and ionic strength (e.g., within a range of medium to high stringency).

In some embodiments, the Cas12i4 polypeptide is encoded by a nucleic acid sequence having at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more sequence identity, but not 100% sequence identity, to a reference nucleic acid sequence, e.g., SEQ ID NO: 2646.

In some embodiments, the Cas12i4 polypeptide of the present invention comprises a polypeptide sequence having at least 50%, 60%, 65%, 70%, 75%, 80%, 85%, 90%, 91%, 92%, 93%, 94%, 95%, 96%, 97%, 98%, 99%, or 100% identity to SEQ ID NO: 2647.

In some embodiments, the present invention describes a Cas12i4 polypeptide having a specified degree of amino acid sequence identity to one or more reference polypeptides, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99%, but not 100%, sequence identity to the amino acid sequence of SEQ ID NO: 2647. Homology or identity can be determined by amino acid sequence alignment, e.g., using a program such as BLAST, ALIGN, or CLUSTAL, as described herein.

Also provided is a Cas12i4 polypeptide of the present invention having enzymatic activity, e.g., nuclease or endonuclease activity, and comprising an amino acid sequence which differs from the amino acid sequences of SEQ ID NO: 2647 by 50, 40, 35, 30, 25, 20, 19, 18, 17, 16, 15, 14, 13, 12, 11, 10, 9, 8, 7, 6, 5, 4, 3, 2, 1, or 0 amino acid residue(s), when aligned using any of the previously described alignment methods.

In some embodiments, the Cas12i4 polypeptide comprises a polypeptide having a sequence of SEQ ID NO: 2648 or SEQ ID NO: 2649.

In some embodiments, the Cas12i4 polypeptide of the present invention comprises a polypeptide sequence having at least 50%, 60%, 65%, 70%, 75%, 80%, 85%, 90%, 91%, 92%, 93%, 94%, 95%, 96%, 97%, 98%, 99%, or 100% identity to SEQ ID NO: 2648 or SEQ ID NO: 2649. In some embodiments, a Cas12i4 polypeptide having at least 50%, 60%, 65%, 70%, 75%, 80%, 85%, 90%, 91%, 92%, 93%, 94%, 95%, 96%, 97%, 98%, 99%, or 100% identity to SEQ ID NO: 2648 or SEQ ID NO: 2649 maintains the amino acid changes (or at least 1, 2, 3 etc, of these changes) that differentiate it from its respective parent/reference sequence.

In some embodiments, the present invention describes a Cas12i4 polypeptide having a specified degree of amino acid sequence identity to one or more reference polypeptides, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99%, but not 100%, sequence identity to the amino acid sequence of SEQ ID NO: 2648 or SEQ ID NO: 2649. Homology or identity can be determined by amino acid sequence alignment, e.g., using a program such as BLAST, ALIGN, or CLUSTAL, as described herein.

Also provided is a Cas12i4 polypeptide of the present invention having enzymatic activity, e.g., nuclease or endonuclease activity, and comprising an amino acid sequence which differs from the amino acid sequences of SEQ ID NO: 2648 or SEQ ID NO: 2649 by 50, 40, 35, 30, 25, 20, 19, 18, 17, 16, 15, 14, 13, 12, 11, 10, 9, 8, 7, 6, 5, 4, 3, 2, 1, or 0 amino acid residue(s), when aligned using any of the previously described alignment methods.

In some embodiments, the composition of the present invention includes a Cas12i1 polypeptide described herein (e.g., a polypeptide comprising SEQ ID NO: 2650). In some embodiments, the Cas12i4 polypeptide comprises at least one RuvC domain.

In some embodiments, the Cas12i1 polypeptide of the present invention comprises a polypeptide sequence having at least 50%, 60%, 65%, 70%, 75%, 80%, 85%, 90%, 91%, 92%, 93%, 94%, 95%, 96%, 97%, 98%, 99%, or 100% identity to SEQ ID NO: 2650.

In some embodiments, the present invention describes a Cas12i1 polypeptide having a specified degree of amino acid sequence identity to one or more reference polypeptides, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99%, but not 100%, sequence identity to the amino acid sequence of SEQ ID NO: 2650. Homology or identity can be determined by amino acid sequence alignment, e.g., using a program such as BLAST, ALIGN, or CLUSTAL, as described herein.

Also provided is a Cas12i1 polypeptide of the present invention having enzymatic activity, e.g., nuclease or endonuclease activity, and comprising an amino acid sequence which differs from the amino acid sequences of SEQ ID NO: 2650 by 50, 40, 35, 30, 25, 20, 19, 18, 17, 16, 15, 14, 13, 12, 11, 10, 9, 8, 7, 6, 5, 4, 3, 2, 1, or 0 amino acid residue(s), when aligned using any of the previously described alignment methods.

In some embodiments, the composition of the present invention includes a Cas12i3 polypeptide described herein (e.g., a polypeptide comprising SEQ ID NO: 2651). In some embodiments, the Cas12i4 polypeptide comprises at least one RuvC domain.

In some embodiments, the Cas12i3 polypeptide of the present invention comprises a polypeptide sequence having at least 50%, 60%, 65%, 70%, 75%, 80%, 85%, 90%, 91%, 92%, 93%, 94%, 95%, 96%, 97%, 98%, 99%, or 100% identity to SEQ ID NO: 2651.

In some embodiments, the present invention describes a Cas12i3 polypeptide having a specified degree of amino acid sequence identity to one or more reference polypeptides, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99%, but not 100%, sequence identity to the amino acid sequence of SEQ ID NO: 2651. Homology or identity can be determined by amino acid sequence alignment, e.g., using a program such as BLAST, ALIGN, or CLUSTAL, as described herein.

Also provided is a Cas12i3 polypeptide of the present invention having enzymatic activity, e.g., nuclease or endonuclease activity, and comprising an amino acid sequence which differs from the amino acid sequences of SEQ ID NO: 2651 by 50, 40, 35, 30, 25, 20, 19, 18, 17, 16, 15, 14, 13, 12, 11, 10, 9, 8, 7, 6, 5, 4, 3, 2, 1, or 0 amino acid residue(s), when aligned using any of the previously described alignment methods.

Although the changes described herein may be one or more amino acid changes, changes to the Cas12i polypeptide may also be of a substantive nature, such as fusion of polypeptides as amino- and/or carboxyl-terminal extensions. For example, the Cas12i polypeptide may contain additional peptides, e.g., one or more peptides. Examples of additional peptides may include epitope peptides for labelling, such as a polyhistidine tag (His-tag), Myc, and FLAG. In some embodiments, the Cas12i polypeptide described herein can be fused to a detectable moiety such as a fluorescent protein (e.g., green fluorescent protein (GFP) or yellow fluorescent protein (YFP)).

In some embodiments, the Cas12i polypeptide comprises at least one (e.g., two, three, four, five, six, or more) nuclear localization signal (NLS). In some embodiments, the Cas12i polypeptide comprises at least one (e.g., two, three, four, five, six, or more) nuclear export signal (NES). In some embodiments, the Cas12i polypeptide comprises at least one (e.g., two, three, four, five, six, or more) NLS and at least one (e.g., two, three, four, five, six, or more) NES.

In some embodiments, the Cas12i polypeptide described herein can be self-inactivating. See, Epstein et al., “Engineering a Self-Inactivating CRISPR System for AAV Vectors,” Mol. Ther., 24 (2016): S50, which is incorporated by reference in its entirety.

In some embodiments, the nucleotide sequence encoding the Cas12i polypeptide described herein can be codon-optimized for use in a particular host cell or organism. For example, the nucleic acid can be codon-optimized for any non-human eukaryote including mice, rats, rabbits, dogs, livestock, or non-human primates. Codon usage tables are readily available, for example, at the “Codon Usage Database” available at www.kazusa.orjp/codon/and these tables can be adapted in a number of ways. See Nakamura et al. Nucl. Acids Res. 28:292 (2000), which is incorporated herein by reference in its entirety. Computer algorithms for codon optimizing a particular sequence for expression in a particular host cell are also available, such as Gene Forge (Aptagen; Jacobus, PA).

### Target Sequence

In some embodiments, the target sequence is within a BCL11A gene or a locus of a BCL11A gene. In some embodiments, the BCL11A gene is a mammalian gene. In some embodiments, the BCL11A gene is a human gene. For example, in some embodiments, the target sequence is within the sequence of SEQ ID NO: 2635 or the reverse complement thereof. In some embodiments, the target sequence is within an exon or enhancer region of the BCL11A gene set forth in SEQ ID NO: 2635 (or the reverse complement thereof), e.g., within a sequence of SEQ ID NO: 2636, 2637, 2638, 2639, or 2640 (or a reverse complement thereof). Target sequences within an exon or enhancer region of the BCL11A gene of SEQ ID NO: 2635 (and the reverse complement thereof) are set forth in Table 5. In some embodiments, the target sequence is within an intron of the BCL11A gene set forth in SEQ ID NO: 2635 or the reverse complement thereof. In some embodiments, the target sequence is within a variant (e.g., a polymorphic variant) of the BCL11A gene sequence set forth in SEQ ID NO: 2635 or the reverse complement thereof. In some embodiments, the BCL11A gene sequence is a homolog of the sequence set forth in SEQ ID NO: 2635 or the reverse complement thereof. For examples, in some embodiments, the BCL11A gene sequence is a non-human BCL11A sequence.

In some embodiments, the target sequence is adjacent to a 5′-NTTN-3′ PAM sequence, wherein N is any nucleotide. The 5′-NTTN-3′ sequence may be immediately adjacent to the target sequence or, for example, within a small number (e.g., 1, 2, 3, 4, or 5) of nucleotides of the target sequence. In some embodiments the 5′-NTTN-3′ sequence is 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′, wherein Y is C or T, B is any nucleotide except for A, D is any nucleotide except for C, and R is A or G. In some embodiments, the 5′-NTTN-3′ sequence is 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′.

In some embodiments, the target sequence is single-stranded (e.g., single-stranded DNA). In some embodiments, the target sequence is double-stranded (e.g., double-stranded DNA). In some embodiments, the target sequence comprises both single-stranded and double-stranded regions. In some embodiments, the target sequence is linear. In some embodiments, the target sequence is circular. In some embodiments, the target sequence comprises one or more modified nucleotides, such as methylated nucleotides, damaged nucleotides, or nucleotides analogs. In some embodiments, the target sequence is not modified. In some embodiments, the RNA guide binds to a first strand of a double-stranded target sequence (e.g., the target strand or the spacer-complementary strand), and the 5′-NTTN-3′ PAM sequence is present in the second, complementary strand (e.g., the non-target strand or the non-spacer-complementary strand). In some embodiments, the RNA guide binds adjacent to a 5′-NAAN-3′ sequence on the target strand (e.g., the spacer-complementary strand).

In some embodiments, the target sequence is present in a cell. In some embodiments, the target sequence is present in the nucleus of the cell. In some embodiments, the target sequence is endogenous to the cell. In some embodiments, the target sequence is a genomic DNA. In some embodiments, the target sequence is a chromosomal DNA. In some embodiments, the target sequence is a protein-coding gene or a functional region thereof, such as a coding region, or a regulatory element, such as a promoter, enhancer, a 5′ or 3′ untranslated region, etc. In some embodiments, the target sequence is a plasmid.

In some embodiments, the target sequence is present in a readily accessible region of the target sequence. In some embodiments, the target sequence is in an exon of a target gene. In some embodiments, the target sequence is across an exon-intron junction of a target gene. In some embodiments, the target sequence is present in a non-coding region, such as a regulatory region of a gene. In some embodiments, wherein the target sequence is exogenous to a cell, the target sequence comprises a sequence that is not found in the genome of the cell.

In some embodiments, the target sequence is exogenous to a cell. In some embodiments, the target sequence is a horizontally transferred plasmid. In some embodiments, the target sequence is integrated in the genome of the cell. In some embodiments, the target sequence is not integrated in the genome of the cell. In some embodiments, the target sequence is a plasmid in the cell. In some embodiments, the target sequence is present in an extrachromosomal array.

In some embodiments, the target sequence is an isolated nucleic acid, such as an isolated DNA or an isolated RNA. In some embodiments, the target sequence is present in a cell-free environment. In some embodiments, the target sequence is an isolated vector, such as a plasmid. In some embodiments, the target sequence is an ultrapure plasmid.

The target sequence is a locus of the BCL11A gene that hybridizes to the RNA guide. In some embodiments, a cell has only one copy of the target sequence. In some embodiments, a cell has more than one copy, such as at least about any one of 2, 3, 4, 5, 10, 100, or more copies of the target sequence.

In some embodiments, a BCL11A target sequence is selected to be edited by a Cas12i polypeptide and an RNA guide using one or more of the following criteria. First, in some embodiments, a target sequence near the 5′ end of the BCL11A coding sequence is selected. For example, in some embodiments, an RNA guide is designed to target a sequence in exon 1 (SEQ ID NO: 2636), exon 2 (SEQ ID NO: 2637), or the enhancer region (SEQ ID NO: 2640). Second, in some embodiments, a target sequence adjacent to a 5′-CTTY-3′ PAM sequence is selected. For example, in some embodiments, an RNA guide is designed to target a sequence adjacent to a 5′-CTTT-3′ or 5′-CTTC-3′ sequence. Third, in some embodiments, a target sequence having low sequence similarity to other genomic sequences is selected. For example, for each target sequence, potential non-target sites can be identified by searching for other genomic sequences adjacent to a PAM sequence and calculating the Levenshtein distance between the target sequence and the PAM-adjacent sequences. The Levenshtein distance (e.g., edit distance) corresponds to the minimum number of edits (e.g., insertions, deletions, or substitutions) required to change one sequence into another (e.g., to change the sequence of a potential non-target locus into the sequence of the on-target locus). Following this analysis, RNA guides are designed for target sequences that do not have potential off-target sequences with a Levenshtein distance of 0 or 1.

### Production

The present invention includes methods for production of the RNA guide, methods for production of the Cas12i polypeptide, and methods for complexing the RNA guide and Cas12i polypeptide.

### RNA Guide

In some embodiments, the RNA guide is made by in vitro transcription of a DNA template. Thus, for example, in some embodiments, the RNA guide is generated by in vitro transcription of a DNA template encoding the RNA guide using an upstream promoter sequence (e.g., a T7 polymerase promoter sequence). In some embodiments, the DNA template encodes multiple RNA guides or the in vitro transcription reaction includes multiple different DNA templates, each encoding a different RNA guide. In some embodiments, the RNA guide is made using chemical synthetic methods. In some embodiments, the RNA guide is made by expressing the RNA guide sequence in cells transfected with a plasmid including sequences that encode the RNA guide. In some embodiments, the plasmid encodes multiple different RNA guides. In some embodiments, multiple different plasmids, each encoding a different RNA guide, are transfected into the cells. In some embodiments, the RNA guide is expressed from a plasmid that encodes the RNA guide and also encodes a Cas12i polypeptide. In some embodiments, the RNA guide is expressed from a plasmid that expresses the RNA guide but not a Cas12i polypeptide. In some embodiments, the RNA guide is purchased from a commercial vendor. In some embodiments, the RNA guide is synthesized using one or more modified nucleotide, e.g., as described above.

### Cas12i Polypeptide

In some embodiments, the Cas12i polypeptide of the present invention can be prepared by (a) culturing bacteria which produce the Cas12i polypeptide of the present invention, isolating the Cas12i polypeptide, optionally, purifying the Cas12i polypeptide, and complexing the Cas12i polypeptide with an RNA guide. The Cas12i polypeptide can be also prepared by (b) a known genetic engineering technique, specifically, by isolating a gene encoding the Cas12i polypeptide of the present invention from bacteria, constructing a recombinant expression vector, and then transferring the vector into an appropriate host cell that expresses the RNA guide for expression of a recombinant protein that complexes with the RNA guide in the host cell. Alternatively, the Cas12i polypeptide can be prepared by (c) an in vitro coupled transcription-translation system and then complexing with an RNA guide.

In some embodiments, a host cell is used to express the Cas12i polypeptide. The host cell is not particularly limited, and various known cells can be preferably used. Specific examples of the host cell include bacteria such as E. coli, yeasts (budding yeast, Saccharomyces cerevisiae, and fission yeast, Schizosaccharomyces pombe), nematodes (Caenorhabditis elegans), Xenopus laevis oocytes, and animal cells (for example, CHO cells, COS cells and HEK293 cells). The method for transferring the expression vector described above into host cells, i.e., the transformation method, is not particularly limited, and known methods such as electroporation, the calcium phosphate method, the liposome method and the DEAE dextran method can be used.

After a host is transformed with the expression vector, the host cells may be cultured, cultivated or bred, for production of the Cas12i polypeptide. After expression of the Cas12i polypeptide, the host cells can be collected and Cas12i polypeptide purified from the cultures etc. according to conventional methods (for example, filtration, centrifugation, cell disruption, gel filtration chromatography, ion exchange chromatography, etc.).

In some embodiments, the methods for Cas12i polypeptide expression comprises translation of at least 5 amino acids, at least 10 amino acids, at least 15 amino acids, at least 20 amino acids, at least 50 amino acids, at least 100 amino acids, at least 150 amino acids, at least 200 amino acids, at least 250 amino acids, at least 300 amino acids, at least 400 amino acids, at least 500 amino acids, at least 600 amino acids, at least 700 amino acids, at least 800 amino acids, at least 900 amino acids, or at least 1000 amino acids of the Cas12i polypeptide.

In some embodiments, the methods for protein expression comprises translation of about 5 amino acids, about 10 amino acids, about 15 amino acids, about 20 amino acids, about 50 amino acids, about 100 amino acids, about 150 amino acids, about 200 amino acids, about 250 amino acids, about 300 amino acids, about 400 amino acids, about 500 amino acids, about 600 amino acids, about 700 amino acids, about 800 amino acids, about 900 amino acids, about 1000 amino acids or more of the Cas12i polypeptide.

A variety of methods can be used to determine the level of production of a Cas12i polypeptide in a host cell. Such methods include, but are not limited to, for example, methods that utilize either polyclonal or monoclonal antibodies specific for the Cas12i polypeptide or a labeling tag as described elsewhere herein. Exemplary methods include, but are not limited to, enzyme-linked immunosorbent assays (ELISA), radioimmunoassays (MA), fluorescent immunoassays (FIA), and fluorescent activated cell sorting (FACS). These and other assays are well known in the art (See, e.g., Maddox et al., J. Exp. Med. 158:1211 [1983]).

The present disclosure provides methods of in vivo expression of the Cas12i polypeptide in a cell, comprising providing a polyribonucleotide encoding the Cas12i polypeptide to a host cell wherein the polyribonucleotide encodes the Cas12i polypeptide, expressing the Cas12i polypeptide in the cell, and obtaining the Cas12i polypeptide from the cell.

### Complexing

In some embodiments, an RNA guide targeting BCL11A is complexed with a Cas12i polypeptide to form a ribonucleoprotein. In some embodiments, complexation of the RNA guide and Cas12i polypeptide occurs at a temperature lower than about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., or 55° C. In some embodiments, the RNA guide does not dissociate from the Cas12i polypeptide at about 37° C. over an incubation period of at least about any one of 10 mins, 15 mins, 20 mins, 25 mins, 30 mins, 35 mins, 40 mins, 45 mins, 50 mins, 55 mins, 1 hr, 2 hr, 3 hr, 4 hr, or more hours.

In some embodiments, the RNA guide and Cas12i polypeptide are complexed in a complexation buffer. In some embodiments, the Cas12i polypeptide is stored in a buffer that is replaced with a complexation buffer to form a complex with the RNA guide. In some embodiments, the Cas12i polypeptide is stored in a complexation buffer.

In some embodiments, the complexation buffer has a pH in a range of about 7.3 to 8.6. In one embodiment, the pH of the complexation buffer is about 7.3. In one embodiment, the pH of the complexation buffer is about 7.4. In one embodiment, the pH of the complexation buffer is about 7.5. In one embodiment, the pH of the complexation buffer is about 7.6. In one embodiment, the pH of the complexation buffer is about 7.7. In one embodiment, the pH of the complexation buffer is about 7.8. In one embodiment, the pH of the complexation buffer is about 7.9. In one embodiment, the pH of the complexation buffer is about 8.0. In one embodiment, the pH of the complexation buffer is about 8.1. In one embodiment, the pH of the complexation buffer is about 8.2. In one embodiment, the pH of the complexation buffer is about 8.3. In one embodiment, the pH of the complexation buffer is about 8.4. In one embodiment, the pH of the complexation buffer is about 8.5. In one embodiment, the pH of the complexation buffer is about 8.6.

In some embodiments, the Cas12i polypeptide can be overexpressed and complexed with the RNA guide in a host cell prior to purification as described herein. In some embodiments, mRNA or DNA encoding the Cas12i polypeptide is introduced into a cell so that the Cas12i polypeptide is expressed in the cell. In some embodiments, the RNA guide is also introduced into the cell, whether simultaneously, separately, or sequentially from a single mRNA or DNA construct, such that the ribonucleoprotein complex is formed in the cell.

### Delivery

Compositions or complexes described herein may be formulated, for example, including a carrier, such as a carrier and/or a polymeric carrier, e.g., a liposome, and delivered by known methods to a cell (e.g., a prokaryotic, eukaryotic, plant, mammalian, etc.). Such methods include, but not limited to, transfection (e.g., lipid-mediated, cationic polymers, calcium phosphate, dendrimers); electroporation or other methods of membrane disruption (e.g., nucleofection), viral delivery (e.g., lentivirus, retrovirus, adenovirus, AAV), microinjection, microprojectile bombardment (“gene gun”), fugene, direct sonic loading, cell squeezing, optical transfection, protoplast fusion, impalefection, magnetofection, exosome-mediated transfer, lipid nanoparticle-mediated transfer, and any combination thereof.

In some embodiments, the method comprises delivering one or more nucleic acids (e.g., nucleic acids encoding the Cas12i polypeptide, RNA guide, donor DNA, etc.), one or more transcripts thereof, and/or a pre-formed RNA guide/Cas12i polypeptide complex to a cell, where a ternary complex is formed. Exemplary intracellular delivery methods, include, but are not limited to: viruses or virus-like agents; chemical-based transfection methods, such as those using calcium phosphate, dendrimers, liposomes, or cationic polymers (e.g., DEAE-dextran or polyethylenimine); non-chemical methods, such as microinjection, electroporation, cell squeezing, sonoporation, optical transfection, impalefection, protoplast fusion, bacterial conjugation, delivery of plasmids or transposons; particle-based methods, such as using a gene gun, magnectofection or magnet assisted transfection, particle bombardment; and hybrid methods, such as nucleofection. In some embodiments, the present application further provides cells produced by such methods, and organisms (such as animals, plants, or fungi) comprising or produced from such cells.

In some embodiments, the Cas12i component and the RNA guide component are delivered together. For example, in some embodiments, the Cas12i component and the RNA guide component are packaged together in a single AAV particle. In another example, in some embodiments, the Cas12i component and the RNA guide component are delivered together via lipid nanoparticles (LNPs). In some embodiments, the Cas12i component and the RNA guide component are delivered separately. For example, in some embodiments, the Cas12i component and the RNA guide are packaged into separate AAV particles. In another example, in some embodiments, the Cas12i component is delivered by a first delivery mechanism and the RNA guide is delivered by a second delivery mechanism.

### Cells

Compositions or complexes described herein can be delivered to a variety of cells. In some embodiments, the cell is an isolated cell. In some embodiments, the cell is in cell culture or a co-culture of two or more cell types. In some embodiments, the cell is ex vivo. In some embodiments, the cell is obtained from a living organism and maintained in a cell culture. In some embodiments, the cell is a single-cellular organism.

In some embodiments, the cell is a prokaryotic cell. In some embodiments, the cell is a bacterial cell or derived from a bacterial cell. In some embodiments, the cell is an archaeal cell or derived from an archaeal cell.

In some embodiments, the cell is a eukaryotic cell. In some embodiments, the cell is a plant cell or derived from a plant cell. In some embodiments, the cell is a fungal cell or derived from a fungal cell. In some embodiments, the cell is an animal cell or derived from an animal cell. In some embodiments, the cell is an invertebrate cell or derived from an invertebrate cell. In some embodiments, the cell is a vertebrate cell or derived from a vertebrate cell. In some embodiments, the cell is a mammalian cell or derived from a mammalian cell. In some embodiments, the cell is a human cell. In some embodiments, the cell is a zebra fish cell. In some embodiments, the cell is a rodent cell. In some embodiments, the cell is synthetically made, sometimes termed an artificial cell.

In some embodiments, the cell is derived from a cell line. A wide variety of cell lines for tissue culture are known in the art. Examples of cell lines include, but are not limited to, 293T, MF7, K562, HeLa, CHO, and transgenic varieties thereof. Cell lines are available from a variety of sources known to those with skill in the art (see, e.g., the American Type Culture Collection (ATCC) (Manassas, Va.)). In some embodiments, the cell is an immortal or immortalized cell.

In some embodiments, the cell is a primary cell. In some embodiments, the cell is a stem cell such as a totipotent stem cell (e.g., omnipotent), a pluripotent stem cell, a multipotent stem cell, an oligopotent stem cell, or an unipotent stem cell. In some embodiments, the cell is an induced pluripotent stem cell (iPSC) or derived from an iPSC. In some embodiments, the cell is a differentiated cell. For example, in some embodiments, the differentiated cell is a muscle cell (e.g., a myocyte), a fat cell (e.g., an adipocyte), a bone cell (e.g., an osteoblast, osteocyte, osteoclast), a blood cell (e.g., a monocyte, a lymphocyte, a neutrophil, an eosinophil, a basophil, a macrophage, a erythrocyte, or a platelet), a nerve cell (e.g., a neuron), an epithelial cell, an immune cell (e.g., a lymphocyte, a neutrophil, a monocyte, or a macrophage), a liver cell (e.g., a hepatocyte), a fibroblast, or a sex cell. In some embodiments, the cell is a terminally differentiated cell. For example, in some embodiments, the terminally differentiated cell is a neuronal cell, an adipocyte, a cardiomyocyte, a skeletal muscle cell, an epidermal cell, or a gut cell. In some embodiments, the cell is an immune cell. In some embodiments, the immune cell is a T cell. In some embodiments, the immune cell is a B cell. In some embodiments, the immune cell is a Natural Killer (NK) cell. In some embodiments, the immune cell is a Tumor Infiltrating Lymphocyte (TIL). In some embodiments, the cell is a mammalian cell, e.g., a human cell or a murine cell. In some embodiments, the murine cell is derived from a wild-type mouse, an immunosuppressed mouse, or a disease-specific mouse model. In some embodiments, the cell is a cell within a living tissue, organ, or organism.

### Methods

The disclosure also provides methods of modifying a target sequence within the BCL11A gene. In some embodiments, the methods comprise introducing a BCL11A-targeting RNA guide and a Cas12i polypeptide into a cell. The BCL11A-targeting RNA guide and Cas12i polypeptide can be introduced as a ribonucleoprotein complex into a cell. The BCL11A-targeting RNA guide and Cas12i polypeptide can be introduced on a nucleic acid vector. The Cas12i polypeptide can be introduced as an mRNA. The RNA guide can be introduced directly into the cell.

In some embodiments, the sequence of the BCL11A gene is set forth in SEQ ID NO: 2635 or the reverse complement thereof. In some embodiments, the target sequence is in an exon of a BCL11A gene, such as an exon having a sequence set forth in any one of SEQ ID NO: 2636, SEQ ID NO: 2637, SEQ ID NO: 2638, or SEQ ID NO: 2639, or a reverse complement thereof, or in an enhancer region of the BCL11A gene, such as an enhancer region having a sequence set forth in SEQ ID NO: 2640, or the reverse complement thereof. In some embodiments, the target sequence is in an intron of a BCL11A gene (e.g., an intron of the sequence set forth in SEQ ID NO: 2635 or the reverse complement thereof). In other embodiments, the sequence of the BCL11A gene is a variant of the sequence set forth in SEQ ID NO: 2635 (or the reverse complement thereof) or a homolog of the sequence set forth in SEQ ID NO: 2635 (or the reverse complement thereof). For example, in some embodiments, the target sequence is polymorphic variant of the BCL11A sequence set forth in SEQ ID NO: 2635 (or the reverse complement thereof) or a non-human form of the BCL11A gene.

In some embodiments, an RNA guide as disclosed herein is designed to be complementary to a target sequence that is adjacent to a 5′-NTTN-3′ PAM sequence. The 5′-NTTN-3′ sequence may be immediately adjacent to the target sequence or, for example, within a small number (e.g., 1, 2, 3, 4, or 5) of nucleotides of the target sequence. In some embodiments the 5′-NTTN-3′ sequence is 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′, wherein Y is C or T, B is any nucleotide except for A, D is any nucleotide except for C, and R is A or G. In some embodiments, the 5′-NTTN-3′ sequence is 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′. In some embodiments, the RNA guide is designed to bind to a first strand of a double-stranded target sequence (e.g., the target strand or the spacer-complementary strand), and the 5′-NTTN-3′ PAM sequence is present in the second, complementary strand (e.g., the non-target strand or the non-spacer-complementary strand). In some embodiments, the RNA guide binds adjacent to a 5′-NAAN-3′ sequence on the target strand (e.g., the spacer-complementary strand).

In some embodiments, the Cas12i polypeptide has enzymatic activity (e.g., nuclease activity). In some embodiments, the Cas12i polypeptide induces one or more DNA double-stranded breaks in the cell. In some embodiments, the Cas12i polypeptide induces one or more DNA single-stranded breaks in the cell. In some embodiments, the Cas12i polypeptide induces one or more DNA nicks in the cell. In some embodiments, DNA breaks and/or nicks result in formation of one or more indels (e.g., one or more deletions).

In some embodiments, an RNA guide disclosed herein forms a complex with the Cas12i polypeptide and directs the Cas12i polypeptide to a target sequence adjacent to a 5′-NTTN-3′ sequence. In some embodiments, the complex induces a deletion (e.g., a nucleotide deletion or DNA deletion) adjacent to the 5′-NTTN-3′ sequence. In some embodiments, the complex induces a deletion adjacent to a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the complex induces a deletion adjacent to a T/C-rich sequence.

In some embodiments, the deletion is downstream of a 5′-NTTN-3′ sequence. In some embodiments, the deletion is downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion is downstream of a T/C-rich sequence.

In some embodiments, the deletion alters expression of the BCL11A gene. In some embodiments, the deletion alters function of the BCL11A gene. In some embodiments, the deletion inactivates the BCL11A gene. In some embodiments, the deletion is a frameshifting deletion. In some embodiments, the deletion is a non-frameshifting deletion. In some embodiments, the deletion leads to cell toxicity or cell death (e.g., apoptosis).

In some embodiments, the deletion starts within about 5 to about 15 nucleotides (e.g., about 3, 4, 5, 6, 7, 8, 9, 10, 11, 12, 13, 14, 15, 16, or 17 nucleotides) of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) of a T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a T/C-rich sequence.

In some embodiments, the deletion starts within about 5 to about 10 nucleotides (e.g., about 3, 4, 5, 6, 7, 8, 9, 10, 11, or 12 nucleotides) of the 5′-NTTN-3′ sequence. In some embodiments, the deletion starts within about 5 to about 10 nucleotides (e.g., about 3, 4, 5, 6, 7, 8, 9, 10, 11, or 12 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 10 nucleotides (e.g., about 3, 4, 5, 6, 7, 8, 9, 10, 11, or 12 nucleotides) of a T/C-rich sequence.

In some embodiments, the deletion starts within about 5 to about 10 nucleotides (e.g., about 3, 4, 5, 6, 7, 8, 9, 10, 11, or 12 nucleotides) downstream of the 5′-NTTN-3′ sequence. In some embodiments, the deletion starts within about 5 to about 10 nucleotides (e.g., about 3, 4, 5, 6, 7, 8, 9, 10, 11, or 12 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 10 nucleotides (e.g., about 3, 4, 5, 6, 7, 8, 9, 10, 11, or 12 nucleotides) downstream of a T/C-rich sequence.

In some embodiments, the deletion starts within about 10 to about 15 nucleotides (e.g., about 8, 9, 10, 11, 12, 13, 14, 15, 16, or 17 nucleotides) of the 5′-NTTN-3′ sequence. In some embodiments, the deletion starts within about 10 to about 15 nucleotides (e.g., about 8, 9, 10, 11, 12, 13, 14, 15, 16, or 17 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 10 to about 15 nucleotides (e.g., about 8, 9, 10, 11, 12, 13, 14, 15, 16, or 17 nucleotides) of a T/C-rich sequence.

In some embodiments, the deletion starts within about 10 to about 15 nucleotides (e.g., about 8, 9, 10, 11, 12, 13, 14, 15, 16, or 17 nucleotides) downstream of the 5′-NTTN-3′ sequence. In some embodiments, the deletion starts within about 10 to about 15 nucleotides (e.g., about 8, 9, 10, 11, 12, 13, 14, 15, 16, or 17 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 10 to about 15 nucleotides (e.g., about 8, 9, 10, 11, 12, 13, 14, 15, 16, or 17 nucleotides) downstream of a T/C-rich sequence.

In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence. In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) of a T/C-rich sequence.

In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-NTTN-3′ sequence. In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) downstream of a T/C-rich sequence.

In some embodiments, the deletion ends within about 20 to about 25 nucleotides (e.g., about 17, 18, 19, 20, 21, 22, 23, 24, 25, 26, 27, or 28 nucleotides) of the 5′-NTTN-3′ sequence. In some embodiments, the deletion ends within about 20 to about 25 nucleotides (e.g., about 17, 18, 19, 20, 21, 22, 23, 24, 25, 26, 27, or 28 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion ends within about 20 to about 25 nucleotides (e.g., about 17, 18, 19, 20, 21, 22, 23, 24, 25, 26, 27, or 28 nucleotides) of a T/C-rich sequence.

In some embodiments, the deletion ends within about 20 to about 25 nucleotides (e.g., about 17, 18, 19, 20, 21, 22, 23, 24, 25, 26, 27, or 28 nucleotides) downstream of the 5′-NTTN-3′ sequence. In some embodiments, the deletion ends within about 20 to about 25 nucleotides (e.g., about 17, 18, 19, 20, 21, 22, 23, 24, 25, 26, 27, or 28 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion ends within about 20 to about 25 nucleotides (e.g., about 17, 18, 19, 20, 21, 22, 23, 24, 25, 26, 27, or 28 nucleotides) downstream of a T/C-rich sequence.

In some embodiments, the deletion ends within about 25 to about 30 nucleotides (e.g., about 22, 23, 24, 25, 26, 27, 28, 29, 30, 31, 32, or 33 nucleotides) of the 5′-NTTN-3′ sequence. In some embodiments, the deletion ends within about 25 to about 30 nucleotides (e.g., about 22, 23, 24, 25, 26, 27, 28, 29, 30, 31, 32, or 33 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion ends within about 25 to about 30 nucleotides (e.g., about 22, 23, 24, 25, 26, 27, 28, 29, 30, 31, 32, or 33 nucleotides) of a T/C-rich sequence.

In some embodiments, the deletion ends within about 25 to about 30 nucleotides (e.g., about 22, 23, 24, 25, 26, 27, 28, 29, 30, 31, 32, or 33 nucleotides) downstream of the 5′-NTTN-3′ sequence. In some embodiments, the deletion ends within about 25 to about 30 nucleotides (e.g., about 22, 23, 24, 25, 26, 27, 28, 29, 30, 31, 32, or 33 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion ends within about 25 to about 30 nucleotides (e.g., about 22, 23, 24, 25, 26, 27, 28, 29, 30, 31, 32, or 33 nucleotides) downstream of a T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of the 5′-NTTN-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a T/C-rich sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of a T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of the 5′-NTTN-3′ sequence and ends within about 20 to about 25 nucleotides (e.g., about 17, 18, 19, 20, 21, 22, 23, 24, 25, 26, 27, or 28 nucleotides) downstream of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence and ends within about 20 to about 25 nucleotides (e.g., about 17, 18, 19, 20, 21, 22, 23, 24, 25, 26, 27, or 28 nucleotides) downstream of the 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a T/C-rich sequence and ends within about 20 to about 25 nucleotides (e.g., about 17, 18, 19, 20, 21, 22, 23, 24, 25, 26, 27, or 28 nucleotides) downstream of the T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of a T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of the 5′-NTTN-3′ sequence and ends within about 25 to about 30 nucleotides (e.g., about 22, 23, 24, 25, 26, 27, 28, 29, 30, 31, 32, or 33 nucleotides) downstream of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence and ends within about 25 to about 30 nucleotides (e.g., about 22, 23, 24, 25, 26, 27, 28, 29, 30, 31, 32, or 33 nucleotides) downstream of the 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a T/C-rich sequence and ends within about 25 to about 30 nucleotides (e.g., about 22, 23, 24, 25, 26, 27, 28, 29, 30, 31, 32, or 33 nucleotides) downstream of the T/C-rich sequence.

In some embodiments, the deletion starts within about 5 to about 10 nucleotides (e.g., about 3, 4, 5, 6, 7, 8, 9, 10, 11, or 12 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence. In some embodiments, the deletion starts within about 5 to about 10 nucleotides (e.g., about 3, 4, 5, 6, 7, 8, 9, 10, 11, or 12 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 10 nucleotides (e.g., about 3, 4, 5, 6, 7, 8, 9, 10, 11, or 12 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a T/C-rich sequence.

In some embodiments, the deletion starts within about 5 to about 10 nucleotides (e.g., about 3, 4, 5, 6, 7, 8, 9, 10, 11, or 12 nucleotides) downstream of the 5′-NTTN-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-NTTN-3′ sequence. In some embodiments, the deletion starts within about 5 to about 10 nucleotides (e.g., about 3, 4, 5, 6, 7, 8, 9, 10, 11, or 12 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 10 nucleotides (e.g., about 3, 4, 5, 6, 7, 8, 9, 10, 11, or 12 nucleotides) downstream of a T/C-rich sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the T/C-rich sequence.

In some embodiments, the deletion starts within about 5 to about 10 nucleotides (e.g., about 3, 4, 5, 6, 7, 8, 9, 10, 11, or 12 nucleotides) and ends within about 20 to about 25 nucleotides (e.g., about 17, 18, 19, 20, 21, 22, 23, 24, 25, 26, 27, or 28 nucleotides) of the 5′-NTTN-3′ sequence. In some embodiments, the deletion starts within about 5 to about 10 nucleotides (e.g., about 3, 4, 5, 6, 7, 8, 9, 10, 11, or 12 nucleotides) and ends within about 20 to about 25 nucleotides (e.g., about 17, 18, 19, 20, 21, 22, 23, 24, 25, 26, 27, or 28 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 10 nucleotides and ends within about 20 to about 25 nucleotides (e.g., about 17, 18, 19, 20, 21, 22, 23, 24, 25, 26, 27, or 28 nucleotides) (e.g., about 3, 4, 5, 6, 7, 8, 9, 10, 11, or 12 nucleotides) of a T/C-rich sequence.

In some embodiments, the deletion starts within about 5 to about 10 nucleotides (e.g., about 3, 4, 5, 6, 7, 8, 9, 10, 11, or 12 nucleotides) downstream of the 5′-NTTN-3′ sequence and ends within about 20 to about 25 nucleotides (e.g., about 17, 18, 19, 20, 21, 22, 23, 24, 25, 26, 27, or 28 nucleotides) downstream of the 5′-NTTN-3′ sequence. In some embodiments, the deletion starts within about 5 to about 10 nucleotides (e.g., about 3, 4, 5, 6, 7, 8, 9, 10, 11, or 12 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence and ends within about 20 to about 25 nucleotides (e.g., about 17, 18, 19, 20, 21, 22, 23, 24, 25, 26, 27, or 28 nucleotides) downstream of the 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 10 nucleotides (e.g., about 3, 4, 5, 6, 7, 8, 9, 10, 11, or 12 nucleotides) downstream of a T/C-rich sequence and ends within about 20 to about 25 nucleotides (e.g., about 17, 18, 19, 20, 21, 22, 23, 24, 25, 26, 27, or 28 nucleotides) downstream of the T/C-rich sequence.

In some embodiments, the deletion starts within about 5 to about 10 nucleotides (e.g., about 3, 4, 5, 6, 7, 8, 9, 10, 11, or 12 nucleotides) and ends within about 25 to about 30 nucleotides (e.g., about 22, 23, 24, 25, 26, 27, 28, 29, 30, 31, 32, or 33 nucleotides) of the 5′-NTTN-3′ sequence. In some embodiments, the deletion starts within about 5 to about 10 nucleotides (e.g., about 3, 4, 5, 6, 7, 8, 9, 10, 11, or 12 nucleotides) and ends within about 25 to about 30 nucleotides (e.g., about 22, 23, 24, 25, 26, 27, 28, 29, 30, 31, 32, or 33 nucleotides) of a T/C-rich sequence.

In some embodiments, the deletion starts within about 5 to about 10 nucleotides (e.g., about 3, 4, 5, 6, 7, 8, 9, 10, 11, or 12 nucleotides) downstream of the 5′-NTTN-3′ sequence and ends within about 25 to about 30 nucleotides (e.g., about 22, 23, 24, 25, 26, 27, 28, 29, 30, 31, 32, or 33 nucleotides) downstream of the 5′-NTTN-3′ sequence. In some embodiments, the deletion starts within about 5 to about 10 nucleotides (e.g., about 3, 4, 5, 6, 7, 8, 9, 10, 11, or 12 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence and ends within about 25 to about 30 nucleotides (e.g., about 22, 23, 24, 25, 26, 27, 28, 29, 30, 31, 32, or 33 nucleotides) downstream of the 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 10 nucleotides (e.g., about 3, 4, 5, 6, 7, 8, 9, 10, 11, or 12 nucleotides) downstream of a T/C-rich sequence and ends within about 25 to about 30 nucleotides (e.g., about 22, 23, 24, 25, 26, 27, 28, 29, 30, 31, 32, or 33 nucleotides) downstream of the T/C-rich sequence.

In some embodiments, the deletion starts within about 10 to about 15 nucleotides (e.g., about 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence. In some embodiments, the deletion starts within about 10 to about 15 nucleotides (e.g., about 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 10 to about 15 nucleotides (e.g., about 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a T/C-rich sequence.

In some embodiments, the deletion starts within about 10 to about 15 nucleotides (e.g., about 8, 9, 10, 11, 12, 13, 14, 15, 16, or 17 nucleotides) downstream of the 5′-NTTN-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-NTTN-3′ sequence. In some embodiments, the deletion starts within about 10 to about 15 nucleotides (e.g., about 8, 9, 10, 11, 12, 13, 14, 15, 16, or 17 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 10 to about 15 nucleotides (e.g., about 8, 9, 10, 11, 12, 13, 14, 15, 16, or 17 nucleotides) downstream of a T/C-rich sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the T/C-rich sequence.

In some embodiments, the deletion starts within about 10 to about 15 nucleotides (e.g., about 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of the 5′-NTTN-3′ sequence. In some embodiments, the deletion starts within about 10 to about 15 nucleotides (e.g., about 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 10 to about 15 nucleotides (e.g., about 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of a T/C-rich sequence.

In some embodiments, the deletion starts within about 10 to about 15 nucleotides (e.g., about 8, 9, 10, 11, 12, 13, 14, 15, 16, or 17 nucleotides) downstream of the 5′-NTTN-3′ sequence and ends within about 20 to about 25 nucleotides (e.g., about 17, 18, 19, 20, 21, 22, 23, 24, 25, 26, 27, or 28 nucleotides) downstream of the 5′-NTTN-3′ sequence. In some embodiments, the deletion starts within about 10 to about 15 nucleotides (e.g., about 8, 9, 10, 11, 12, 13, 14, 15, 16, or 17 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence and ends within about 20 to about 25 nucleotides (e.g., about 17, 18, 19, 20, 21, 22, 23, 24, 25, 26, 27, or 28 nucleotides) downstream of the 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 10 to about 15 nucleotides (e.g., about 8, 9, 10, 11, 12, 13, 14, 15, 16, or 17 nucleotides) downstream of a T/C-rich sequence and ends within about 20 to about 25 nucleotides (e.g., about 17, 18, 19, 20, 21, 22, 23, 24, 25, 26, 27, or 28 nucleotides) downstream of the T/C-rich sequence.

In some embodiments, the deletion starts within about 10 to about 15 nucleotides (e.g., about 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of the 5′-NTTN-3′ sequence. In some embodiments, the deletion starts within about 10 to about 15 nucleotides (e.g., about 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 10 to about 15 nucleotides (e.g., about 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of a T/C-rich sequence.

In some embodiments, the deletion starts within about 10 to about 15 nucleotides (e.g., about 8, 9, 10, 11, 12, 13, 14, 15, 16, or 17 nucleotides) downstream of the 5′-NTTN-3′ sequence and ends within about 25 to about 30 nucleotides (e.g., about 22, 23, 24, 25, 26, 27, 28, 29, 30, 31, 32, or 33 nucleotides) downstream of the 5′-NTTN-3′ sequence. In some embodiments, the deletion starts within about 10 to about 15 nucleotides (e.g., about 8, 9, 10, 11, 12, 13, 14, 15, 16, or 17 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence and ends within about 25 to about 30 nucleotides (e.g., about 22, 23, 24, 25, 26, 27, 28, 29, 30, 31, 32, or 33 nucleotides) downstream of the 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 10 to about 15 nucleotides (e.g., about 8, 9, 10, 11, 12, 13, 14, 15, 16, or 17 nucleotides) downstream of a T/C-rich sequence and ends within about 25 to about 30 nucleotides (e.g., about 22, 23, 24, 25, 26, 27, 28, 29, 30, 31, 32, or 33 nucleotides) downstream of the T/C-rich sequence.

In some embodiments, the deletion is up to about 50 nucleotides in length (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides). In some embodiments, the deletion is up to about 40 nucleotides in length (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotides). In some embodiments, the deletion is between about 4 nucleotides and about 40 nucleotides in length (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotides). In some embodiments, the deletion is between about 4 nucleotides and about 25 nucleotides in length (e.g., about 3, 4, 5, 6, 7, 8, 9, 10, 11, 12, 13, 14, 15, 16, 17, 18, 19, 20, 21, 22, 23, 24, 25, 26, 27, or 28 nucleotides). In some embodiments, the deletion is between about 10 nucleotides and about 25 nucleotides in length (e.g., about 7, 8, 9, 10, 11, 12, 13, 14, 15, 16, 17, 18, 19, 20, 21, 22, 23, 24, 25, 26, 27, or 28 nucleotides). In some embodiments, the deletion is between about 10 nucleotides and about 15 nucleotides in length (e.g., about 7, 8, 9, 10, 11, 12, 13, 14, 15, 16, or 17 nucleotides).

In some embodiments, the methods described herein are used to engineer a cell comprising a deletion as described herein in a BCL11A gene.

Compositions, vectors, nucleic acids, RNA guides and cells disclosed herein may be used in therapy. Compositions, vectors, nucleic acids, RNA guides and cells disclosed herein may be used in methods of treating a disease or condition in a subject. Any suitable delivery or administration method known in the art may be used to deliver compositions, vectors, nucleic acids, RNA guides and cells disclosed herein. Such methods may involve contacting a target sequence with a composition, vector, nucleic acid, or RNA guide disclosed herein. Such methods may involve a method of editing a BCL11A sequence as disclosed herein. In some embodiments, a cell engineered using an RNA guide disclosed herein is used for ex vivo gene therapy. In some embodiments, the compositions, vectors, nucleic acids, RNA guides and cells disclosed herein are used in the treatment of sickle cell anemia. In some embodiments, the compositions, vectors, nucleic acids, RNA guides and cells disclosed herein are used in the treatment of beta-thalassemia. In some embodiments, wherein one or more RNA guides targets the enhancer region of BCL11A (SEQ ID NO: 2640), the one or more RNA guides are used in the treatment of sickle cell anemia or beta-thalassemia.

### Kits

The invention also provides kits or systems that can be used, for example, to carry out a method described herein. In some embodiments, the kits or systems include an RNA guide and a Cas12i polypeptide. In some embodiments, the kits or systems include a polynucleotide that encodes such a Cas12i polypeptide, and optionally the polynucleotide is comprised within a vector, e.g., as described herein. In some embodiments, the kits or systems include a polynucleotide that encodes an RNA guide disclosed herein. The Cas12i polypeptide and the RNA guide (e.g., as a ribonucleoprotein) can be packaged within the same or other vessel within a kit or system or can be packaged in separate vials or other vessels, the contents of which can be mixed prior to use. The kits or systems can additionally include, optionally, a buffer and/or instructions for use of the RNA guide and Cas12i polypeptide.

All references and publications cited herein are hereby incorporated by reference.

### EXAMPLES

The following examples are provided to further illustrate some embodiments of the present invention but are not intended to limit the scope of the invention; it will be understood by their exemplary nature that other procedures, methodologies, or techniques known to those skilled in the art may alternatively be used.

### Example 1—Editing of BCL11A in a Mammalian Cell

This example describes generation of modified CD34+ hematopoietic stem/progenitor cells (HSPC) with variant Cas12i2. For this study, human primary CD34+ HSPCs were transfected with BCL11A intronic erythroid enhancer-targeting RNPs comprising variant Cas12i2 of SEQ ID NO: 2642 and RNA guide. The modified CD34+ HSPCs were analyzed by FACS staining and indel assessment at the BCL11A intronic erythroid enhancer target.

Two frozen human bone marrow CD34+ cell vials per cell lot were thawed (Day 0), washed and assessed for cell number and viability by acridine orange/propidium iodide (AO/PI) staining using a cell counter. CD34+ cells were cultured in serum-free expansion media (from StemCell Technologies) with the appropriate supplement for approximately 48 hours.

RNP Complexation Reactions:

Variant Cas12i2 RNP complexes were prepared by mixing purified variant Cas12i2 of SEQ ID NO: 2642 (400 μM) with different RNA guides (1 mM in 250 mM NaCl) at a 1:1 Cas12i2 effector:RNA guide volume ratio (corresponding to 2.5:1 RNA guide:Cas12i2 effector molar ratio). SpCas9 RNP complexes were prepared by mixing purified SpCas9 (62 μM) with single guide RNA (sgRNA) (1 mM in water) at a 6.45:1 SpCas9 effector: sgRNA volume ratio (corresponding to 2.5:1 sgRNA: SpCas9 effector molar ratio). SpCas9 protein was purchased from Aldevron. Sequences of RNA guides and sgRNA are shown in Table 6.

TABLE 6
Sequences of BCL11A intronic erythroid enhancer-targeting RNA guides (for variant
Cas12i2) and sgRNA (for SpCas9) used for RNP complexes

| | | | | DNA | |
|---|---|---|---|---|---|
| Guide Name | Gene | Effector | PAM | Strand | RNA guide |
| Cas12i2_BCL11A_ | BCL11A | Cas12i2 | CTTT | Antise | AGAAAUCCGUCUUUCAUUGACGGG |
| enh_T1 | enhancer | | | nse | AAGCUAGUCUAGUGCAAGC (SEQ ID |
| | | | | | NO: 2677) |
| Cas12i2_BCL11A_ | BCL11A | Cas12i2 | CTTC | Sense | AGAAAUCCGUCUUUCAUUGACGGC |
| enh_T4 | enhancer | | | | UGGAGCCUGUGAUAAAAGC (SEQ ID |
| | | | | | NO: 2678) |
| Cas12i2_BCL11A_ | BCL11A | Cas1212 | CTTC | Sense | AGAAAUCCGUCUUUCAUUGACGGU |
| enh_T5 | enhancer | | | | ACCCCACCCACGCCCCCAC (SEQ ID |
| | | | | | NO: 2679) |
| SpCas9_BCL11A_ | BCL11A | SpCas9 | AGG | Antise | mC*mU*mA*ACAGUUGCUUUUAUCA |
| enh_T1 | enhancer | | | nse | CGUUUUAGAGCUAGAAAUAGCAAG |
| | | | | | UUAAAAUAAGGCUAGUCCGUUAUC |
| | | | | | AACUUGAAAAAGUGGCACCGAGUC |
| | | | | | GGUGCmU*mU*mU*U (SEQ ID NO: |
| | | | | | 2680) |

*-phosphorothioated
m-2′ O-methyl

For effector only controls, variant Cas12i2 or SpCas9 were mixed with protein storage buffer (25 mM Tris, pH 7.5, 250 mM NaCl, 1 mM TCEP, 50% glycerol) at the same volume ratio as the RNA guide or sgRNA, respectively. Complexations were incubated at 37 degrees Celsius for 30-60 minutes. Following incubation, RNPs were diluted to 18.75 μM, 50 μM, 100 μM, or 160 μM effector concentration for variant Cas12i2 and 18.75 μM or 50 μM for SpCas9. For multiplexing, separate RNPs were mixed together prior to electroporation.

On Day 2, approximately 1e5 cells per electroporation reaction, plus 20% extra, were harvested and counted. Cells were washed once with PBS and resuspended in buffer+supplement (from Lonza #VXP-3032)+1 mM transfection enhancer oligo (to bring concentration to 4.28 μM in P3 buffer). Concentration of resuspended cells was approximately 5,555 cells/μL. 18 μL of resuspended cells (˜1e5 cells) were mixed with 2 μL of individual or multiplexed RNP complexes to bring final concentration of variant Cas12i2 RNPs to 1.875 PM, 5 PM, 10 μM or 16 PM. Final concentration of SpCas9 RNPs was 1.875 μM or 5 μM. The following controls were set up: unelectroporated cells only, cells in protein storage buffer only. The plate was electroporated using an electroporation device, excluding the unelectroporated conditions. Each electroporation reaction was transferred into 24-well culture plate well containing pre-warmed serum-free media and the appropriate supplement. Cultures were incubated at 37 degrees Celsius, 5% CO₂ for 3 days.

A portion of cell samples (approximately 20 μL) from each test condition was collected at 24, 48, and 72 h post electroporation. Viability was evaluated using AO/PI stain on a cell counter.

On Day 3, cell pellets were prepared from cells remaining after viability testing. Approximately 5e4 cells from each sample were harvested and transferred to a microcentrifuge tube. Cells were pelleted at 1500 rpm for 5 min. Supernatants were removed and pellets were frozen at −80° C.

For genomic DNA extraction, pellets were thawed to room temperature and resuspended in appropriate volume of DNA extraction buffer (from Lucigen) to give final concentration of 1000 cells/μL. Samples were then cycled in PCR machine at 65° C. for 15 min, 68° C. for 15 min, 98° C. for 10 min. Samples were then frozen at −20° C.

Samples for Next Generation Sequencing (NGS) were prepared by rounds of PCR. The first round (PCR I) was used to amplify the genomic regions flanking the target site and add NGS adapters. The second round (PCR II) was used to add NGS indexes. Reactions were then pooled, purified by column purification, and quantified on a fluorometer (Qubit). Sequencing runs were done using a 300 or 150 cycle NGS instrument (NextSeq v2.5) mid or high output kit and run on an NGS instrument (NextSeq 550).

For NGS analysis, the indel mapping function used a sample's fastq file, the amplicon reference sequence, and the forward primer sequence. For each read, a kmer-scanning algorithm was used to calculate the edit operations (match, mismatch, insertion, deletion) between the read and the reference sequence. In order to remove small amounts of primer dimer present in some samples, the first 30 nucleotides of each read were required to match the reference and reads where over half of the mapping nucleotides are mismatches were filtered out as well. Up to 50,000 reads passing those filters were used for analysis, and reads were counted as an indel read if they contained an insertion or deletion. The indel % was calculated as the number of indel-containing reads divided by the number of reads analyzed (reads passing filters up to 50,000). The QC standard for the minimum number of reads passing filters was 10,000. Indels were further assessed for disruption of the GATAA motif sequence by searching for TTATC (reverse complement of GATAA sequence, on the forward strand) sequence in each indel.

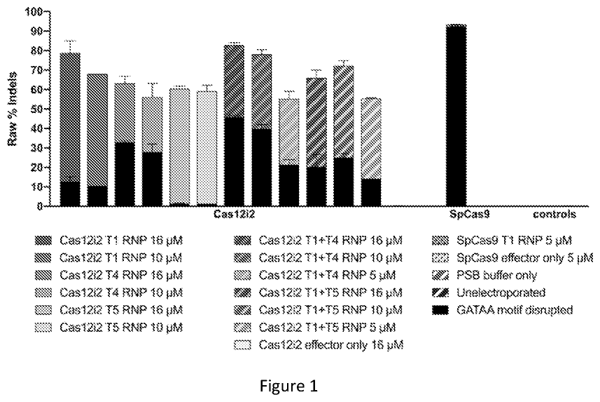

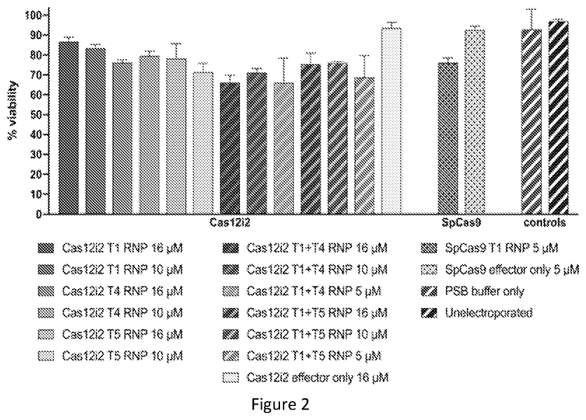

FIG. 1 and FIG. 2 demonstrate the results of this example. As shown in FIG. 1, BCL11A intronic erythroid enhancer-targeting RNP complexes comprising variant Cas12i2 and RNA guide resulted in indel activity in primary CD34+ HSPCs. The data showed that at least 50% of variant Cas12i2-induced indels partially or fully disrupted the GATAA motif of BCL11A intronic erythroid enhancer region.

FIG. 2 illustrates that modified CD34+ HSPCs generated with variant Cas12i2 editing of BCL11A intronic erythroid enhance were viable at least 72 hours after treatment of primary CD34+ HSPCs with variant Cas12i2 RNP complexes.

This example demonstrated that Cas12i2 complexed with the tested RNA guides comprised robust indel activity. Variant Cas12i2 RNPs that targeted BCL11A intronic erythroid enhancer region-targeting were used to generate modified CD34+ HSPCs and resulted in at least about 50% partial or complete disruption of the GATAA motif in the modified cells. The results also show that more than one RNA guide (e.g., multiplexed RNA guides) can be used to introduce indels into BCL11A.

| | | |
|---|---|---|
| Nucleotide | atgagcagcg cgatcaaaag ctacaagagc gttctgcgtc cgaacgagcg taagaaccaa | 60 |
| sequence | ctgctgaaaa gcaccattca gtgcctggaa gacggtagcg cgttcttttt caagatgctg | 120 |
| encoding | caaggcctgt ttggtggcat caccccggag attgttcgtt tcagcaccga acaggagaaa | 180 |
| Cas12i2- | cagcaacagg atatcgcgct gtggtgcgcg gttaactggt tccgtccggt gagccaagac | 240 |
| SEQ ID NO: | agcctgaccc acaccattgc gagcgataac ctggtggaga agtttgagga atactatggt | 300 |
| 2633 | ggcaccgcga gcgacgcgat caaacagtac ttcagcgcga gcattggcga aagctactat | 360 |
| | tggaacgact gccgtcaaca gtactatgat ctgtgccgtg agctgggtgt tgaggtgagc | 420 |
| | gacctgaccc atgatctgga gatcctgtgc cgtgaaaagt gcctggcggt tgcgaccgag | 480 |
| | agcaaccaga acaacagcat cattagcgtt ctgtttggca ccggcgaaaa agaggaccgt | 540 |
| | agcgtgaaac tgcgtatcac caagaaaatt ctggaggcga tcagcaacct gaaagaaatc | 600 |
| | ccgaagaacg ttgcgccgat tcaagagatc attctgaacg tggcgaaagc gaccaaggaa | 660 |
| | accttccgtc aggtgtatgc gggtaacctg ggtgcgccga gcaccctgga gaaatttatc | 720 |
| | gcgaaggacg gccaaaaaga gttcgatctg aagaaactgc agaccgacct gaagaaagtt | 780 |
| | attcgtggta aaagcaagga gcgtgattgg tgctgccagg aagagctgcg tagctacgtg | 840 |
| | gagcaaaaca ccatccagta tgacctgtgg gcgtggggcg aaatgttcaa caaagcgcac | 900 |
| | accgcgctga aaatcaagag cacccgtaac tacaactttg cgaagcaacg tctggaacag | 960 |
| | ttcaaagaga ttcagagcct gaacaacctg ctggttgtga agaagctgaa cgactttttc | 1020 |
| | gatagcgaat ttttcagcgg cgaggaaacc tacaccatct gcgttcacca tctgggggc | 1080 |
| | aaggacctga gcaaactgta taaggcgtgg gaggatgatc cggcggaccc ggaaaacgcg | 1140 |
| | attgtggttc tgtgcgacga tctgaaaaac aactttaaga aagagccgat ccgtaacatt | 1200 |
| | ctgcgttaca tcttcaccat tcgtcaagaa tgcagcgcgc aggacatcct ggcggcggcg | 1260 |
| | aagtacaacc aacagctgga tcgttataaa agccaaaagg cgaacccgag cgttctgggt | 1320 |
| | aaccagggct ttacctggac caacgcggtg atcctgccgg agaaggcgca gcgtaacgac | 1380 |
| | cgtccgaaca gcctggatct gcgtatttgg ctgtacctga aactgcgtca cccggacggt | 1440 |
| | cgttggaaga aacaccatat cccgttctac gatacccgtt tcttccaaga aatttatgcg | 1500 |
| | gcgggcaaca gcccggttga cacctgccag tttcgtaccc cgcgtttcgg ttatcacctg | 1560 |
| | ccgaaactga ccgatcagac cgcgatccgt gttaacaaga aacatgtgaa agcggcgaag | 1620 |
| | accgaggcgc gtattcgtct ggcgatccaa cagggcaccc tgccggtgag caacctgaag | 1680 |
| | atcaccgaaa ttagcgcgac catcaacagc aaaggtcaag tgcgtattcc ggttaagttt | 1740 |
| | gacgtgggtc gtcaaaaagg caccctgcag atcggtgacc gtttctgcgg ctacgatcaa | 1800 |
| | aaccagaccg cgagccacgc gtatagcctg tgggaagtgg ttaaagaggg tcaataccat | 1860 |
| | aaagagctgg gctgctttgt tcgtttcatc agcagcggtg acatcgtgag cattaccgag | 1920 |
| | aaccgtggca accaatttga tcagctgagc tatgaaggtc tggcgtaccc gcaatatgcg | 1980 |
| | gactggcgta agaaagcgag caagttcgtg agcctgtggc agatcaccaa gaaaaacaag | 2040 |
| | aaaaaggaaa tcgtgaccgt tgaagcgaaa gagaagtttg acgcgatctg caagtaccag | 2100 |
| | ccgcgtctgt ataaattcaa caaggagtac gcgtatctgc tgcgtgatat tgttcgtggc | 2160 |
| | aaaagcctgg tggaactgca acagattcgt caagagatct ttcgtttcat tgaacaggac | 2220 |
| | tgcggtgtta cccgtctggg cagcctgagc ctgagcaccc tggaaaccgt gaaagcggtt | 2280 |
| | aagggtatca tttacagcta ttttagcacc gcgctgaacg cgagcaagaa caacccgatc | 2340 |
| | agcgacgaac agcgtaaaga gtttgatccg gaactgttcg cgctgctgga aaagctggag | 2400 |
| | ctgattcgta cccgtaaaaa gaaacaaaaa gtggaacgta tcgcgaacag cctgattcag | 2460 |
| | acctgcctgg agaacaacat caagttcatt cgtggtgaag gcgacctgag caccaccaac | 2520 |
| | aacgcgacca agaaaaaggc gaacagccgt agcatggatt ggttggcgcg tggtgttttt | 2680 |
| | aacaaaatcc gtcaactggc gccgatgcac aacattaccc tgttcggttg cggcagcctg | 2640 |
| | tacaccagcc accaggaccc gctggtgcat cgtaacccgg ataaagcgat gaagtgccgt | 2700 |
| | tgggcggcga tcccggttaa ggacattggc gattgggtgc tgcgtaagct gagccaaaac | 2760 |
| | ctgcgtgcga aaaacatcgg caccggcgag tactatcacc aaggtgttaa agagttcctg | 2820 |
| | agccattatg aactgcagga cctggaggaa gagctgctga agtggcgtag cgatcgtaaa | 2880 |
| | agcaacattc cgtgctgggt gctgcagaac cgtctggcgg agaagctggg caacaaagaa | 2940 |
| | gcggtggttt acatcccggt tcgtggtggc cgtatttatt ttgcgaccca caaggtggcg | 3000 |
| | accggtgcgg tgagcatcgt tttcgaccaa aaacaagtgt gggtttgcaa cgcggatcat | 3060 |
| | gttgcggcgg cgaacatcgc gctgaccgtg aagggtattg gcgaacaaag cagcgacgaa | 3120 |
| | gagaacccgg atggtagccg tatcaaactg cagctgacca gc | 3162 |

| | |
|---|---|
| Cas12i2 | MSSAIKSYKSVLRPNERKNQLLKSTIQCLEDGSAFFFKMLQGLEGGITPEIVRESTEQEK |
| amino acid | QQQDIALWCAVNWFRPVSQDSLTHTIASDNLVEKFEEYYGGTASDAIKQYFSASIGESYY |
| sequence- | WNDCRQQYYDLCRELGVEVSDLTHDLEILCREKCLAVATESNQNNSIISVLFGTGEKEDR |
| SEQ ID NO: | SVKLRITKKILEAISNLKEIPKNVAPIQEIILNVAKATKETFRQVYAGNLGAPSTLEKFI |
| 2634 | AKDGQKEFDLKKLQTDLKKVIRGKSKERDWCCQEELRSYVEQNTIQYDLWAWGEMENKAH |
| | TALKIKSTRNYNFAKQRLEQFKEIQSLNNLLVVKKLNDFFDSEFFSGEETYTICVHHLGG |
| | KDLSKLYKAWEDDPADPENAIVVLCDDLKNNFKKEPIRNILRYIFTIRQECSAQDILAAA |
| | KYNQQLDRYKSQKANPSVLGNQGFTWTNAVILPEKAQRNDRPNSLDLRIWLYLKLRHPDG |
| | RWKKHHIPFYDTRFFQEIYAAGNSPVDTCQFRTPRFGYHLPKLTDQTAIRVNKKHVKAAK |
| | TEARIRLAIQQGTLPVSNLKITEISATINSKGQVRIPVKFDVGRQKGTLQIGDRFCGYDQ |
| | NQTASHAYSLWEVVKEGQYHKELGCFVRFISSGDIVSITENRGNQFDQLSYEGLAYPQYA |
| | DWRKKASKFVSLWQITKKNKKKEIVTVEAKEKFDAICKYQPRLYKENKEYAYLLRDIVRG |
| | KSLVELQQIRQEIFRFIEQDCGVTRLGSLSLSTLETVKAVKGIIYSYFSTALNASKNNPI |
| | SDEQRKEFDPELFALLEKLELIRTRKKKQKVERIANSLIQTCLENNIKFIRGEGDLSTIN |
| | NATKKKANSRSMDWLARGVFNKIRQLAPMHNITLFGCGSLYTSHQDPLVHRNPDKAMKCR |
| | WAAIPVKDIGDWVLRKLSQNLRAKNIGTGEYYHQGVKEFLSHYELQDLEEELLKWRSDRK |
| | SNIPCWVLQNRLAEKLGNKEAVVYIPVRGGRIYFATHKVATGAVSIVFDQKQVWVCNADH |
| | VAAANIALTVKGIGEQSSDEENPDGSRIKLQLTS |
| BCL11A- | GTCTCTGTCCATCCAGACTCCTGACGTTCAAGTTCGCAGGGACGTCACGTCCGCACTTGAACTTG |
| SEQ ID NO: | CAGCTCAGGGGGGCTTTTGCCATTTTTTTCATCTCTCTCTCTCTCTCTCCCTCTATCTCTCTTCT |
| 2635 | CTCTCTCTCCCTCTTTTTTTTTTTTTTTTTTTTTTTTTTTTTGCTTAAAAAAAAGCCATGACGGC |
| | TCTCCCACAATTCATCTTCCCTGCGCCATCTTTGTATTATTTCTAATTTATTTTGGATGTCAAAA |
| | GGCACTGATGAAGATATTTTCTCTGGAGTCTCCTTCTTTCTAACCCGGCTCTCCCGATGTGAACC |
| | GAGCCGTCGTCCGCCCGCCGCCGCCGCCGCCGCCGCCGCCGCCCGCCCCGCAGCCCACCATGTCT |
| | CGCCGCAAGCAAGGCAAACCCCAGCACTTAAGCAAACGGGAATTCTCGCGTAAGTAACCCAATAA |
| | TAGTAATAATAATTATTAATAATCACGAGAGCGCGCAGGACTAGAAGCAAAAGCGAGGGGGAGAG |
| | AGGGGTGTGTGCATGCATTTTTAAATTTTTCACGAGAAAAACCTCCGAGAGTCGAGGTAAAAGAG |
| | ATAAAGGGGGAAAAAACCCTCATCCCATCTGGAACCATTGCCGTGTATGCACTTTTGAGACAGCA |
| | CGCACCTTTTAATTTTATTTAATTTTACAAAAATTTGACTCCTCCTCTTTCCTCCTTTCCGCCGC |
| | TTTATTTCTCTTTTCGAAAAGGAATGCAATGATTCCACTCCCCCCCCGCCCCGCCAGTTTTGCAA |
| | AATAATGAACAATGCTAAGGTTGCGAACAACTCACATGCAAACCTGGGGGTGGGAGCTGGTGGGG |
| | AAAGGGAGGTTGCTTCCCACTCACCGTAAGAAAATGGGGGGGTAGGGAGGGAGTGAGTACAAGTC |
| | TAAAAAACGATTCCCGGGGAGAAAAGAGGTGAGACTGGCTTTTGGACACCAGCGCGCTCACGGTC |
| | AAGTGTGCAGCGGGAGGAAAGTAGTCATCCCCACAATAGTGAGAAAGTGGCACTGTGGAAAGGGG |
| | CCCCCGGCGCTCCTGAGTCCGCGGAGTCGGGAGAGGGGCCGCGGCGACGGGGAGAGCCGTGGGAC |
| | CGGGAAGGACGGGAGACGCGGCCGGCACTGCCGCCTTTTGTTCCGGCCAGAGGTGGGTGTTTGTC |
| | CCGCTGCCTTTTGTGCCGGCTCCTCGCGCTTGCCCTCCCGCGCCGCCGCCGCCGCCGCCGCCGAA |
| | GGGCAGGAGCTAGGGCCGGGGGAGGAGGCGGCCGGGGGCACGCGGGAGAGGGAGGGAGGGAGCCC |
| | GGACTGCTGCCTCCTGGGTTGCCGCTGCCCTCCCCTCCCGACCGAACCTCAGAGGCAGCAAGGAG |
| | AAGACTGGCACATAAATAAATAAATACATAAAAATAAAATAAATAAAACAAGGCAAGAGAATGTA |
| | CAATTTCTTGCCCCCAAACCGAAGCCAAACGCTCTGCCAACCCTTTTCTGTGACGGCCTTCTCTT |
| | TGACTCCCCCACCAGCCCCCCTGCAAAAATCTCACAATCTCTCATCTAGAAAAAAATTTACAATC |
| | ACCCTCTTCCCCCAAACCCCTTCAGTTGCAAACTTAGGGCGCCGACGGCACGGAGAGGGAGAGAG |
| | GAACTCCCTCCTCTTACTATTTTTTGGAAGATTTTCAAAAAAAGTGGAAGTGGATTTTGATTGGG |
| | AAAAATCTCGTGTCTGAATGTTTACAAGCACCGCGTGTGCGGGAGCCTCCTGCCAACAAACAGAC |
| | AGAGGACCGAGCGCGGCGCGGCAGCCCCGGAGCAGGCGGCGGCGGCGGCCTGGCCTCGCCCGGGC |
| | CTTGCCCGACCTCGCCGCGCCCCAGCCCAGCCCCGGATCGCCCACCCGGCGCCCGGCGCCCACCC |
| | GCCAGGCACGGCGGCAGGCCACGCAGTGTCTCCGCGCCAGCCTGGCCCGTGGTCCTGGTCCGCCC |
| | CCAGCACAATGCCGAGACCTCTTCTCGACCTCCCAGACTGCGAAATCGGCTGGGTGAAACTCGGC |
| | TTTGCAAAGCATTTTTATTTTGCAGGGCAACTGTAAAAGCGCGTTCTGCGCCTCCCCTCCCCTCC |
| | GCCCTGGGTACTTTCTCAGACGTCTCTTGTCCACAGCTCGGGACCGCGAGGAGGTCACCACGTGC |
| | TTTCCCTGCCCACCCCCCACCCCGCCCGGTCTGGTCACCAGTCCCCCTGCGCCCCGAGCACGACT |
| | AGGCCAGGTGGCGGGGTTGCCGGGGAAGGGCAGGGGAGAAGTGTGTTTGAGTGTGCATTTTAAGG |
| | GCGCTGGATCCTGGGACCCCGAGCACTTCACCCTTTGGGTCTTGCCCTCTTCCCCAGGCCTGGCT |
| | TGGTCTGAGGTTCTTGTGAGTCTGTATAAGAGCTGGTGGTGGTGGCTGTCTCCCGCTGACTGCGC |
| | CTGGAAAGGCGGGCGGTGGGCACTTAGGAAAGTTTGGCAGCAAGGGAAAGAAAGGCGTGAGGGCC |
| | CACATTCCCCCCCTACTCAATTTATGATTCTTACTTTAAATTTTTGATGCAGTTTTAAAGGACCA |
| | CCTATACTTGGTTCTGTGTTTTTTTAAAGGGGTGGTGGTGGGGGGTGCGCACAGGGAATTTAATT |
| | TTTCACTGGGCCCTGGAACTTGTCACACACTTCGGAAGCTCCCCCACCCCGGCACGTTGCGCGGC |
| | CCTCCCTCTCCCCACCCCCTCGCATCCCTCCCTCGCCGCTCTCCCCCGCCCCCAACTCCCCCGGC |
| | CGCGCCGGATGCGGATCAGACGCGGCGCGCGGCGGTGTGAAGTTACAGCCCGGCCAGGTACCGGC |
| | GGGAAGGAAGGGCAGTGTTCGCAGGACTCGGGAAAGTCAGGCCCTTCTTCGGAAGGATGCAGTGG |
| | GGGCTCAAAGGACAGACCGGGGCGCGCAGTCCAGGCTGCTCCCTCGTACCCCTCTCCCTCCTGGG |
| | TCCATCTTGGGACACTCTAGGCTGGGAGGGTTAGTACCCCCTCCTCCTGTCCCGGGGTTAAAGGG |
| | CCAGTTTGGGAGGGGGTGAGGGGGCCACTTCTTTCTGTCCTCATTTTCTGGGTGCTCAGAGGGGG |
| | CAGGAGCCATCCCGGTCCTCAGTACCACCCCCCCGCCCCCCGCCTCTGCTATGTGGGCTGAATGA |
| | GCCATTCGGTCGCTAGGAGGCAGAACAAGATCAAGAAAGCTCAGCGAACTTGAACCTGTACCAGA |
| | GCCTCCCCCACCTCCTGCCCGGCGATTCTCGTCCGGGGAGGAACGAGCTTTGCGAGGGTGGGGGT |
| | GGGGGGAAAGAACGGTTAGGCAGAATTCCCTTTCTCTCCCCCATCACCCCGTATGTCTTTGTTCT |
| | TCATTTTGACTTTAAAAATGCTTCTGGCCGGGGCCGCGGAGAAGCGACCGGGCGCGCGGCCGACA |
| | CCCCCGTGCGCGAGCTGAGACCAGCGCGCGCCGGGCTCGGAGCACGGTGCAGTTTTCGCTTTCTT |
| | TCGGGGCCGGCATTTTTGGTAGGGAGGAACCGGGAGTGCGCGCTCTAGGGCTTCGGGGCATGGCC |
| | GAAGAGGGGGATATGGCAAGTTTGCACTTGGTCTCCAGCCTCACTTCTTCCACCCCCTCACCCCC |
| | ATGCAAAGCACAGACCTCGGTGGCCTCGGCTGGCTTGCTGGGCGGCTCTGCAGCCCGACACCCCC |
| | CCTCTCGCCTCGGAGCTCGGAATCACAACAATAGTAATAGTTATCATCATAATGATGCGGGCAGG |
| | CAGCGTCATTAATAATGAATAACCGCAGCCGCCGCCGCGCACACCCAGTGCCCAGAATTGCGGGG |
| | GAAATGCATTTGCAGAGATCCCCCAAAGTAAAAAGTGTAAGCTTGTGGACACAGAATGAATCTCA |
| | GGACCCGCGCTTGAGGTGTGTGCGGAGATACTGAGACTGCACCAGGTTAACCAGCCGGGTTTTCC |
| | AAACCTCACTTCCTTTTTCACCAACTGGCAGGCCCAGGGAACCGTCACCCCGCGGCCGAGCTGGC |
| | CGAGCTGGACGGGCATGGAGGCAGCAGTCAGGGCCCCTGGCTGCCCTCCGTCTCCGGGCCCCCGG |
| | GCCCCAAGGCCCCGCGGCCGCTGCTGCACGTGTTCGCAGCAAGCGCGGCGGGAGCCTGCAGCCAG |
| | CACGCTGCTCGCTTTGTGCCTCAGAGTCCCCGCGCCCAACTTCACTTTCTGCACGGTCCACCCTT |
| | GCCGGGGCCCCTGCCCCGGGCCTGTAGCCCCCGGCTTTGCTTTTGTTTCTTTGCTTTTCCTCTCT |
| | GAATTTCAGCCTCCGTTTGCTTCTTTACCCTGTTAAGACAATCAAGGAGAAGGACTTGGAAAGCA |
| | AACTTGAAGACACATCTCCCTTTCCCCCTCCCCCTCCGCTCCCCGGCAGCTCTCGTTTTGCTCGC |
| | TCCTTACCAACATTTCCTATAAGGATTATTTTTTTCCCTTAAATTTATTCTTTTGCAACTACACA |
| | GAGAGGAAAGAGATCTCAGTCTGTCACTGAGACATTGAGACGTTCCAGGCTGTCTTGCTGTTTGA |
| | ACGTAGAAGCATTTTATTTTCTATTTCTTCCTCCCCTCGTAGAGAGAATTCGCGGCTAATTATTA |
| | TGATTATTTGCCCACTCCCTTCCACTTCAATCGAGGACTCCCTGCTTTGTAGCCGGAGTTTAGGC |
| | CGGAGCTTAGAAATGTTGGTATTGTTGGGGCGAAGGAGGATGGAGTTGAATTGAGGGAGGGGGTA |
| | AATGGCTGAGGGTTAGGAAGGTTTTTAGGGAAAGGGGAATTTGCATTAAAATGCAGAGAAATTAT |
| | CAGATGCCCAGAAAGGAAATGTTGATTGCCACTGAGAAAAGATGTCAATGCAAATCAGTAGACTA |
| | CACCATGAGAATTGTATTTTCATATTTTCTTTGTGTCCCACTTTGTCTGATTTTTAATAATATAC |
| | CAGCAATGATAAAAACACGTTTTGGTATTTCTCTGAACACCACTAGCCAAATGTTTTGCAAGGAG |
| | ACCGATGTTAAACGTATTTCATACATTAGAATATAATTCTTGTTAATTAGCAATAATTTACGTTA |
| | AGAGCATAGAAAATGTTGAGGTTACAGGTTTTATATCTGTACATTTGATCATCTTGTTATTTTCA |
| | AGAACTTTGCCTCCTATAAAATTAATTAGGTGAAATGTGGAGGTGTAATCAGCAACCTCTGAATT |
| | ACCACTTCATTTCCCGGTTTTGATTGTAAATCAGTTCAGTCACTACATTTAGAAGACTTTAACCA |
| | AGTCTGTTTTGAACCACATTACCTTTAACTATTTGATACCTAGGAGAATATTTCCTTTTGCACCT |
| | AAATAATATTCCCACTTTTAGAAATGTGTCAGACCTTGGGAACAAAAAAAAAAAAAAAAGAATCT |
| | TAACGGTGGAAATAAAAAATTTTTTTTTTTGCAAAGGTTCTATGTACTAGTAAGTTTGATAAAAT |
| | ATTTTCCTAAGTCTTCCTTCAGTCTGTAAACCTCAGAACTTGTAGCTAATGCTAAACAAAAAAGC |
| | CACATTTATCAATGTGTACTTAAAATCCTTAATTCAGACAACAGGAATATTTTGAGAATGAGTTC |
| | CCTATTCCTCACTTGGTCAAAATGGAAGCAAATGTAAGAGAAGAATGACATTAAGGCACAATGCA |
| | GAGGCACTTCTGTTTGTCTTCTTTTATTTGAAAAGTATGCATATGTATTCTGTATTTATCTTTTG |
| | GCCAGTATGTTGGGCAAAGAAACATAAGTGCTTACTTTACTGTCTTTATTAGTAGGAATATAACC |
| | TTCATATTCCTGTGGTGACCTTATGTTAAATTAGGAGGAGTACCAGAGGCTAGAAATTATGAGAT |
| | GTCCTACTTGAGCACAGGTGCAGCTAGGCAGGGCTCTCTCAATATTATTTCACCTAGCACATCTG |
| | GGAGTTACTCCAGATCTTCCCCCTCAATATTCAGCCTGGGTAGGGTTGAAATAAATTTAACCTGA |
| | GTTCACTGGATTTTTGCACTTTATCAAAATCTGTTCCAATATTCTACACTCAAATTAAAATCTAT |
| | TTTTTGATTCTCTGTGGCTTTAAGTTCATTAAATGTAAAATTGGCAGCTTGCTAAAGAAGGTCAG |
| | ACTGATTAACTGTTTAAGACTTGTACATTTTCTGCTTCAGTTTTATTAACTGGCAGCATCCTGGA |
| | TGTTTTGTATTTTGTGATTTTTTTTTTTTTTTTGATAGAGCAAGCATAAGATTTCACAAGCAGAG |
| | ACTTACCAACTCTCTTTTCCCCTTTGGAAGCTTAAAAAATGATAGAAGCTGGTAAAGTAGATGCT |
| | GGAGTATTTTAGTACAAAGTTAAAAAAAAAAGCAAACAGGAAAGAAAGACATGTCTACCTTGTTA |
| | TACCATCCGCTGGTGATTATGTGTGCAGAAATAGTCTCATAATGAAGCATTTTGGAGCTCATTCA |
| | GAAAATTAGTCCACTTTGACAACATTAGGCGAAGTATTTCAAGTCTAAAGAAAGGACTTCTCAGC |
| | CTTGCTCTGAAATGTGGTGTTTGCTTGACCATTCTGATTTTTATATCATAGATGCCACCAAGTGC |
| | AAACATGTTTAGAATATTATAGGCATTCCATTTCTCAGAATAAAAAAAAAATGACTAATTGGCTT |
| | ATTTTCTTAAGTACTCAAAAGTATCCCATTTAGCTAATGTGTCTGAGAAATACTGCCCGTGCATT |
| | TGGTATTTCTTTGATTTTGTGGCACTGCTGAGAGTGAGAGCAGAAAGGTTTTTGGCAGTGTGAAT |
| | TATGCTGCGACATGATTATTATTTAGATCCGTTTCATAGGTGCATGCAGTCGTTTTCTTATTACA |
| | GCAGTGTAAATGTGGCACATTTTTCATGTGACATAGTAGCTTTCTAATTTATGAAGCCATGTCTG |
| | TTTACTTAGGAGTATATACATTCACACACAAAGGGTGTGTGTGTTTATTCACCTCTCCTTTCATT |
| | CTTTGGCACAATGGACAACTTGGTGTATAGGAAAAAAGAAACAAATTTGGTTTCTATCCACTTTT |
| | TTTTTTAACCAGTTTTTCTTGTAGTTATTATTTAAGCTTTCTTTATGTTCCCTGTGTTAACTATT |
| | TAAGTAGCATTCTTTCTAAACTTACAAACCAGACACATTTGTTGCTGTGGGTGTGTGCATGGGTA |
| | TATGTGTGTGTGTGTGTTCTCTGGAGTTATGCAAGGAAGACTGTTTTCTTTACATATGTGATGAT |
| | TTGCCTCATTGACAAATTTGCTCTCTGGTTGATAACCTTCACATCCTTGTACTTTTTGTATGCTC |
| | ACATTTTCTGGGTATTATATAGAGAAGCCTAGAAACACTTTACATGATGTGGTGGGATGGCATGG |
| | GGTTGAGATGTGCTTCTCCCCTTTCTGTCCTCTCTGGCACTCTAATAATTGTGCTTTTGTTTCTC |
| | CAACCACAGCCGAGCCTCTTGAAGCCATTCTTACAGATGATGAACCAGACCACGGCCCGTTGGGA |
| | GCTCCAGAAGGGGATCATGACCTCCTCACCTGTGGGCAGTGCCAGATGAACTTCCCATTGGGGGA |
| | CATTCTTATTTTTATCGAGCACAAACGGAAACAATGCAATGGCAGCCTCTGCTTAGAAAAAGCTG |
| | TGGATAAGCCACCTTCCCCTTCACCAATCGAGATGAAAAAAGCATCCAATCCCGTGGAGGTTGGC |
| | ATCCAGGTCACGCCAGAGGATGACGATTGTTTATCAACGTCATCTAGAGGAATTTGCCCCAAACA |
| | GGAACACATAGCAGGTAAATGAGAAGCAAGGAGAAAAGCTGTTTGCATGTTTTCTTTTCATTTTC |
| | AGAGGTGCTGTAGCCAAGCAGTAAGGAGTTGTGAAGTGCTTTCTCTATTACTCTATGTGACTGTC |
| | CATGACAGCCCTGTAATGTTAAAATAATCATTTCTGTTGCTTACGTCCAGAACACAGAAAAATAA |
| | ATATTTTCCACCTCACTGAATCAGATGTAGGCAGGATAGGTACACACATCAGACACCTTCTCTCT |
| | GGATCTGTCGATTTTGGATTTCTTTTCTTCCCCATCCCCACCTTCTCATTTTGAAGTATTGAGCT |
| | TTACTACACCTAGTCCAGCTTCCATTGTCCATTTCCAGCCTTGGTGACGTGTCAGAGGCAAAGTG |
| | GCCATATAGGCATTTGCAGTTCAGCCAATGACTTGTTTGACTCAGAACATCTGGCCAGGCCTCCT |
| | TAGGGGTTCAGCTCGTTCTCAAGGCTTCCCTGAAGTAGAGTGGGCTGGCAGGGTAGTTGGAGGTG |
| | GTGGAAAGAGTTAACTGAGCTTCAGGGCTAGCCTTGGATCCATATTGGCTGTCAGCCCGGATGGG |
| | GCTGTAATTAAACACAGCCCCGTGGTGGGATGACACCATGACCTTGACTTTAAGATGCCATTTTC |
| | GACTGGCCAGGCCAGAGTAGAGAGGGCAGTTGCTGAAGCGCACAGACATGCTTACTCGAAAAGTT |
| | TAAGGGCATGTTGGAAATTTCAAAAGGTTGGTTTGACAGGAACGGCTGCTCCCTGCAGCCTGCCT |
| | CCTCAGCTAAATGATAAATGCTTCTCTGTGCTCTCTCTTGTCTCTGATGTGGTTTTGACAGATGT |
| | ATCTTGATTTTGTTTGTGGTTTACACAGCCACATGTCACCCTTACAAATGTCCAGTCCAGACTCC |
| | ACTGTTTCTGCTATAACACAATGTAAAAATTTTCTTGGAAAAATACACACACGTATTCAACAGCC |
| | CTCCCTCCTTTGGTTAATTTTAGCAGGGAGGCAGCTAGGTGTGTGGGTTTCTCGGCAGCTCAAGG |
| | GAAAAGGAATTAAAGGCTAGCAGTGGGACTTAAATTCCCTTCTCTAAGTGATAAACAGTAACACT |
| | ATATAGTGACCCTCAAAACATTTTTTGCTTGAGCATGTTAGACAAAAGTCAATGCAGATTCTGTG |
| | ATGACAGACATGCCATGCCTGTTGGTGGATCGCTTTCTTCCATCTACCTACCACCCAGCTCCCGA |
| | AAGGCAAGAGGTTTGTTCAGTTTTAGGAAAGGTAGTGCATATCATGAATTGATTCACTGGAACTT |
| | GTCTCTCCGACCTAGTTTGACCACAAAGTTGAACCATAATAGGTCAGTGGTCTAGAGGGGATTAA |
| | ATGTCATATTATTTCTCCTCTCCCCCTCTAGAATTTGATCATTAAAACCAAACATGGCATTTTCT |
| | TTCTTTTTTTAGTGCTTTCTGTGATAGCACTCAGATACTTTCCCTTTAGTGAAATGGGAAATCTG |
| | CTGCTAGGGAAGCTGCATTTGTGGAGTGTATTTCTTGAATCCACCACATTTACCTTATGTGACAT |
| | GTAGGTGAAGATTTTATCTCCCCTACCCCCCAGCAGGATGTGGGAATGACCATTTCCATGTGTTG |
| | TCTTGTGACTGGAAGGAAAATGAACAGAAGTGTAAGGCATGATTAATGAAGCAAGAGCAGGCGGA |
| | AGGGGATTTGTCGTCTTCGGAGATCCAAAGCCTTGCTAAATCACCAAATATGGAGTAACACTTGC |
| | GTGATGTAACATCGTATTTACATATCGAGCTGCTCGTTTAAAAGACAAAACACAGTGTCTGTCAA |
| | GCAAGAATTAAAACCACACTTCTTACTGAGGTCCCAAATAGGTTATTCAGTCTTAGATTAACCAG |
| | CTCAAAAATTCTGTGCCTCTGTATTTAGAGGAGGAATCTAAATGCTGGGGGGAAGGCCTTACATA |
| | TAGTTAAGACTTTTACTGCTATAGITGTGAATCTATGTAGGGAAATAAGAGATATTTGCTTGAAC |
| | TCCCTGGTTGTCTAAAGGTTCTGTTATTATTTTTTTAAAGAACAAGTATAATAGCAGAGCCTAGA |
| | GAAGCCAAAACCAAAAGCAAATTTAAAATATATTTTATAGCGCTAATAATCAATCATTTAACTGA |
| | GACGAAAAGCTCTCTAAGATGTCTAAGATATTCAATGGGCGCACAACAAGTGCTGTGACCCAGGT |
| | GAGGTAAACCTTTCGTGCATGAATAATTACAAAGTCTTGATTTCTTTCATTGTGTTTAATCACCT |
| | GTTCCCACCCTGGAACTGGCTGAACATAAATAGTGTGGTCACATCTCAAAGTGAGATGTCAGTAA |
| | CTAGAATCACGACTTCTCATAATTCACAGTAATGAATTAAGAGTTTCCTATGGTGAAGTTAACAT |
| | TCTACCATTGCACATAAATTCCGACGCTCTGGCCCTCAGGTGCCCCTGAAGCGAAGTTCTGGAAG |
| | ACGGCTGTGTGTGTACCCCCAGCCCATTTCTCTAAAGCACGTCTGCACAATTCCAAGTCTGCTTT |
| | TCTTTTTATGATGAGGAAGGAAACAATAACAGTAATCATTCAGTAGATATTTGAATTGTGTCACA |
| | AAAAGAAAGGAGAAGCAATGCCTTGTATTAAGGAAAGAGATATATTGATGAATCTCTAGAAGAAT |
| | GTGTTTGGCAACCACATAAAAGGTAGTCATTTAAGCGTGCTGGGTAGGAAAGGCTTTATTAAAGT |
| | GATGTAAGTTGGATTTGAGTTCACTGTGAGCCTGTACTATTTTATAGGCAGGAAGCAAGAATAAA |
| | ACAGTGACAGATCTTCTTCCTAAGATAAATAAAGCTTAGAATTCGGGACTTTCAGATAGGAGAAT |
| | AAGGCAGAGTTCTTTAAATCTTGAGTAAAATGGTATGCATTTTCACTGTACTCAGGCCTCTCCAA |
| | GCTGAGTTTTTTTTTTTTTTTTTTTTTTTTAGACAGAGTTTTGCTCTTGTTGCCCAGGCTGGAGT |
| | GCAGTGGCATGATTTTGGCTCACTGCAACCTCTGCCTCCTAGGTTCAAGCGATTCTCCTGCCTCA |
| | GCCTCCCAAGTAGCTGGGATTACAGGCGTGTGCCACTACGCCCAGCTAGTTTTTTGTATTTTCAG |
| | TAGAGGCAGGGTTTCACCATGTTGGCCAGGCTGGTCTTGAACTCCTGACCTCAGGTGATCCACCT |
| | GGCTTGGCCTCCCAAAGTGCTGGGATTACAGGCTTGAGCGACCGCACCTGGCCCAAACTGAGTAT |
| | TTTTTAGAGGATTCTTTTTACCTGGTGAATAGATGGGTATGTGCTCCCCCACTTCATCCACGTAC |
| | ACATATGAGCTGTACACATAGATGTTAATCAGGTTGCTTTTTTCATTTTATTTAAATATTCAAAA |
| | TATTGTGTGATTGTGATCCATTGAGATCATTGAAGTAGTATTAATTTAGCTGGGAATTAGCAGCT |
| | TATGTTGTGTGGGCCCAGTTCATCAATATGTGTGTCAAATATCCACCCTCAGATATCAGCAGCCT |
| | TTGACTTGCAGTCAGAGCTTTCAATAGGGGTTTTGTTTTGTTTTGTTTTCTTTTTTTTTAGTCTC |
| | ATATTTTATGATGGGGGGAAATATTCATGGAAGATTACTGAATGTGAAACTGCTGTTCCCTTATA |
| | GAAAAGACCCAAACATTATCCCATCATGTCAGATTTGAGGTTTTGGCTTATTGCACATAGGAATC |
| | TTTAAATTAGGTTTGGGCTGTCATAAAGTTAGCTTTTTGAGAGACTAGAGAAAAAATATGACAAG |
| | TCCTATAAAATGTAGTAAAGTCTGCTCCTGTTGATATATAACATTTTTTCTCTTTAAAATCATGA |
| | ACATAGACATTTATATCAGGGTATTTAAAATTATTTGCCTAGACAGTATAGCTGGTATTTAGATA |
| | ATGATATATCAGACAGATACTCTAAAGAAGGGAGATATAGTAATTCAAAATGGAAATACATTAGC |
| | ATGCTCTTAAAAGTAAATCAAATACAATATGCTTTTCAGAATTTAGAAAATGAACTTACCTTTTC |
| | TTTTTGTATCCATTCTGATTCTCTCCTAGCTCAGACTGAACTGGAGGATGTATTTGTGTACCTTA |
| | TGGTGTAATTGTAAATGAGACTATAAATTATAGTATGCTATTATGACTACTTATTTTTCTTTTCT |
| | CTTTATTTATGTGATTCTGAATAAAAACGACTGACTTCTGAAGTGAATTTTCAGCATGGCAGTTA |
| | AAACAAAAAAACACCACTACCACAAAAAACAATATACTGGTGAACATTTTACTTACCTTCAAAAT |
| | CCAGAGAGTAGAGAATATTGTTTTCTAATAAGTACCTAGGATTTTCATAGGGAGCCTATGTTTGT |
| | GAGGCCACTATCCAAAGACTAATGTTCATGAAACTGGGAGTCCTATATACAAGCTGTATTTGTAA |
| | ATAAGACAGAGAAAGGTTGTGAACTGGCAGCTTGGATGTTTTGTCAAAGTACAATGTCAGAGAAA |
| | CTCTTTCTAAGACAAATTGTAAATAGAACTGTCACAGTGATTGCATGATTGAGCCGCAGAAGTGT |
| | CCTACAATTGTTGGCATTGTCAGGACTTTTGAAAGCTTAATTAAACACAGTGGCCCGCATGGCTG |
| | CTAAACTTACTAAGGAAAGCCAAAAGGAAAAAAAAAATATATCTAAAATGTAGAAGATCCTAAAG |
| | ATCCCAAACTTCTTCAGACATTACCCTTGTGGTGACACAGAAAAGATGCATTGTGCATTTTCCTA |
| | GTCATCTTTTATAAATAATGTTTTGAGAATAGGTCCACGTGATATGAAGATCATCCTGTGTGTGA |
| | TGTGGAGATTGTGTTAGTTTTTCTGTCTCCTGCTGTTACAGACAGTTAATGTAAAAATGGCCTTT |
| | TATTGGAAACACAAATATGTATTCACTTCAAGAACAGGAGCAGAGGGGGACAAAGTTTCCCTCCC |
| | GTTCTCTGTGTTTATTCTGTGAAAGCTTAAAAAAACAAAAAAACAAAATAACCACACACACACAC |
| | ACACACACACACACACACACACACACACACACAAAACCAGTGCTGTAATTTCTTAAATCACCCCA |
| | TTCCTTGTTGTGTTGCAAGTIGTGCCTTTACTATAAAGGATCTGAAATATGTTTTGCACACCTTT |
| | CTCTTAGTGAATGTGGCATATAATTGTGGAGCTGCACATGGGCTGGAAAATGCAACTGGGTGTAG |
| | AAACGTGCAGGCAGGGTCAGGACAGGCAACCTTCAGCTCAGGGAGGAGGCGAGTTGGATGCTTAT |
| | TAATGGCATCTTTTAGAGTCCTGGAAAATCATTAATTTCACACTGCAGCTTCCATGTAGTTTGGC |
| | TAATGTGGGAGTACTAAATTGGGGTACATAAAAAACATGCAAATCCTAGGGAAACATTTTTTAGA |
| | TTTTTGTGATTTCTCAATGAAAATGTTTAATAAAGGAGAATAGGTGAATGGTGGCTTTGCGTGCT |
| | GGTCAGTAAGAAAGGAAACAAATTTTGGCTTTCCTTTGGGGGGAAATATTAAATTTAACCGAAGT |
| | AGAAAGCACAGCTAGGGAGATGCACCAAGATTGCGTCACTGGATGTTAATTTAATCTACTTTGGT |
| | TGGTCTGTTCACACCCTTTATTGCACAAAGAAGTCATTTGACAAAATTTACCCCAAGCCCAAGTC |
| | TGTTTTTACATACAGTAGTACCAGCTTTGTGCAATAAAAAGCACTTAGCATCAAGCTGGACCCAG |
| | CCTGGCACCTTGCCACTTTTTTAGCATGAGATTTAATCACCAAGCATCTTTAGTACCTTTCTGCT |
| | TGTTCAGATTTCATTTGGGTCATGGCTATGTCAGCAGTGTTGTTATTTCAAGGATTAAAAAAAAA |
| | GATTCTAACTTAGAGCCCACTTTTAACATTATTTCAAATCAAGCAGGTTTTTATGTTATGTATCA |
| | ATTTGGATGACATTAATGAAGTTCATAAAATATGTTCAGACTATATAATTTAATGGATAATGACT |
| | ACTATTTTTATTTGTAATACAAATAGGAAATTGACTGTTGTCTCCCTCCCTCCTTGGTTCTTTTC |
| | TCCTACCTAGGTAAATGGGCATCCTTAAACAGCTTCTCCCCTTTACGACCAGGGTATAGAGCACT |
| | GGCCAATATCAGTAAATTTACCTTTGTAATTTGCCACGTAGTTTTTACAACATGACCTAATTAAT |
| | TTGAGCACGAACCATATTATTGCTACTGGAGTCATTTTCTGTCACAAACTTAATTTCCAGGAAAT |
| | GTAACCTGACAAATAAGAATTCTTTAGCTCTCTACATGTGCTCCTAGAGACCAAAGGCAGATTTA |
| | AAATAATAATAATTTTAAAAGTGCCAGCATTATTAAAGCCAGTATACTTGATGCCAAACTCAATT |
| | TGAAGCCAGTAAACATCAGACTGTATTTCTAATCAGTTTTAAAATGTAACTTATTCCATATTGGG |
| | TTCATTGGAATTTGTCTCCCTGCTTTTTACTGGCCAGCTGCACTCCCTATGCATTTTTAAAACAT |
| | TTCAGCAAAGGCTTTTGCTGTTCTTAGCAGGGTTAGTAACTTGGGGTCTATTTCTGAGCTCATTC |
| | GTCATTCTGCAATGGCATTGAGTTAGGTTGGCAAGGGAAGGATTTGAGGCATGGGGGGTGGTGAG |
| | GTCACTTCTGATCCCAGCAGGGAATAGGTGAGCTTCATTTGCCTTTACAATAGGCGCACAGTTAC |
| | TGCACCTTGGAGGAGCTCTCAGGTGCCGCTCAGATGGGCGCATGTAAATGCCCTGTCAGATGCGG |
| | AGCTGGAATATTAATGCTTCTTCCACCACCACACCATAATAAAGCTGTACACAGCAAGCTTAATA |
| | TGCAGCTAGTCTGGGGAATTGTATAAACTTAGATAGCCCAGTGTGAAAGACAGCAATGGAAAAAT |
| | GCTCGATATGCCACAGTTTCCATTCTTGTTCTCCTTTGATCTATAGCGAAATGAAAACATCATCC |
| | TTTTCTTCTCTTGGAGTTGTCTCCCCAACTCTGCCACCTCCCAATTCACCACCAGAATTTTTTTG |
| | CATGTGCTGGTATGGAGAAATGCAACATAATTTGTTTCATATGGTTAATTACAGGCGTTTGAATA |
| | TTTAAAATTTTAAATAGCCACAGTTCCAGCTTTTCCCGTTAAAAGTATGTGATTTGAACAAAGGG |
| | AGTACAGTGTAAATATTTGTGGTGATTTGCAACACCCCTCTTTCCCCATTACACACACACACACA |
| | CACACACACACACACACACACACACACACAGAGAGAGAGAGAGAGAGAGACATTCAGTTTAAATC |
| | TAGTACTGATCTAAAGGACTTCTGTACCTTCATTTGTACTTTTTTTAAAAAACAACTTTCAACTG |
| | GAATTCCATTAAAATATTTCACCATTATATTTTTGAGTCCACATTGCTTGAGGTTTTAAAGAAGA |
| | TTTTTATAATGTGTTCCTTTAACAGAATCAACAGCTGTCAGAAGCAGTTGTGGTAGATCCACAAA |
| | ACGTATAAAGAAAAATACACGTTTCCTGAAACAAAACACTTGGAAAAATAAGCTGCTAAGAACTG |
| | GCGAATTAGAAGTTTGCAGACAGGGAACTGAAGTGTCATCTCTTGGTTCACCCTGGAGACTGATG |
| | TGAGTGGATCTGATGCAATGCTGCTGGAAGATTTACCTGCACAGGTTGCTCCTCTAAGGCAACGC |
| | GCAGTGCACGATTGACGTATGCACCAGAGCTAGGCTGGGCCTCAGCTCGCTCCATCTTTTGCCCT |
| | TTTTGATCTCTTTAGATAGATAAAATACCAAGTTCTCAGAGTGCTAAACAACAAATTATATATAC |
| | CTAAAGGTGAGATGATCAGGTTTAAACTTCCTGTAAAAGAGGCGAGAAGGCGCCTTGCACACCCT |
| | TTTCCAGATAGGGCTGGCAGCATGTTATTCAGAACTGAATCAGTCTCTGCCAGAGAATTCCCAGT |
| | GGGAACCTGGAGCAGGTATGTTGATGAAGGGGATACCTGGGGACCTTTGGTCACTCACAATGAGT |
| | TTTTGTTTGTATTCTCACTGTTGTTAGCATTGCCAATGAACAATTGCACCTACAATTTGATTTTA |
| | GTTTTAGATAGAGGCGAATCCACTGATTAAAAACTCCCATTAAAATAAAGAATGGAATTATTGTG |
| | AAACCTGCAAGGGTGCCTTCAAAAAGAAAACCAGTGCTGTTGTATACCTACCTCGCCTTTCTATT |
| | TGCTTTTTGAACTTTCTAAAAAACACAAGGAAGCTTTTTGCTAAGCATCAGGGCATTTAAATTTA |
| | TATTCATCAGTTGTTCATTTTCTTAATATGTAATGATGCATAAAAAGGCTGCAAGGAAATCACAT |
| | CTGTTAATTTTTAGGGAAATAAAGTGTAGCTTGGATTCTTATGTTGGAGCACAAAGCACTATGTG |
| | CCAAGTCTGTTCCTGTACATTTTAAATATAGAGTTTTAATATTTGGCCAATCCCTGCACCTCCTC |
| | AAACAAAAACAAACCTCAAAAAACTAAGAGAACCAAACCTGAAGTATTCTCCTTCACCAACTCAA |
| | GGTATACCATGATTTTATGATTTATTTACATTTAGGGGGGAACCCCTCAGTGAACCATTTACTCC |
| | CCATTTTAACTCCCCTGCCCCGATCCTTTCAGTTTCCAGTTAAAACAATGCATTAACCAATGTTA |
| | AATCTTAAATCTCGTGAGTTTCTCTCCATCACACCCTAATATTTTAAAAAAATTATTCCTTTACA |
| | TTTAAAACTGAACATTGGCTACTGAAGAATGATTTAAAGGCTGAAAAAAATTTTAATAATAAATC |
| | GTAACCTTCTCATGTTATGTTTTTGTTATGTTAAGGAGAAAAAAATCAATAAGGAAAAATTTAAT |
| | TCTGATAAAGATACTCTTGGATCTTTGAAAACAACTGCTGTCCTTTTAACTAAAACATTTGAGCA |
| | GCTTCAAAGACTATGTATTTCTTCTGATCTTGGAGCTGTGTGACTGGTAGCAAGAAAGAAAAAAA |
| | ATCTTATTCTACATACAAGTGGATTGCTTAACAAGTCAGCACAGACACGTACTTGTTTGTACAAT |
| | AGAGATAAAAATTCCTGTATAAAAATAATTCAGCTGCTGACAGCAGGCATTGTTGTTGGACCTGT |
| | CTTTTGTGCTTGTCCCAGCTCTGGGTCCCCCTCCCCTCCTATCTGCTTGGGGCAGCCTGCTGCCT |
| | GCACACTGCTGACCAGAAGTTAATTGCTATATATTAAGTATATAGGTATTGTATTTAAAGAGGAA |
| | TATCTCAAGGCTTCCTATATGCATTCCACTTTACTTTCTGATGTGATTGCGGTGTTGCCAGCAGG |
| | GGGGTGGCAGGCAAACGCTCTAATAGGGAAAATCACTTGAAGGCAGTTAGGGGAAATTTGGCCTT |
| | CAAGTCCCATTTGCTCTGTAGTGTAGCATTGGTTTCTAAACTTTTGTTTTTAATCTAATTCTGAT |
| | TTGCCCTGTCACATCCCATATCAACCCTCATTGAACTCTACTCATGTAGAGTAACATTAGTGTCA |
| | AACGGAATTGGTCAGGACTGTGGACCTGTGGCTCATACAGATGGTTGTGGATGTGGGTTCCATGC |
| | AGCTCTGCATCCTATCCTTTCTAATAAATGTTAAAATGTGGCACATTTCTGAGCAGGGCCCAAGG |
| | ATAAGAGAGTTAAGAAATCAGGGGGTAGTACCTGAGATTTTTCTCCCTTCTCTTTCCGATTTCCT |
| | TGATAACATCCACATTTCCGGTAAGATCAACTCTAGGAGAAAGTCTGAGGCTGGGGGAGAGAGGG |
| | GGAGAAGGGTGCGGAGAGAGGTTCTTGGAATATTCTTCGATAGCAGTTCAAATGAAATCCCCACA |
| | GCAGAGAGCTTTTGGGTCTAGCAGTGGAGCGGTAAGCTGGGACACGTGGCCTTTCGAAGCTGTTA |
| | TTCTCAGTCTGACTTGCACACCAGCTGAGATAGGACTTAACATATACTTTCTTGCTTTCACCTGG |
| | GTTGGAGAGGTTGGGGTTGGGAGGAAGAGGAGGAGTTCATTGGGAATTCTGTCACTAGAATTTTT |
| | TAAATGTCAGGAGGTTAGCAAGGTGTGAGTTAGCATTCAAGCAAAGGATTCTTCTCCAGACTAGT |
| | AATTGGAAAGCCTGCAAATCCAGGTTCCCACGATACTCTCTAATAACTGGGGTGGGATGGTGGTG |
| | GTGGGTGGACACCACAACTTTCTGAATGTCAGCTGATGTCTGCATGACCCGTTCACCATGGATTA |
| | AATGCGGCTGGTGCCGAATGGAGGAAATCAGAAAGGCAAATCTCAAGCAACAGGATTTGCACTCC |
| | TCAGAAGTAAACCAGACCTTGCTCCTCTCCCTCCTGTGCTTCTCCTTTCTTGCTGGTTTCCCTTT |
| | GGAAGCAGAAACTTCTAAAATTAATGCCACTCCAAGCCAATGAAAAAGCTGTTTTTATACCACAG |
| | TGGATGTTTACACAGGAGAGACAACTTGAGGGGGAAAAGGCTTTTTGGAAGGGTGGAGGGACTCG |
| | TGTTAATCTGTTCTGTTGGAGGACTATGCAGTATTGCCTATGAGCGACTCTGGGCTGTTTTTGAT |
| | AAATTACCATGTTTAGAGATAGGTTTGGCTCTTAAGGGCTTAGTTTTATGAACAAAGTCCGTGAC |
| | GATGTTTGCAGCCTCTGTTTGTATCTTAGCCCCTTTGGCTTGACTAGAAGCTCTATGTTTAGTTT |
| | AAGCTCAGTCTGGAAGATATTACAATTTTGCATTAAAAAAATGAGGAAATCATAGGAAGAAAAAC |
| | CCTTTGCTTTTTGGATGAATCTTACTGATAATTTGCTAAAGCTCATTTGAATTTTAAGCACTTCT |
| | TTAATCTTCAAAGGCTAAATTGCTTTATGAATATGCATGGTGTGGGCAGACTTCAGTTCATTACC |
| | TAGTTGTAAATTCTAATGACCATTAGGTCCTTCCAGTAATTGCGAATTGTTTTGCATTTTTGATT |
| | GGCCTATTAACATGTACATTCGGTGCACATCAGGCTGGCCTGTCAGCCTGCTGAAGGAGAAAAAA |
| | AAGGTGAAAATTGTTTATAGCACCAAGATTCTTAGATTTTCAATCTTGCAAAATTGATGATGTAA |
| | AAAAATTAAAGCAGTGTTTTTTCTTCTCAAGATTAAAAGTTCACCAAGAGATTTGACATATTTAA |
| | TTTACATGATGACTTTGCACTCCTTCATTAATGTAATTTGCATATGAAGCTGTTGTTAATCACTT |
| | TTGATCATGTTTTGTGTATTAGCTGCCTCAGTGGCTCTCCTCCTCAGATGCCCCAGTAGAAAGGA |
| | GCAAAATGATGCATCTTCTTGCCAAGTTTCCTTTAGTGAATTGAGGAATTAGAAGTCTAACCTTG |
| | AGTAATTACATATGTTTTATCCGTTTTCTTTTAACGTTAAGTACAGTTTGTGAACGTGTTGGCTG |
| | GAAATCGTTCTCATTTGGGGAGAAGACTGTAAAATTTAAGTATATGATTGAGGCACTTCCAGATA |
| | CATAGAGAAATATGTATTGCCTGTTTCTGTTCCCCACGAACATTGCAGGGCAGTTTTATTGTTAG |
| | CAGTTTGATGGCAGGAAGCCTTGGCTATTATAGTGTATTAAGACATCAGGTTCCTCCTTTGGAGG |
| | AGGGAAGGCTACAGAACTACAAACCTTTCTAACAATGCTTTAGGTTTCTTCTTTAGATAGATGGC |
| | TGGCACCTAAAGGACTTGGGCCTGGGTTTGGCTGACTCTTTTATCTTTTAGATCAAGTAAGTTTT |
| | CTCATTCAGCTGCTGCTCTGAGCTACAATGTGTCCTCCCCTCATCACCAAAGTATATCCTGGTCT |
| | CCAGGCTCCCTGGGCTCCCAGTGTCTCCCTCAAGGTACACGAGTGCCCTGGTGGTGAAAACAAGG |
| | TGCTAACTAACGGTTTCCGATTTTTGAGAGCCTGTGATTTTGGTGTTTGCCTTTGCTGTTGAATA |
| | ACCTGTGCTGTATTATTGATGTTCATCTTTGGTTTATGAGTTTATCACTGGTTAACAAGCAGAAT |
| | CAGAACAGTGTAACTGATATTCTGATTAAAACGAATGTTTAATGAAAGAAAATAAATTGTGATGG |
| | AAAATGAACAGTGTGTAAGAAACATAACTATAATTTTAACCTCCGAGGGACCTAGCACTGCCCTA |
| | CCGTGACTTCCATCCATACCATGCTAAAAGCATGCTTCAGTTTAAAGTTGTTAATATTCAGCTGG |
| | GAAACAGTATCCAGAACACAAATAAATTATTAAGTGCATGAACTTTTTAGGCAGTAAGATGAACT |
| | GATGGGGTCCATCTGTGAGATCCAGGGGCTTTTTATTTGTGTGTGTCGAGCGATTCTGCCCTCTC |
| | CGACTTCACAGCCTTTGGTCTCCGGCCAACTGCATGCATAATTGATTCCACACGCACTATCATTT |
| | TCTTGATGTAATTGCTTTACTAAGATATGATGAAATCTAATGGATAATTTGCTATTTGAAAATGG |
| | TCAAAAAAAATCTTCATACTTTATGTGGGGCTGAGTGGGCAGTGGAGAAAGGGGTATTCAGCTGA |
| | CCCGGTATTTAAGAAAACAAAACAAGCAACACTAACTTATGCATGCTGCTTCAGTCGCGTTGGCT |
| | GTGGATAGGAAGGTCTTTGTGACATATGGAAGCCAGTGTATAAATCTCTCTCCTTCTATCTTGCA |
| | TCACCCCCTTCATTCCTTCTCTCTTTCTCTCCTCTCTCTCTCCCCCAAACTTTACAAGAAAGGGA |
| | TCCTAACAAGGTAAAAAGTAAACAATTTAGTCATCACAAGCCTTATTATTCAGTCTATCCAGGAG |
| | TTTTGCCATGTCGGTTTATTTAACTTCCAGGAATGTAAACACTGACACAGCCCTAGAAGCAGCAA |
| | GAAAGATTACAGTATTAGAGTTAAAAACGTGAGCATGGAGGAGCTGTGCTTTATACTCTGCTATA |
| | ATAACACTTTACATTGAAACATAATGGTAAGTCAAAAGTGACTGGAAACTTCTGCTTATATGGAG |
| | TACAAATTTCATTCTAATAGATTGGCATAATCTAGTGTACCCAGGGTAGATTGTTATATAATGGA |
| | GAAACTGTATAAATGTCAAGTACACAAATAATTCTACAGGAAGTAAATAAAAAGTATTAGAATTT |
| | CTTAAGTCACCATTAAATTTTGGTGGTGGGACAATCTCATTAGCTCCTTCAAAATCATGTGGCTT |
| | TGCATAAGTCTTTTGAAAATGTATTTTCAGGGAATTTACAGATGGTGAAACATTGTTTTAATCCA |
| | AACCAGTTAATGCTTTAAATCTACCTTTAAAAAAATTGTACTGTTTTTCGAAGTACTTAAAGGGA |
| | GTGGAGGGGTAGAAAGCATATAAGTGAATCCATCTCACTGTGGCAAACTGTTTTTCAAGTAAAGT |
| | CATAATAATGAACAACACATGATCTGAAATTTGATCAGCAAACATATCCTTATGCCAAGGAATTT |
| | TCTTTTTTTCTTTCCTTTTTTTTCTTTTTCGCCATTCACATACCAAGGTTCTGTAAATCAGTAAA |
| | CCAGGCAGAGAGTAACTATTGTAAGGGGGAAACCAAATCATAATACCCAGAGTGGCCCAGAAGCT |
| | GTCTTTCTGAAGAAACATTAACGCCACCACCACCAAAAAAAGAAAAACAAAAAAACAAAAAACAA |
| | AGCAAAACAAAACAAAACCTTTTTAAAAAACTGGAAATGACAGAATAGTTTTAAAAGGAAAAAAA |
| | AAAAAACCCAAAAACCAAAAAGCAACAACCACCTTCTGACGCTCAAAACTTCAAACTATTAATAG |
| | ACCACCAGTGAGATAGACTGTCTTTGTGCCTTGAAATGCAAAATGAGGGAAATAATTAGCAGAGG |
| | AACAAAATTCTCAAAATTTGAAGAACTTCTGTGATTACTGGGGGTACAGTGAAAAGAAAATGCAA |
| | ATTTCTTCCTGATCTTAATTAGATTCGATTGTGCGGTGGGTGTGTTGGATTTGGGGGGAGGGGCA |
| | GAGGCAGGGAGTGCTGGGGTGAGGCGTGAGGCTGAGTGTTGTGGAGACAGGTTAGCAGGGGCCCG |
| | GCGGTGTGGCAGGAACAAAGGCAGCTTCCAACGCTGGTGCAGGATTCCGAGCCTTAACCCAGATG |
| | CTCATGGTGCCCTAGTCTTGAGTTCTTCATTTAGGTGGGCTTATTTCCCACTGGGTCTGGGGGAT |
| | TTCATTTGTCCTTTGAGGGGCAGGGTGGACACTGACAGAACAGCTGCGGCCGGCAGAGAGGGTGG |
| | TTAGGAAGAGGGAAGCAGCCTGTGGGTAACTTCCCGACCACATGGAAAGGCTGAATAAGACGTTA |
| | TGGACCCTGCCTTGGGTACTGGGGTCAGCGTCTCCTGGTGGTGTCTGCACAGGGCCCCCCAATGC |
| | CAGGGCACTGCCAAAACACGCTCTTGAGTTTAATGGTAGTGGTTGGTCTGAGTCCTGCCAAAGTG |
| | TATGGAGCAAGTTTCATTGGCTGGACTTTCCCCTTGCATGAAATAATAAAAGCCCTGGCCAAGGC |
| | TTATGAATCTATTTTTGTTTCATTAATATTATTTATTATGTATTTTATTAATATTTTTTGGAGGG |
| | ACCTTGCTCTCATTTGACCATTTGTAGTTATAATTAATGCATTCCGTACTGGTTGTAAAAAGTGT |
| | GCTTGCATTTAATTGCAAGTCAGGGTAAATTAATGGATATGATTTAAAACAAAACTCACTTAAAA |
| | TATTCTTGCAGAACGCAAAGGAGGGGGCAGTCCCAGTATTTAATTTATTTTCTGGTTTAGTGTTA |
| | GTGTGAGAGGGTCGAAAAGATTCTGTGGGTCCAACGGGATTTGTGTCTGTGTGTGCAGGACCGTC |
| | GGGCAACACAGAGGGAGGAGAAAAACCTGGACCGGAGTAGGGTAGCCAGGAGCTCTTTTTTTTTT |
| | TTTCTCTAATTTCTGAGGTTGCCAGGAGGGGCTTAAGCAAAGTGGTCAAGTCCATCTGCTCCGGA |
| | GAAGGTGGTAAAGAAAAGAGGTTAGTGGCAAGAGGGAAGGAGCACAAAGGGAAAATTGTACATTG |
| | GGAGCGTTACTCTCCCTGGCCATGGTGTAGCCAGACTGGTTTAGCAGACAGAATGATAGATTGTT |
| | TTGTCAGGGGTCCCAGGGTGCGCCCTGAACTTGAAGCACTTTGTTTATCTTGAATAGAAAGGGAA |
| | AAGCGCAGACATAATCGATGTCTAGTTTTTAGGAGCTCGAAAGAGGTAGGAGAACAGAGAAGACT |
| | CAGGAGGGGTAGTGGGAGGTGGGGGAGGTGCAGGCCCTGGTTGTGGTTGTCCATTAACAGATGAA |
| | CTTGGCCGAGGGCCAGGCTTTAGATGAGAGCGTGTCAGGGCCCCAGTGCAGCCAAGCCTTTTCAG |
| | TGTTTTTTTTTTCCTTTTTCTTTCTTTCTTTTTTAAATACCTGCTGACTGTACATCAAATGCTCC |
| | CTGGTCTTTTGGCTAAAGGCAAAAAAATAAAAAATAAAAAAAAAAAGAGACGCACAGCTCAATTT |
| | TTTCCCTCCTCTGAACCAGTTGAGGCCAGTCTTTTGGCTACATATGCGGGTTCTATCATCTTTCC |
| | TGGCTTGCCGTTGGGAAAAAAAGTTGTGATAACGCCAGTAACCCGAGGGCCAGATGGGAAGGGTT |
| | TGGTTGTGTTCAGGCGACCAGGTGTGAGAGCTCGTGGTGCAGTGGGGTGGGGCGTGGCCGGCGTG |
| | CCTGCGTGTGCAGGTAAGAAATCAGTGGAAACTCTTTTTTTTTTTTTTTTTTAAATGGCTGAAGT |
| | TTAACTTGTTGGAAGGGCCTGTGAATTAAGCTGTCGGTGGCTGAGAACGATAATATGCAAGGAAG |
| | GCTCAAGGAAGGCTCAAGAAAGGCCAGGGGTGGGGAAAAGGTGCTCTTGTTAGAGGCGCAGCCTT |
| | TCCTGGGCAGGACCCAGGACCGATGGCAAACCCATGTGTTTGGGCTTGTTTTGTTCTCGATTTTC |
| | TTATCTTCTTGGCCTCTTCCTGTGTTTTTTAGTTTATTGTGACATTATGCATTCATATATGAATG |
| | TTGGCAAGCAGGAGTCATCATCCCAATAACTTCCTGACATTTTTAGCTCTTTTAATGTGCAGTCT |
| | TTGCCCTCCTGCCACAAGTGGCGAAGTAATTGAATTTCCCTGTTACTAACTGGCAGGAGGCATGT |
| | TCTAGTTCCCACCAGAGGAGCTGCTGGGGCTAAAGCTGGGTTCATAGAATCCCACCTAGGGGACA |
| | CCAGGGCTTTCAAGTGGTTTGGGGACCTGTCTGAAATGATATTCACACAATAAAAAATATTTTTC |
| | CCATCATAGACTTGAAAAGGCACCATTGTGCACATCTATATAAAATGTGATAAAATCACATTTAC |
| | TTCCCCTGGCTAGGCCTCATAAGGGAGGCAGGATTTCCTTCTCCTTTTCTAGTAGCAAATAAAAA |
| | CTGGGAAAATTTGGGGGCCTCTGGGTTTATCCCATGGATACCTGCCCCCGCTCCCGCCCGCCAAC |
| | TCAGCCAAGCCCTTAGAGGCAGTCTTCTCTCCCACCTAGATGTCTTTGTAACCTGAGCTGGTAAG |
| | AAAGGGAGGAGGGACAGAAAGAGGGGAAATATGCCCTTGACATATGATGTATCTTCTTTCTTTTC |
| | TTCTTCTCTTTGATTACACGAAATAAAATGGTTTAGGCTGAGGGTAAAGAAGTAATACCATTTCT |
| | AGTTGTGCAACCTTGGGCAGATTTCATTCCCTAAGCCTCCGCTTCCTCAATCTGTAAAGTGGGGA |
| | GAATCACGGGGCTTGCCTCATAGGGCCTTTGAGCATCCTATGAGAGCATGTGGGGGCGCTGGGCT |
| | CAGTGCTGGGCACATGGTAAAACATGTCACAAAAGCTCATTACTATTACGGTTATGACTCATGGC |
| | TTGGAACTGTGTGCTCCTGGGGTCTCAAAGTAGTTCCCCCATTATGGGGTGAGCAGGTTGGGATG |
| | AGAGAAGAGCAGGGCAGGTGGGGGTCTAAAGAGCTCAGGGTCTCATTATGTTTCTGGTGGCAGCT |
| | CCCTCGTGGGTGGGAGTCCCCTCTCCCCATAGACGTGTGTTGCCTTACGAGAGGCTTGTGCCTGC |
| | CTGGGTGTGTGACACAGTTACTCTGGGTTCAGATTTCTATGTTACTGCTAGCTGGTTGGGAGAGT |
| | CTGAGGGAATCATTTCACCTCTCTGTGAAATGGAGATAACTCAAGGTCCCTTACCTCATAGAGTC |
| | CATGTGAGAAGTAAATGAGGGAAAGCACAGACATTACTCGCTCCGGGGGCTGCACCTCCAGAATT |
| | GCTGTTGTCATTATTACCATGTGTCTGACACATTGATATTCCATCCCACAACAACCTCGGAAAGG |
| | AAACACTCCCATTAGCCTCATTTGGTAGAGGAGGAAATTGGGGTTCATCAAGGGTTAAATGACTT |
| | CCCTGAGGGTCCACAGTTGTTCAATCCTTTGGCCTGCGGCCGCCACCCTCTGCTACCTCTTCAGT |
| | ACGTTTGCAGCTTTCTTCAGCGGTGCCAGGCAACAACTGGGCAGGAAGCTCTGGTGCTGGACAGT |
| | TGTCCCTCCCATGGGTTCTGTGGTCAAGTTTTTCAATCTTCTGGGAAAGAGAAGAATGTTCCCCT |
| | CCAGTTCTGGGCATATTGAAGGAGCACGGAGCTGTTGGGAAAAGTTGCAATGTAAGGAATCCTGC |
| | TTTGCAAGTAGTCATTTCCCCATCTGTCCAGAATGAGCCTGAAATCAAGTGAGGGTCCTGAGAAA |
| | CAGAGGGAGGAGGTTTTACTGTTTGTGTGTGGCTTGGTCAGGAGACTGCAGTGGGCTGAATGAGA |
| | AACTAAGCTCGGACTTTTAAGAAGTGGTGAGGCTTGGCCTGCAGCAGTTCTGTGTGTTGTCTCTG |
| | TGGCATTTACTTCTCGGATCGTACCTTCAAAGGCTGGGGAGAATCAGAATTATACAGGGAGGGAG |
| | AGACTGAGTGTGAGTGAGTGTGCGTGGCAGTGGTGTTTCTTAGGACGATGGGTTCTGGGGGGTCA |
| | TAATCTGCTTCGAGGAGGTTTTCATTTCTGGCTGAACAAGGCTGTGGTAAGGCAAGTCCGGAAGG |
| | CATGCTGGAAACTTGAGGGAAGTTTTGAATGGAAACTGCAGTCAACAGCTCCATATGATCCGCAT |
| | GTGGCTTCCCCAGAGGCAAGTTTTCAGCTGCGTGGTGGCCTCTCCCAGTCACTCCACAGGCTGCC |
| | CTGACGCTATTAATATTTGCTGAAGCAAGACCTGAGGTTCGTTGCAGATGGATTACACAATGTAT |
| | TCCAAAACCAAATGTTACTGTTTTCCTGTATTCTCCATCCTTTCAAATTGGCCAGGCTAACATAG |
| | ACCTCCACTGAGAGAATTTCAGAATCATTTGGTAGTTGAGAAGCGCCTACTTCATGCGGAGGCCC |
| | CGTGGGAGGAGTGGAAGAGTTGGCCTCAGCACTGGCGAGTATCGGATGGGAGCTCTGCTCACTTG |
| | GTAAGTCCTTCTGCTAGAACCAAGGGAGGCTGTTCAGATCCATCACAAAGAAGTTGTCGGTCACA |
| | TCCAGGTTGTCTTCTGAGTTTGAGGTGGGATGGAGGTGGCTGCTGAGAATCCATGTGGGTCAAGA |
| | GCTCCAAAGCTTCACTTTTACTTCGCACTCTGTCCCGGGGCATGGACGTCCTCAATGGAGGTCAT |
| | GCAAGCCCTTCCCCCTCACCCCTTCTCTTGGCCCTCTTCATTGTCTCTACATACCCTTGGGTCAA |
| | GAGTGTAGTGGTTCTCCCTTGTCACCCTGGAAGAGAAGCTCTTAGTTTTATTTGCTGGGTCTCCT |
| | AGACTGAAATGATAAAGCTGAAATGATAAAAGGCGTATCATGGCTTTAGAACCCTTCTTATTTCC |
| | CTCGCTCGCACCCCCTAGTTTTCCTTCTCTTCCCTTGAAAATCAGTGAAAATCAGGCCACATCTC |
| | TGATGATGGCCTTTTGTTTCTTTTTCTTTTTCTGTTTCTGCCTTCGTTAGGTAAGCACAAATTTG |
| | ATGTCCCAAGAGGCAGGCCGGTGACCCTTCAGGCCAAGTGCCTGGATGTGGCAAAGCTACAATAA |
| | ATATCGAATGGTGAGAGCAATGGAAATTTAGCAAAGCCATAACCGGGGAGACCTCAGAGGGGCAG |
| | TGGACTGGTTAAGAGGCTGTTGGATGAGCCGGGTAGTATTTCTACTTCAACCTGATTGAAATGTC |
| | GACTAAAAATCAATGCTGTTGACTAGTGATAATTTACAACGTTCCTGGTGCTAAGTAGTTCCCCG |
| | CTTAAGAATGCGTTGGCTGGGCAGAGATTAGCGCAGGGAGTTGTGTGTGTCACAATGAATCAGAC |
| | GCATTATAGGTCAGCCCTTTATTTGTTTCATCATGACTTTTACACAGTTGTCATGTAATTTATGG |
| | CTGCTTTCACGTTGTCAAACATTTTCATTGCATCTTCTTCTTTAACACCCTCCTGACATAGACAC |
| | ACTGCACTTGAAGGCTTGGTATTGTTTCATAATCCGAGAGGAGGCCTATAAACCATCAAATTACA |
| | CTATCTTTGGGCTAATCTAAATGCGCTGCAGATTAAAATCAGAGCTCATTTGTCCCTGATGCAAA |
| | TTATTAAGTTCTAATTATAAATACCCATTTAATTACCCGACACATTTTTATTTTGCGGACCCTTT |
| | TGAGCACTGCTGTCTGCGATGCAGAGGGGGTGGGGGGAGATGCATAGGAGACAATCTGCAGTAAT |
| | TAATGTACACTTCCCAAATGGTAAAGGATAAACATATGCTGCTTTGTTTGTCTTATTTATTTATT |
| | GATTAGATGTATAGAGACTTTGGCGTGGGCACAATCTGAAGTTGAAATCCTTTTAAAGATGAAAA |
| | CTATTTAAAAATCTTTTGGGGAAGAAAGAGCAAAATATAGCCAACCAATAGCTTTCTGCTAGAAC |
| | ACATCATCCCAAAATATGGGATTCTGAATTTGATCAAATCACCAGTTTCTGAATTTGATCAAATC |
| | TAGATTTTGCAGAAGTTCAGGGTGAGAGAAACCATGCCTGTTTTATATCTAGAAAGTGAAATCAT |
| | TGTTATAGAAAAAACCTACTGTGGTTAGAAAAAAACCACATTCTTTTTTCCCAGCCCTGCTGCCA |
| | TCCTCTACCAGAAAATAACAGTATCTGCCTGTAGTATGAAGACCTTCCAATTGAGAGCATTATGA |
| | TAAACTATTTTTGATTACCAAACACGAATGAAGGAAGAAGATAACATAAAAATTAGTAAAGGCCT |
| | TCCAAGTAGACATTTACCCTTCTGTGAAAGCCATGGAGAAATTACCAAGACTGGTTTGGGGGGAG |
| | GGCATTTAAGGTCTTTTGGGCATTACAGATTTTCCAGAACCAAACTTTGACTTTTAGTGTTAACA |
| | GAGAGACACTGATCTGAAAACCAGGACACCTGGGTTCTGACCCTTATTGTATCATGCTGTGAGAT |
| | TTTGGGCTCCCTTCACCTGTTAGCATTTGTTTCCTTGTCTTGTAAAGTAGGTAAAATAGATAGTT |
| | TGGACTGGGTGGGTCTCTAAGTCCCCATGATGTTCTAGCATAGTATGAACACCACTGACCAGTTT |
| | TCTCCCTGCTATTTTTTGGAATCTAGTTGCTGAATGGGGCTCACCTGCAAAGACAGCAGAATATT |
| | ATTTTCTTGATTTGCCTCAAAGATGGAAGCTATGGTGGAGATTAAGGCTTGGATTCGTGATTCCC |
| | CAACAGAAAGCTTAAAGGCATCTTTCAAATTGCTGGAAGCAAAATTGAAGTGCAGTATAATGGAA |
| | TGGTGATAATTCACAGAAGTTTCCAGCCTTATAAGATTTCTCCATCTTTTAATTGTTGCAAGCTG |
| | TTTTTTTTGAAAAACTCCAAAGAATGTAATGTGTATTTTCTCCAAGTTTGCTTTTTTGGGCAAAT |
| | GTAACTACATCAAAATAGAAGTACGTTTTTGAAAAAGAAATAGTTGAATTCAAACAACCAGGTAT |
| | TTTAAATTCAATTAACTGACTGAATTCAGTGATATTTTCCTCCTTCCTCCTCCCAAAAGCTGGTT |
| | TCTCTGTATGGACATAGCCTACATATGCTGAGTCCCTGGAGTTAGGAATTTTTGCTTGTTAAAGG |
| | CATCCGATGCAACATGTTTAGAAGAACTCTCCCTCTGTTAGTGTTGAAGACAGCATAAATTGAGG |
| | GAAAATGTTCTTTTTTTATTCATCATGTAGGTAAAAGCATATGGCCTGTTCTGGGACATGCGATC |
| | TTTGCAATCCATTTTTTAAACTTGGTGTTTACCATTGGCTTTTAGCACGGATGTTTCTGTTTTCC |
| | ACACTGTCCAGCAAATACCATTTATATGTGGCATTGAATGAGATATGAAATGTTTTCAGAAGCAT |
| | GCTGAAAAAGGGCATTCAAAGTTATCCTTTGGATAATGATGATCTAAAACTTTCTTTTATTATCC |
| | CATGTGCTCAGAGTAAGGGGCAAATGAATCAGTTGTGAAATATGTGTTCCTTGTAGGACACAGGC |
| | ACTCTTGAGATCTATAGCTTCAATAAAAAGGTAATTTATTTAAATTACTGCCTCTTTAATTTATA |
| | ATGTTTTGGGGATTTTTAATAGGCATGCTCTGTAAGGGCACTGGTAATCAGCTGTTTCTGATTTT |
| | GCATGCTCTTCTATCTCTGGTAACAAAATAAAATCTTAAAAAACAAGAAAAAAGAAAAAAAAACA |
| | AAAACAAAAACAAGGAACATAAAGTTTAGCCCTAACCCAACCCAAAAGCAAATAACAGGCCGAAT |
| | GAATGGCAGCCCCCCAGAGGCTCTACTTTCCCCTTCCATTATTACCTGAAATAAAAGCATGATAA |
| | CATTCATGCCAGAGATAGGTGACAAAATTATGTATTCAGACATGAAGTTTAGGATTTCATAGCCC |
| | AATGTTCTCTCTTCTCCCCCACCTCTTATTGTGTTGTGCAAATGTATCAGCCGTTGTATTGTTAA |
| | TGCATGATAGGAAGCTGCCGCTAGGACAGTCTTGGCTCACTAATGCGGTCAGCTGTGTCACAATG |
| | TGATATATAGATTATATTTACCATGGCATATTTTGTTTGCGAAATGGGAGCGGATGATAAATGAA |
| | GATACCCTCCAGTTTTCACACTAGITCCTGTGGTCCGGAGTCTCTCAAACAATAAAGCACCCCTG |
| | ATAATGGAGAGGTATTTATGGGAACATAATTGACTTCAAAGTTTTAGATCTCTGGCTGAAGTTTA |
| | AGATGGGATAGTCCATTACATTAATGTCTGTGCTTAAAGCTCCTATTTGGCTTAAATAAATTATT |
| | TAGGGTTTACTGCTTAAACCTTGGTCAATTCTTGAACGTTTGGGCTAGTTAAGTAATTTTCCAGT |
| | GACTTTCTGTGCCTTGGTGATTCATTTACTTGATTGAGCTCCTGTGTGCTCGTATGATTTCTAAA |
| | TGTATTTCTCAAGTTTTGCCTGGCAATGAATGATTTTGCTTACTGGAGTCTTGTGTGGTACACCT |
| | ATAAAAGGCTTATTAACTCTTTTTGAAAAAAAAAAAAATCCCCAAACACATCAACACTGTCATCA |
| | TAAGATAAAGCATATATACATATGCATCTATATACACACATACATATGTACATACTACATATATA |
| | CATACGTATATGCATGTATGAATATATATATAGTTGTGTGCCTGTGTGTGTGTAGAAAGGGAGAG |
| | AGAGAGAATAGGAAAGTCTTTAGAATTCACCATGATTCCATCAAATCAATATAGAAGTTTTTGAA |
| | AGCTATCCATGTAGAAACCACTTTTCATCAAAATCTGACTTAAGCAAATTATCTCCATACTATTT |
| | ATCTGAAAGTCTGTTGTTCACATAGCGCTGGATTGAGGATCATAGTGGCAAATTTAGGAGCAACA |
| | GTCCCAAGCAGGAATCCTGGATGGCAGGCTGTCCTTTGTGCCTCCCCTGAGTTGAGAAGACTGGT |
| | GTTTATTCTTTCTCTAGGTTGCAACACGTGTTGCCTTGAAATCTCCCTTCTTTACGGTTCTGCCA |
| | TGAGTGTATTTTCTGTGACCTGCCTCTGCATCTGGTTAAATGGACTTCAGTAATCTGTACACAGT |
| | TACTTCTTACTTATTTTATATCCTGAAAGATATTAAGTCCAACAAGCTTTTACCCACAGAGTCTA |
| | CAGAGAAAACGGCCAGGCAATTTTTGTTTCAATCTCTGTGTCTCTCTGGAGCACTAGTTCCAGAG |
| | GCTGATCAATAGGTTTTATTGTAGACCTCACTGTCTCTAAAAGCATTTTGACCTTATCCTGTCTA |
| | AAAATAGTATTTGCTCTTGCCTGCAGAACCTTGACCTGTGAAAACCCATTTGGAACATAACTGAC |
| | ATATCTAGTCAGCTGTATATCCAAGACATGCTCTGTGAATGAATTCTGTGCAGAACCGTCCAGGA |
| | GAACACTTTCTTCCAAGACAAATGAATTCCAGTTCTGAACACTGGGAGTGCACCTGCTTGTCGGA |
| | TGTGGTGATGGGCCACATGGTGGGGAGTGAGGGAGACTCAGGGCCTGTGGGGCAGTCGATGTGGG |
| | AGGACTGTCACAGAGACTCTCAGAGGGTGCATTCAGCCCTGAACAGGGCAAAGGACTGCAAGGGG |
| | CAGGAGCTTGGGCTGACATGCAAGGTGGCTTTACACAAGGCCCTTTTTAGAGAGTGTGATTCTCT |
| | GAAGCTTTTCTTGGCAGCTTCAGTCTTGAACCTCACTGGAAGGGATCCTCCAAAACATGACCCAG |
| | ATGGAAAGAAGTATTTCTGAGTTTAAAATAACTCCCCTATTTGGTAATACGGGACTTTATTTGTG |
| | ACTTTATTATTTTTAGGTGTGATAATGGTTTTGCAGTTGTATTTAAAAGAAAAAAAACGAGTTCC |
| | TATGTTTAAAAAATACATACAGAGGTGTTTACTGATGAAATGATATGACGTCTGGGATCAACTTA |
| | AATAATAAAATGGGCTAGGGAGGCGATAGGGTTACAGAAGACAAGAATGACTGTGAGCTGTGGTG |
| | GTTGGAGCTGGAAGATGTGGACTTGGGGACTGATTTATAACATTCTCTCTACTTTTGTAGTATTT |
| | GAGATTTTTCCAGAAAATAAAGGTATTGCCTGACTGGTGGAGAGCAGTATGGCCTTGTTTAGTCG |
| | GTGTTGTTTCTTCACCAAGGGTTTGGCTCAGAGGTAGCAAGGGGACAAGTGTCCTATGGGCAAGA |
| | AAGTACCTGTGAGCTCAAGTCTTGTATCTGGGAAGTTCATTGTGAAGGGGTCATTTAAGGGTCTG |
| | TACTGTGCACTGTCCCCCATTCTCCTGGAAGAACAGAGATCCCTTGTCTTTTTCAGTGCATGAGG |
| | CAGAGTCAGATGTGGCGTTTGCTTGAGTTTCAGCACAGGTGCCTCTGTGCCTCGTGGTGAGGGTC |
| | AGGAAGAAGCAGCTGGGACGTGCTCACGTGGCTGGTAGTGTTATGAAGACAAGGCTTTGGGACCT |
| | TTCTTTGGCCATTTGAGCCCTGGCTATTAGAGAAAGATGATTTGCCTGAGAGGAGATTGACCACA |
| | CTCTCAGAAAGAAGGGGACAAAGAACACGTCAAGGGTTAAGCAGCCTTCCCTTTAAGGGAGGACT |
| | GGGGCACAAGATGGAAGATGAAAGGGAGCAGAGTGGCAATTGCAGAGCTGGAAAGGGGAATTTTG |
| | TTCTTCTAGATAGCAAAAGCCAGGACTGTCGCTGTGTGACTTGAAAGCTAGGTCACTGGTGGGCT |
| | TCGTGCAGCCCGTCACAGGGGAGCCATGGTGGGCCTCGTCTCTGCCGTATCTGCTGCCTGGAAGC |
| | TGAGACTGGCCTAACCACATCACACCATTCCCAGACCCAGGCCCAGGCCCAGGCCCGGGTCCCTC |
| | TGGTTTTACAAAATGTCCGCTCTCTCTCGCTTCACACAGAGGCTATTATTAGCAAGTGTCACTCA |
| | GTTATCTGAGAGTGGCGCTTTTAGCTGCCATCTAAGTGCCTGATACTTGGGTTTACAGCAGATTA |
| | AATTAAATTTTAGGCTGGTTTGGCTTCACTGGCAGTAGACAATGGAAGGCAGCTGTTGTAGAAAT |
| | GTAACCTGGCACCCTCAAGGATTTGTGTGAGTGTGTGTGTGTGTGTGTGTGTGAGTGTGTGTGTG |
| | TGTGTGTGTGTGTGTGTGTGTGCTGACCACTAGGCTACACTTCCTTTTCCTTTCCTCTCCATTTC |
| | ATCCCTTTCCAAAAAGTGTTTAGACAAATAGTTTCCCAGACTTGGTTTTATCATGCTGGGTTGAC |
| | AAAGGTTGTGTACAGAGCTGGAATAATTTTTTCTTCTTTCTACTGTTGGCACATCAATATCTTTT |
| | TTTCTGC |
| BCL11A- | GTCTCTGTCCATCCAGACTCCTGACGTTCAAGTTCGCAGGGACGTCACGTCCGCACTTGAACTTG |
| Exon 1- | CAGCTCAGGGGGGCTTTTGCCATTTTTTTCATCTCTCTCTCTCTCTCTCCCTCTATCTCTCTTCT |
| SEQ ID NO: | CTCTCTCTCCCTCTTTTTTTTTTTTTTTTTTTTTTTTTTTTTGCTTAAAAAAAAGCCATGACGGC |
| 2636 | TCTCCCACAATTCATCTTCCCTGCGCCATCTTTGTATTATTTCTAATTTATTTTGGATGTCAAAA |
| | GGCACTGATGAAGATATTTTCTCTGGAGTCTCCTTCTTTCTAACCCGGCTCTCCCGATGTGAACC |
| | GAGCCGTCGTCCGCCCGCCGCCGCCGCCGCCGCCGCCGCCGCCCGCCCCGCAGCCCACCATGTCT |
| | CGCCGCAAGCAAGGCAAACCCCAGCACTTAAGCAAACGGGAATTCTCGCGTAAGTAACCCAATAA |
| | TAGTAATAATAATTATTAATAATCACGAGAGCGC |
| BCL11A- | CTGTCCTCTCTGGCACTCTAATAATTGTGCTTTTGTTTCTCCAACCACAGCCGAGCCTCTTGAAG |
| Exon 2- | CCATTCTTACAGATGATGAACCAGACCACGGCCCGTTGGGAGCTCCAGAAGGGGATCATGACCTC |
| SEQ ID NO: | CTCACCTGTGGGCAGTGCCAGATGAACTTCCCATTGGGGGACATTCTTATTTTTATCGAGCACAA |
| 2637 | ACGGAAACAATGCAATGGCAGCCTCTGCTTAGAAAAAGCTGTGGATAAGCCACCTTCCCCTTCAC |
| | CAATCGAGATGAAAAAAGCATCCAATCCCGTGGAGGTTGGCATCCAGGTCACGCCAGAGGATGAC |
| | GATTGTTTATCAACGTCATCTAGAGGAATTTGCCCCAAACAGGAACACATAGCAGGTAAATGAGA |
| | AGCAAGGAGAAAAGCTGTTTGCATGTTTTCTTTTCATTTT |
| BCL11A- | GATGCACGTTGTTTGTAGCTGTAGTGCTTGATTTTGGGTTTCTTTCACAGATAAACTTCTGCACT |
| Exon 3- | GGAGGGGCCTCTCCTCCCCTCGTTCTGCACATGGAGCTCTAATCCCCACGCCTGGGATGAGTGCA |
| SEQ ID NO: | GAATATGCCCCGCAGGGTATTTGTAAGTTGAGCCTTATTTCTTCTACAAATGTCCATGTGTATAG |
| 2638 | AGATGAG |
| BCL11A | TGCCCGCCTCAGTGATTAAACATTGATGTTGGTGTTGTATTATTTTGCAGGTAAAGATGAGCCCA |
| Exon 4- | GCAGCTACACATGTACAACTTGCAAACAGCCATTCACCAGTGCATGGTTTCTCTTGCAACACGCA |
| SEQ ID NO: | CAGAACACTCATGGATTAAGAATCTACTTAGAAAGCGAACACGGAAGTCCCCTGACCCCGCGGGT |
| 2639 | TGGTATCCCTTCAGGACTAGGTGCAGAATGTCCTTCCCAGCCACCTCTCCATGGGATTCATATTG |
| | CAGACAATAACCCCTTTAACCTGCTAAGAATACCAGGATCAGTATCGAGAGAGGCTTCCGGCCTG |
| | GCAGAAGGGCGCTTTCCACCCACTCCCCCCCTGTTTAGTCCACCACCGAGACATCACTTGGACCC |
| | CCACCGCATAGAGCGCCTGGGGGCGGAAGAGATGGCCCTGGCCACCCATCACCCGAGTGCCTTTG |
| | ACAGGGTGCTGCGGTTGAATCCAATGGCTATGGAGCCTCCCGCCATGGATTTCTCTAGGAGACTT |
| | AGAGAGCTGGCAGGGAACACGTCTAGCCCACCGCTGTCCCCAGGCCGGCCCAGCCCTATGCAAAG |
| | GTTACTGCAACCATTCCAGCCAGGTAGCAAGCCGCCCTTCCTGGCGACGCCCCCCCTCCCTCCTC |
| | TGCAATCCGCCCCTCCTCCCTCCCAGCCCCCGGTCAAGTCCAAGTCATGCGAGTTCTGCGGCAAG |
| | ACGTTCAAATTTCAGAGCAACCTGGTGGTGCACCGGCGCAGCCACACGGGCGAGAAGCCCTACAA |
| | GTGCAACCTGTGCGACCACGCGTGCACCCAGGCCAGCAAGCTGAAGCGCCACATGAAGACGCACA |
| | TGCACAAATCGTCCCCCATGACGGTCAAGTCCGACGACGGTCTCTCCACCGCCAGCTCCCCGGAA |
| | CCCGGCACCAGCGACTTGGTGGGCAGCGCCAGCAGCGCGCTCAAGTCCGTGGTGGCCAAGTTCAA |
| | GAGCGAGAACGACCCCAACCTGATCCCGGAGAACGGGGACGAGGAGGAAGAGGAGGACGACGAGG |
| | AAGAGGAAGAAGAGGAGGAAGAGGAGGAGGAGGAGCTGACGGAGAGCGAGAGGGTGGACTACGGC |
| | TTCGGGCTGAGCCTGGAGGCGGCGCGCCACCACGAGAACAGCTCGCGGGGCGCGGTCGTGGGCGT |
| | GGGCGACGAGAGCCGCGCCCTGCCCGACGTCATGCAGGGCATGGTGCTCAGCTCCATGCAGCACT |
| | TCAGCGAGGCCTTCCACCAGGTCCTGGGCGAGAAGCATAAGCGCGGCCACCTGGCCGAGGCCGAG |
| | GGCCACAGGGACACTTGCGACGAAGACTCGGTGGCCGGCGAGTCGGACCGCATAGACGATGGCAC |
| | TGTTAATGGCCGCGGCTGCTCCCCGGGCGAGTCGGCCTCGGGGGGCCTGTCCAAAAAGCTGCTGC |
| | TGGGCAGCCCCAGCTCGCTGAGCCCCTTCTCTAAGCGCATCAAGCTCGAGAAGGAGTTCGACCTG |
| | CCCCCGGCCGCGATGCCCAACACGGAGAACGTGTACTCGCAGTGGCTCGCCGGCTACGCGGCCTC |
| | CAGGCAGCTCAAAGATCCCTTCCTTAGCTTCGGAGACTCCAGACAATCGCCTTTTGCCTCCTCGT |
| | CGGAGCACTCCTCGGAGAACGGGAGTTTGCGCTTCTCCACACCGCCCGGGGAGCTGGACGGAGGG |
| | ATCTCGGGGCGCAGCGGCACGGGAAGTGGAGGGAGCACGCCCCATATTAGTGGTCCGGGCCCGGG |
| | CAGGCCCAGCTCAAAAGAGGGCAGACGCAGCGACACTTGTGAGTACTGTGGGAAAGTCTTCAAGA |
| | ACTGTAGCAATCTCACTGTCCACAGGAGAAGCCACACGGGCGAAAGGCCTTATAAATGCGAGCTG |
| | TGCAACTATGCCTGTGCCCAGAGTAGCAAGCTCACCAGGCACATGAAAACGCATGGCCAGGTGGG |
| | GAAGGACGTTTACAAATGTGAAATTTGTAAGATGCCTTTTAGCGTGTACAGTACCCTGGAGAAAC |
| | ACATGAAAAAATGGCACAGTGATCGAGTGTTGAATAATGATATAAAAACTGAATAGAGGTATATT |
| | AATACCCCTCCCTCACTCCCACCTGACACCCCCTTTTTCACCACTCCCCTTCCCCATCGCCCTCC |
| | AGCCCCACTCCCTGTAGGATTTTTTTCTAGTCCCATGTGATTTAAACAAACAAACAAACAAACAG |
| | AAGTAACGAAGCTAAGAATATGAGAGTGCTTGTCACCAGCACACCTGTTTTTTTTCTTTTTCTTT |
| | TTCTTTTTTCTTTTTCCTTTTTTTTTTTTTTCCTTTATGTTCTCACCGTTTGAATGCATGATCTG |
| | TATGGGGCAATACTATTGCATTTTACGCAAACTTTGAGCCTTTCTCTTGTGCAATAATTTACATG |
| | TTGTGTATGTTTTTTTTTAAACTTAGACAGCATGTATGGTATGTTATGGCTATTTTAAATTGTCC |
| | CTAATTCGTTGCTGAGCAAACATGTTGCTGTTTCCAGTTCCGTTCTGAGAGAAAAAGAGAGAGAG |
| | AGAGAAAAAGACCATGCTGCATACATTCTGTAATACATATCATGTACAGTTTTATTTTATAACGT |
| | GAGGAGGAAAAACAGTCTTTGGATTAACCCTCTATAGACAGAATAGATAGCACTGAAAAAAAATC |
| | TCTATGAGCTAAATGTCTGTCTCTAAAGGGTTAAATGTATCAATTGGAAAGGAAGAAAAAAGGCC |
| | TTGAATTGACAAATTAACAGAAAAACAGAACAAGTTTATTCTATCATTTGGTTTTAAAATATGAG |
| | TGCCTTGGATCTATTAAAACCACATCGATGGTTCTTTCTACTTGTTATAAACTTGTAGCTTAATT |
| | CAGCATTGGGTGAGGTAATAAACCTTAGGAACTAGCATATAATTCTATATTGTATTTCTCACAAC |
| | AATGGCTACCTAAAAAGATGACCCATTATGTCCTAGTTAATCATCATTTTTCCTTTAGTTTAATT |
| | TTATAAACAAAACTGATTATACCAGTATAAAAGCTACTTTGCTCCTGGTGAGAGCTTAAAAGAAA |
| | TGGGCTGTTTTGCCCAAAGTTTTATTTTTTTTAAACAATGATTAAATTGAATGTGTAATGTGCAA |
| | AAGCCCTGGAACGCAATTAAATACACTAGTAAGGAGTTCATTTTATGAAGATATTTGCTTTAATA |
| | ATGTCTTTTTAAAAATACTGGCACCAAAAGAAATAGATCCAGATCTACTTGGTTGTCAAGTGGAC |
| | AATCAAATGATAAACTTTAAGACCTTGTATACCATATTGAAAGGAAGAGGCTGACAATAAGGTTT |
| | GACAGAGGGGAACAGAAGAAAATAATATGATTTATTAGCACAACGTGGTACTATTTGCCATTTAA |
| | AACTAGAACAGGTATATAAGCTAATATTGATACAATGATGATTAACTATGAATTCTTAAGACTTG |
| | CATTTAAATGTGACATTCTTAAAAAAAGAAGAGAAAGAATTTTAAGAGTAGCAGTATATATGTCT |
| | GTGCTCCCTAAAAGTTGTACTTCATTTCTTTTCCATACACTGTGTGCTATTTGTGTTAACATGGA |
| | AGAGGATTCATTGTTTTTATTTTTATTTTTTTAATTTTTTCTTTTTTATTAAGCTAGCATCTGCC |
| | CCAGTTGGTGTTCAAATAGCACTTGACTCTGCCTGTGATATCTGTATCTTTTCTCTAATCAGAGA |
| | TACAGAGGTTGAGTATAAAATAAACCTGCTCAGATAGGACAATTAAGTGCACTGTACAATTTTCC |
| | CAGTTTACAGGTCTATACTTAAGGGAAAAGTTGCAAGAATGCTGAAAAAAAATTGAACACAATCT |
| | CATTGAGGAGCATTTTTTAAAAACTAAAAAAAAAAAAACTTTGCCAGCCATTTACTTGACTATTG |
| | AGCTTACTTACTTGGACGCAACATTGCAAGCGCTGTGAATGGAAACAGAATACACTTAACATAGA |
| | AATGAATGATTGCTTTCGCTTCTACAGTGCAAGGATTTTTTTGTACAAAACTTTTTTAAATATAA |
| | ATGTTAAGAAAAATTTTTTTTAAAAAACACTTCATTATGTTTAGGGGGGAACTGCATTTTAGGGT |
| | TCCATTGTCTTGGTGGTGTTACAAGACTTGTTATCCATTTAAAAATGGTAGTGGAAATTCTATGC |
| | CTTGGATACACACCGCTCTTCAGGTTGTAAAAAAAAAAAACATACATTGGGGAAAGGTTTAAGAT |
| | TATATAGTACTTAAATATAGGAAAATGCACACTCATGTTGATTCCTATGCTAAAATACATTTATG |
| | GTCTTTTTTCTGTATTTCTAGAATGGTATTTGAATTAAATGTTCATCTAGTGTTAGGCACTATAG |
| | TATTTATATTGAAGCTTGTATTTTTAACTGTTGCTTGTTCTCTTAAAAGGTATCAATGTACCTTT |
| | TTTGGTAGTGGAAAAAAAAAAGACAGGCTGCCACAGTATATTTTTTTAATTTGGCAGGATAATAT |
| | AGTGCAAATTATTTGTATGCTTCAAAAAAAAAAAAAAGAGAGAAACAAAAAAGTGTGACATTACA |
| | GATGAGAAGCCATATAATGGCGGTTTGGGGGAGCCTGCTAGAATGTCACATGGATGGCTGTCATA |
| | GGGGTTGTACATATCCTTTTTTGTTCCTTTTTCCTGCTGCCATACTGTATGCAGTACTGCAAGCT |
| | AATAACGTTGGTTTGTTATGTAGTGTGCTTTTTGTCCCTTTCCTTCTATCACCCTACATTCCAGC |
| | ATCTTACCTTCATATGCAGTAAAAGAAAGAAAGAAAAAAAAAGGAAAAAAAAAAAAAAACCAATG |
| | TTTTGCAGTTTTTTTCATTGCCAAAAACTAAATGGTGCTTTATATTTAGATTGGAAAGAATTTCA |
| | TATGCAAAGCATATTAAAGAGAAAGCCCGCTTTAGTCAATACTTTTTTGTAAATGGCAATGCAGA |
| | ATATTTTGTTATTGGCCTTTTCTATTCCTGTAATGAAAGCTGTTTGTCGTAACTTGAAATTTTAT |
| | CTTTTACTATGGGAGTCACTATTTATTATTGCTTATGTGCCCTGTTCAAAACAGAGGCACTTAAT |
| | TTGATCTTTTATTTTTCTTTGTTTTTATTTTTTTTTTTATTTAGATGACCAAAGGTCATTACAAC |
| | CTGGCTTTTTATTGTATTTGTTTCTGGTCTTTGTTAAGTTCTATTGGAAAAACCACTGTCTGTGT |
| | TTTTTTGGCAGTTGTCTGCATTAACCTGTTCATACACCCATTTTGTCCCTTTATTGAAAAAATAA |
| | AAAAAATTAAAGTACACATTGTAAGCTTCTTGTGTCCTCATTTGACACACTCTGTAAATTACTTG |
| | C |
| BCL11A- | CATCTACTCTTAGACATAACACACCAGGGTCAATACAACTTTGAAGCTAGTCTAGTGCAAGCTAA |
| Enhancer | CAGTTGCTTTTATCACAGGCTCCAGGAAGGGTTTGGCCTCTGATTAGGGTGGGGGCGTGGGTGGG |
| region- | GTAGAAGAGGACTGGCAGA |
| SEQ ID NO: | |
| 2640 | |
| SEQ ID NO: | MSSAIKSYKS VLRPNERKNQ LLKSTIQCLE DGSAFFFKML QGLEGGITPE IVRFSTEQEK |
| 2641 | QQQDIALWCA VNWFRPVSQD SLTHTIASDN LVEKFEEYYG GTASDAIKQY FSASIGESYY |
| (Variant | WNDCRQQYYD LCRELGVEVS DLTHDLEILC REKCLAVATE SNQNNSIISV LFGTGEKEDR |
| Cas12i2 of | SVKLRITKKI LEAISNLKEI PKNVAPIQEI ILNVAKATKE TFRQVYAGNL GAPSTLEKFI |
| SEQ ID NO: 3 | AKDGQKEFDL KKLQTDLKKV IRGKSKERDW CCQEELRSYV EQNTIQYDLW AWGEMENKAH |
| of | TALKIKSTRN YNFAKQRLEQ FKEIQSLNNL LVVKKLNDFF DSEFFSGEET YTICVHHLGG |
| PCT/US2021/ | KDLSKLYKAW EDDPADPENA IVVLCDDLKN NFKKEPIRNI LRYIFTIRQE CSAQDILAAA |
| 025257) | KYNQQLDRYK SQKANPSVLG NQGETWTNAV ILPEKAQRND RPNSLDLRIW LYLKLRHPDG |
| | RWKKHHIPFY DTRFFQEIYA AGNSPVDTCQ FRTPRFGYHL PKLTDQTAIR VNKKHVKAAK |
| | TEARIRLAIQ QGTLPVSNLK ITEISATINS KGQVRIPVKF RVGRQKGTLQ IGDRFCGYDQ |
| | NQTASHAYSL WEVVKEGQYH KELGCFVRFI SSGDIVSITE NRGNQFDQLS YEGLAYPQYA |
| | DWRKKASKFV SLWQITKKNK KKEIVTVEAK EKFDAICKYQ PRLYKENKEY AYLLRDIVRG |
| | KSLVELQQIR QEIFRFIEQD CGVTRLGSLS LSTLETVKAV KGIIYSYFST ALNASKNNPI |
| | SDEQRKEFDP ELFALLEKLE LIRTRKKKQK VERIANSLIQ TCLENNIKFI RGEGDLSTIN |
| | NATKKKANSR SMDWLARGVF NKIRQLAPMH NITLFGCGSL YTSHQDPLVH RNPDKAMKCR |
| | WAAIPVKDIG RWVLRKLSQN LRAKNRGTGE YYHQGVKEFL SHYELQDLEE ELLKWRSDRK |
| | SNIPCWVLQN RLAEKLGNKE AVVYIPVRGG RIYFATHKVA TGAVSIVEDQ KQVWVCNADH |
| | VAAANIALTG KGIGEQSSDE ENPDGSRIKL QLTS |
| SEQ ID NO: | MSSAIKSYKS VLRPNERKNQ LLKSTIQCLE DGSAFFFKML QGLEGGITPE IVRESTEQEK |
| 2642 | QQQDIALWCA VNWFRPVSQD SLTHTIASDN LVEKFEEYYG GTASDAIKQY FSASIGESYY |
| (Variant | WNDCRQQYYD LCRELGVEVS DLTHDLEILC REKCLAVATE SNQNNSIISV LFGTGEKEDR |
| Cas12i2 of | SVKLRITKKI LEAISNLKEI PKNVAPIQEI ILNVAKATKE TFRQVYAGNL GAPSTLEKFI |
| SEQ ID NO: 4 | AKDGQKEFDL KKLQTDLKKV IRGKSKERDW CCQEELRSYV EQNTIQYDLW AWGEMENKAH |
| of | TALKIKSTRN YNFAKQRLEQ FKEIQSLNNL LVVKKLNDFF DSEFFSGEET YTICVHHLGG |
| PCT/US2021/ | KDLSKLYKAW EDDPADPENA IVVLCDDLKN NFKKEPIRNI LRYIFTIRQE CSAQDILAAA |
| 025257) | KYNQQLDRYK SQKANPSVLG NQGFTWTNAV ILPEKAQRND RPNSLDLRIW LYLKLRHPDG |
| | RWKKHHIPFY DTRFFQEIYA AGNSPVDTCQ FRTPRFGYHL PKLTDQTAIR VNKKHVKAAK |
| | TEARIRLAIQ QGTLPVSNLK ITEISATINS KGQVRIPVKF RVGRQKGTLQ IGDRFCGYDQ |
| | NQTASHAYSL WEVVKEGQYH KELGCFVRFI SSGDIVSITE NRGNQFDQLS YEGLAYPQYA |
| | DWRKKASKFV SLWQITKKNK KKEIVTVEAK EKFDAICKYQ PRLYKENKEY AYLLRDIVRG |
| | KSLVELQQIR QEIFRFIEQD CGVTRLGSLS LSTLETVKAV KGIIYSYFST ALNASKNNPI |
| | SDEQRKEFDP ELFALLEKLE LIRTRKKKQK VERIANSLIQ TCLENNIKFI RGEGDLSTTN |
| | NATKKKANSR SMDWLARGVF NKIRQLAPMH NITLFGCGSL YTSHQDPLVH RNPDKAMKCR |
| | WAAIPVKDIG DWVLRKLSQN LRAKNRGTGE YYHQGVKEFL SHYELQDLEE ELLKWRSDRK |
| | SNIPCWVLQN RLAEKLGNKE AVVYIPVRGG RIYFATHKVA TGAVSIVEDQ KQVWVCNADH |
| | VAAANIALTG KGIGEQSSDE ENPDGSRIKL QLTS |
| SEQ ID NO: | MSSAIKSYKS VLRPNERKNQ LLKSTIQCLE DGSAFFFKML QGLEGGITPE IVRESTEQEK |
| 2643 | QQQDIALWCA VNWFRPVSQD SLTHTIASDN LVEKFEEYYG GTASDAIKQY FSASIGESYY |
| (Variant | WNDCRQQYYD LCRELGVEVS DLTHDLEILC REKCLAVATE SNQNNSIISV LFGTGEKEDR |
| Cas12i2 of | SVKLRITKKI LEAISNLKEI PKNVAPIQEI ILNVAKATKE TFRQVYAGNL GAPSTLEKFI |
| SEQ ID NO: 5 | AKDGQKEFDL KKLQTDLKKV IRGKSKERDW CCQEELRSYV EQNTIQYDLW AWGEMENKAH |
| of | TALKIKSTRN YNFAKQRLEQ FKEIQSLNNL LVVKKLNDFF DSEFFSGEET YTICVHHLGG |
| PCT/US2021/ | KDLSKLYKAW EDDPADPENA IVVLCDDLKN NFKKEPIRNI LRYIFTIRQE CSAQDILAAA |
| 025257) | KYNQQLDRYK SQKANPSVLG NQGFTWTNAV ILPEKAQRND RPNSLDLRIW LYLKLRHPDG |
| | RWKKHHIPFY DTRFFQEIYA AGNSPVDTCQ FRTPRFGYHL PKLTDQTAIR VNKKHVKAAK |
| | TEARIRLAIQ QGTLPVSNLK ITEISATINS KGQVRIPVKF RVGRQKGTLQ IGDRFCGYDQ |
| | NQTASHAYSL WEVVKEGQYH KELGCFVRFI SSGDIVSITE NRGNQFDQLS YEGLAYPQYA |
| | DWRKKASKFV SLWQITKKNK KKEIVTVEAK EKFDAICKYQ PRLYKENKEY AYLLRDIVRG |
| | KSLVELQQIR QEIFRFIEQD CGVTRLGSLS LSTLETVKAV KGIIYSYFST ALNASKNNPI |
| | SDEQRKEFDP ELFALLEKLE LIRTRKKKQK VERIANSLIQ TCLENNIKFI RGEGDLSTIN |
| | NATKKKANSR SMDWLARGVF NKIRQLAPMH NITLFGCGSL YTSHQDPLVH RNPDKAMKCR |
| | WAAIPVKDIG DWVLRKLSQN LRAKNRGTGE YYHQGVKEFL SHYELQDLEE ELLKWRSDRK |
| | SNIPCWVLQN RLAEKLGNKE AVVYIPVRGG RIYFATHKVA TGAVSIVEDQ KQVWVCNADH |
| | VAAANIALTG KGIGEQSSDE ENPDGGRIKL QLTS |
| SEQ ID NO: | MSSAIKSYKS VLRPNERKNQ LLKSTIQCLE DGSAFFFKML QGLFGGITPE IVRESTEQEK |
| 2644 | QQQDIALWCA VNWFRPVSQD SLTHTIASDN LVEKFEEYYG GTASDAIKQY FSASIGESYY |
| (Variant | WNDCRQQYYD LCRELGVEVS DLTHDLEILC REKCLAVATE SNQNNSIISV LFGTGEKEDR |
| Cas12i2 of | SVKLRITKKI LEAISNLKEI PKNVAPIQEI ILNVAKATKE TFRQVYAGNL GAPSTLEKFI |
| SEQ ID NO: | AKDGQKEFDL KKLQTDLKKV IRGKSKERDW CCQEELRSYV EQNTIQYDLW AWGEMENKAH |
| 495 of | TALKIKSTRN YNFAKQRLEQ FKEIQSLNNL LVVKKLNDFF DSEFFSGEET YTICVHHLGG |
| PCT/US2021/ | KDLSKLYKAW EDDPADPENA IVVLCDDLKN NFKKEPIRNI LRYIFTIRQE CSAQDILAAA |
| 025257) | KYNQQLDRYK SQKANPSVLG NQGFTWTNAV ILPEKAQRND RPNSLDLRIW LYLKLRHPDG |
| | RWKKHHIPFY DTRFFQEIYA AGNSPVDTCQ FRTPRFGYHL PKLTDQTAIR VNKKHVKAAK |
| | TEARIRLAIQ QGTLPVSNLK ITEISATINS KGQVRIPVKF RVGRQKGTLQ IGDRFCGYDQ |
| | NQTASHAYSL WEVVKEGQYH KELRCRVRFI SSGDIVSITE NRGNQFDQLS YEGLAYPQYA |
| | DWRKKASKFV SLWQITKKNK KKEIVTVEAK EKFDAICKYQ PRLYKENKEY AYLLRDIVRG |
| | KSLVELQQIR QEIFRFIEQD CGVTRLGSLS LSTLETVKAV KGIIYSYFST ALNASKNNPI |
| | SDEQRKEFDP ELFALLEKLE LIRTRKKKQK VERIANSLIQ TCLENNIKFI RGEGDLSTIN |
| | NATKKKANSR SMDWLARGVF NKIRQLAPMH NITLFGCGSL YTSHQDPLVH RNPDKAMKCR |
| | WAAIPVKDIG DWVLRKLSQN LRAKNRGIGE YYHQGVKEFL SHYELQDLEE ELLKWRSDRK |
| | SNIPCWVLQN RLAEKLGNKE AVVYIPVRGG RIYFATHKVA TGAVSIVEDQ KQVWVCNADH |
| | VAAANIALTG KGIGRQSSDE ENPDGGRIKL QLTS |
| SEQ ID NO: | MSSAIKSYKS VLRPNERKNQ LLKSTIQCLE DGSAFFFKML QGLFGGITPE IVRESTEQEK |
| 2645 | QQQDIALWCA VNWFRPVSQD SLTHTIASDN LVEKFEEYYG GTASDAIKQY FSASIGESYY |
| (Variant | WNDCRQQYYD LCRELGVEVS DLTHDLEILC REKCLAVATE SNQNNSIISV LFGTGEKEDR |
| Cas12i2 of | SVKLRITKKI LEAISNLKEI PKNVAPIQEI ILNVAKATKE TFRQVYAGNL GAPSTLEKFI |
| SEQ ID NO: | AKDGQKEFDL KKLQTDLKKV IRGKSKERDW CCQEELRSYV EQNTIQYDLW AWGEMENKAH |
| 496 of | TALKIKSTRN YNFAKQRLEQ FKEIQSLNNL LVVKKLNDFF DSEFFSGEET YTICVHHLGG |
| PCT/US2021/ | KDLSKLYKAW EDDPADPENA IVVLCDDLKN NFKKEPIRNI LRYIFTIRQE CSAQDILAAA |
| 025257) | KYNQQLDRYK SQKANPSVLG NQGFTWTNAV ILPEKAQRND RPNSLDLRIW LYLKLRHPDG |
| | RWKKHHIPFY DTRFFQEIYA AGNSPVDTCQ FRTPRFGYHL PKLTDQTAIR VNKKHVKAAK |
| | TEARIRLAIQ QGTLPVSNLK ITEISATINS KGQVRIPVKF RVGRQKGTLQ IGDRFCGYDQ |
| | NQTASHAYSL WEVVKEGQYH KELRCRVRFI SSGDIVSITE NRGNQFDQLS YEGLAYPQYA |
| | DWRKKASKFV SLWQITKKNK KKEIVTVEAK EKFDAICKYQ PRLYKENKEY AYLLRDIVRG |
| | KSLVELQQIR QEIFRFIEQD CGVTRLGSLS LSTLETVKAV KGIIYSYFST ALNASKNNPI |
| | SDEQRKEFDP ELFALLEKLE LIRTRKKKQK VERIANSLIQ TCLENNIKFI RGEGDLSTIN |
| | NATKKKANSR SMDWLARGVF NKIRQLATMH NITLFGCGSL YTSHQDPLVH RNPDKAMKCR |
| | WAAIPVKDIG DWVLRKLSQN LRAKNRGTGE YYHQGVKEFL SHYELQDLEE ELLKWRSDRK |
| | SNIPCWVLQN RLAEKLGNKE AVVYIPVRGG RIYFATHKVA TGAVSIVEDQ KQVWVCNADH |
| | VAAANIALTG KGIGRQSSDE ENPDGGRIKL QLTS |
| SEQ ID NO: | ATGGCTTCCATCTCTAGGCCATACGGCACCAAGCTGCGACCGGACGCACGGAAGAAGGAGATGCT |
| 2646 | CGATAAGTTCTTTAATACACTGACTAAGGGTCAGCGCGTGTTCGCAGACCTGGCCCTGTGCATCT |
| (Nucleotide | ATGGCTCCCTGACCCTGGAGATGGCCAAGTCTCTGGAGCCAGAAAGTGATTCAGAACTGGTGTGC |
| sequence | ACAGCCCCAGCTCCGACAAGTACGTGTGGATCGATTGCAGGCAGAAATTCCTGAGGTTTCAGCGC |
| encoding | GCTATTGGGTGGTTTCGGCTGGTGGACAAGACCATCTGGTCCAAGGATGGCATCAAGCAGGAGAA |
| Cas12i4) | TCTGGTGAAACAGTACGAAGCCTATTCCGGAAAGGAGGCTTCTGAAGTGGTCAAAACATACCTGA |
| | GAGCTCGGCACTCGCAACCTGTCCGAGGACTTCGAATGTATGCTCTTTGAACAGTACATTAGACT |
| | GACCAAGGGCGAGATCGAAGGGTATGCCGCTATTTCAAATATGTTCGGAAACGGCGAGAAGGAAG |
| | ACCGGAGCAAGAAAAGAATGTACGCTACACGGATGAAAGATTGGCTGGAGGCAAACGAAAATATC |
| | ACTTGGGAGCAGTATAGAGAGGCCCTGAAGAACCAGCTGAATGCTAAAAACCTGGAGCAGGTTGT |
| | GGCCAATTACAAGGGGAACGCTGGCGGGGCAGACCCCTTCTTTAAGTATAGCTTCTCCAAAGAGG |
| | GAATGGTGAGCAAGAAAGAACATGCACAGCAGCTCGACAAGTTCAAAACCGTCCTGAAGAACAAA |
| | GCCCGGGACCTGAATTTTCCAAACAAGGAGAAGCTGAAGCAGTACCTGGAGGCCGAAATCGGCAT |
| | TCCGGTCGACGCTAACGTGTACTCCCAGATGTTCTCTAACGGGGTGAGTGAGGTCCAGCCTAAGA |
| | CCACACGGAATATGTCTTTTAGTAACGAGAAACTGGATCTGCTCACTGAACTGAAGGACCTGAAC |
| | AAGGGCGATGGGTTCGAGTACGCCAGAGAAGTGCTGAACGGGTTCTTTGACTCCGAGCTCCACAC |
| | TACCGAGGATAAGTTTAATATCACCTCTAGGTACCTGGGAGGCGACAAATCAAACCGCCTGAGCA |
| | AACTCTATAAGATCTGGAAGAAAGAGGGTGTGGACTGCGAGGAAGGCATTCAGCAGTTCTGTGAA |
| | GCCGTCAAAGATAAGATGGGCCAGATCCCCATTCGAAATGTGCTGAAGTACCTGTGGCAGTTCCG |
| | GGAGACAGTCAGTGCCGAGGATTTTGAAGCAGCCGCTAAGGCTAACCATCTGGAGGAAAAGATCA |
| | GCCGGGTGAAAGCCCACCCAATCGTGATTAGCAATAGGTACTGGGCTTTTGGGACTTCCGCACTG |
| | GTGGGAAACATTATGCCCGCAGACAAGAGGCATCAGGGAGAGTATGCCGGTCAGAATTTCAAAAT |
| | GTGGCTGGAGGCTGAACTGCACTACGATGGCAAGAAAGCAAAGCACCATCTGCCTTTTTATAACG |
| | CCCGCTTCTTTGAGGAAGTGTACTGCTATCACCCCTCTGTCGCCGAGATCACTCCTTTCAAAACC |
| | AAGCAGTTTGGCTGTGAAATCGGGAAGGACATTCCAGATTACGTGAGCGTCGCTCTGAAGGACAA |
| | TCCGTATAAGAAAGCAACCAAACGAATCCTGCGTGCAATCTACAATCCCGTCGCCAACACAACTG |
| | GCGTTGATAAGACCACAAACTGCAGCTTCATGATCAAACGCGAGAATGACGAATATAAGCTGGTC |
| | ATCAACCGAAAAATTTCCGTGGATCGGCCTAAGAGAATCGAAGTGGGCAGGACAATTATGGGGTA |
| | CGACCGCAATCAGACAGCTAGCGATACTTATTGGATTGGCCGGCTGGTGCCACCTGGAACCCGGG |
| | GCGCATACCGCATCGGAGAGTGGAGCGTCCAGTATATTAAGTCCGGGCCTGTCCTGTCTAGTACT |
| | CAGGGAGTTAACAATTCCACTACCGACCAGCTGGTGTACAACGGCATGCCATCAAGCTCCGAGCG |
| | GTTCAAGGCCTGGAAGAAAGCCAGAATGGCTTTTATCCGAAAACTCATTCGTCAGCTGAATGACG |
| | AGGGACTGGAATCTAAGGGTCAGGATTATATCCCCGAGAACCCTTCTAGTTTCGATGTGCGGGGC |
| | GAAACCCTGTACGTCTTTAACAGTAATTATCTGAAGGCCCTGGTGAGCAAACACAGAAAGGCCAA |
| | GAAACCTGTTGAGGGGATCCTGGACGAGATTGAAGCCTGGACATCTAAAGACAAGGATTCATGCA |
| | GCCTGATGCGGCTGAGCAGCCTGAGCGATGCTTCCATGCAGGGAATCGCCAGCCTGAAGAGTCTG |
| | ATTAACAGCTACTTCAACAAGAATGGCTGTAAAACCATCGAGGACAAAGAAAAGTTTAATCCCGT |
| | GCTGTATGCCAAGCTGGTTGAGGTGGAACAGCGGAGAACAAACAAGCGGTCTGAGAAAGTGGGAA |
| | GAATCGCAGGTAGTCTGGAGCAGCTGGCCCTGCTGAACGGGGTTGAGGTGGTCATCGGCGAAGCT |
| | GACCTGGGGGAGGTCGAAAAAGGAAAGAGTAAGAAACAGAATTCACGGAACATGGATTGGTGCGC |
| | AAAGCAGGTGGCACAGCGGCTGGAGTACAAACTGGCCTTCCATGGAATCGGTTACTTTGGAGTGA |
| | ACCCCATGTATACCAGCCACCAGGACCCTTTCGAACATAGGCGCGTGGCTGATCACATCGTCATG |
| | CGAGCACGTTTTGAGGAAGTCAACGTGGAGAACATTGCCGAATGGCACGTGCGAAATTTCTCAAA |
| | CTACCTGCGTGCAGACAGCGGCACTGGGCTGTACTATAAGCAGGCCACCATGGACTTCCTGAAAC |
| | ATTACGGTCTGGAGGAACACGCTGAGGGCCTGGAAAATAAGAAAATCAAGTTCTATGACTTTAGA |
| | AAGATCCTGGAGGATAAAAACCTGACAAGCGTGATCATTCCAAAGAGGGGGGGGCGCATCTACAT |
| | GGCCACCAACCCAGTGACATCCGACTCTACCCCGATTACATACGCCGGCAAGACTTATAATAGGT |
| | GTAACGCTGATGAGGTGGCAGCCGCTAATATCGTTATTTCTGTGCTGGCTCCCCGCAGTAAGAAA |
| | AACGAGGAACAGGACGATATCCCTCTGATTACCAAGAAAGCCGAGAGTAAGTCACCACCGAAAGA |
| | CCGGAAGAGATCAAAAACAAGCCAGCTGCCTCAGAAA |
| SEQ ID NO: | MASISRPYGTKLRPDARKKEMLDKFFNTLTKGQRVFADLALCIYGSLTLEMAKSLEPESDSELVC |
| 2647 | AIGWFRLVDKTIWSKDGIKQENLVKQYEAYSGKEASEVVKTYLNSPSSDKYVWIDCRQKFLRFQR |
| Cas12i4 amino | ELGTRNLSEDFECMLFEQYIRLTKGEIEGYAAISNMFGNGEKEDRSKKRMYATRMKDWLEANENI |
| acid sequence | TWEQYREALKNQLNAKNLEQVVANYKGNAGGADPFFKYSFSKEGMVSKKEHAQQLDKFKTVLKNK |
| of SEQ ID | ARDLNFPNKEKLKQYLEAEIGIPVDANVYSQMFSNGVSEVQPKTTRNMSFSNEKLDLLTELKDLN |
| NO: 14 of | KGDGFEYAREVLNGFFDSELHTTEDKFNITSRYLGGDKSNRLSKLYKIWKKEGVDCEEGIQQFCE |
| U.S. Pat. No. | AVKDKMGQIPIRNVLKYLWQFRETVSAEDFEAAAKANHLEEKISRVKAHPIVISNRYWAFGTSAL |
| 10,808,245) | VGNIMPADKRHQGEYAGQNFKMWLEAELHYDGKKAKHHLPFYNARFFEEVYCYHPSVAEITPFKT |
| | KQFGCEIGKDIPDYVSVALKDNPYKKATKRILRAIYNPVANTTGVDKTTNCSFMIKRENDEYKLV |
| | INRKISVDRPKRIEVGRTIMGYDRNQTASDTYWIGRLVPPGTRGAYRIGEWSVQYIKSGPVLSST |
| | QGVNNSTTDQLVYNGMPSSSERFKAWKKARMAFIRKLIRQLNDEGLESKGQDYIPENPSSFDVRG |
| | ETLYVENSNYLKALVSKHRKAKKPVEGILDEIEAWTSKDKDSCSLMRLSSLSDASMQGIASLKSL |
| | INSYFNKNGCKTIEDKEKFNPVLYAKLVEVEQRRTNKRSEKVGRIAGSLEQLALLNGVEVVIGEA |
| | DLGEVEKGKSKKQNSRNMDWCAKQVAQRLEYKLAFHGIGYFGVNPMYTSHQDPFEHRRVADHIVM |
| | RARFEEVNVENIAEWHVRNFSNYLRADSGTGLYYKQATMDFLKHYGLEEHAEGLENKKIKFYDER |
| | KILEDKNLTSVIIPKRGGRIYMATNPVTSDSTPITYAGKTYNRCNADEVAAANIVISVLAPRSKK |
| | NEEQDDIPLITKKAESKSPPKDRKRSKTSQLPQK |
| SEQ ID NO: | MASISRPYGT KLRPDARKKE MLDKFFNTLT KGQRVFADLA LCIYGSLTLE MAKSLEPESD |
| 2648 | SELVCAIGWF RLVDKTIWSK DGIKQENLVK QYEAYSGKEA SEVVKTYLNS PSSDKYVWID |
| (Variant | CRQKFLRFQR ELGTRNLSED FECMLFEQYI RLTKGEIEGY AAISNMFGNG EKEDRSKKRM |
| Cas12i4) | YATRMKDWLE ANENITWEQY REALKNQLNA KNLEQVVANY KGNAGGADPF FKYSFSKEGM |
| | VSKKEHAQQL DKFKTVLKNK ARDLNFPNKE KLKQYLEAEI GIPVDANVYS QMFSNGVSEV |
| | QPKTTRNMSF SNEKLDLLTE LKDLNKGDGF EYAREVLNGF FDSELHTTED KFNITSRYLG |
| | GDKSNRLSKL YKIWKKEGVD CEEGIQQFCE AVKDKMGQIP IRNVLKYLWQ FRETVSAEDF |
| | EAAAKANHLE EKISRVKAHP IVISNRYWAF GTSALVGNIM PADKRHQGEY AGQNFKMWLE |
| | AELHYDGKKA KHHLPFYNAR FFEEVYCYHP SVAEITPFKT KQFGCEIGKD IPDYVSVALK |
| | DNPYKKATKR ILRAIYNPVA NTTGVDKTTN CSFMIKREND EYKLVINRKI SRDRPKRIEV |
| | GRTIMGYDRN QTASDTYWIG RLVPPGTRGA YRIGEWSVQY IKSGPVLSST QGVNNSTTDQ |
| | LVYNGMPSSS ERFKAWKKAR MAFIRKLIRQ LNDEGLESKG QDYIPENPSS FDVRGETLYV |
| | FNSNYLKALV SKHRKAKKPV EGILDEIEAW TSKDKDSCSL MRLSSLSDAS MQGIASLKSL |
| | INSYFNKNGC KTIEDKEKEN PVLYAKLVEV EQRRINKRSE KVGRIAGSLE QLALLNGVEV |
| | VIGEADLGEV EKGKSKKQNS RNMDWCAKQV AQRLEYKLAF HGIGYFGVNP MYTSHQDPFE |
| | HRRVADHIVM RARFEEVNVE NIAEWHVRNF SNYLRADSGT GLYYKQATMD FLKHYGLEEH |
| | AEGLENKKIK FYDFRKILED KNLTSVIIPK RGGRIYMATN PVTSDSTPIT YAGKTYNRCN |
| | ADEVAAANIV ISVLAPRSKK NREQDDIPLI TKKAESKSPP KDRKRSKTSQ LPQK |
| SEQ ID NO: | MASISRPYGT KLRPDARKKE MLDKFENTLT KGQRVFADLA LVIYHDLYLR MAKSLEPESD |
| 2649 | SELVCAIGWF RLVDKTIWSK DGIKQENLVK QYEAYSGKEA SEVVKTYLNS PSSDKYVWID |
| (Variant | CRQKFLRFQR ELGTRNLSED FECMLFEQYI RLTKGEIEGY AAISNMFGNG EKEDRSKKRM |
| Cas12i4) | YATRMKDWLE ANENITWEQY REALKNQLNA KNLEQVVANY KGNAGGADPF FKYSFSKEGM |
| | VSKKEHAQQL DKFKTVLKNK ARDLNFPNKE KLKQYLEAEI GIPVDANVYS QMFSNGVSEV |
| | QPKTTRNMSF SNEKLDLLTE LKDLNKGDGF EYAREVLNGF FDSELHTTED KFNITSRYLG |
| | GDKSNRLSKL YKIWKKEGVD CEEGIQQFCE AVKDKMGQIP IRNVLKYLWQ FRETVSAEDF |
| | EAAAKANHLE EKISRVKAHP IVISNRYWAF GTSALVGNIM PADKRHQGEY AGQNFKMWLR |
| | AELHYDGKKA KHHLPFYNAR FFEEVYCYHP SVAEITPFKT KQFGCEIGKD IPDYVSVALK |
| | DNPYKKATKR ILRAIYNPVA NTTRVDKTIN CSFMIKREND EYKLVINRKI SRDPRKRIEV |
| | GRTIMGYDRN QTASDTYWIG RLVPPGTRGA YRIGEWSVQY IKSGPVLSST QGVNNSTTDQ |
| | LVYNGMPSSS ERFKAWKKAR MAFIRKLIRQ LNDEGLESKG QDYIPENPSS FDVRGETLYV |
| | FNSNYLKALV SKHRKAKKPV EGILDEIEAW TSKDKDSCSL MRLSSLSDAS MQGIASLKSL |
| | INSYFNKNGC KTIEDKEKEN PVLYAKLVEV EQRRINKRSE KVGRIAGSLE QLALLNGVEV |
| | VIGEADLGEV EKGKSKKQNS RNMDWCAKQV AQRLEYKLAF HGIGYFGVNP MYTSHQDPFE |
| | HRRVADHIVM RARFEEVNVE NIAEWHVRNF SNYLRADSGT GLYYKQATMD FLKHYGLEEH |
| | AEGLENKKIK FYDERKILED KNLTSVIIPK RGGRIYMATN PVTSDSTPIT YAGKTYNRCN |
| | ADEVAAANIV ISVLAPRSKK NREQDDIPLI TKKAESKSPP KDRKRSKTSQ LPQK |
| SEQ ID NO: | MSNKEKNASETRKAYTTKMIPRSHDRMKLLGNFMDYLMDGTPIFFELWNQFGGGIDRDI |
| 2650 | ISGTANKDKISDDLLLAVNWFKVMPINSKPQGVSPSNLANLFQQYSGSEPDIQAQEYFA |
| (Cas12i1 of | SNFDTEKHQWKDMRVEYERLLAELQLSRSDMHHDLKLMYKEKCIGLSLSTAHYITSVMF |
| SEQ ID NO: 3 | GTGAKNNRQTKHQFYSKVIQLLEESTQINSVEQLASIILKAGDCDSYRKLRIRCSRKGA |
| of U.S. Pat. | TPSILKIVQDYELGINHDDEVNVPSLIANLKEKLGRFEYECEWKCMEKIKAFLASKVGP |
| No. | YYLGSYSAMLENALSPIKGMTTKNCKFVLKQIDAKNDIKYENEPFGKIVEGFFDSPYFE |
| 10,808,245) | SDTNVKWVLHPHHIGESNIKTLWEDLNAIHSKYEEDIASLSEDKKEKRIKVYQGDVCQT |
| | INTYCEEVGKEAKTPLVQLLRYLYSRKDDIAVDKIIDGITFLSKKHKVEKQKINPVIQK |
| | YPSFNFGNNSKLLGKIISPKDKLKHNLKCNRNQVDNYIWIEIKVLNTKTMRWEKHHYAL |
| | SSTRFLEEVYYPATSENPPDALAARFRTKINGYEGKPALSAEQIEQIRSAPVGLRKVKK |
| | RQMRLEAARQQNLLPRYTWGKDFNINICKRGNNFEVTLATKVKKKKEKNYKVVLGYDAN |
| | IVRKNTYAAIEAHANGDGVIDYNDLPVKPIESGFVTVESQVRDKSYDQLSYNGVKLLYC |
| | KPHVESRRSFLEKYRNGTMKDNRGNNIQIDFMKDFEAIADDETSLYYFNMKYCKLLQSS |
| | IRNHSSQAKEYREEIFELLRDGKLSVLKLSSLSNLSFVMFKVAKSLIGTYFGHLLKKPK |
| | NSKSDVKAPPITDEDKQKADPEMFALRLALEEKRLNKVKSKKEVIANKIVAKALELRDK |
| | YGPVLIKGENISDTTKKGKKSSTNSFLMDWLARGVANKVKEMVMMHQGLEFVEVNPNFT |
| | SHQDPFVHKNPENTFRARYSRCTPSELTEKNRKEILSFLSDKPSKRPTNAYYNEGAMAF |
| | LATYGLKKNDVLGVSLEKFKQIMANILHQRSEDQLLFPSRGGMFYLATYKLDADATSVN |
| | WNGKQFWVCNADLVAAYNVGLVDIQKDFKKK |
| SEQ ID NO: | MSISNNNILPYNPKLLPDDRKHKMLVDTFNQLDLIRNNLHDMIIALYGALKYDNIKQFA |
| 2651 | SKEKPHISADALCSINWFRLVKTNERKPAIESNQIISKFIQYSGHTPDKYALSHITGNH |
| (Cas12i3 of | EPSHKWIDCREYAINYARIMHLSFSQFQDLATACLNCKILILNGTLTSSWAWGANSALF |
| SEQ ID NO: | GGSDKENFSVKAKILNSFIENLKDEMNTTKFQVVEKVCQQIGSSDAADLFDLYRSTVKD |
| 14 of U.S. | GNRGPATGRNPKVMNLFSQDGEISSEQREDFIESFQKVMQEKNSKQIIPHLDKLKYHLV |
| Pat. No. | KQSGLYDIYSWAAAIKNANSTIVASNSSNLNTILNKTEKQQTFEELRKDEKIVACSKIL |
| 10,808,245) | LSVNDTLPEDLHYNPSTSNLGKNLDVFFDLLNENSVHTIENKEEKNKIVKECVNQYMEE |
| | CKGLNKPPMPVLLTFISDYAHKHQAQDFLSAAKMNFIDLKIKSIKVVPTVHGSSPYTWI |
| | SNLSKKNKDGKMIRTPNSSLIGWIIPPEEIHDQKFAGQNPIIWAVLRVYCNNKWEMHHF |
| | PFSDSRFFTEVYAYKPNLPYLPGGENRSKRFGYRHSTNLSNESRQILLDKSKYAKANKS |
| | VLRCMENMTHNVVFDPKTSLNIRIKTDKNNSPVLDDKGRITFVMQINHRILEKYNNTKI |
| | EIGDRILAYDQNQSENHTYAILQRTEEGSHAHQFNGWYVRVLETGKVTSIVQGLSGPID |
| | QLNYDGMPVTSHKFNCWQADRSAFVSQFASLKISETETFDEAYQAINAQGAYTWNLFYL |
| | RILRKALRVCHMENINQFREEILAISKNRLSPMSLGSLSQNSLKMIRAFKSIINCYMSR |
| | MSFVDELQKKEGDLELHTIMRLTDNKLNDKRVEKINRASSELTNKAHSMGCKMIVGESD |
| | LPVADSKTSKKQNVDRMDWCARALSHKVEYACKLMGLAYRGIPAYMSSHQDPLVHLVES |
| | KRSVLRPRFVVADKSDVKQHHLDNLRRMLNSKTKVGTAVYYREAVELMCEELGIHKTDM |
| | AKGKVSLSDFVDKFIGEKAIFPQRGGRFYMSTKRLTTGAKLICYSGSDVWLSDADEIAA |
| | INIGMFVVCDQTGAFKKKKKEKLDDEECDILPFRPM |

What is claimed is:

1. A composition comprising an RNA guide, wherein the RNA guide comprises (i) a spacer sequence that is substantially complementary to a target sequence within a BCL11A gene and (ii) a direct repeat sequence; wherein the target sequence is adjacent to a protospacer adjacent motif (PAM) comprising the sequence 5′-NTTN-3′.

2. The composition of claim 1, wherein the target sequence is within exon 1, exon 2, exon 3, exon 4, or the enhancer region of the BCL11A gene.

3. The composition of claim 1 or 2, wherein the BCL11A gene comprises the sequence of SEQ ID NO: 2635, the reverse complement of SEQ ID NO: 2635, a variant of SEQ ID NO: 2635, or the reverse complement of a variant of SEQ ID NO: 2635.

4. The composition of any one of claims 1 to 3, wherein the spacer sequence comprises:
a. nucleotide 1 through nucleotide 16 of a sequence that is at least 90% identical to a sequence of any one of SEQ ID NOs: 1322-2632;
b. nucleotide 1 through nucleotide 17 of a sequence that is at least 90% identical to a sequence of any one of SEQ ID NOs: 1322-2632;
c. nucleotide 1 through nucleotide 18 of a sequence that is at least 90% identical to a sequence of any one of SEQ ID NOs: 1322-2632;
d. nucleotide 1 through nucleotide 19 of a sequence that is at least 90% identical to a sequence of any one of SEQ ID NOs: 1322-2632;
e. nucleotide 1 through nucleotide 20 of a sequence that is at least 90% identical to a sequence of any one of SEQ ID NOs: 1322-2632;
f. nucleotide 1 through nucleotide 21 of a sequence that is at least 90% identical to a sequence of any one of SEQ ID NOs: 1322-2632;
g. nucleotide 1 through nucleotide 22 of a sequence that is at least 90% identical to a sequence of any one of SEQ ID NOs: 1322-2632;
h. nucleotide 1 through nucleotide 23 of a sequence that is at least 90% identical to a sequence of any one of SEQ ID NOs: 1322-2632;
i. nucleotide 1 through nucleotide 24 of a sequence that is at least 90% identical to a sequence of any one of SEQ ID NOs: 1322-2632;
j. nucleotide 1 through nucleotide 25 of a sequence that is at least 90% identical to a sequence of any one of SEQ ID NOs: 1322-2632;
k. nucleotide 1 through nucleotide 26 of a sequence that is at least 90% identical to a sequence of any one of SEQ ID NOs: 1322-2632;
l. nucleotide 1 through nucleotide 27 of a sequence that is at least 90% identical to a sequence of any one of SEQ ID NOs: 1322-2632;
m. nucleotide 1 through nucleotide 28 of a sequence that is at least 90% identical to a sequence of any one of SEQ ID NOs: 1322-2632;
n. nucleotide 1 through nucleotide 29 of a sequence that is at least 90% identical to a sequence of any one of SEQ ID NOs: 1322-1425 and 1427-2632; or
o. nucleotide 1 through nucleotide 30 of a sequence that is at least 90% identical to a sequence of any one of SEQ ID NOs: 1322-1425 and 1427-2632.

5. The composition of any one of claims 1 to 4, wherein the spacer sequence comprises:
a. nucleotide 1 through nucleotide 16 of any one of SEQ ID NOs: 1322-2632;
b. nucleotide 1 through nucleotide 17 of any one of SEQ ID NOs: 1322-2632;
c. nucleotide 1 through nucleotide 18 of any one of SEQ ID NOs: 1322-2632;
d. nucleotide 1 through nucleotide 19 of any one of SEQ ID NOs: 1322-2632;
e. nucleotide 1 through nucleotide 20 of any one of SEQ ID NOs: 1322-2632;
f. nucleotide 1 through nucleotide 21 of any one of SEQ ID NOs: 1322-2632;
g. nucleotide 1 through nucleotide 22 of any one of SEQ ID NOs: 1322-2632;
h. nucleotide 1 through nucleotide 23 of any one of SEQ ID NOs: 1322-2632;
i. nucleotide 1 through nucleotide 24 of any one of SEQ ID NOs: 1322-2632;
j. nucleotide 1 through nucleotide 25 of any one of SEQ ID NOs: 1322-2632;
k. nucleotide 1 through nucleotide 26 of any one of SEQ ID NOs: 1322-2632;
l. nucleotide 1 through nucleotide 27 of any one of SEQ ID NOs: 1322-2632;
m. nucleotide 1 through nucleotide 28 of any one of SEQ ID NOs: 1322-2632;
n. nucleotide 1 through nucleotide 29 of any one of SEQ ID NOs: 1322-1425 and 1427-2632; or
o. nucleotide 1 through nucleotide 30 of any one of SEQ ID NOs: 1322-1425 and 1427-2632.

6. The composition of any one of claims 1 to 5, wherein the direct repeat comprises:
a. nucleotide 1 through nucleotide 36 of a sequence that is at least 90% identical to a sequence of any one of SEQ ID NOs: 1-8;
b. nucleotide 2 through nucleotide 36 of a sequence that is at least 90% identical to a sequence of any one of SEQ ID NOs: 1-8;
c. nucleotide 3 through nucleotide 36 of a sequence that is at least 90% identical to a sequence of any one of SEQ ID NOs: 1-8;
d. nucleotide 4 through nucleotide 36 of a sequence that is at least 90% identical to a sequence of any one of SEQ ID NOs: 1-8;
e. nucleotide 5 through nucleotide 36 of a sequence that is at least 90% identical to a sequence of any one of SEQ ID NOs: 1-8;
f. nucleotide 6 through nucleotide 36 of a sequence that is at least 90% identical to a sequence of any one of SEQ ID NOs: 1-8;
g. nucleotide 7 through nucleotide 36 of a sequence that is at least 90% identical to a sequence of any one of SEQ ID NOs: 1-8;
h. nucleotide 8 through nucleotide 36 of a sequence that is at least 90% identical to a sequence of any one of SEQ ID NOs: 1-8;
i. nucleotide 9 through nucleotide 36 of a sequence that is at least 90% identical to a sequence of any one of SEQ ID NOs: 1-8;
j. nucleotide 10 through nucleotide 36 of a sequence that is at least 90% identical to a sequence of any one of SEQ ID NOs: 1-8;
k. nucleotide 11 through nucleotide 36 of a sequence that is at least 90% identical to a sequence of any one of SEQ ID NOs: 1-8;
l. nucleotide 12 through nucleotide 36 of a sequence that is at least 90% identical to a sequence of any one of SEQ ID NOs: 1-8;
m. nucleotide 13 through nucleotide 36 of a sequence that is at least 90% identical to a sequence of any one of SEQ ID NOs: 1-8;
n. nucleotide 14 through nucleotide 36 of a sequence that is at least 90% identical to a sequence of any one of SEQ ID NOs: 1-8;
o. nucleotide 1 through nucleotide 34 of a sequence that is at least 90% identical to a sequence of SEQ ID NO: 9;
p. nucleotide 2 through nucleotide 34 of a sequence that is at least 90% identical to a sequence of SEQ ID NO: 9;
q. nucleotide 3 through nucleotide 34 of a sequence that is at least 90% identical to a sequence of SEQ ID NO: 9;
r. nucleotide 4 through nucleotide 34 of a sequence that is at least 90% identical to a sequence of SEQ ID NO: 9;
s. nucleotide 5 through nucleotide 34 of a sequence that is at least 90% identical to a sequence of SEQ ID NO:9;
t. nucleotide 6 through nucleotide 34 of a sequence that is at least 90% identical to a sequence of SEQ ID NO: 9;
u. nucleotide 7 through nucleotide 34 of a sequence that is at least 90% identical to a sequence of SEQ ID NO: 9;
v. nucleotide 8 through nucleotide 34 of a sequence that is at least 90% identical to a sequence of SEQ ID NO: 9;
w. nucleotide 9 through nucleotide 34 of a sequence that is at least 90% identical to a sequence of SEQ ID NO: 9;
x. nucleotide 10 through nucleotide 34 of a sequence that is at least 90% identical to a sequence of SEQ ID NO: 9;
y. nucleotide 11 through nucleotide 34 of a sequence that is at least 90% identical to a sequence of SEQ ID NO: 9;
z. nucleotide 12 through nucleotide 34 of a sequence that is at least 90% identical to a sequence of SEQ ID NO: 9; or
aa. a sequence that is at least 90% identical to a sequence of SEQ ID NO: 10 or a portion thereof.

7. The composition of any one of claims 1 to 6, wherein the direct repeat comprises:
a. nucleotide 1 through nucleotide 36 of any one of SEQ ID NOs: 1-8;
b. nucleotide 2 through nucleotide 36 of any one of SEQ ID NOs: 1-8;
c. nucleotide 3 through nucleotide 36 of any one of SEQ ID NOs: 1-8;
d. nucleotide 4 through nucleotide 36 of any one of SEQ ID NOs: 1-8;
e. nucleotide 5 through nucleotide 36 of any one of SEQ ID NOs: 1-8;
f. nucleotide 6 through nucleotide 36 of any one of SEQ ID NOs: 1-8;
g. nucleotide 7 through nucleotide 36 of any one of SEQ ID NOs: 1-8;
h. nucleotide 8 through nucleotide 36 of any one of SEQ ID NOs: 1-8;
i. nucleotide 9 through nucleotide 36 of any one of SEQ ID NOs: 1-8;
j. nucleotide 10 through nucleotide 36 of any one of SEQ ID NOs: 1-8;
k. nucleotide 11 through nucleotide 36 of any one of SEQ ID NOs: 1-8;
l. nucleotide 12 through nucleotide 36 of any one of SEQ ID NOs: 1-8;
m. nucleotide 13 through nucleotide 36 of any one of SEQ ID NOs: 1-8;
n. nucleotide 14 through nucleotide 36 of any one of SEQ ID NOs: 1-8;
o. nucleotide 1 through nucleotide 34 of SEQ ID NO: 9;
p. nucleotide 2 through nucleotide 34 of SEQ ID NO: 9;
q. nucleotide 3 through nucleotide 34 of SEQ ID NO: 9;
r. nucleotide 4 through nucleotide 34 of SEQ ID NO: 9;
s. nucleotide 5 through nucleotide 34 of SEQ ID NO: 9;
t. nucleotide 6 through nucleotide 34 of SEQ ID NO: 9;
u. nucleotide 7 through nucleotide 34 of SEQ ID NO: 9;
v. nucleotide 8 through nucleotide 34 of SEQ ID NO: 9;
w. nucleotide 9 through nucleotide 34 of SEQ ID NO: 9;
x. nucleotide 10 through nucleotide 34 of SEQ ID NO: 9;
y. nucleotide 11 through nucleotide 34 of SEQ ID NO: 9;
z. nucleotide 12 through nucleotide 34 of SEQ ID NO: 9; or
aa. SEQ ID NO: 10 or a portion thereof.

8. The composition of any one of claims 1 to 5, wherein the direct repeat comprises:
a. nucleotide 1 through nucleotide 36 of a sequence that is at least 90% identical to a sequence of any one of SEQ ID NOs: 2652-2669;
b. nucleotide 2 through nucleotide 36 of a sequence that is at least 90% identical to a sequence of any one of SEQ ID NOs: 2652-2669;
c. nucleotide 3 through nucleotide 36 of a sequence that is at least 90% identical to a sequence of any one of SEQ ID NOs: 2652-2669;
d. nucleotide 4 through nucleotide 36 of a sequence that is at least 90% identical to a sequence of any one of SEQ ID NOs: 2652-2669;
e. nucleotide 5 through nucleotide 36 of a sequence that is at least 90% identical to a sequence of any one of SEQ ID NOs: 2652-2669;
f. nucleotide 6 through nucleotide 36 of a sequence that is at least 90% identical to a sequence of any one of SEQ ID NOs: 2652-2669;
g. nucleotide 7 through nucleotide 36 of a sequence that is at least 90% identical to a sequence of any one of SEQ ID NOs: 2652-2669;
h. nucleotide 8 through nucleotide 36 of a sequence that is at least 90% identical to a sequence of any one of SEQ ID NOs: 2652-2669;
i. nucleotide 9 through nucleotide 36 of a sequence that is at least 90% identical to a sequence of any one of SEQ ID NOs: 2652-2669;
j. nucleotide 10 through nucleotide 36 of a sequence that is at least 90% identical to a sequence of any one of SEQ ID NOs: 2652-2669;
k. nucleotide 11 through nucleotide 36 of a sequence that is at least 90% identical to a sequence of any one of SEQ ID NOs: 2652-2669;
l. nucleotide 12 through nucleotide 36 of a sequence that is at least 90% identical to a sequence of any one of SEQ ID NOs: 2652-2669;
m. nucleotide 13 through nucleotide 36 of a sequence that is at least 90% identical to a sequence of any one of SEQ ID NOs: 2652-2669;
n. nucleotide 14 through nucleotide 36 of a sequence that is at least 90% identical to a sequence of any one of SEQ ID NOs: 2652-2669; or
o. a sequence that is at least 90% identical to a sequence of SEQ ID NO: 2670 or a portion thereof.

9. The composition of any one of claims 1 to 5 or 8, wherein the direct repeat comprises:
a. nucleotide 1 through nucleotide 36 of any one of SEQ ID NOs: 2652-2669;
b. nucleotide 2 through nucleotide 36 of any one of SEQ ID NOs: 2652-2669;
c. nucleotide 3 through nucleotide 36 of any one of SEQ ID NOs: 2652-2669;
d. nucleotide 4 through nucleotide 36 of any one of SEQ ID NOs: 2652-2669;
e. nucleotide 5 through nucleotide 36 of any one of SEQ ID NOs: 2652-2669;
f. nucleotide 6 through nucleotide 36 of any one of SEQ ID NOs: 2652-2669;
g. nucleotide 7 through nucleotide 36 of any one of SEQ ID NOs: 2652-2669;
h. nucleotide 8 through nucleotide 36 of any one of SEQ ID NOs: 2652-2669;
i. nucleotide 9 through nucleotide 36 of any one of SEQ ID NOs: 2652-2669;
j. nucleotide 10 through nucleotide 36 of any one of SEQ ID NOs: 2652-2669;
k. nucleotide 11 through nucleotide 36 of any one of SEQ ID NOs: 2652-2669;
l. nucleotide 12 through nucleotide 36 of any one of SEQ ID NOs: 2652-2669;
m. nucleotide 13 through nucleotide 36 of any one of SEQ ID NOs: 2652-2669;
n. nucleotide 14 through nucleotide 36 of any one of SEQ ID NOs: 2652-2669; or
o. SEQ ID NO: 2670 or a portion thereof.

10. The composition of any one of claims 1 to 5, wherein the direct repeat comprises:
a. nucleotide 1 through nucleotide 36 of a sequence that is at least 90% identical to SEQ ID NO: 2671;
b. nucleotide 2 through nucleotide 36 of a sequence that is at least 90% identical to SEQ ID NO: 2671;
c. nucleotide 3 through nucleotide 36 of a sequence that is at least 90% identical to SEQ ID NO: 2671;
d. nucleotide 4 through nucleotide 36 of a sequence that is at least 90% identical to SEQ ID NO: 2671;
e. nucleotide 5 through nucleotide 36 of a sequence that is at least 90% identical to SEQ ID NO: 2671;
f. nucleotide 6 through nucleotide 36 of a sequence that is at least 90% identical to SEQ ID NO: 2671;
g. nucleotide 7 through nucleotide 36 of a sequence that is at least 90% identical to SEQ ID NO: 2671;
h. nucleotide 8 through nucleotide 36 of a sequence that is at least 90% identical to SEQ ID NO: 2671;
i. nucleotide 9 through nucleotide 36 of a sequence that is at least 90% identical to SEQ ID NO: 2671;
j. nucleotide 10 through nucleotide 36 of a sequence that is at least 90% identical to SEQ ID NO: 2671;
k. nucleotide 11 through nucleotide 36 of a sequence that is at least 90% identical to SEQ ID NO: 2671;
l. nucleotide 12 through nucleotide 36 of a sequence that is at least 90% identical to SEQ ID NO: 2671;
m. nucleotide 13 through nucleotide 36 of a sequence that is at least 90% identical to SEQ ID NO: 2671;
n. nucleotide 14 through nucleotide 36 of a sequence that is at least 90% identical to SEQ ID NO: 2671; or
o. a sequence that is at least 90% identical to a sequence of SEQ ID NO: 2672 or SEQ ID NO: 2673 or a portion thereof.

11. The composition of any one of claims 1 to 5 or 10, wherein the direct repeat comprises:
a. nucleotide 1 through nucleotide 36 of SEQ ID NO: 2671;
b. nucleotide 2 through nucleotide 36 of SEQ ID NO: 2671;
c. nucleotide 3 through nucleotide 36 of SEQ ID NO: 2671;
d. nucleotide 4 through nucleotide 36 of SEQ ID NO: 2671;
e. nucleotide 5 through nucleotide 36 of SEQ ID NO: 2671;
f. nucleotide 6 through nucleotide 36 of SEQ ID NO: 2671;
g. nucleotide 7 through nucleotide 36 of SEQ ID NO: 2671;
h. nucleotide 8 through nucleotide 36 of SEQ ID NO: 2671;
i. nucleotide 9 through nucleotide 36 of SEQ ID NO: 2671;
j. nucleotide 10 through nucleotide 36 of SEQ ID NO: 2671;
k. nucleotide 11 through nucleotide 36 of SEQ ID NO: 2671;
l. nucleotide 12 through nucleotide 36 of SEQ ID NO: 2671;
m. nucleotide 13 through nucleotide 36 of SEQ ID NO: 2671;
n. nucleotide 14 through nucleotide 36 of SEQ ID NO: 2671; or
o. SEQ ID NO: 2672 or SEQ ID NO: 2673 or a portion thereof.

12. The composition of any one of claims 1 to 5, wherein the direct repeat comprises:
a. nucleotide 1 through nucleotide 36 of a sequence that is at least 90% identical to a sequence of SEQ ID NO: 2674 or SEQ ID NO: 2675;
b. nucleotide 2 through nucleotide 36 of a sequence that is at least 90% identical to a sequence of SEQ ID NO: 2674 or SEQ ID NO: 2675;
c. nucleotide 3 through nucleotide 36 of a sequence that is at least 90% identical to a sequence of SEQ ID NO: 2674 or SEQ ID NO: 2675;
d. nucleotide 4 through nucleotide 36 of a sequence that is at least 90% identical to a sequence of SEQ ID NO: 2674 or SEQ ID NO: 2675;
e. nucleotide 5 through nucleotide 36 of a sequence that is at least 90% identical to a sequence of SEQ ID NO: 2674 or SEQ ID NO: 2675;
f. nucleotide 6 through nucleotide 36 of a sequence that is at least 90% identical to a sequence of SEQ ID NO: 2674 or SEQ ID NO: 2675;
g. nucleotide 7 through nucleotide 36 of a sequence that is at least 90% identical to a sequence of SEQ ID NO: 2674 or SEQ ID NO: 2675;
h. nucleotide 8 through nucleotide 36 of a sequence that is at least 90% identical to a sequence of SEQ ID NO: 2674 or SEQ ID NO: 2675;
i. nucleotide 9 through nucleotide 36 of a sequence that is at least 90% identical to a sequence of SEQ ID NO: 2674 or SEQ ID NO: 2675;
j. nucleotide 10 through nucleotide 36 of a sequence that is at least 90% identical to a sequence of SEQ ID NO: 2674 or SEQ ID NO: 2675;
k. nucleotide 11 through nucleotide 36 of a sequence that is at least 90% identical to a sequence of SEQ ID NO: 2674 or SEQ ID NO: 2675;
l. nucleotide 12 through nucleotide 36 of a sequence that is at least 90% identical to a sequence of SEQ ID NO: 2674 or SEQ ID NO: 2675;
m. nucleotide 13 through nucleotide 36 of a sequence that is at least 90% identical to a sequence of SEQ ID NO: 2674 or SEQ ID NO: 2675;
n. nucleotide 14 through nucleotide 36 of a sequence that is at least 90% identical to a sequence of SEQ ID NO: 2674 or SEQ ID NO: 2675;
o. nucleotide 15 through nucleotide 36 of a sequence that is at least 90% identical to a sequence of SEQ ID NO: 2674 or SEQ ID NO: 2675; or
p. a sequence that is at least 90% identical to a sequence of SEQ ID NO: 2676 or a portion thereof.

13. The composition of any one of claims 1 to 5 or 12, wherein the direct repeat comprises:
a. nucleotide 1 through nucleotide 36 of SEQ ID NO: 2674 or SEQ ID NO: 2675;
b. nucleotide 2 through nucleotide 36 of SEQ ID NO: 2674 or SEQ ID NO: 2675;
c. nucleotide 3 through nucleotide 36 of SEQ ID NO: 2674 or SEQ ID NO: 2675;
d. nucleotide 4 through nucleotide 36 of SEQ ID NO: 2674 or SEQ ID NO: 2675;
e. nucleotide 5 through nucleotide 36 of SEQ ID NO: 2674 or SEQ ID NO: 2675;
f. nucleotide 6 through nucleotide 36 of SEQ ID NO: 2674 or SEQ ID NO: 2675;
g. nucleotide 7 through nucleotide 36 of SEQ ID NO: 2674 or SEQ ID NO: 2675;
h. nucleotide 8 through nucleotide 36 of SEQ ID NO: 2674 or SEQ ID NO: 2675;
i. nucleotide 9 through nucleotide 36 of SEQ ID NO: 2674 or SEQ ID NO: 2675;
j. nucleotide 10 through nucleotide 36 of SEQ ID NO: 2674 or SEQ ID NO: 2675;
k. nucleotide 11 through nucleotide 36 of SEQ ID NO: 2674 or SEQ ID NO: 2675;
l. nucleotide 12 through nucleotide 36 of SEQ ID NO: 2674 or SEQ ID NO: 2675;
m. nucleotide 13 through nucleotide 36 of SEQ ID NO: 2674 or SEQ ID NO: 2675;
n. nucleotide 14 through nucleotide 36 of SEQ ID NO: 2674 or SEQ ID NO: 2675;
o. nucleotide 15 through nucleotide 36 of SEQ ID NO: 2674 or SEQ ID NO: 2675; or
p. SEQ ID NO: 2676 or a portion thereof.

14. The composition of any one of claims 1 to 13, wherein the spacer sequence is substantially complementary to the complement of a sequence of any one of SEQ ID NOs: 11-1321.

15. The composition of claim 1, wherein the PAM comprises the sequence 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′.

16. The composition of claim 1 or 15, wherein the target sequence is immediately adjacent to the PAM sequence.

17. The composition of any one of claims 1 to 16, wherein the composition further comprises a Cas12i polypeptide.

18. The composition of claim 17, wherein the Cas12i polypeptide is:
a. a Cas12i2 polypeptide comprising a sequence that is at least 90% identical to the sequence of SEQ ID NO: 2634, SEQ ID NO: 2641, SEQ ID NO: 2642, SEQ ID NO: 2643, SEQ ID NO: 2644, or SEQ ID NO: 2645;
b. a Cas12i4 polypeptide comprising a sequence that is at least 90% identical to the sequence of SEQ ID NO: 2647, SEQ ID NO: 2648, or SEQ ID NO: 2649;
c. a Cas12i1 polypeptide comprising a sequence that is at least 90% identical to the sequence of SEQ ID NO: 2650; or
d. a Cas12i3 polypeptide comprising a sequence that is at least 90% identical to the sequence of SEQ ID NO: 2651.

19. The composition of claim 18, wherein the Cas12i polypeptide is:
a. a Cas12i2 polypeptide comprising a sequence of SEQ ID NO: 2634, SEQ ID NO: 2641, SEQ ID NO: 2642, SEQ ID NO: 2643, SEQ ID NO: 2644, or SEQ ID NO: 2645;
b. a Cas12i4 polypeptide comprising a sequence of SEQ ID NO: 2647, SEQ ID NO: 2648, or SEQ ID NO: 2649;
c. a Cas12i1 polypeptide comprising a sequence of SEQ ID NO: 2650; or
d. a Cas12i3 polypeptide comprising a sequence of SEQ ID NO: 2651.

20. The composition of any one of claims 17 to 19, wherein the RNA guide and the Cas12i polypeptide form a ribonucleoprotein complex.

21. The composition of claim 20, wherein the ribonucleoprotein complex binds a target nucleic acid.

22. The composition of claim 20 or 21, wherein the composition is present within a cell.

23. The composition of any one of claims 17 to 22, wherein the RNA guide and the Cas12i polypeptide are encoded in a vector, e.g., expression vector.

24. The composition of claim 23, wherein the RNA guide and the Cas12i polypeptide are encoded in a single vector or the RNA guide is encoded in a first vector and the Cas12i polypeptide is encoded in a second vector.

25. An RNA guide comprising (i) a spacer sequence that is substantially complementary to a target sequence within a BCL11A gene and (ii) a direct repeat sequence.

26. The RNA guide of claim 25, wherein the target sequence is within exon 1, exon 2, exon 3, exon 4, or the enhancer region of the BCL11A gene.

27. The RNA guide of claim 25 or 26, wherein the BCL11A gene comprises the sequence of SEQ ID NO: 2635, the reverse complement of SEQ ID NO: 2635, a variant of SEQ ID NO: 2635, or the reverse complement of SEQ ID NO: 2635.

28. The RNA guide of any one of claims 25 to 27, wherein the spacer sequence comprises:
a. nucleotide 1 through nucleotide 16 of a sequence that is at least 90% identical to a sequence of any one of SEQ ID NOs: 1322-2632;
b. nucleotide 1 through nucleotide 17 of a sequence that is at least 90% identical to a sequence of any one of SEQ ID NOs: 1322-2632;
c. nucleotide 1 through nucleotide 18 of a sequence that is at least 90% identical to a sequence of any one of SEQ ID NOs: 1322-2632;
d. nucleotide 1 through nucleotide 19 of a sequence that is at least 90% identical to a sequence of any one of SEQ ID NOs: 1322-2632;
e. nucleotide 1 through nucleotide 20 of a sequence that is at least 90% identical to a sequence of any one of SEQ ID NOs: 1322-2632;
f. nucleotide 1 through nucleotide 21 of a sequence that is at least 90% identical to a sequence of any one of SEQ ID NOs: 1322-2632;
g. nucleotide 1 through nucleotide 22 of a sequence that is at least 90% identical to a sequence of any one of SEQ ID NOs: 1322-2632;
h. nucleotide 1 through nucleotide 23 of a sequence that is at least 90% identical to a sequence of any one of SEQ ID NOs: 1322-2632;
i. nucleotide 1 through nucleotide 24 of a sequence that is at least 90% identical to a sequence of any one of SEQ ID NOs: 1322-2632;
j. nucleotide 1 through nucleotide 25 of a sequence that is at least 90% identical to a sequence of any one of SEQ ID NOs: 1322-2632;
k. nucleotide 1 through nucleotide 26 of a sequence that is at least 90% identical to a sequence of any one of SEQ ID NOs: 1322-2632;
l. nucleotide 1 through nucleotide 27 of a sequence that is at least 90% identical to a sequence of any one of SEQ ID NOs: 1322-2632;
m. nucleotide 1 through nucleotide 28 of a sequence that is at least 90% identical to a sequence of any one of SEQ ID NOs: 1322-2632;
n. nucleotide 1 through nucleotide 29 of a sequence that is at least 90% identical to a sequence of any one of SEQ ID NOs: 1322-1425 and 1427-2632; or
o. nucleotide 1 through nucleotide 30 of a sequence that is at least 90% identical to a sequence of any one of SEQ ID NOs: 1322-1425 and 1427-2632.

29. The RNA guide of any one of claims 25 to 28, wherein the spacer sequence comprises:
a. nucleotide 1 through nucleotide 16 of any one of SEQ ID NOs: 1322-2632;
b. nucleotide 1 through nucleotide 17 of any one of SEQ ID NOs: 1322-2632;
c. nucleotide 1 through nucleotide 18 of any one of SEQ ID NOs: 1322-2632;
d. nucleotide 1 through nucleotide 19 of any one of SEQ ID NOs: 1322-2632;
e. nucleotide 1 through nucleotide 20 of any one of SEQ ID NOs: 1322-2632;
f. nucleotide 1 through nucleotide 21 of any one of SEQ ID NOs: 1322-2632;
g. nucleotide 1 through nucleotide 22 of any one of SEQ ID NOs: 1322-2632;
h. nucleotide 1 through nucleotide 23 of any one of SEQ ID NOs: 1322-2632;
i. nucleotide 1 through nucleotide 24 of any one of SEQ ID NOs: 1322-2632;
j. nucleotide 1 through nucleotide 25 of any one of SEQ ID NOs: 1322-2632;
k. nucleotide 1 through nucleotide 26 of any one of SEQ ID NOs: 1322-2632;
l. nucleotide 1 through nucleotide 27 of any one of SEQ ID NOs: 1322-2632;
m. nucleotide 1 through nucleotide 28 of any one of SEQ ID NOs: 1322-2632;
n. nucleotide 1 through nucleotide 29 of any one of SEQ ID NOs: 1322-1425 and 1427-2632; or
o. nucleotide 1 through nucleotide 30 of any one of SEQ ID NOs: 1322-1425 and 1427-2632.

30. The RNA guide of any one of claims 25 to 29, wherein the direct repeat comprises:
a. nucleotide 1 through nucleotide 36 of a sequence that is at least 90% identical to a sequence of any one of SEQ ID NOs: 1-8;
b. nucleotide 2 through nucleotide 36 of a sequence that is at least 90% identical to a sequence of any one of SEQ ID NOs: 1-8;
c. nucleotide 3 through nucleotide 36 of a sequence that is at least 90% identical to a sequence of any one of SEQ ID NOs: 1-8;
d. nucleotide 4 through nucleotide 36 of a sequence that is at least 90% identical to a sequence of any one of SEQ ID NOs: 1-8;
e. nucleotide 5 through nucleotide 36 of a sequence that is at least 90% identical to a sequence of any one of SEQ ID NOs: 1-8;
f. nucleotide 6 through nucleotide 36 of a sequence that is at least 90% identical to a sequence of any one of SEQ ID NOs: 1-8;
g. nucleotide 7 through nucleotide 36 of a sequence that is at least 90% identical to a sequence of any one of SEQ ID NOs: 1-8;
h. nucleotide 8 through nucleotide 36 of a sequence that is at least 90% identical to a sequence of any one of SEQ ID NOs: 1-8;
i. nucleotide 9 through nucleotide 36 of a sequence that is at least 90% identical to a sequence of any one of SEQ ID NOs: 1-8;
j. nucleotide 10 through nucleotide 36 of a sequence that is at least 90% identical to a sequence of any one of SEQ ID NOs: 1-8;
k. nucleotide 11 through nucleotide 36 of a sequence that is at least 90% identical to a sequence of any one of SEQ ID NOs: 1-8;
l. nucleotide 12 through nucleotide 36 of a sequence that is at least 90% identical to a sequence of any one of SEQ ID NOs: 1-8;
m. nucleotide 13 through nucleotide 36 of a sequence that is at least 90% identical to a sequence of any one of SEQ ID NOs: 1-8;
n. nucleotide 14 through nucleotide 36 of a sequence that is at least 90% identical to a sequence of any one of SEQ ID NOs: 1-8;
o. nucleotide 1 through nucleotide 34 of a sequence that is at least 90% identical to a sequence of SEQ ID NO:9;
p. nucleotide 2 through nucleotide 34 of a sequence that is at least 90% identical to a sequence of SEQ ID NO: 9;
q. nucleotide 3 through nucleotide 34 of a sequence that is at least 90% identical to a sequence of SEQ ID NO: 9;
r. nucleotide 4 through nucleotide 34 of a sequence that is at least 90% identical to a sequence of SEQ ID NO: 9;
s. nucleotide 5 through nucleotide 34 of a sequence that is at least 90% identical to a sequence of SEQ ID NO: 9;
t. nucleotide 6 through nucleotide 34 of a sequence that is at least 90% identical to a sequence of SEQ ID NO: 9;
u. nucleotide 7 through nucleotide 34 of a sequence that is at least 90% identical to a sequence of SEQ ID NO: 9;
v. nucleotide 8 through nucleotide 34 of a sequence that is at least 90% identical to a sequence of SEQ ID NO: 9;
w. nucleotide 9 through nucleotide 34 of a sequence that is at least 90% identical to a sequence of SEQ ID NO:9;
x. nucleotide 10 through nucleotide 34 of a sequence that is at least 90% identical to a sequence of SEQ ID NO: 9;
y. nucleotide 11 through nucleotide 34 of a sequence that is at least 90% identical to a sequence of SEQ ID NO: 9;
z. nucleotide 12 through nucleotide 34 of a sequence that is at least 90% identical to a sequence of SEQ ID NO: 9; or
aa. a sequence that is at least 90% identical to a sequence of SEQ ID NO: 10 or a portion thereof.

31. The RNA guide of any one of claims 25 to 30, wherein the direct repeat comprises:
a. nucleotide 1 through nucleotide 36 of any one of SEQ ID NOs: 1-8;
b. nucleotide 2 through nucleotide 36 of any one of SEQ ID NOs: 1-8;
c. nucleotide 3 through nucleotide 36 of any one of SEQ ID NOs: 1-8;
d. nucleotide 4 through nucleotide 36 of any one of SEQ ID NOs: 1-8;
e. nucleotide 5 through nucleotide 36 of any one of SEQ ID NOs: 1-8;
f. nucleotide 6 through nucleotide 36 of any one of SEQ ID NOs: 1-8;
g. nucleotide 7 through nucleotide 36 of any one of SEQ ID NOs: 1-8;
h. nucleotide 8 through nucleotide 36 of any one of SEQ ID NOs: 1-8;
i. nucleotide 9 through nucleotide 36 of any one of SEQ ID NOs: 1-8;
j. nucleotide 10 through nucleotide 36 of any one of SEQ ID NOs: 1-8;
k. nucleotide 11 through nucleotide 36 of any one of SEQ ID NOs: 1-8;
l. nucleotide 12 through nucleotide 36 of any one of SEQ ID NOs: 1-8;
m. nucleotide 13 through nucleotide 36 of any one of SEQ ID NOs: 1-8;
n. nucleotide 14 through nucleotide 36 of any one of SEQ ID NOs: 1-8;
o. nucleotide 1 through nucleotide 34 of SEQ ID NO: 9;
p. nucleotide 2 through nucleotide 34 of SEQ ID NO: 9;
q. nucleotide 3 through nucleotide 34 of SEQ ID NO: 9;
r. nucleotide 4 through nucleotide 34 of SEQ ID NO: 9;
s. nucleotide 5 through nucleotide 34 of SEQ ID NO: 9;
t. nucleotide 6 through nucleotide 34 of SEQ ID NO: 9;
u. nucleotide 7 through nucleotide 34 of SEQ ID NO: 9;
v. nucleotide 8 through nucleotide 34 of SEQ ID NO: 9;
w. nucleotide 9 through nucleotide 34 of SEQ ID NO: 9;
x. nucleotide 10 through nucleotide 34 of SEQ ID NO: 9;
y. nucleotide 11 through nucleotide 34 of SEQ ID NO: 9;
z. nucleotide 12 through nucleotide 34 of SEQ ID NO: 9; or
aa. SEQ ID NO: 10 or a portion thereof.

32. The RNA guide of any one of claims 25 to 31, wherein the direct repeat comprises:
a. nucleotide 1 through nucleotide 36 of a sequence that is at least 90% identical to a sequence of any one of SEQ ID NOs: 2652-2669;
b. nucleotide 2 through nucleotide 36 of a sequence that is at least 90% identical to a sequence of any one of SEQ ID NOs: 2652-2669;
c. nucleotide 3 through nucleotide 36 of a sequence that is at least 90% identical to a sequence of any one of SEQ ID NOs: 2652-2669;
d. nucleotide 4 through nucleotide 36 of a sequence that is at least 90% identical to a sequence of any one of SEQ ID NOs: 2652-2669;
e. nucleotide 5 through nucleotide 36 of a sequence that is at least 90% identical to a sequence of any one of SEQ ID NOs: 2652-2669;
f. nucleotide 6 through nucleotide 36 of a sequence that is at least 90% identical to a sequence of any one of SEQ ID NOs: 2652-2669;
g. nucleotide 7 through nucleotide 36 of a sequence that is at least 90% identical to a sequence of any one of SEQ ID NOs: 2652-2669;
h. nucleotide 8 through nucleotide 36 of a sequence that is at least 90% identical to a sequence of any one of SEQ ID NOs: 2652-2669;
i. nucleotide 9 through nucleotide 36 of a sequence that is at least 90% identical to a sequence of any one of SEQ ID NOs: 2652-2669;
j. nucleotide 10 through nucleotide 36 of a sequence that is at least 90% identical to a sequence of any one of SEQ ID NOs: 2652-2669;
k. nucleotide 11 through nucleotide 36 of a sequence that is at least 90% identical to a sequence of any one of SEQ ID NOs: 2652-2669;
l. nucleotide 12 through nucleotide 36 of a sequence that is at least 90% identical to a sequence of any one of SEQ ID NOs: 2652-2669;
m. nucleotide 13 through nucleotide 36 of a sequence that is at least 90% identical to a sequence of any one of SEQ ID NOs: 2652-2669;
n. nucleotide 14 through nucleotide 36 of a sequence that is at least 90% identical to a sequence of any one of SEQ ID NOs: 2652-2669; or
o. a sequence that is at least 90% identical to a sequence of SEQ ID NO: 2670 or a portion thereof.

33. The RNA guide of any one of claims 25 to 29 or 32, wherein the direct repeat comprises:
a. nucleotide 1 through nucleotide 36 of any one of SEQ ID NOs: 2652-2669;
b. nucleotide 2 through nucleotide 36 of any one of SEQ ID NOs: 2652-2669;
c. nucleotide 3 through nucleotide 36 of any one of SEQ ID NOs: 2652-2669;
d. nucleotide 4 through nucleotide 36 of any one of SEQ ID NOs: 2652-2669;
e. nucleotide 5 through nucleotide 36 of any one of SEQ ID NOs: 2652-2669;
f. nucleotide 6 through nucleotide 36 of any one of SEQ ID NOs: 2652-2669;
g. nucleotide 7 through nucleotide 36 of any one of SEQ ID NOs: 2652-2669;
h. nucleotide 8 through nucleotide 36 of any one of SEQ ID NOs: 2652-2669;
i. nucleotide 9 through nucleotide 36 of any one of SEQ ID NOs: 2652-2669;
j. nucleotide 10 through nucleotide 36 of any one of SEQ ID NOs: 2652-2669;
k. nucleotide 11 through nucleotide 36 of any one of SEQ ID NOs: 2652-2669;
l. nucleotide 12 through nucleotide 36 of any one of SEQ ID NOs: 2652-2669;
m. nucleotide 13 through nucleotide 36 of any one of SEQ ID NOs: 2652-2669;
n. nucleotide 14 through nucleotide 36 of any one of SEQ ID NOs: 2652-2669; or
o. SEQ ID NO: 2670 or a portion thereof.

34. The RNA guide of any one of claims 25 to 29, wherein the direct repeat comprises:
a. nucleotide 1 through nucleotide 36 of a sequence that is at least 90% identical to SEQ ID NO: 2671;
b. nucleotide 2 through nucleotide 36 of a sequence that is at least 90% identical to SEQ ID NO: 2671;
c. nucleotide 3 through nucleotide 36 of a sequence that is at least 90% identical to SEQ ID NO: 2671;
d. nucleotide 4 through nucleotide 36 of a sequence that is at least 90% identical to SEQ ID NO: 2671;
e. nucleotide 5 through nucleotide 36 of a sequence that is at least 90% identical to SEQ ID NO: 2671;
f. nucleotide 6 through nucleotide 36 of a sequence that is at least 90% identical to SEQ ID NO: 2671;
g. nucleotide 7 through nucleotide 36 of a sequence that is at least 90% identical to SEQ ID NO: 2671;
h. nucleotide 8 through nucleotide 36 of a sequence that is at least 90% identical to SEQ ID NO: 2671;
i. nucleotide 9 through nucleotide 36 of a sequence that is at least 90% identical to SEQ ID NO: 2671;
j. nucleotide 10 through nucleotide 36 of a sequence that is at least 90% identical to SEQ ID NO: 2671;
k. nucleotide 11 through nucleotide 36 of a sequence that is at least 90% identical to SEQ ID NO: 2671;
l. nucleotide 12 through nucleotide 36 of a sequence that is at least 90% identical to SEQ ID NO: 2671;
m. nucleotide 13 through nucleotide 36 of a sequence that is at least 90% identical to SEQ ID NO: 2671;
n. nucleotide 14 through nucleotide 36 of a sequence that is at least 90% identical to SEQ ID NO: 2671; or
o. a sequence that is at least 90% identical to a sequence of SEQ ID NO: 2672 or SEQ ID NO: 2673 or a portion thereof.

35. The RNA guide of any one of claims 25 to 29 or 34, wherein the direct repeat comprises:
a. nucleotide 1 through nucleotide 36 of SEQ ID NO: 2671;
b. nucleotide 2 through nucleotide 36 of SEQ ID NO: 2671;
c. nucleotide 3 through nucleotide 36 of SEQ ID NO: 2671;
d. nucleotide 4 through nucleotide 36 of SEQ ID NO: 2671;
e. nucleotide 5 through nucleotide 36 of SEQ ID NO: 2671;
f. nucleotide 6 through nucleotide 36 of SEQ ID NO: 2671;
g. nucleotide 7 through nucleotide 36 of SEQ ID NO: 2671;
h. nucleotide 8 through nucleotide 36 of SEQ ID NO: 2671;
i. nucleotide 9 through nucleotide 36 of SEQ ID NO: 2671;
j. nucleotide 10 through nucleotide 36 of SEQ ID NO: 2671;
k. nucleotide 11 through nucleotide 36 of SEQ ID NO: 2671;
l. nucleotide 12 through nucleotide 36 of SEQ ID NO: 2671;
m. nucleotide 13 through nucleotide 36 of SEQ ID NO: 2671;
n. nucleotide 14 through nucleotide 36 of SEQ ID NO: 2671; or
o. SEQ ID NO: 2672 or SEQ ID NO: 2673 or a portion thereof.

36. The RNA guide of any one of claims 25 to 29, wherein the direct repeat comprises:
a. nucleotide 1 through nucleotide 36 of a sequence that is at least 90% identical to a sequence of SEQ ID NO: 2674 or SEQ ID NO: 2675;
b. nucleotide 2 through nucleotide 36 of a sequence that is at least 90% identical to a sequence of SEQ ID NO: 2674 or SEQ ID NO: 2675;
c. nucleotide 3 through nucleotide 36 of a sequence that is at least 90% identical to a sequence of SEQ ID NO: 2674 or SEQ ID NO: 2675;
d. nucleotide 4 through nucleotide 36 of a sequence that is at least 90% identical to a sequence of SEQ ID NO: 2674 or SEQ ID NO: 2675;
e. nucleotide 5 through nucleotide 36 of a sequence that is at least 90% identical to a sequence of SEQ ID NO: 2674 or SEQ ID NO: 2675;
f. nucleotide 6 through nucleotide 36 of a sequence that is at least 90% identical to a sequence of SEQ ID NO: 2674 or SEQ ID NO: 2675;
g. nucleotide 7 through nucleotide 36 of a sequence that is at least 90% identical to a sequence of SEQ ID NO: 2674 or SEQ ID NO: 2675;
h. nucleotide 8 through nucleotide 36 of a sequence that is at least 90% identical to a sequence of SEQ ID NO: 2674 or SEQ ID NO: 2675;
i. nucleotide 9 through nucleotide 36 of a sequence that is at least 90% identical to a sequence of SEQ ID NO: 2674 or SEQ ID NO: 2675;
j. nucleotide 10 through nucleotide 36 of a sequence that is at least 90% identical to a sequence of SEQ ID NO: 2674 or SEQ ID NO: 2675;
k. nucleotide 11 through nucleotide 36 of a sequence that is at least 90% identical to a sequence of SEQ ID NO: 2674 or SEQ ID NO: 2675;
l. nucleotide 12 through nucleotide 36 of a sequence that is at least 90% identical to a sequence of SEQ ID NO: 2674 or SEQ ID NO: 2675;
m. nucleotide 13 through nucleotide 36 of a sequence that is at least 90% identical to a sequence of SEQ ID NO: 2674 or SEQ ID NO: 2675;
n. nucleotide 14 through nucleotide 36 of a sequence that is at least 90% identical to a sequence of SEQ ID NO: 2674 or SEQ ID NO: 2675;
o. nucleotide 15 through nucleotide 36 of a sequence that is at least 90% identical to a sequence of SEQ ID NO: 2674 or SEQ ID NO: 2675; or
p. a sequence that is at least 90% identical to a sequence of SEQ ID NO: 2676 or a portion thereof.

37. The RNA guide of any one of claims 25 to 29 or 36, wherein the direct repeat comprises:
a. nucleotide 1 through nucleotide 36 of SEQ ID NO: 2674 or SEQ ID NO: 2675;
b. nucleotide 2 through nucleotide 36 of SEQ ID NO: 2674 or SEQ ID NO: 2675;
c. nucleotide 3 through nucleotide 36 of SEQ ID NO: 2674 or SEQ ID NO: 2675;
d. nucleotide 4 through nucleotide 36 of SEQ ID NO: 2674 or SEQ ID NO: 2675;
e. nucleotide 5 through nucleotide 36 of SEQ ID NO: 2674 or SEQ ID NO: 2675;
f. nucleotide 6 through nucleotide 36 of SEQ ID NO: 2674 or SEQ ID NO: 2675;
g. nucleotide 7 through nucleotide 36 of SEQ ID NO: 2674 or SEQ ID NO: 2675;
h. nucleotide 8 through nucleotide 36 of SEQ ID NO: 2674 or SEQ ID NO: 2675;
i. nucleotide 9 through nucleotide 36 of SEQ ID NO: 2674 or SEQ ID NO: 2675;
j. nucleotide 10 through nucleotide 36 of SEQ ID NO: 2674 or SEQ ID NO: 2675;
k. nucleotide 11 through nucleotide 36 of SEQ ID NO: 2674 or SEQ ID NO: 2675;
l. nucleotide 12 through nucleotide 36 of SEQ ID NO: 2674 or SEQ ID NO: 2675;
m. nucleotide 13 through nucleotide 36 of SEQ ID NO: 2674 or SEQ ID NO: 2675;
n. nucleotide 14 through nucleotide 36 of SEQ ID NO: 2674 or SEQ ID NO: 2675;
o. nucleotide 15 through nucleotide 36 of SEQ ID NO: 2674 or SEQ ID NO: 2675; or
p. SEQ ID NO: 2676 or a portion thereof.

38. The RNA guide of any one of claims 25 to 37, wherein the spacer sequence is substantially complementary to the complement of a sequence of any one of SEQ ID NOs: 11-1321.

39. The RNA guide of any one of claims 25 to 38, wherein the target sequence is adjacent to a protospacer adjacent motif (PAM) comprising the sequence 5′-NTTN-3′, wherein N is any nucleotide.

40. The RNA guide of claim 39, wherein the PAM comprises the sequence 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′.

41. The RNA guide of claim 39 or 40, wherein the target sequence is immediately adjacent to the PAM sequence.

42. A nucleic acid encoding an RNA guide of any one of claims 25 to 41.

43. A vector comprising the nucleic acid of claim 42.

44. A vector system comprising one or more vectors encoding (i) the RNA guide as defined in any of claims 1 to 41 and (ii) a Cas12i polypeptide, optionally wherein the vector system comprises a first vector encoding the RNA guide and a second vector encoding the Cas12i polypeptide.

45. A cell comprising the composition of any one of claims 1 to 24, the RNA guide of any one of claims 25 to 41, the nucleic acid of claim 42, the vector of claim 43, or the vector system of claim 44.

46. The cell of claim 45, wherein the cell is a eukaryotic cell, an animal cell, a mammalian cell, a human cell, a primary cell, a cell line, a stem cell, or a T cell.

47. A kit comprising the composition of any one of claims 1 to 24, the RNA guide of any one of claims 25 to 41, the nucleic acid of claim 42, the vector of claim 43, or the vector system of claim 44.

48. A method of editing a BCL11A sequence, the method comprising contacting a BCL11A sequence with a composition of any one of claims 1 to 24 or an RNA guide of any one of claims 25 to 41.

49. The method of claim 48, wherein the BCL11A sequence is in a cell.

50. The method of claim 48 or 49, wherein the composition or the RNA guide induces a deletion in the BCL11A sequence.

51. The method of claim 50, wherein the deletion is adjacent to a 5′-NTTN-3′ sequence, wherein N is any nucleotide.

52. The method of claim 50 or 51, wherein the deletion is downstream of the 5′-NTTN-3′ sequence.

53. The method of any one of claims 50 to 52, wherein the deletion is up to about 50 nucleotides in length.

54. The method of any one of claims 50 to 53, wherein the deletion is up to about 40 nucleotides in length.

55. The method of any one of claims 50 to 54, wherein the deletion is from about 4 nucleotides to 40 nucleotides in length.

56. The method of any one of claims 50 to 55, wherein the deletion is from about 4 nucleotides to 25 nucleotides in length.

57. The method of any one of claims 50 to 56, wherein the deletion is from about 10 nucleotides to 25 nucleotides in length.

58. The method of any one of claims 50 to 57, wherein the deletion is from about 10 nucleotides to 15 nucleotides in length.

59. The method of any one of claims 50 to 58, wherein the deletion starts within about 5 nucleotides to about 15 nucleotides of the 5′-NTTN-3′ sequence.

60. The method of any one of claims 50 to 59, wherein the deletion starts within about 5 nucleotides to about 10 nucleotides of the 5′-NTTN-3′ sequence.

61. The method of any one of claims 50 to 60, wherein the deletion starts within about 10 nucleotides to about 15 nucleotides of the 5′-NTTN-3′ sequence.

62. The method of any one of claims 50 to 61, wherein the deletion starts within about 5 nucleotides to about 15 nucleotides downstream of the 5′-NTTN-3′ sequence.

63. The method of any one of claims 50 to 62, wherein the deletion starts within about 5 nucleotides to about 10 nucleotides downstream of the 5′-NTTN-3′ sequence.

64. The method of any one of claims 50 to 63, wherein the deletion starts within about 10 nucleotides to about 15 nucleotides downstream of the 5′-NTTN-3′ sequence.

65. The method of any one of claims 50 to 64, wherein the deletion ends within about 20 nucleotides to about 30 nucleotides of the 5′-NTTN-3′ sequence.

66. The method of any one of claims 50 to 65, wherein the deletion ends within about 20 nucleotides to about 25 nucleotides of the 5′-NTTN-3′ sequence.

67. The method of any one of claims 50 to 66, wherein the deletion ends within about 25 nucleotides to about 30 nucleotides of the 5′-NTTN-3′ sequence.

68. The method of any one of claims 50 to 67, wherein the deletion ends within about 20 nucleotides to about 30 nucleotides downstream of the 5′-NTTN-3′ sequence.

69. The method of any one of claims 50 to 68, wherein the deletion ends within about 20 nucleotides to about 25 nucleotides downstream of the 5′-NTTN-3′ sequence.

70. The method of any one of claims 50 to 69, wherein the deletion ends within about 25 nucleotides to about 30 nucleotides downstream of the 5′-NTTN-3′ sequence.

71. The method of any one of claims 50 to 70, wherein the deletion starts within about 5 nucleotides to about 15 nucleotides downstream of the 5′-NTTN-3′ sequence and ends within about 20 nucleotides to about 30 nucleotides downstream of the 5′-NTTN-3′ sequence.

72. The method of any one of claims 50 to 71, wherein the deletion starts within about 5 nucleotides to about 15 nucleotides downstream of the 5′-NTTN-3′ sequence and ends within about 20 nucleotides to about 25 nucleotides downstream of the 5′-NTTN-3′ sequence.

73. The method of any one of claims 50 to 72, wherein the deletion starts within about 5 nucleotides to about 15 nucleotides downstream of the 5′-NTTN-3′ sequence and ends within about 25 nucleotides to about 30 nucleotides downstream of the 5′-NTTN-3′ sequence.

74. The method of any one of claims 50 to 73, wherein the deletion starts within about 5 nucleotides to about 10 nucleotides downstream of the 5′-NTTN-3′ sequence and ends within about 20 nucleotides to about 30 nucleotides downstream of the 5′-NTTN-3′ sequence.

75. The method of any one of claims 50 to 74, wherein the deletion starts within about 5 nucleotides to about 10 nucleotides downstream of the 5′-NTTN-3′ sequence and ends within about 20 nucleotides to about 25 nucleotides downstream of the 5′-NTTN-3′ sequence.

76. The method of any one of claims 50 to 75, wherein the deletion starts within about 5 nucleotides to about 10 nucleotides downstream of the 5′-NTTN-3′ sequence and ends within about 25 nucleotides to about 30 nucleotides downstream of the 5′-NTTN-3′ sequence.

77. The method of any one of claims 50 to 76, wherein the deletion starts within about 10 nucleotides to about 15 nucleotides downstream of the 5′-NTTN-3′ sequence and ends within about 20 nucleotides to about 30 nucleotides downstream of the 5′-NTTN-3′ sequence.

78. The method of any one of claims 50 to 77, wherein the deletion starts within about 10 nucleotides to about 15 nucleotides downstream of the 5′-NTTN-3′ sequence and ends within about 20 nucleotides to about 25 nucleotides downstream of the 5′-NTTN-3′ sequence.

79. The method of any one of claims 50 to 78, wherein the deletion starts within about 10 nucleotides to about 15 nucleotides downstream of the 5′-NTTN-3′ sequence and ends within about 25 nucleotides to about 30 nucleotides downstream of the 5′-NTTN-3′ sequence.

80. The method of any one of claims 50 to 79, wherein the 5′-NTTN-3′ sequence is 5′-CTTT-3′, 5′-CTTC-3′, 5′-GTTT-3′, 5′-GTTC-3′, 5′-TTTC-3′, 5′-GTTA-3′, or 5′-GTTG-3′.

81. The method of any one of claims 50 to 80, wherein the deletion overlaps with a mutation in the BCL11A sequence.

82. The method of any one of claims 50 to 81, wherein the deletion overlaps with an insertion in the BCL11A sequence.

83. The method of any one of claims 50 to 82, wherein the deletion removes a repeat expansion of the BCL11A sequence or a portion thereof.

84. The method of any one of claims 50 to 83, wherein the deletion disrupts one or both alleles of the BCL11A sequence.

85. The method of any one of claims 50 to 84, wherein the deletion disrupts a GATAA motif of an enhancer region of the BCL11A gene.

86. The composition, RNA guide, nucleic acid, vector, cell, kit or method of any one of the previous claims, wherein the composition, RNA guide, nucleic acid, vector, cell, kit or method disrupts a GATAA motif of an enhancer region of the BCL11A gene.

87. The composition, cell, kit or method of any one of the previous claims, wherein the composition, cell, kit or method comprises at least two RNA guides targeting a GATAA motif of an enhancer region of the BCL11A gene.

88. The composition, cell, kit or method of claim 87, wherein the at least two RNA guides comprise at least 90% identity to:
(SEQ ID NO: 2677)
AGAAAUCCGUCUUUCAUUGACGGGAAGCUAGUCUAGUGCAAGC;
(SEQ ID NO: 2678)
AGAAAUCCGUCUUUCAUUGACGGCUGGAGCCUGUGAUAAAAGC;
and/or
(SEQ ID NO: 2679)
AGAAAUCCGUCUUUCAUUGACGGUACCCCACCCACGCCCCCAC.

89. The composition, cell, kit or method of claim 88, wherein the at least two RNA guides comprise at least 95% identity to:
(SEQ ID NO: 2677)
AGAAAUCCGUCUUUCAUUGACGGGAAGCUAGUCUAGUGCAAGC;
(SEQ ID NO: 2678)
AGAAAUCCGUCUUUCAUUGACGGCUGGAGCCUGUGAUAAAAGC;
and/or
(SEQ ID NO: 2679)
AGAAAUCCGUCUUUCAUUGACGGUACCCCACCCACGCCCCCAC.

90. The composition, cell, kit or method of claim 89, wherein the at least two RNA guides comprise at least two sequences of:
(SEQ ID NO: 2677)
AGAAAUCCGUCUUUCAUUGACGGGAAGCUAGUCUAGUGCAAGC;
(SEQ ID NO: 2678)
AGAAAUCCGUCUUUCAUUGACGGCUGGAGCCUGUGAUAAAAGC;
and/or
(SEQ ID NO: 2679)
AGAAAUCCGUCUUUCAUUGACGGUACCCCACCCACGCCCCCAC.

91. The composition, RNA guide, nucleic acid, vector, cell, kit or method of any one of the previous claims, wherein the RNA guide consists of the sequence of:
(SEQ ID NO: 2677)
AGAAAUCCGUCUUUCAUUGACGGGAAGCUAGUCUAGUGCAAGC;
(SEQ ID NO: 2678)
AGAAAUCCGUCUUUCAUUGACGGCUGGAGCCUGUGAUAAAAGC;
and/or
(SEQ ID NO: 2679)
AGAAAUCCGUCUUUCAUUGACGGUACCCCACCCACGCCCCCAC.

92. The composition, RNA guide, nucleic acid, vector, cell, kit or method of any one of the previous claims, wherein the RNA guide does not consist of the sequence of:
(SEQ ID NO: 2677)
AGAAAUCCGUCUUUCAUUGACGGGAAGCUAGUCUAGUGCAAGC;
(SEQ ID NO: 2678)
AGAAAUCCGUCUUUCAUUGACGGCUGGAGCCUGUGAUAAAAGC;
and/or
(SEQ ID NO: 2679)
AGAAAUCCGUCUUUCAUUGACGGUACCCCACCCACGCCCCCAC.
